SEQUENCE LISTING (55 sequences)

SEQ ID NO: 1 - protein, 176 aa, Bos indicus x Bos taurus
MGTFNLWTDYLGLARLVGAQREEEEPETRLDRQPEAVPEPGGQRPSPESSPAPERLCSFCKHNGESRAIYQSHVLKDEAGRVLCPILRDYVCPQCGATRERAHTRRFCPLTGQGYTSVYSYTTRNSAGKKLVRSDKARTQDPGHGPRRGGGACAGSKGARKSSGTPPSSCCPSTSA

SEQ ID NO: 2 - DNA, 3207 bp, Bos indicus x Bos taurus
  source 1..3207: mol_type = genomic DNA
gccgcccctggagggagggactggggaccgggtttgagggtgaagaaatggggaagagcattaacggggtaagcctcgtgtagttatgcgcttgggcccccgtctgatccgacaagggcccgagtttggaagcccgggaccctctgcgatcctctagcttcgcccttgtccaaccggcaggtggacccacaaggcgggctaggcagcggccccacctcggggctcgaatttgcaaagtgcagactcagacaaccctccccccaaccaccttgggttgttgtgattcataaaccattgtgtccggaacacggtgaagctcacttaggtattacattgtattaaaatgacttgtttatctctccgttgcatccatgcccccggggccagaaccacttggcctccagacctcttggggcctctcggaatccctcctctgcctctgcctctagctaagggtgccctctgttctggcctgtctcccaaactgataattggaagaaatatgcaccgttgagggcccttttgagaatgctttgactaaatgggttagaagcccagcgcccgctgctgctatatttgcatagcaaaggtgacagaagtatctgctgatattattacttagatttatctcctttttccctgtcctggagcagagttggctccttcctgctatctgttccctgacttaatagattctctaagtctctcattcccttcccctccctcaccctacccggttccttgacccaccccgccccccagcctccactccctgccccccaaggagttgccaagggtttgggggaacattcaacctgtcggtgagtttgggcagctcaggcaaaccatcgaccgttgagtggaccccgaggcctggaactgccgtccacccacccacccatcacgacccccaactttcagatctggggtaggggcaggggatcccgaacacatcccctcccttaggccacagcgaaggtcacaatcaacattcattgttgtcggtgggttgtgacagagaccagacccaccgagggatgaatgtcactgtggctgggccagacacaatcctggactccccccctcccgccccccaaaactgctcagccagaacctgaccctgaccctggcctttcacccctcgaggagggctggtgtctggggtacttaaagacacaggctagatttgggggcatcaatcctggagggctgtggacaggaattacaagtttaggactgggcagctgaaaaacctttctgaaagggattagggggccctgcttccagaaggctcagtgaagctttcttgaatgaatgaatgaatgaggtgtgtaggcggcacgtcacctcttctctgagttccagtcttgggccctgctttctcaccctttttacctggtacctgcagacccctcctttaccttcagttgcccacctagcacctgatgcccgttgatcacctgccagtctgtgtcccacctgggtgactcgggggcacaccgcatcctcctgagatggagcgcaggtctcatttgagagggcaatcaaggacctggccaatctaggggtctcccctctgccccgttagccccacctgtgcctgtgctctcttccccataatcctcagtctcaaacccttttccaccccaggacctggagagactgactccacaacacctaaggctcctgtaactggtgggggaggcaggctttgttgcctttgtgaataaccccagggcaggtgacttcaaacccgtttgttcatcagctaaaaggaggttccactgacaaggggtgtgaaagctccctgagggtgaccagaggtaggggccttggtccttgtccccccccaccataagacaggcccttcctccttccaaagtcagctggaaggtcagtggctccccctcccccctcccccagtcctggagaaggaagaaagaagttactaagttactgactacagcactgctagtctttggggtggggcttccaatgcccccacctgcatcactctggttctcctggaggagtagacaagggcagccctctcagtgccctctgggtggggtgtgtggctgcttattgctggtaccccctgcagcctgtgtcttgtcacgccccctcacccttagcctacccagaggccatgcagccccgtggcaggtgcatttctggggggagctgcagcaagccccctgtggcaatagggaacctcctacagcctgctcctccctcttcacacccccttggagtataaggagggaactgacagcccagactcctcggctccagagaggggaagggaagggagattaggcagaagtagagagaccagcttgggggcggctgcgtttcccctgtcttctgcccctccacctggcacacggggcccagccatggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggaggtgcctgtgcaggttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgccccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatgaatggatcagcgtca

SEQ ID NO: 3 - protein, 171 aa, Bos indicus x Bos taurus
MGTFNLWTDYLGLARLVGAQREEEEPETRLDRQPEAVPEPGGQRPSPESSPAPERLCSFCKHNGESRAIYQSHVLKDEAGRVLCPILRDYVCPQCGATRERAHTRRFCPLTGQGYTSVYSYTTRNSAGKKLVRSDKARTQDPGHGPRRGGGSKGARKSSGPPSSCCPSTSA

SEQ ID NO: 4 - DNA, 3196 bp, Bos indicus x Bos taurus
  source 1..3196: mol_type = genomic DNA
agccgcccctggagggagggactggggaccgggtttgagggtgaagaaatggggaagagcattaacggggtaagcctcgtgtagttatgcgcttgggcccccgtctgatccgacaagggcccgagtttggaagcccgggaccctctgcgatcctctagcttcgcccttgtccaaccggcaggtggacccacaaggcgggctaggcagcggccccacctcggggctcgaatttgcaaagtgcagactcagacaaccctccccccaaccaccttgggttgttgtgattcataaaccattgtgtccggaacacggtgaagctcacttaggtattacattgtattaaaatgacttgtttatctctccgttgcatccatgcccccggggccagaaccacttggcctccagacctcttggggcctctcggaatccctcctctgcctctgcctctagctaagggtgccctctgttctggcctgtctcccaaactgataattggaagaaatatgcaccgttgagggcccttttgagaatgctttgactaaatgggttagaagcccagcgcccgctgctgctatatttgcatagcaaaggtgacagaagtatctgctgatattattacttagatttatctcctttttccctgtcctggagcagagttggctccttcctgctatctgttccctgacttaatagattctctaagtctctcattcccttcccctccctcaccctacccggttccttgacccaccccgccccccagcctccactccctgccccccaaggagttgccaagggtttgggggaacattcaacctgtcggtgagtttgggcagctcaggcaaaccatcgaccgttgagtggaccccgaggcctggaactgccgtccacccacccacccatcacgacccccaactttcagatctggggtaggggcaggggatcccgaacacatcccctcccttaggccacagcgaaggtcacaatcaacattcattgttgtcggtgggttgtgacagagaccagacccaccgagggatgaatgtcactgtggctgggccagacacaatcctggactccccccctcccgccccccaaaactgctcagccagaacctgaccctgaccctggcctttcacccctcgaggagggctggtgtctggggtacttaaagacacaggctagatttgggggcatcaatcctggagggctgtggacaggaattacaagtttaggactgggcagctgaaaaacctttctgaaagggattagggggccctgcttccagaaggctcagtgaagctttcttgaatgaatgaatgaatgaggtgtgtaggcggcacgtcacctcttctctgagttccagtcttgggccctgctttctcaccctttttacctggtacctgcagacccctcctttaccttcagttgcccacctagcacctgatgcccgttgatcacctgccagtctgtgtcccacctgggtgactcgggggcacaccgcatcctcctgagatggagcgcaggtctcatttgagagggcaatcaaggacctggccaatctaggggtctcccctctgccccgttagccccacctgtgcctgtgctctcttccccataatcctcagtctcaaacccttttccaccccaggacctggagagactgactccacaacacctaaggctcctgtaactggtgggggaggcaggctttgttgcctttgtgaataaccccagggcaggtgacttcaaacccgtttgttcatcagctaaaaggaggttccactgacaaggggtgtgaaagctccctgagggtgaccagaggtaggggccttggtccttgtccccccccaccataagacaggcccttcctccttccaaagtcagctggaaggtcagtggctccccctcccccctcccccagtcctggagaaggaagaaagaagttactaagttactgactacagcactgctagtctttggggtggggcttccaatgcccccacctgcatcactctggttctcctggaggagtagacaagggcagccctctcagtgccctctgggtggggtgtgtggctgcttattgctggtaccccctgcagcctgtgtcttgtcacgccccctcacccttagcctacccagaggccatgcagccccgtggcaggtgcatttctggggggagctgcagcaagccccctgtggcaatagggaacctcctacagcctgctcctccctcttcacacccccttggagtataaggagggaactgacagcccagactcctcggctccagagaggggaagggaagggagattaggcagaagtagagagaccagcttgggggcggctgcgtttcccctgtcttctgcccctccacctggcacacggggcccagccatggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggaggttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgccccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatgaatggatcagcgtca

SEQ ID NO: 5 - DNA, 283 bp, Bos indicus x Bos taurus
  source 1..283: mol_type = genomic DNA
tgggaggcggcggccgcgggttcgagccggcgccggagccccgcggtcccctccccctgcccgcggcctggggagcccccgcccagccccggagccgccaaaatgcaatttcccgtgccggcgcctcgcggctcggggggcttttccgggcgggttttggacagaagagggggaaacaaggcggcggccccaaaacgaggttccaaaggtgccaggaagtcttctggaactcctccctcttcctgctgcccctcaacttctgcctaaggagactggcgtgggc

SEQ ID NO: 6 - protein, 176 aa, Bos indicus; identical to SEQ ID NO: 1

SEQ ID NO: 7 - DNA, 2116 bp, Bos indicus
  source 1..2116: mol_type = genomic DNA
tccccccctcccgccccccaaaactgctcagccagaacctgaccctgaccctggcctttcacccctcgaggagggctggtgtctggggtacttaaagacacaggctagatttgggggcatcaatcctggagggctgtggacaggaattacaagtttaggactgggcagctgaaaaacctttctgaaagggattagggggccctgcttccagaaggctcagtgaagctttcttgaatgaatgaatgaatgaggtgtgtaggcggcacgtcacctcttctctgagttccagtcttgggccctgctttctcacccttcttacctggtacctgcagacccctcctttaccttcagttgcccacctagcacctgatgcccgttgatcacctgccagtctgtgtcccacctgggtgactcgggggcacaccgcatcctcctgagatggagcgcaggtctcatttgagagggcaatcaaggwcctggccaatctaggggtctcccctctgccccgttagccccacctgtgcctgtgctctcttccccataatcctcagtctcaaacccttttccaccccaggacctggagagactgactccacaacacctaaggctcctgtaactggtgggggaggcaggctttgttgccttcgtgaataaccccagggcaggtgacttcaaacccgtttgttcatcagctaaaaggaggttccactgacaaggggtgtgaaagctccctgagggtgaccagaggtaggggccttggtccttgtccccccccaccataagacaggcccttcctccttccaaagtcagctggaaggtcagtggctccccctcccccctcccccagtcctggagaaggaagaaagaagttactaagttactgactacagcactgctagtctttggggtggggcttccaatgcccccacctgcatcactctggttctcctggaggagtagacaagggcagccctctcagtgccctctgggtggggtgtgtggctgcttattgctggtaccccctgcagcctgtgtcttgtcacgccccctcacccttagcctacccagaggccatgcagccccgtggcaggtgcatttctggggggagctgcagcaagccccctgtggcaatagggaacctcctacagcctgctcctccctcttcacacccccttggagtataaggagggaactgacagcccagactcctcggctccagagaggggaagggaagggagattaggcagaagtagagagaccagcttgggggcggctgcgtttctcctgtcttctgcccctccacctggcacacggggcccagccatggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggaggtgcctgtgcaggttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgccccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatg

SEQ ID NO: 8 - DNA, 2633 bp, Bos sp.
  source 1..2633: mol_type = genomic DNA
atggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggaggtgcctgtgcaggtggctggggggactcgctgcagaggggggtctgccctgggctgacttagagcctctgagaagtgggttaacccctgggctgacccacttcagagggttgggtgggggagagaatccatacacaatagaaggcttagaaccatgttctagaactgttgccctagtgggtaagctggggctggggttccctgtgtgaccttgggcaagacactctccttctctgggcccatagagggtatattgttttcaggcgcagggtgagctagcagggaggccgtggggactagtgatggggtctggcaacaggtacagcaggggctgcaatcttgtgcggaatgcaggtgccacgtgaccagagggaaacagcctcacttgtccactcattcgccaggtggtttgtgaggcttgtcctgtgccaagcactgggctgggcttggccttcagtggtagatgggacagtgacagttataacagaaacgggggagtctaaagggagcactcaagctagacaaagaggtccaggagggcttcctggaggaagtgactcctggaaacctcattcagtttggtgaaatgctgaagggggtctgggcactggggctcactagggaacttggttaaattccgtgcgtctgggatgggagtatgcgtgcatgaaggatggaggggaaggtgtcagaaatttcggggaagggctgcttgctgatcgccttgaagagtttggacttgagtactagggagctgagaaggggacacgagtaggtctgtgtattaagggtgtgttagactgtgcgtgtgttagactgtggctaatgggttgggctgtgggggtggatttgagggggttggaagaagttggtcctccaggcaggtgttggagttggcatcagtttggagggaaagaaccatgagtttgctgtgtgtgggtcaccccccgggagggtggctggaggctccctaaggtctgcacagccacagagggttccctccatcttgctcatcaaattcggagtccagttttgcaggcctgagtgaggccagagcatctgtacttctaactagatccccttgacctccgtagccggcaggcttcgagtagctaggcttacgtatcagcagttactattacagactcgaaacacctccgcttctgcaggtttataagagaaatccgttttagcaactttggggaagtcagccaacagagggaccggtttgacggggcggtaccccattactgcctctctcagactgggggcccctcgggaagtcaccagaagggaggaccccttcccatcccggcggctgtgcggagaacacgttagaagggttgtgagactgcctgcgggagaggatagtattcctgtgaggtctgacaaggtccaactgggagagggaggaagcggaagcgacacagagacgatggtctcagaatcttcttttttcccctagggaacaggaaaacttgactcaggggtggggtggggtcggggagccctcactacgatattggcacggaaaggctgcctgaggctgtcctcccaggaaactttctgagtgtaacaaatccggggttcccggagccctgcggccgcgagggggcagtacggcaggacagagttgggagtttccgttgctttgtgtctggctctgggtcccacctgggactgcccggggctgcgaatagcacaggggtgctagtccggaaggagcctgcaggtggagccagagccccggcagctctgcaggttgagtcggaacttccggatcatcgaatcatccttattactaaatgcttttcccctccccccaactctgcttttaaaatctaggttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgcccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatgaatggatca

SEQ ID NO: 9 - DNA, 790 bp, Bos sp.
  source 1..790: mol_type = genomic DNA
atggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggaggttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgccccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatgaatggatca

SEQ ID NO: 10 - DNA, 451 bp, Bos sp.
  source 1..451: mol_type = genomic DNA
atggggaccttcaacctgtggacagactacttgggtttggcacgcctggttggggctcagcgtgaagaagaggagccggagaccaggctggatcgccagccagaagcagtgcccgaaccggggggtcagcgacccagccctgaatcctcaccagctcccgagcgcctgtgttctttctgcaaacacaacggcgagtcccgggccatctaccagtcccacgtgctcaaggatgaagcgggccgggtgctgtgccccatcctccgcgactacgtgtgcccccagtgcggggccacccgcgagcgcgcccacacccgccgcttctgcccgctcaccggccagggctacacctccgtctacagctacaccacccggaactcggccggcaagaagctggtccgctcggacaaggcgaggacgcaggaccctggacacggaccgcgccgaggaggag

SEQ ID NO: 11 - DNA, 339 bp, Bos sp.
  source 1..339: mol_type = genomic DNA
gttccaaaggtgccaggaagtcttctggaactcctccctcttcctgctgcccctcaacttctgcctaaggagactggcgtgggcaggatgacgccttcacctggggatggggacccaggctcagtggaggctgggtttcagggaagacccaccctccgaggatccgccccctagacggtgcctccagcctgggggcttggcaaaggagcccggtctgggaccaccgcccaaagcgcgcccgcccctgtcactgaagggggtggtcctcaggcacccctgcccttcttccccaacgctgagcaaccagtcagcgctcaataaatgtttatgaatggatca

SEQ ID NO: 12 - protein, 7 aa, Simian virus 40
PKKKRKV

SEQ ID NO: 13 - protein, 10 aa, Simian virus 40
PKKKRKVEAS

SEQ ID NO: 14 - protein, 16 aa, synthetic construct
  REGION 1..16: note = Synthetic
KRPAATKKAGQAKKKK

SEQ ID NO: 15 - protein, 9 aa, Homo sapiens
PAAKRVKLD

SEQ ID NO: 16 - protein, 11 aa, Homo sapiens
RQRRNELKRSP

SEQ ID NO: 17 - protein, 38 aa, Homo sapiens
NQSSNFGPMKGGNFGGRSSGPYGGGGQYFAKPRNQGGY

SEQ ID NO: 18 - protein, 42 aa, Homo sapiens
RMRIZFKNKGKDTAELRRRRVEVSVELRKAKKDEQILKRRNV

SEQ ID NO: 19 - protein, 8 aa, Homo sapiens
VSRKRPRP

SEQ ID NO: 20 - protein, 8 aa, Homo sapiens
PPKKARED

SEQ ID NO: 21 - protein, 8 aa, Homo sapiens
PQPKKKPL

SEQ ID NO: 22 - protein, 12 aa, Mus sp.
SALIKKKKKMAP

SEQ ID NO: 23 - protein, 5 aa, Influenza virus
DRLRR

SEQ ID NO: 24 - protein, 7 aa, Influenza virus
PKQKKRK

SEQ ID NO: 25 - protein, 10 aa, Hepatitis D virus
RKLKKKIKKL

SEQ ID NO: 26 - protein, 10 aa, Mus sp.
REKKKFLKRR

SEQ ID NO: 27 - protein, 20 aa, Homo sapiens
KRKGDEVDGVDEVAKKKSKK

SEQ ID NO: 28 - protein, 17 aa, Homo sapiens
RKCLQAGMNLEARKTKK

SEQ ID NO: 29 - protein, 10 aa, synthetic construct
  REGION 1..10: note = Synthetic
  VARIANT 3: note = misc_feature - Xaa can be any naturally occuring amino acid
  VARIANT 2: note = misc_feature - Xaa can be any naturally occuring amino acid
  VARIANT 4: note = misc_feature - Xaa can be any naturally occuring amino acid
  VARIANT 6: note = misc_feature - Xaa can be any naturally occuring amino acid
  VARIANT 7: note = misc_feature - Xaa can be any naturally occuring amino acid
  VARIANT 9: note = misc_feature - Xaa can be any naturally occuring amino acid
LXXXLXXLXL

SEQ ID NO: 30 - protein, 4 aa, synthetic construct
  REGION 1..4: note = Synthetic
KDEL

SEQ ID NO: 31 - protein, 15 aa, synthetic construct
  REGION 1..15: note = Synthetic
GGGGGGGGGGGGGGG

SEQ ID NO: 32 - protein, 15 aa, synthetic construct
  REGION 1..15: note = Synthetic
GGGGSGGGGSGGGGS

SEQ ID NO: 33 - protein, 8 aa, synthetic construct
  REGION 1..8: note = Synthetic
GPLGIAGQ

SEQ ID NO: 34 - RNA, 23 nt, synthetic construct
  source 1..23: mol_type = other RNA
gtggacagactacttgggtttgg

SEQ ID NO: 35 - RNA, 23 nt, synthetic construct
  source 1..23: mol_type = other RNA
ctacttgggtttggcacgcctgg

SEQ ID NO: 36 - RNA, 23 nt, synthetic construct
  source 1..23: mol_type = other RNA
gttcgggcactgcttctggctgg

SEQ ID NO: 37 - RNA, 23 nt, synthetic construct
  source 1..23: mol_type = other RNA
agaagcagtgcccgaaccggggg

SEQ ID NO: 38 - RNA, 23 nt, synthetic construct
  source 1..23: mol_type = other RNA
cgcttcatccttgagcacgtggg

SEQ ID NO: 39 - RNA, 23 nt, synthetic construct
  source 1..23: mol_type = other RNA
tggtccgctcggacaaggcgagg

SEQ ID NO: 40 - RNA, 23 nt, synthetic construct
  source 1..23: mol_type = other RNA
ctcggacaaggcgaggacgcagg

SEQ ID NO: 41 - RNA, 23 nt, synthetic construct
  source 1..23: mol_type = other RNA
actggacacggatcgcgccgagg

SEQ ID NO: 42 - RNA, 23 nt, synthetic construct
  source 1..23: mol_type = other RNA
actgggggcacacgtagtcgcgg

SEQ ID NO: 43 - RNA, 23 nt, synthetic construct
  source 1..23: mol_type = other RNA
tggtccgctcggacaaggcgagg

SEQ ID NO: 44 - RNA, 23 nt, synthetic construct
  source 1..23: mol_type = other RNA
acacgtagtcgcggaggatgggg

SEQ ID NO: 45 - RNA, 23 nt, synthetic construct
  source 1..23: mol_type = other RNA
gcaaacacaacggcgagtcccgg

SEQ ID NO: 46 - RNA, 20 nt, synthetic construct
  source 1..20: mol_type = other RNA
cctcaactgacggggaagtc

SEQ ID NO: 47 - RNA, 20 nt, synthetic construct
  source 1..20: mol_type = other RNA
agtgttgggtggctgttgtt

SEQ ID NO: 48 - DNA, 34 bp, Bos sp.
  source 1..34: mol_type = genomic DNA
agaagcagtgcccgaaccggggggtcagcgaccc

SEQ ID NO: 49 - DNA, 31 bp, Bos sp.
  source 1..31: mol_type = genomic DNA
agaagcagtgcccgaactgggtcagcgaccc

SEQ ID NO: 50 - DNA, 31 bp, Bos sp.; identical to SEQ ID NO: 49
  source 1..31: mol_type = genomic DNA

SEQ ID NO: 51 - DNA, 33 bp, Bos sp.
  source 1..33: mol_type = genomic DNA
ccgctcggacaaggcgaggacgcaggaccctgg

SEQ ID NO: 52 - protein, 150 aa, Bos sp.
MGTFKLWTDYLGLARLVGAQREEEEPETRLDRQPEAVPEPGGQRPSPESSPAPERLCSFCKHNGESRAIYQSHVLKDEAGRVLCPILRDYVCPQCGATRERAHTRRFCPLTGQGYTSVYSYTTRNSAGKKLVRSDKARTQDPGHGPRRGG

SEQ ID NO: 53 - protein, 146 aa, Bos sp.
MGTFKLWTDYLGLARLVGAQREEEEPETRLDRQPEAVPELGQRPPESSPAPERLCSFCKHNGESRAIYQSHVLKDEAGRVLCPILRDYVCPQCGATRERAHTRRFCPLTGQGYTSVYSYTTRNSAGKKLVRSDKAQDPGHGPRRGG

SEQ ID NO: 54 - DNA, 12 bp, Bos sp.
  source 1..12: mol_type = genomic DNA
ttctcagaatag

SEQ ID NO: 55 - DNA, 26 bp, Bos sp.
  source 1..26: mol_type = genomic DNA
ttaactcactgacagtgagtcgacag

(19) United States
(12) Patent Application Publication
(10) Pub. No.: US 2026/0101880 A1
(43) Pub. Date: Apr. 16, 2026

(54) GENETIC COMPLEMENTATION COMPOSITIONS AND METHODS

(71) Applicant: The Regents of the University of California, Oakland, CA (US)

(72) Inventors: Maci L. MUELLER, Davis, CA (US); Alba Veronica LEDESMA, Davis, CA (US); Pablo Juan ROSS, College Station, TX (US); Alison Louise Van EENENNAAM, Davis, CA (US)

(21) Appl. No.: 18/852,108

(22) PCT Filed: Apr. 4, 2023
(86) PCT No.: PCT/US2023/065338

§ 371 Date: Sep. 27, 2024

Related U.S. Application Data
(60) Provisional application No. 63/327,168, filed on Apr. 4, 2022.

(51) Int. Cl. A01K 67/0276; C12N 5/073; C12N 9/22; C12N 15/11; C12N 15/90

(52) U.S. Cl. CPC A01K 67/0276; C12N 5/0604; C12N 9/226; C12N 15/11; C12N 15/907; A01K 2217/075; A01K 2227/101; A01K 2227/103; C12N 2310/20

(57) ABSTRACT

Described in several exemplary embodiments herein are germline complementation methods and compositions, particularly NANOS3 deficient cells and non-human animals. In some embodiments, the NANOS3 deficient non-human animals are germline ablated.

### CROSS-REFERENCE TO RELATED APPLICATIONS

This application is a U.S. National Phase Application Under 371 of International Application PCT/US2023/065338 filed Apr. 4, 2023 and claims the benefit of and priority to U.S. Provisional Patent Application No. 63/327,168, filed on Apr. 4, 2022, entitled “Genetic Complementation Compositions and Methods,” the contents of each of which are incorporated by reference herein in their entireties.

### SEQUENCE LISTING

This application contains a sequence listing filed in electronic form as an xml file entitled revised_081906-129621_ST26.xml, created on Oct. 27, 2025, and having size of 67,315 bytes. The content of the sequence listing is incorporated herein in its entirety.

### TECHNICAL FIELD

The subject matter disclosed herein is generally directed to genetically germline ablated non-human animals and uses thereof.

### BACKGROUND

Conventional genetic selection and breeding programs have generated populations of elite genetic seedstock. However, there exists a lag in the genetic improvement between the elite nucleus seedstock population and commercial animals. This lag is due to the generation intervals of conventional breeding programs. As such, there exists a need for approaches to improve the rate of genetic improvement, particularly at the commercial animal level.

Citation or identification of any document in this application is not an admission that such a document is available as prior art to the present invention.

### SUMMARY

Described in certain example embodiments herein are engineered non-human animal cells or populations thereof comprising a NANOS3 gene modification, wherein the NANOS3 gene modification reduces or eliminates the expression of a NANOS3 gene product.

In certain example embodiments, the NANOS3 gene modification is an insertion of one or more nucleotides; a deletion of one or more nucleotides; a substitution of one or more nucleotides; or any combination thereof. In certain example embodiments, the NANOS3 gene modification is in exon 1 of the NANOS3 gene, optionally in the zinc finger domain of the NANOS3 gene.

In certain example embodiments, the engineered non-human animal cell or population thereof is a bovine, equine, porcine, ovine, caprine, camelid, cervine, canine, feline, murine, leporine, or a cavine cell.

In certain example embodiments, one or both NANOS3 alleles are modified. In certain example embodiments, the engineered non-human animal cell or population thereof is monoallelic for the NANOS3 gene modification. In certain example embodiments, the engineered non-human animal cell or population thereof is biallelic for the NANOS3 gene modification. In certain example embodiments, the engineered non-human animal cell population thereof does not express a functional NANOS3 gene or gene product.

In certain example embodiments, the engineered non-human animal cell is heterozygous or homozygous for the NANOS3 gene modification, wherein the NANOS gene modification is optionally a NANOS3 gene knockout.

In certain example embodiments, the engineered non-human animal cell or population thereof is an engineered male cell or population thereof. In certain example embodiments, the engineered non-human animal cell is an engineered female cell or cell population.

In certain example embodiments, the engineered non-human animal cell or population thereof is an engineered somatic cell or population thereof. In certain example embodiments, the engineered non-human animal cell or population thereof is an engineered germ cell or population thereof. In certain example embodiments, the engineered germ cell or population thereof is an engineered gamete or population thereof. In certain example embodiments, the engineered gamete or population thereof is an engineered spermatozoon or population thereof or an engineered ovum or population thereof. In certain example embodiments, the engineered germ cell or population thereof is an engineered immature germ cell or population thereof. In certain example embodiments, the engineered immature germ cell or population thereof is an engineered spermatid or population thereof or an engineered oocyte or population thereof. In certain example embodiments, the engineered non-human animal cell is an engineered embryonic cell population thereof, optionally wherein the engineered embryonic cell is a zygote. In certain example embodiments, wherein the engineered non-human animal cell population thereof is an engineered blastocyst cell or population thereof, optionally an engineered inner cell mass cell or population thereof. In certain example embodiments, the engineered non-human animal cell or population thereof is an engineered stem cell or population thereof, optionally an engineered embryonic stem cell or population thereof or an induced pluripotent stem cell or population thereof. In certain example embodiments, the engineered non-human animal cell or cell population is an engineered spermatogonial stem cell or population thereof or an engineered oogonial stem cell or population thereof. In certain example embodiments, the engineered non-human animal cell or cell population cell is a primordial germ cell or population thereof or an engineered primordial germ cell-like cell or population thereof. In certain example embodiments, the engineered non-human animal cell or population thereof is an engineered self-renewing cell or population thereof. In certain example embodiments, the engineered non-human animal cell is pluripotent, totipotent, or multipotent.

Described in certain example embodiments herein are engineered non-human animals, embryos, or progeny thereof comprising an engineered non-human animal cell or population thereof as in any of the preceding paragraphs or as described elsewhere herein.

In certain example embodiments, the engineered non-human animal, embryo, or progeny thereof is a chimera. In certain example embodiments, the engineered non-human animal, embryo, or progeny thereof is a mosaic. In certain example embodiments, the engineered non-human animal, embryo, or progeny thereof is not chimeric. In certain example embodiments, the engineered non-human animal, embryo, or progeny is not a mosaic. In certain example embodiments, at least 1 cell of or at least 0.0001 percent to 100 percent of all cells of the engineered non-human animal, embryo, or progeny thereof is an engineered non-human animal cell as in any of the preceding paragraphs or as described elsewhere herein.

In certain example embodiments, the engineered non-human animal, embryo, or progeny thereof is a male. In certain example embodiments, the engineered non-human animal, embryo, or progeny thereof is a female. In certain example embodiments, the engineered non-human animal is a bovine, equine, porcine, ovine, caprine, camelid, cervine, canine, feline, murine, leporine, or a cavine.

In certain example embodiments, the engineered non-human animal, embryo, or progeny thereof further comprises a second population of cells comprising one or more cells, wherein the second population of cells does not comprise engineered non-human animal cells of any one of the preceding paragraphs or as described elsewhere herein and wherein the second population of cells are germline competent cells, germ cells, or gametes. In certain example embodiments, the second population of cells comprises or consists of one or more embryonic cells, optionally a zygote or inner cell mass cells; stem cells, optionally embryonic stem cells or induced pluripotent stem cells; spermatogonial stem cells or oogonial stem cells; primordial germ cells; or primordial germ cell like cells. In certain example embodiments, the second population of cells comprises or consists of one or more spermatids or one or more oocytes. In certain example embodiments, the second population of cells comprises or consists of spermatozoa or ova. In certain example embodiments, the second population of cells comprises or consists of one or more engineered cells comprising one or more genetic modifications in one or more target genes and wherein the one or more target genes are not NANOS3. In certain example embodiments, the second population of cells does not comprise or consist of an engineered cell or population thereof. In certain example embodiments, the second population of cells comprises or consists of an elite genome, a genomically selected genome, or both.

Described in certain example embodiments herein are complemented non-human animal or embryo comprising or consisting of a first population of cells comprising one or more cells, wherein the first population of cells consists of an engineered non-human animal cell or population thereof of any one of the preceding paragraphs and/or as described elsewhere herein; and a second population of cells comprising one or more cells, wherein the second population cells are not an engineered non-human cell or population thereof any one of the preceding paragraphs and/or as described elsewhere herein.

In certain example embodiments, the second population of cells comprises or consist of one or more engineered cell population comprising one or more genetic modifications in one or more target genes and wherein the one or more target genes are not NANOS3. In certain example embodiments, the second population of cells is not an engineered cell or population thereof. In certain example embodiments, the second population of cells comprises an elite genome, a genomically selected genome, or both. In certain example embodiments, the second population of cells comprises or consists of one or more embryonic cells, optionally a zygote or inner cell mass cells; stem cells, optionally embryonic stem cells or induced pluripotent stem cells; spermatogonial stem cells or oogonial stem cells; primordial germ cells; or primordial germ cell like cells. In certain example embodiments, the second population of cells are self-renewing cells. In certain example embodiments, the second population of cells is pluripotent, totipotent, or multipotent. In certain example embodiments, the second population of cells is germline competent.

In certain example embodiments, the complemented embryo is a preimplantation embryo, optionally a zygote, 2 cell, 4 cell, an 8 cell, 16 cell, a blastocyst, or a morula. In certain example embodiments, the first population of cells makes up a percentage of cells of the complemented non-human animal or embryo ranging from about 25 percent to any percent up to but not including 100 percent. In certain example embodiments, the complemented non-human animal or embryo comprises at least one cell of the second population of cells, optionally wherein the second population of cells makes up a percentage of cells of the engineered non-human animal or embryo ranging from any non-zero percent to about 75 percent. In certain example embodiments, the complemented embryo is a day 3 post fertilization embryo, a day 4 post fertilization embryo, a day 5 post fertilization embryo, or a day 6 post fertilization day embryo. In certain example embodiments, the day 3 post fertilization complemented embryo comprises about 5 cells from the second population of cells; the day 4 post fertilization complemented embryo comprises about 5 cells from the second population of cells the day 5 post fertilization complemented embryo comprises about 8-10 cells from the second population of cells; and the day 6 post fertilization complemented embryo comprises about 10-20 cells from the second population of cells.

In certain example embodiments, the complemented embryo is a morula. In certain example embodiments, the complemented non-human animal or embryo is a male. In certain example embodiments, the complemented non-human animal or embryo is a female. In certain example embodiments, the complemented non-human animal or embryo is a bovine, equine, porcine, ovine, caprine, camelid, cervine, canine, feline, murine, leporine, or cavine.

Described in certain example embodiments herein are non-human animals developed or generated from the complemented non-human animal or embryo of any one of the preceding paragraphs or as described elsewhere herein. In certain example embodiments, one or more germ cells of the engineered animal originated from the second population of cells. In certain example embodiments, about 0.001 percent to 100 percent of the germ cells originated from the second population of cells. In certain example embodiments, the non-human animal is a male. In certain example embodiments, the non-human animal is female.

Described in certain example embodiments herein are progeny of one or more complemented non-human animals or non-human animals of any one of the preceding paragraphs or as described elsewhere herein.

Described in certain example embodiments are methods of generating a NANOS3 modified non-human animals or embryos, the method comprising introducing one or more NANOS3 gene modifications to a non-human animal cell, wherein the NANOS3 gene modification reduces or eliminates the expression of a NANOS3 gene product; and one or more of the following techniques: somatic cell nuclear transfer, oocyte pronuclear DNA microinjection, zygote microinjection, or embryo microinjection, intracytoplasmic sperm injection, in vitro fertilization, embryo transfer, in vitro embryo culture, or any combination thereof.

In certain example embodiments, NANOS3 gene modification is an insertion of one or more nucleotides; a deletion of one or more nucleotides; a substitution of one or more nucleotides; or any combination thereof. In certain example embodiments, the NANOS3 gene modification is in exon 1 of the NANOS3 gene, optionally in the zinc finger domain of the NANOS3 gene.

In certain example embodiments, one or both of the NANOS3 alleles are modified. In certain example embodiments, the non-human animal or embryo is monoallelic for the NANOS3 gene modification. In certain example embodiments, the non-human animal or embryo is biallelic for the NANOS3 gene modification. In certain example embodiments, the engineered non-human animal or embryo does not express a functional NANOS3 gene or gene product. In certain example embodiments, the non-human animal or embryo is a heterozygous or homozygous NANOS3 gene knockout. In certain example embodiments, the non-human animal or embryo is germline ablated.

In certain example embodiments, the non-human animal or embryo is a bovine, equine, porcine, ovine, caprine, camelid, cervine, canine, feline, murine, leporine, or a cavine. In certain example embodiments, the non-human animal or embryo is a male. In certain example embodiments, the non-human animal or embryo is a female.

In certain example embodiments, introducing one or more NANOS3 gene modifications to the non-human animal cell comprises CRISPR-Cas mediated gene modification, Zinc Finger Nuclease gene modification, TALEN mediated gene modification, recombinase mediated gene modification, prime editing mediated gene modification, meganuclease mediated gene modification, transposase/transposon mediated gene modification, or any combination thereof.

In certain example embodiments, introducing one or more NANOS3 gene modifications to the non-human animal cell comprises use of a CRISPR-Cas system and wherein the guide RNA for the CRISPR-Cas system targets exon 1 of the NANOS3 gene, optionally in the zinc finger region, and are optionally selected from any one of SEQ ID NOs: 39-45, or any combination thereof.

Described in certain example embodiments herein are methods of non-human animal embryo complementation comprising introducing a self-renewing exogenous population of cells into a non-human animal preimplantation embryo, optionally at about day 3, 4, 5, or 6 post fertilization; optionally washing the non-human animal preimplantation embryo in HEPES or other suitable buffer; and culturing the non-human preimplantation embryo in a suitable culture media optionally consisting of a 1:1 ratio by volume of a suitable bovine culture media that is at least supplemented with N2, B27, FGF, and IWR-1.

In certain example embodiments, the number of exogenous cells introduced is about 1 to about 25 cells or about 30-50 percent of the total number of cells present in the embryo prior to introducing the exogenous cells. In certain example embodiments, the number of exogenous cells introduced at 3 days or 4 days post fertilization is about 5 cells. In certain example embodiments, the number of exogenous cells introduced at 5 days post fertilization is 8 cells. In certain example embodiments, the number of exogenous cells introduced at 5 days post fertilization is 9 cells. In certain example embodiments, the number of exogenous cells introduced at 5 days post fertilization is 10 cells. In certain example embodiments, the number of exogenous cells introduced at 6 days post fertilization is about 10-20 cells.

In certain example embodiments, self-renewing exogenous cells are embryonic stem cells, expanded embryonic stem cells, pluripotent stem cells, induced pluripotent stem cells, multipotent stem cells, totipotent stem cells, primordial germ cells, primordial germ cell-like cells, totipotent cells, or a combination thereof.

In certain example embodiments, the non-human animal embryo is genetically germline ablated. In certain example embodiments, the non-human animal embryo comprises or consists of one or more engineered cells of any one of the preceding paragraphs or as described elsewhere herein. In certain example embodiments, the self-renewing exogenous cells are germline competent. In certain example embodiments, the self-renewing exogenous cells are engineered cells comprising one or more gene modifications in one or more target genes and wherein the one or more target genes are not NANOS3. In certain example embodiments, the self-renewing exogenous cells are not genetically modified. In certain example embodiments, the self-renewing exogenous cells comprise an elite genome, a genomically selected genome, or both.

In certain example embodiments, the non-human animal is a bovine, equine, porcine, ovine, caprine, camelid, cervine, canine, feline, murine, leporine, or a cavine.

Described in certain example embodiments herein are complemented non-human embryo produced from a method of embryo complementation of any one of the preceding paragraphs or as described elsewhere herein.

Described in certain example embodiments are non-human animals produced from the embryo of any one of the preceding paragraphs and progeny thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a schematic of exemplary surrogate sire production systems. Light grey (“host”) represents steps to generate the host animal. Pathways A and B represent potential alternative sources and steps for generating donor cells. The germline complementation steps are identified as such for each of the donor cell generation pathways. Dark grey represents the surrogate sire and it use to develop offspring of the donor germline.

FIG. 2 shows a diagram CRISPR-Cas target sites within the NANOS3 gene.

FIG. 3 shows a diagram of bovine NANOS3 exon 1 with selected dgRNA_4+7 genomic locations.

FIGS. 4A-4F demonstrate the production of NANOS3−/− live calves. FIGS. 4A-4B show images of CRISPR-Cas9 NANOS3 targeted bovine embryos transferred into recipient cows. FIGS. 4C-4D show images of 1-day-old calves produced from the embryos of FIGS. 4A-4B. FIG. 4E shows images of the calves at 2-months of age. FIG. 4F shows results from PCR for NANOS3 performed using DNA from the calves. Letters represent different alleles present in each animal.

FIGS. 5A-5D show results from a genotype analysis of the third live NANOS3 gene edited calf, named Frodo. FIG. 5A shows an image of Frodo at 1-week-old. FIG. 5B shows results from a PCR analysis for NANOS3 using DNA obtained from Frodo. FIGS. 5C1-5C3 show a diagram of bovine NANOS3 exon 1 with selected dual gRNA_4+7 genomic locations and Sanger sequencing results showing bi-allelic, homozygous in-frame mutations (SEQ ID NOs: 48-51). FIG. 5C2 demonstrates that gRNA4 resulted in a single base pair (bp) substitution (C to T) and a 3 bp deletion (SEQ ID NOs: 48-49). FIG. 5C3 demonstrates that gRNA7 resulted in a 6 bp deletion (SEQ ID NOs: 50-51). FIG. 5D shows a comparison of bovine wild type NANOS3 exon 1 protein sequence (SEQ ID NO: 52) to Frodo's predicted protein sequence (SEQ ID NO: 53). The amino acid substitution is highlighted in grey and italicized (P to L). The three deleted amino acids are represented * in the wild type sequence and dashes in Frodo's sequence. The highly conserved Zinc Finger binding domain is underlined.

FIG. 6 shows a structural annotation of ovine NANOS3 Exon 1 in dark grey with the critical domains in light grew, sgRNA binding sites in dark grey arrows, PAM sites in light grey bars, and primer binding sites in dark grey bars.

FIG. 7 shows results from an in vitro cleavage assay demonstrating CRISPR-Cas9 ovine NANOS3 cleavage. Lanes are as follows: Invitrogen 1 kb+ ladder labelled “L”, sgRNAs 1-5, and H2O negative control labelled “—”. The 749 bp NANOS3 PCR amplified genomic DNA is cut minimally by sgRNA 1, highly efficiently by sgRNAs 2, 3, and 4, and medium-lowly efficiently by sgRNA 5.

FIGS. 8A-8B show images of the EGFP-ESC cell line used to optimize the conditions to achieve embryo chimerism. FIG. 8A shows a colony of EGFP-ESC in culture. FIG. 8B shows EGFP-ESC after harvest and dissociation. 20× magnification. The second ESCs line was derived from a female embryo at the early blastocyst stage, and cultured in N2B27 media previously described, these cells were platted in a different matrix, vitronectin, free from mouse embryonic fibroblast (MEF) feeder cells, with the aim of obtain a pure line of bovine cells.

FIGS. 9A-9D show early stage embryos after injection. FIG. 9A shows a representative brightfield image of GFP-positive bovine embryos. FIG. 9B shows a representative fluorescent microscopy image showing GFP fluorescence (green, represented in greyscale). FIG. 9C shows a merge of the images in FIGS. 9A-9B. These embryos can be used to derive ESCs for use in embryo complementation. In other words, these embryos can be used to derive ESCs to inject into host embryos. FIG. 9D shows embryos at the morula stage injected with a GFP expressing embryonic stem cells, such as those produced from the embryos shown in FIGS. 9A-9C.

FIG. 10 shows representative immunofluorescence images of ESC stained with DAPI as a nuclear marker (blue, represented in greyscale), an anti-OCT4 antibody (red, represented in greyscale), an anti-SOX2 antibody (green, represented in greyscale) and an overlay of the three markers. Images are at 20× magnification.

FIG. 11 shows a sequence of injection of ESC into embryos using a microinjection system.

FIGS. 12A-12D show ESCs incubated in a fluorescent dye and their injection into host embryos. FIG. 12A shows images of bovine ESC after incubation with PKH26 red fluorescent dye and embryos. 20× magnification, color fluorescence is represented in greyscale. FIG. 12B shows images taken in the confocal cellular imaging system of embryos at the blastocyst stage injected with PKH26 (red) ESCs. Red fluorescing cells were detected in the ICM. FIG. 12C shows representative images of embryos at the blastocyst stage injected with PKH26 (red) ESC. Embryos were fixed and stained with DAPI (blue, represented in greyscale) to detect cell nucleus. Cells that fluoresced red were detected in the ICM. Images are under 20× magnification. FIG. 12D shows representative immunofluorescence images of embryos at the blastocyst stage, injected with PKH26 (red dye) ESCs. Embryos were fixed and stained with DAPI (blue fluorescence, represented in greyscale) to detect cell nucleus and an anti-SOX2 antibody (green fluorescence, represented in greyscale) as a pluripotent and ICM marker. Red fluorescing cells were detected in the ICM and showed pluripotency 20× magnification.

FIGS. 13A-13D show representative immunofluorescence images of embryos at day 8 of developmental stage following injection of ESCs at day 6 post fertilization stained with (FIG. 13A) DAPI as a nuclear marker, (FIG. 13B) an anti-SOX2 green fluorescent antibody, (FIG. 13C) PHK26 and (FIG. 13D) overlayed image. Color fluorescence is represented in greyscale. Images are under 20× magnification.

FIGS. 14A-14D show representative immunofluorescence images of embryos at day 8 of developmental stage following injection of ESCs at day 6 post fertilization stained with (FIG. 14A) DAPI as a nuclear marker, (FIG. 14B) an anti-SOX2 green fluorescent antibody, (FIG. 14C) PHK26 and (FIG. 14D) overlayed image. Color fluorescence is represented in greyscale. Images are under 20× magnification.

FIGS. 15A-15D show representative immunofluorescence images of embryos at day 8 of developmental stage following injection of ESCs at day 6 post fertilization stained with (FIG. 15A) DAPI as a nuclear marker, (FIG. 15B) an anti-SOX2 green fluorescent antibody, (FIG. 15C) PHK26 and (FIG. 15D) overlayed image. Color fluorescence is represented in greyscale. Images are under 20× magnification.

FIG. 16 shows a plasmid map of the FUW plasmid (Addgene plasmid #14882).

FIG. 17 shows a brightfield and fluorescent microscopy image merge demonstrating clonal outgrowth of male Jersey embryonic stem cells transduced with an EGFP lentivirus.

FIGS. 18A-18B show (FIG. 18A) NANOS3 PCR on DNA extracted from 90-day fetal tails. Images of 90-day fetal testes from (FIG. 18B) #3987, and (FIG. 18C) #5069.

FIG. 19 shows a UMAP plot of different cell populations of the fetal testis. Clusters were identified based on expression of well conserved marker genes. PGCs represented 9% of all cells.

FIG. 20 shows a UMAP plot of individual samples (n=4).

FIG. 21 shows a UMAP plot of samples by treatment showing that only control (CT) samples are present in the PGC cluster.

FIGS. 22A-22F show expression of well conserved pluripotency, early PGC and late PGC markers indicating the majority of 90 d PGCs are in the late stage (FIG. 22A—POUF (OCT4), FIG. 22B—NANOG, FIG. 22C—NANOS3, FIG. 22D—KIT, FIG. 22E—DAZL, FIG. 22F—DDX4 (VASA)).

FIG. 23A-23B shows a violin expression plot of late PGC markers showing the lack of germ cell marker expression in NANOS3 KO samples compared to the control (CT) samples.

FIG. 24 shows the general steps in an in vitro embryo production method to generate NANOS3 KO embryos using CRISPR-Cas9.

FIG. 25 shows a sample collection and analysis schedule for scRNA-Seq analysis of gonads from NANOS3 KO animals.

FIG. 26 shows NANOS3 KO efficiency using different gRNAs targeting NANOS3.

FIG. 27A-27B shows images of fetal testes from two different NANOS3 KO fetuses.

FIG. 28 shows results from PCR to detect NANOS3 in DNA extracted from blood from NANOS3 KO fetuses.

FIGS. 29A-29D—NANOS3 KO bull was germline ablated, but otherwise had normal reproductive development. (FIG. 29A) NANOS3 PCR on DNA extracted from bull #838 (“Fauci”) blood. Letters A-D represent different alleles. #838 was a mosaic KO, with 4+ alleles, including 1 large deletion, and no wildtype. (FIG. 29B) 1-day-old NANOS3 KO bull #838. (FIG. 29C) 12-months-old NANOS3 KO bull #838. (FIG. 29D) 12-month Breeding Soundness Exam (BSE) results for NANOS3 KO bull #838.

FIGS. 30A-30C—Physiological Characterization of NANOS3 KO bull #838. (FIG. 30A) Image of bull #838 at 15-months-old. (FIG. 30B) Image of bull #838's reproductive tract. (FIG. 30C) Representative images of H&E stained testis cross-sections from an age matched, wildtype (NANOS3+/+), bull (left panel) compared to bull #838 (NANOS3−/−) (right panel). Both samples have Sertoli cells lining the seminiferous tubules, but bull #838 lacks any spermatogenesis.

FIGS. 31A-31B—Physiological Characterization of bull #3964. (FIG. 31A) Image of bull #3964 at 15-months-old. (FIG. 31B) Image of bull #3964's reproductive tract. FIGS. 32A-32F—Physiological Characterization of NANOS3 KO heifer #854. (FIG. 32A) Image of heifer #854 at 15-months-old. (FIG. 32B) Image of heifer #854's reproductive tract. (FIGS. 32C-32D) Images of heifer #854's left ovary (FIG. 32C) and right putative primitive streak (FIG. 32D). (FIGS. 32E-32F) Representative images at different magnifications of H&E stained ovary cross-sections showing a complete lack of oogenesis.

FIG. 32E are from ovary shown in FIG. 32C. FIG. 32F are from ovary shown in FIG. 32D.

FIG. 33—PCR of DDX3 for sex confirmation of the fetus. A single band indicates a female and a double band at 184 and 208 bp indicates a male. Cosmo, a bull, was a positive control.

FIG. 34—PCR to detect GPF. The absence of a band at 425 bp indicates no GFP in fetuses' samples. Cosmo DNA was used as a positive control.

FIG. 35—Genotype analysis of CRISPR/CAS9 NANOS3 targeted bovine samples. NANOS3 long-range PCR results. The wild type (+) band size is 6,274 bp. Bands smaller than the wild type indicate a large (>500 bp) deletion in NANOS3.

FIG. 36—Summary of representation of the pluripotency state of murine, bovine, porcine and equine stem cells derived under different culture conditions. mESC: mouse embryonic stem cells (Naïve). FTW-mESC: formative mouse embryonic stem cells Yu et al. (2021). EpiSC: epiblast stem cells (Primed). bEPSC: bovine expanded potential stem cells (Zhao et al., 2021) bESC: bovine embryonic stem cells (Bogliotti et al., 2018, Proceedings of the National Academy of Sciences, 115, 2090-2095). pEPSC: porcine expanded potential stem cells (Zhao et al., 2021, Proceedings of the National Academy of Sciences 118, 9.) FTW-equi: formative equine stem cells (Yu et al. Cell Stem Cell 28, 550-567. (2021)).

FIG. 37—Representative images of presumptive NANOS/knockout embryos complemented with red (as represented in greyscale) bEPSC stained with DAPI as a nuclear marker (blue, as represented in greyscale), and anti-SOX2 antibody (green, as represented in greyscale). Overlay of the three channels. 20× magnification.

FIG. 38—Representative 7-day blastocysts with red-stained (as represented in greyscale) ESCs on the day of embryo transfer.

FIG. 39—Representative images of two recovered embryos under a stereoscope, arrows are pointing the embryonic disc.

FIG. 40A-40B—qPCR assay for ESCs carrying the EF1a-tdTomato marker in the first DNA extraction from elongated embryo placental tissues.

FIG. 41A-41B—qPCR assay for ESCs carrying the EF1a-tdTomato marker in the second DNA extraction from elongated embryo placental tissues.

FIG. 42—Representative immunofluorescence images of tdTomato expressing cells stained with an anti-TdTomato protein (green, as represented in greyscale). 20× magnification.

FIG. 43—Representative image of ESCs cells in the ICM of a blastocyst following injection of 5 cells ESC into a 5-day morula. There is clear evidence of expansion of these 5 ESC in the ICM of the expanded blastocyst as shown with red fluorescence (as represented in greyscale and denoted by black arrows).

FIG. 44—Representative image of embryos collected at blastocyst stage and transferred to recipients. 20× magnification.

FIG. 45—qPCR assay for ESCs carrying the EF1a-tdTomato marker in DNA extracted from placental tissue from three elongated embryos injected with ESC expressing TdTomato (from recipients 1076 and 1125) and seven embryos injected with ESC carrying the green fluorescent marker (from recipients #1074 and #1078).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater′ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); Marten H. Hofker and Jan van Deursen, Transgenic Mouse Methods and Protocols, 2^{nd} edition (2011); and Primrose and Twyman. Principles of Gene Manipulation and Genomics 2006, published by Blackwell Publishers.

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd}ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity (e.g., an individual). A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “blastocyst” means an early developmental stage of embryo comprising of inner cell mass (from which embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000).

As used herein, the terms “encoding” or “encoded”, with respect to a specified nucleic acid, refers to the information for transcription into RNA and, in some cases, translation into the specified protein. A nucleic acid encoding a protein can comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

As used herein, the term “early stage embryo” means any embryo at embryonic stages between fertilized ovum and blastocyst. Typically, eight cell stage and morula stage embryos are referred to as early stage embryos.

As used herein, “embryonic stem cells” or “ES cells” means cultured cells derived from inner cell mass of early stage embryo, which are amenable to genetic modification and which retain their totipotency and can contribute to all organs of resulting chimeric animal if injected into host embryo. “Developmental Biology”, sixth edition, ed. by Scott F. Gilbert, Sinauer Associates, Inc., Publishers, Sunderland, Mass. (2000).

As used herein, “primordial germ cells” means those cells arising early in the embryonic development that give rise to the spermatogenic lineage via a gonocyte intermediate or female germline via an oogonia intermediate.

As used herein, “self-renewing” refers to the capacity of an undifferentiated cell to divide while maintaining an undifferentiated state in at least one of the progeny cells so as maintain or expand the undifferentiated cell population, while optionally giving rise to a differentiated cell or cell population. Thus, “self-renewing cells”, as the term is used herein, are undifferentiated cells that have the capacity to divide and optionally differentiate, where upon division, at least one of the progeny cells retain an undifferentiated state so as to allow for maintenance or expansion of the undifferentiated cell population.

As used herein, “pluripotent” refers to the capacity of a cell to differentiate into any cell of the three germ layers (endoderm, mesoderm, and ectoderm). Thus, “pluripotent cells”, as the term is used herein, are cells that have the capacity to differentiate into or give rise to any cell of the three germ layers. Pluripotent cells thus have the capacity to divide into most cells of an organism but cannot develop a complete organism on their own.

As used herein, “totipotent” refers to the capacity of a cell or cell population to differentiate into any cell type (of e.g., a blastomere) or a complete embryo or animal (inclusive of a placenta). Thus, “totipotent cells”, as the term is used herein, are cells that have the capacity to differentiate into or give rise to any cell type (e.g., of a blastomere) or a complete embryo or animal (inclusive of a placenta). In other words, totipotent cells can develop a complete organism on their own. For example, zygotes are totipotent. Totipotent cells have the capacity to divide until the entire embryo or animal is formed.

As used herein, “genome selected” or “genotype selected” refers to cells, tissues, animals, and/or the like that have been selected based upon one or more DNA sequences of their genome. Techniques for determining the sequence of a genome and genotype at any particular locus are generally known in the art and are inclusive of all molecular biology methods of genome and DNA analysis, population genetics based approaches based on principles of inheritance, and combinations thereof.

As used herein, “zygote” refers to a single-cell embryo.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “allergen” refers to an antigen, microorganism, plant, or product thereof that produces an abnormal immune response in which the immune system fights off a perceived threat that would otherwise be harmless to the body. Allergens can be found in a variety of sources (e.g., animal products (e.g., meat, milk, and products produced therefrom), foods, insects, mold spores, plants, and chemicals). Allergens can include, but are not limited to dust mite, pollen, spores, poison ivy, poison oak, pet dander, royal jelly, peanuts (a legume), nuts, insect bites or stings, seafood, and shellfish.

As used herein, “culturing” can refer to maintaining cells under conditions in which they can proliferate and avoid senescence as a group of cells. “Culturing” can also include conditions in which the cells also or alternatively differentiate. Culturing can include one or more steps or conditions, and include in one or more steps passaging, transfer of cells, media changing, incubation temperature changes, atmospheric gas changes, and/or the like.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically, or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein. The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased expression” refers to a modification in a gene which affects the expression level of that or another gene such that expression of that or another gene is increased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “gene product” refers to any polynucleotide that is transcribed (in vivo or in vitro) into an RNA molecule. The term “gene product” also refers to polypeptides that are translated from an RNA gene product.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×1¹⁰ cells.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. For example, in some embodiments, the methods described herein include determining the disassociation constant for the targeting moiety and target. In some embodiments, the targeting moiety has a specificity the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 or more fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

The terms “subject,” and “individual,” are used interchangeably herein to refer to a vertebrate, preferably a mammal, more preferably a bovine. Mammals include, but are not limited to, murines, simians, humans, farm animals, sport animals, and pets. Tissues, cells and their progeny of a biological entity obtained in vivo or cultured in vitro are also encompassed by such terms.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Conventional genetic selection and breeding programs have generated populations of elite genetic seedstock. However, there exists a lag in the genetic improvement between the elite nucleus seedstock population and commercial animals. This lag is due to the generation intervals of conventional breeding programs. As such, there exists a need for approaches to improve the rate of genetic improvement, particularly at the commercial animal level.

Germline complementation, which involves the use of germline-deficient hosts, is an approach that can be used to efficiently disseminate animals with superior genetics and/or traits. One method to generate germline-deficient hosts is via treatment with chemotoxic drugs (e.g., busulfan) or local irradiation, but these methods are not efficient in livestock because they either fail to completely eliminate the endogenous germline, or the treatment has undesirable side effects on animal health. A promising alternative is to use GnEd to knockout in a zygote a gene (e.g., NANOS2 or DAZL) that is necessary for that animal's own germ cell production. NANOS2 is predominantly expressed in male germ cells and is required for the maintenance of the spermatogonial stem cell population.

The NANOS gene family, including NANOS3, is required for germ cell development, although the processes regulated vary among species and among different homologs. NANOS3 is found in migrating primordial germ cells (PGCs) of both sexes and homozygous deficiency of NANOS3 results in the complete loss of male and female germ cells in mice (see e.g., Tsuda et al., Science. 2003. 301(5637):1239-1241). More recently, female NANOS3 knockout embryos were generated using a somatic cell nuclear transfer strategy and were observed to contained fetal ovaries that lacked germ cells (Ideta et al., 2016. Sci. Rep. 6:24983). However, there have been no reports of a knockout male bovines or germline depleted male bovines or any approach to knock out NANOS3 in bovine using a gene editing approach.

With that said, embodiments disclosed herein can provide methods and compositions of germline complementation and for use in a germline complementation strategy, such as NANOS3 deficient cells and/or non-human animals, and more particularly, genetically germline ablated non-human animals (such as bovine). Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Germline Complementation

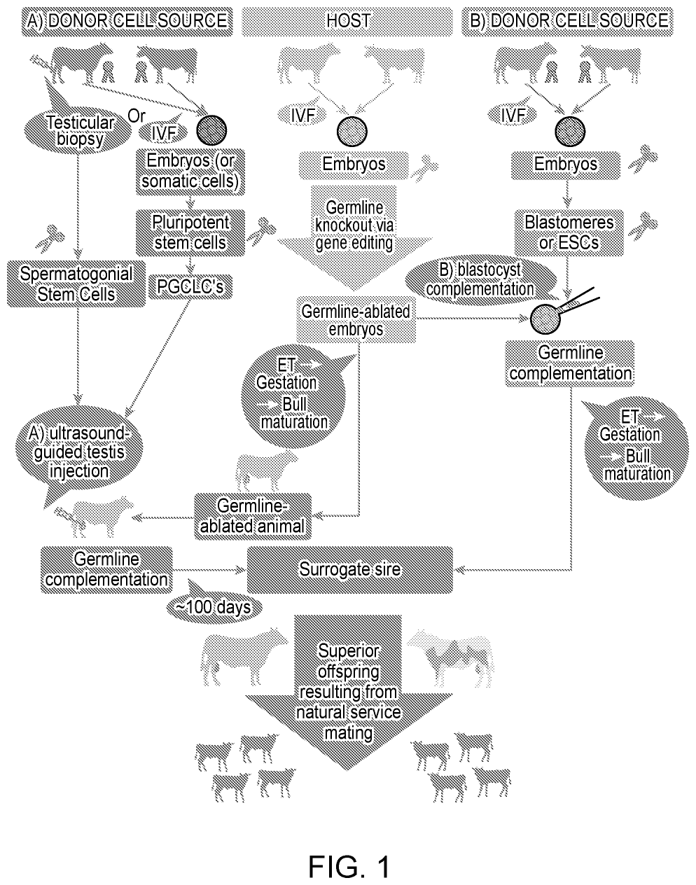

Described in several exemplary embodiments herein are methods of germline complementation utilizing host animals and/or cells that are NANOS3 deficient and suitable donor cells. Host and donor cells and/or animals for use in the germline complementation methods are described in greater detail elsewhere herein. A general strategy for germline complementation to generate surrogate sires is shown in FIG. 1. Although not shown, a similar approach and strategy can be used to generate surrogate dams. Generally, host animals/embryos that are NANOS3 deficient such that they do not contain germ cells (also said herein to be germline ablated, genetically germline ablated, germline defective, or germline deficient) or have the capacity to produce germ cells. Germline complementation with a suitable donor cell is then used to supplement the host cells with germ cells (or the capacity to produce the missing germ cells) from the donor cell/animal. This can produce the surrogate sires and/or dams that can be used in natural service mating or conventional A.I. breeding to produce commercial offspring with the donor cell genetics.

Complementation of cells occur by injecting donor cells (exogenous cells to the host) into the gonads of a host animal or into a preimplantation host embryo during host embryo development (see e.g., FIG. 1). In some embodiments, a method of non-human animal embryo complementation includes introducing a self-renewing exogenous population of cells into a non-human animal preimplantation embryo, optionally at about day 3, 4, 5, or 6 post fertilization; optionally washing the non-human animal preimplantation embryo in HEPES or other suitable buffer; and culturing the non-human preimplantation embryo in a suitable culture media optionally consisting of a 1:1 ratio by volume of a suitable bovine culture media, such as one that is at least supplemented with N2, B27, FGF, and IWR-1. The suitable embryo culture medium can include one or more salts (e.g., sodium chloride, potassium chloride, calcium chloride, monopotassium phosphate, magnesium sulphate), one or more buffers (e.g., sodium bicarbonate), one or more energy substrates (e.g., glucose, sodium lactate, and/or sodium pyruvate), non-essential amino acids or mixes (e.g., NEAA's 8, NEAA's 9), one or more glutamine dipeptides (e.g., alanyl-glutamine), one or more essential amino acids or mixes (e.g., EAA's 2, EAAs 11, and/or the like), one or more chelators (e.g., EDTA), one or more macromolecules (e.g., hyaluronan, HAS, and/or the like), one or more fatty acids (e.g., lipoic acid and/or the like), one or more vitamins (e.g., A, E, D, C, K, B, folate), one or more antibiotics and/or antifungals, or any combination thereof. Other exemplary embryo culture media includes, without limitation, M2 medium, cleavage K-SCIM medium, Blastocyst K-SIBM medium, Quinns Advantage Cleavage media, Quinns Advantage Blastocyst media, FERTICUK IVF medium, FERTICULT G3 medium, IVC-TWO medium, IVC-THREE medium, ECM medium, MultiBlast medium, EmbryoAssist medium, BlastAssist medium, ISM1 medium, ISM2 medium, G-1PLUS medium, G-2PLUS medium, IVF medium, CCM medium, BO-IVF medium (ivf bioscience), those described in e.g., Thompson and Peterson. 2000. Hum Reprod. December; 15 Suppl 5:59-67. doi: 10.1093/humrep/15.suppl_5.59; Santana et al., Mol Reprod Dev. 2014 October; 81(10):918-27. doi: 10.1002/mrd.22387; Gandhi et al., Hum Reprod. 2000. 15(2):395-401; Rizos et al. 2003. Biol. Reprod. 68(1):236-243, and/or the like. In some embodiments the cell culture media for embryo culture (including but not limited to any of those previously described) is supplemented with supplementation with N2, B27, FGF, and IWR-1. In some embodiments, the culture media is as described in Bogliotti et al. PNAS. 2018. 115(9), doi.org/10.1073/pnas.1716161115.

In certain example embodiments, the number of donor exogenous cells introduced into the host is about 1 to about 25 cells or about 30-50 percent of the total number of cells present in the embryo prior to introducing the exogenous cells. In certain example embodiments, the number of exogenous cells introduced at 3 days or 4 days post fertilization is about 5 cells. In certain example embodiments, the number of exogenous cells introduced at 5 days post fertilization is 8 cells. In certain example embodiments, the number of exogenous cells introduced at 5 days post fertilization is 9 cells. In certain example embodiments, the number of exogenous cells introduced at 5 days post fertilization is 10 cells. In certain example embodiments, the number of exogenous cells introduced at 6 days post fertilization is about 10-20 cells.

In certain example embodiments, self-renewing exogenous donor cells are embryonic stem cells, expanded embryonic stem cells, pluripotent stem cells, induced pluripotent stem cells, multipotent stem cells, totipotent stem cells, primordial germ cells, primordial germ cell-like cells, totipotent cells, or a combination thereof.

In certain example embodiments, the host non-human animal embryo into which the exogenous cells are introduced is genetically germline ablated. In certain example embodiments, the non-human animal embryo comprises or consists of one or more engineered host cells of any as described elsewhere herein. In certain example embodiments, the self-renewing exogenous cells are germline competent. In certain example embodiments, the self-renewing exogenous cells are engineered cells comprising one or more gene modifications in one or more target genes and wherein the one or more target genes are not NANOS3. In certain example embodiments, the self-renewing exogenous cells are not genetically modified. In certain example embodiments, the self-renewing exogenous cells comprise an elite genome, a genomically selected genome, or both.

### Complemented Embryos and Animals

Animals containing both host (a first cell or cell population) and donor (a second cell or cell population) cells produced by a complementation technique previously described are referred to herein as complemented animals. Such a term encompasses specific cell type complementation (e.g., germ cells or other tissue type cells) and genetic complementation (e.g., specific genome or genotype complementation). It will be appreciated that in some contexts complemented embryos, animals, and/or progeny thereof may be considered non-natural or engineered and that in other contexts complemented embryos, animals, and/or progeny thereof may be considered natural or not engineered. Such contexts can be influenced about the cell type and/or genetics being complemented. The complemented animals can be used as surrogate sires or damns that can produce progeny. Progeny can be obtained by any suitable method or technique including natural mating, in vitro fertilization, artificial insemination, embryo transfer, and/or the like.

Described in certain example embodiments herein are complemented non-human animals or embryos comprising or consisting of a first population of cells (host cells) comprising one or more cells, wherein the first population of cells consists of an engineered non-human animal cell or population thereof as described elsewhere herein; and a second population of cells (donor cells) comprising one or more cells, wherein the second population cells are not an engineered deficient non-human cell or population thereof any one of the preceding paragraphs and/or as described elsewhere herein. In certain example embodiments, the second population of cells comprises or consist of one or more engineered cell population comprising one or more genetic modifications in one or more target genes and wherein the one or more target genes are not NANOS3. In certain example embodiments, the second population of cells is not an engineered cell or population thereof. In certain example embodiments, the second population of cells comprises an elite genome, a genomically selected genome, or both. In certain example embodiments, the second population of cells comprises or consists of one or more embryonic cells, optionally a zygote or inner cell mass cells; stem cells, optionally embryonic stem cells or induced pluripotent stem cells; spermatogonial stem cells or oogonial stem cells; primordial germ cells; or primordial germ cell like cells. In certain example embodiments, the second population of cells are self-renewing cells. In certain example embodiments, the second population of cells is pluripotent, totipotent, or multipotent. In certain example embodiments, the second population of cells is germline competent.

In certain example embodiments, the complemented embryo is a morula. In certain example embodiments, the complemented non-human animal or embryo is a male. In certain example embodiments, the complemented non-human animal or embryo is a female. In certain example embodiments, the complemented non-human animal or embryo is a bovine, equine, porcine, ovine, caprine, camelid, cervine, canine, feline, murine, leporine, or cavine.

Described in certain example embodiments herein are non-human animals developed or generated from the complemented non-human animal or embryo as described elsewhere herein. In certain example embodiments, one or more germ cells of the non-human animal originated from the second population of cells (donor cells). In certain example embodiments, about 0.001 percent to 100 percent of the germ cells originated from the second population of cells. In certain example embodiments, the non-human animal is a male. In certain example embodiments, the non-human animal is female.

In certain example embodiments, the complemented embryo is a preimplantation embryo, optionally a zygote, 2 cell, 4 cell, an 8 cell, 16 cell, a blastocyst, or a morula. In certain example embodiments, the first population of cells makes up a percentage of cells of the complemented non-human animal or embryo ranging from about 25 percent to any percent up to but not including 100 percent. In certain example embodiments, the complemented non-human animal or embryo comprises at least one cell of the second population of cells, optionally wherein the second population of cells makes up a percentage of cells of the non-human animal or embryo ranging from any non-zero percent to about 75 percent. In certain example embodiments, the complemented embryo is a day 3 post fertilization embryo, a day 4 post fertilization embryo, a day 5 post fertilization embryo, or a day 6 post fertilization day embryo. In certain example embodiments, the day 3 post fertilization complemented embryo comprises about 5 cells from the second population of cells; the day 4 post fertilization complemented embryo comprises about 5 cells from the second population of cells the day 5 post fertilization complemented embryo comprises about 8-10 (e.g., 8, 9, or 10 cells) cells from the second population of cells; and the day 6 postfertilization complemented embryo comprises about 10-20 cells from the second population of cells.

In some embodiments, germline complementation results in a complemented embryo and/or animal that is an engineered NANOS3 deficient non-human animal or embryo that, in addition to NANOS3 deficient cells(s), further includes a second population of cells comprising one or more cells, wherein the second population of cells does not comprise engineered non-human animal cells of any one of the preceding paragraphs or as described elsewhere herein and wherein the second population of cells are germline competent cells, germ cells, or gametes. In certain example embodiments, the second population of cells comprises or consists of one or more embryonic cells, optionally a zygote or inner cell mass cells; stem cells, optionally embryonic stem cells or induced pluripotent stem cells; spermatogonial stem cells or oogonial stem cells; primordial germ cells; or primordial germ cell like cells. In certain example embodiments, the second population of cells comprises or consists of one or more spermatids or one or more oocytes. In certain example embodiments, the second population of cells comprises or consists of spermatozoa or ova. In certain example embodiments, the second population of cells comprises or consists of one or more engineered cells comprising one or more genetic modifications in one or more target genes and wherein the one or more target genes are not NANOS3. In certain example embodiments, the second population of cells do not comprise or consist of an engineered cell or population thereof. In certain example embodiments, the second population of cells comprises or consists of an elite genome, a genomically selected genome, or both.

Also provided herein are progeny of the complemented embryos and/or animals.

### Host and Donor Cells for Germline Complementation

As shown in FIG. 1, germline complementation utilizes a germline deficient or germline ablated host animals/cells and germline or genetic donor cells/animals. The germline ablated host animals/cells can be NANOS3 deficient and are further described in greater detail below. The donor cells contain the desired genetics to ultimately pass on in the complementation strategy via the surrogate sires and damns. In some embodiments, donor animals/cells are genetically modified to contain a desired genotype and/or transgene. In some embodiments, the donor animals/cells are not genetically modified. In some embodiments, the donor cells contain elite genetics. Donor animals and cells are described in greater detail below.

### Engineered NANOS3 Deficient Host Cells and Organisms

Germline complementation allows for the reduction of generation interval for an increased rate of genetic improvement, using unmodified and modified genetic seedstock, even at the commercial animal level. See e.g., FIG. 1. As shown and described in e.g., FIG. 1, a key component of a germline complementation strategy is a host organism or embryo that is germline depleted. Described in various embodiments herein are non-human animals, particularly bovine, cells thereof and progeny thereof that are deficient in or lacking a functional NANOS3 gene and/or gene product such that they are rendered genetically germline deficient/ablated. In general, such non-human animals contain one or more genetic modifications that result in NANOS3 gene and/or gene product that is deficient or eliminated, and ultimately lead to a lack of sufficient function of the NANOS3 gene and/or gene product so as to result in a non-human that is germline ablated, depleted, deficient and/or incompetent. Non-human animals and cells with genetic modifications to the NANOS3 gene and/or gene product such that the NANOS3 gene and/or gene product are eliminated, depleted, deficient in, and/or non-functional are generally referred to herein as NANOS3 deficient cells and organisms (e.g., non-human animals). Thus, in some embodiments, NANOS3 deficient organism, such as non-human animals, are genetically germline deficient/ablated. Without being bound by theory, the genetic germline ablation/deficiency results from the modifications to the NANOS3 gene and/or gene product that render the NANOS3 gene and/or gene product non-functional. The NANOS3 deficient cells and/or organisms can be used to produce NANOS3 deficient cells, embryos, and/or adult animals suitable for germline complementation by a suitable complementation strategy (see e.g., FIG. 1). Further embodiments, features, and advantages of such modifications, cells, and organism are now described in greater detail.

Described in several exemplary embodiments herein are engineered NANOS3 deficient cells and organisms. In some embodiments, the cells are bovine cells. In some embodiments, the NANOS3 deficient organisms are bovine. In some embodiments, the NANOS3 deficient organism is a male. In some embodiments, the NANOS3 deficient organism is a female. In some embodiments, the NANOS3 deficient organism is a male bovine. In some embodiments, the NANOS3 deficient organism is a female bovine. Such cells can be used in an embryo complementation strategy to complement a NANOS3 deficient embryo with allogenic donor cells, particularly germline allogenic donor cells. In other embodiments, NANOS3 deficient organisms can be complemented with allogenic cells that are depleted or absent in the NANOS3 deficient organism, such as germline cells or germline progenitor cells, or germline competent embryonic cells. In some embodiments, the NANOS3 deficient organisms can be complemented with allogenic cells that capable of producing the cells that are absent or depleted in the NANOS3 deficient organism, such as progenitor or stem cells capable of producing the cells that are absent or depleted in the NANOS3 deficient organism. In some embodiments, the cells that are depleted or absent in the NANOS3 deficient organism are germline and/or germline progenitor cells. In the context of FIG. 1, NANOS3 deficient organisms are “Host” animals and the allogenic cells which can be introduced to the NANOS3 deficient organisms are “donor cells” which can be obtained from a “donor cell source” and optionally modified as described elsewhere herein.

In certain example embodiments, the engineered non-human animal cell or population thereof (e.g., a host NANOS3 deficient cell or population thereof) is a bovine, equine, porcine, ovine, caprine, camelid, cervine, canine, feline, murine, leporine, or a cavine cell. In certain example embodiments, the engineered non-human animal cell or population thereof is an engineered somatic cell or population thereof. In certain example embodiments, the engineered non-human animal cell or population thereof is an engineered germ cell or population thereof. In certain example embodiments, the engineered germ cell or population thereof is an engineered gamete or population thereof. In certain example embodiments, the engineered gamete or population thereof is an engineered spermatozoon or population thereof or an engineered ovum or population thereof. In certain example embodiments, the engineered germ cell or population thereof is an engineered immature germ cell or population thereof. In certain example embodiments, the engineered immature germ cell or population thereof is an engineered spermatid or population thereof or an engineered oocyte or population thereof. In certain example embodiments, the engineered non-human animal cell is an engineered embryonic cell population thereof, optionally wherein the engineered embryonic cell is a zygote. In certain example embodiments, wherein the engineered non-human animal cell population thereof is an engineered blastocyst cell or population thereof, optionally an engineered inner cell mass cell or population thereof. In certain example embodiments, the engineered non-human animal cell or population thereof is an engineered stem cell or population thereof, optionally an engineered embryonic stem cell or population thereof or an induced pluripotent stem cell or population thereof. In certain example embodiments, the engineered non-human animal cell or cell population is an engineered spermatogonial stem cell or population thereof or an engineered oogonial stem cell or population thereof. In certain example embodiments, the engineered non-human animal cell or cell population cell is a primordial germ cell or population thereof or an engineered primordial germ cell-like cell or population thereof. In certain example embodiments, the engineered non-human animal cell or population thereof is an engineered self-renewing cell or population thereof. In certain example embodiments, the engineered non-human animal cell is pluripotent, totipotent, or multipotent. In some embodiments, the engineered non-human animal cell is a pluripotent cell as described in International Pat. App. Pub WO 2019/140260.

In certain example embodiments, one or both of the NANOS3 alleles are modified. In certain example embodiments, the engineered non-human animal cell or population thereof is monoallelic for the NANOS3 gene modification. In certain example embodiments, the engineered non-human animal cell or population thereof is biallelic for the NANOS3 gene modification. In certain example embodiments, the engineered non-human animal cell population thereof does not express a functional NANOS3 gene or gene product.

In certain example embodiments, the engineered non-human animal cell is heterozygous or homozygous for the NANOS3 gene modification, wherein the NANOS gene modification is optionally a NANOS3 gene knockout.

In certain example embodiments, the engineered non-human animal cell or population thereof (e.g., a NANOS3 deficient non-human animal cell or population thereof) is an engineered male cell or population thereof. In certain example embodiments, the engineered non-human animal cell is an engineered female cell or cell population.

Also provided herein are engineered non-human animal embryos, engineered non-human animals, and progeny thereof that are NANOS3 deficient and/or have one or more engineered non-human animal NANOS3 deficient cells. The engineered NANOS3 deficient non-human animal can include one or more engineered NANOS3 deficient non-human animal cells described herein. Also described herein are progeny of the NANOS3 deficient non-human animals. Progeny can be obtained by any suitable method or technique including natural mating, in vitro fertilization, artificial insemination, embryo transfer, and/or the like.

In certain example embodiments, the engineered non-human animal, embryo, or progeny thereof is a chimera. In certain example embodiments, the engineered non-human animal, embryo, or progeny thereof is a mosaic. In certain example embodiments, the engineered non-human animal, embryo, or progeny thereof is not chimeric. In certain example embodiments, the engineered non-human animal, embryo, or progeny is not a mosaic. In certain example embodiments, at least 1 cell of or at least 0.0001 percent to 100 percent of all cells of the engineered non-human animal, embryo, or progeny thereof is an engineered non-human animal cell (e.g., an engineered NANOS3 deficient non-human animal cell) as described elsewhere herein.

In some embodiments, 0.0001%-100%, 0.0001%-0.001%, 0.001%-0.01%, 0.01%-0.1%, 0.1%-1%, 1%-10%, 10%-20%, 20%-30%, 30%-40%, 40%-50%, 50%-60%, 60%-70%, 70%-80%, 80%-90%, or 90%-100% of all cells of the engineered non-human animal, embryo, or progeny thereof is an engineered non-human animal cell (e.g., an engineered NANOS3 deficient non-human animal cell).

In certain example embodiments, the engineered NANOS3 deficient non-human animal, embryo, or progeny thereof is a male. In certain example embodiments, the engineered NANOS3 deficient non-human animal, embryo, or progeny thereof is a female. In certain example embodiments, the engineered NANOS3 deficient non-human animal is a bovine, equine, porcine, ovine, caprine, camelid, cervine, canine, feline, murine, leporine, or a cavine.

The engineered NANOS3 deficient non-human animal, embryo, or progeny thereof can be used as a host animal in a germline complementation strategy as described in greater detail elsewhere herein.

In some embodiments, a method of generating a NANOS3 modified non-human animals or embryos, comprises introducing one or more NANOS3 gene modifications to a non-human animal cell, wherein the NANOS3 gene modification reduces or eliminates the expression of a NANOS3 gene product; and one or more of the following techniques: somatic cell nuclear transfer, oocyte pronuclear DNA microinjection, zygote microinjection, or embryo microinjection, intracytoplasmic sperm injection, in vitro fertilization, embryo transfer, in vitro embryo culture, or any combination thereof. NANOS3 gene modifications are described elsewhere herein. In certain example embodiments, introducing one or more NANOS3 gene modifications to the non-human animal cell comprises CRISPR-Cas mediated gene modification, Zinc Finger Nuclease gene modification, TALEN mediated gene modification, recombinase mediated gene modification, prime editing mediated gene modification, meganuclease mediated gene modification, transposase/transposon mediated gene modification, or any combination thereof. In certain example embodiments, introducing one or more NANOS3gene modifications to the non-human animal cell comprises use of a CRISPR-Cas system and wherein the guide RNA for the CRISPR-Cas system targets exon 1 of the NANOS3 gene, optionally in the zinc finger region, and are optionally selected from any one of SEQ ID NOs: 39-45, or any combination thereof. Other methods and techniques of introducing gene modifications, such as NANOS3 are described in greater detail elsewhere herein.

### NANOS3 Modifications

In some embodiments, one or more copies or alleles of NANOS3 can be modified such that expression of the NANOS3 gene and/or gene product is reduced (e.g., reduced below detectable or functional levels), and/or eliminated. Any suitable gene or genetic modification system can be used to modify the NANOS3 gene. Exemplary genetic modification systems are described in greater detail elsewhere herein.

The NANOS3 deficient cells and organisms can be generated using any suitable genetic modification method and/or system. Exemplary suitable systems are described in greater detail below and the Working Examples elsewhere herein.

Modification of the NANOS3 encoding polynucleotide (e.g., a gene and/or transcribed gene product) to generate a germline ablated host can be accomplished by utilization of a genetic modification system and can occur at any appropriate stage for the system utilized. For example, modification can occur by modification of a polynucleotide (such as a genome) in vitro or ex vivo in zygotes, developing embryos, early embryos, blastocysts, blastomeres, morulas, embryonic stem cells, primordial germ cells, primordial germ cell like cells, pluripotent stem cells (including but not limited to those described in In some embodiments the cell is a pluripotent embryonic stem cell as described International Pat. App. Pub WO 2019/140260), induced pluripotent stem cells (such as those reprogrammed from somatic cells), spermatogonial or oogonial stem cells, and/or the like using any suitable system, such as a CRISPR-Cas, transposon, ZFN, TALEN, and/or the like. Exemplary suitable systems for genetic modification of the polynucleotides generally and, more particularly, the genome are described in greater detail elsewhere herein.

The modification in the NANOS3 encoding polynucleotide can be an insertion, deletion, insertion and deletion (indel), substitution, or any combination thereof.

In certain example embodiments, the NANOS3 gene modification is an insertion of one or more nucleotides; a deletion of one or more nucleotides; a substitution of one or more nucleotides; or any combination thereof. In certain example embodiments, the NANOS3 gene modification(s) is/are in exon 1, exon 2, or both of the NANOS3 gene. In certain example embodiments, the NANOS3 gene modification(s) is/are in exon 1 of the NANOS3 gene, optionally in the zinc finger domain of the NANOS3 gene.

In some embodiments, the NANOS3 polynucleotide (gene) that is modified has a sequence that is 80-100 percent identical; at least 85; at least 90; at least 95; at least 96; at least 96; at least 97; at least 98; at least 99; 85-100; 90-100; 91; 92; 93; 94; 95; 96; 97; 98; 99; or 100 percent identical to any one of SEQ ID NOs: 8, 10 or 11 or region thereof comprising at least 20 contiguous nucleotides.

>Bovine NANOS3 gene
>ENSBTAG00000000399
SEQ ID NO: 8
ATGGGGACCTTCAACCTGTGGACAGACTACTTGGGTTTGGCACGC
CTGGTTGGGGCTCAGCGTGAAGAAGAGGAGCCGGAGACCAGGCTG
GATCGCCAGCCAGAAGCAGTGCCCGAACCGGGGGGTCAGCGACCC
AGCCCTGAATCCTCACCAGCTCCCGAGCGCCTGTGTTCTTTCTGC
AAACACAACGGCGAGTCCCGGGCCATCTACCAGTCCCACGTGCTC
AAGGATGAAGCGGGCCGGGTGCTGTGCCCCATCCTCCGCGACTAC
GTGTGCCCCCAGTGCGGGGCCACCCGCGAGCGCGCCCACACCCGC
CGCTTCTGCCCGCTCACCGGCCAGGGCTACACCTCCGTCTACAGC
TACACCACCCGGAACTCGGCCGGCAAGAAGCTGGTCCGCTCGGAC
AAGGCGAGGACGCAGGACCCTGGACACGGACCGCGCCGAGGAGGA
GGTGCCTGTGCAGGTGGCTGGGGGGACTCGCTGCAGAGGGGGGTC
TGCCCTGGGCTGACTTAGAGCCTCTGAGAAGTGGGTTAACCCCTG
GGCTGACCCACTTCAGAGGGTTGGGTGGGGGAGAGAATCCATACA
CAATAGAAGGCTTAGAACCATGTTCTAGAACTGTTGCCCTAGTGG
GTAAGCTGGGGCTGGGGTTCCCTGTGTGACCTTGGGCAAGACACT
CTCCTTCTCTGGGCCCATAGAGGGTATATTGTTTTCAGGCGCAGG
GTGAGCTAGCAGGGAGGCCGTGGGGACTAGTGATGGGGTCTGGCA
ACAGGTACAGCAGGGGCTGCAATCTTGTGCGGAATGCAGGTGCCA
CGTGACCAGAGGGAAACAGCCTCACTTGTCCACTCATTCGCCAGG
TGGTTTGTGAGGCTTGTCCTGTGCCAAGCACTGGGCTGGGCTTGG
CCTTCAGTGGTAGATGGGACAGTGACAGTTATAACAGAAACGGGG
GAGTCTAAAGGGAGCACTCAAGCTAGACAAAGAGGTCCAGGAGGG
CTTCCTGGAGGAAGTGACTCCTGGAAACCTCATTCAGTTTGGTGA
AATGCTGAAGGGGGTCTGGGCACTGGGGCTCACTAGGGAACTTGG
TTAAATTCCGTGCGTCTGGGATGGGAGTATGCGTGCATGAAGGAT
GGAGGGGAAGGTGTCAGAAATTTCGGGGAAGGGCTGCTTGCTGAT
CGCCTTGAAGAGTTTGGACTTGAGTACTAGGGAGCTGAGAAGGGG
ACACGAGTAGGTCTGTGTATTAAGGGTGTGTTAGACTGTGCGTGT
GTTAGACTGTGGCTAATGGGTTGGGCTGTGGGGGTGGATTTGAGG
GGGTTGGAAGAAGTTGGTCCTCCAGGCAGGTGTTGGAGTTGGCAT
CAGTTTGGAGGGAAAGAACCATGAGTTTGCTGTGTGTGGGTCACC
CCCCGGGAGGGTGGCTGGAGGCTCCCTAAGGTCTGCACAGCCACA
GAGGGTTCCCTCCATCTTGCTCATCAAATTCGGAGTCCAGTTTTG
CAGGCCTGAGTGAGGCCAGAGCATCTGTACTTCTAACTAGATCCC
CTTGACCTCCGTAGCCGGCAGGCTTCGAGTAGCTAGGCTTACGTA
TCAGCAGTTACTATTACAGACTCGAAACACCTCCGCTTCTGCAGG
TTTATAAGAGAAATCCGTTTTAGCAACTTTGGGGAAGTCAGCCAA
CAGAGGGACCGGTTTGACGGGGCGGTACCCCATTACTGCCTCTCT
CAGACTGGGGGCCCCTCGGGAAGTCACCAGAAGGGAGGACCCCTT
CCCATCCCGGCGGCTGTGCGGAGAACACGTTAGAAGGGTTGTGAG
ACTGCCTGCGGGAGAGGATAGTATTCCTGTGAGGTCTGACAAGGT
CCAACTGGGAGAGGGAGGAAGCGGAAGCGACACAGAGACGATGGT
CTCAGAATCTTCTTTTTTCCCCTAGGGAACAGGAAAACTTGACTC
AGGGGTGGGGGGGGTCGGGGAGCCCTCACTACGATATTGGCACGG
AAAGGCTGCCTGAGGCTGTCCTCCCAGGAAACTTTCTGAGTGTAA
CAAATCCGGGGTTCCCGGAGCCCTGCGGCCGCGAGGGGGCAGTAC
GGCAGGACAGAGTTGGGAGTTTCCGTTGCTTTGTGTCTGGCTCTG
GGTCCCACCTGGGACTGCCCGGGGCTGCGAATAGCACAGGGGTGC
TAGTCCGGAAGGAGCCTGCAGGTGGAGCCAGAGCCCCGGCAGCTC
TGCAGGTTGAGTCGGAACTTCCGGATCATCGAATCATCCTTATTA
CTAAATGCTTTTCCCCTCCCCCCAACTCTGCTTTTAAAATCTAGG
TTCCAAAGGTGCCAGGAAGTCTTCTGGAACTCCTCCCTCTTCCTG
CTGCCCCTCAACTTCTGCCTAAGGAGACTGGCGTGGGCAGGATGA
CGCCTTCACCTGGGGATGGGGACCCAGGCTCAGTGGAGGCTGGGT
TTCAGGGAAGACCCACCCTCCGAGGATCCGCCCCCTAGACGGTGC
CTCCAGCCTGGGGGCTTGGCAAAGGAGCCCGGTCTGGGACCACCG
CCCAAAGCGCGCCCGCCCCTGTCACTGAAGGGGGTGGTCCTCAGG
CACCCCTGCCCTTCTTCCCCAACGCTGAGCAACCAGTCAGCGCTC
AATAAATGTTTATGAATGGATCA
>Bovine NANOS3 Exon 1
>ENSBTAE00000003992
SEQ ID NO: 10
ATGGGGACCTTCAACCTGTGGACAGACTACTTGGGTTTGGCACGC
CTGGTTGGGGCTCAGCGTGAAGAAGAGGAGCCGGAGACCAGGCTG
GATCGCCAGCCAGAAGCAGTGCCCGAACCGGGGGGTCAGCGACCC
AGCCCTGAATCCTCACCAGCTCCCGAGCGCCTGTGTTCTTTCTGC
AAACACAACGGCGAGTCCCGGGCCATCTACCAGTCCCACGTGCTC
AAGGATGAAGCGGGCCGGGTGCTGTGCCCCATCCTCCGCGACTAC
GTGTGCCCCCAGTGCGGGGCCACCCGCGAGCGCGCCCACACCCGC
CGCTTCTGCCCGCTCACCGGCCAGGGCTACACCTCCGTCTACAGC
TACACCACCCGGAACTCGGCCGGCAAGAAGCTGGTCCGCTCGGAC
AAGGCGAGGACGCAGGACCCTGGACACGGACCGCGCCGAGGAGGA
G
>bovine NANOS3 Exon 2
>ENSBTAE00000411199
SEQ ID NO: 11
GTTCCAAAGGTGCCAGGAAGTCTTCTGGAACTCCTCCCTCTTCCT
GCTGCCCCTCAACTTCTGCCTAAGGAGACTGGCGTGGGCAGGATG
ACGCCTTCACCTGGGGATGGGGACCCAGGCTCAGTGGAGGCTGGG
TTTCAGGGAAGACCCACCCTCCGAGGATCCGCCCCCTAGACGGTG
CCTCCAGCCTGGGGGCTTGGCAAAGGAGCCCGGTCTGGGACCACC
GCCCAAAGCGCGCCCGCCCCTGTCACTGAAGGGGGTGGTCCTCAG
GCACCCCTGCCCTTCTTCCCCAACGCTGAGCAACCAGTCAGCGCT
CAATAAATGTTTATGAATGGATCA

In some embodiments, the NANOS3 gene transcript (i.e. mRNA) that is expressed from the NANOS3 polynucleotide (gene) that is modified has a sequence that is 80-100 percent identical; at least 85; at least 90; at least 95; at least 96; at least 96; at least 97; at least 98; at least 99; 85-100; 90-100; 91; 92; 93; 94; 95; 96; 97; 98; 99; or 100 percent identical to any one of SEQ ID NOs: 2, 4, 5, 7, or 9 or region thereof comprising at least 20 contiguous nucleotides.

```
NCBI Reference Sequence: XM_027547963.1
>XM_027547963.1 PREDICTED:
Bos indicus x Bos taurus nanos C2HC-type zinc finger 3
(NANOS3), transcript variant X1, mRNA polynucleotide sequence
SEQ ID NO: 2
GCCGCCCCTGGAGGGAGGGACTGGGGACCGGGTTTGAGGGTGAAGAAATGGGGAAGAGCATTAAC
GGGGTAAGCCTCGTGTAGTTATGCGCTTGGGCCCCCGTCTGATCCGACAAGGGCCCGAGTTTGGA
AGCCCGGGACCCTCTGCGATCCTCTAGCTTCGCCCTTGTCCAACCGGCAGGTGGACCCACAAGGC
GGGCTAGGCAGCGGCCCCACCTCGGGGCTCGAATTTGCAAAGTGCAGACTCAGACAACCCTCCCC
CCAACCACCTTGGGTTGTTGTGATTCATAAACCATTGTGTCCGGAACACGGTGAAGCTCACTTAG
GTATTACATTGTATTAAAATGACTTGTTTATCTCTCCGTTGCATCCATGCCCCCGGGGCCAGAAC
CACTTGGCCTCCAGACCTCTTGGGGCCTCTCGGAATCCCTCCTCTGCCTCTGCCTCTAGCTAAGG
GTGCCCTCTGTTCTGGCCTGTCTCCCAAACTGATAATTGGAAGAAATATGCACCGTTGAGGGCCC
TTTTGAGAATGCTTTGACTAAATGGGTTAGAAGCCCAGCGCCCGCTGCTGCTATATTTGCATAGC
AAAGGTGACAGAAGTATCTGCTGATATTATTACTTAGATTTATCTCCTTTTTCCCTGTCCTGGAG
CAGAGTTGGCTCCTTCCTGCTATCTGTTCCCTGACTTAATAGATTCTCTAAGTCTCTCATTCCCT
TCCCCTCCCTCACCCTACCCGGTTCCTTGACCCACCCCGCCCCCCAGCCTCCACTCCCTGCCCCC
CAAGGAGTTGCCAAGGGTTTGGGGGAACATTCAACCTGTCGGTGAGTTTGGGCAGCTCAGGCAAA
CCATCGACCGTTGAGTGGACCCCGAGGCCTGGAACTGCCGTCCACCCACCCACCCATCACGACCC
CCAACTTTCAGATCTGGGGTAGGGGCAGGGGATCCCGAACACATCCCCTCCCTTAGGCCACAGCG
AAGGTCACAATCAACATTCATTGTTGTCGGTGGGTTGTGACAGAGACCAGACCCACCGAGGGATG
AATGTCACTGTGGCTGGGCCAGACACAATCCTGGACTCCCCCCCTCCCGCCCCCCAAAACTGCTC
AGCCAGAACCTGACCCTGACCCTGGCCTTTCACCCCTCGAGGAGGGCTGGTGTCTGGGGTACTTA
AAGACACAGGCTAGATTTGGGGGCATCAATCCTGGAGGGCTGTGGACAGGAATTACAAGTTTAGG
ACTGGGCAGCTGAAAAACCTTTCTGAAAGGGATTAGGGGGCCCTGCTTCCAGAAGGCTCAGTGAA
GCTTTCTTGAATGAATGAATGAATGAGGTGTGTAGGCGGCACGTCACCTCTTCTCTGAGTTCCAG
TCTTGGGCCCTGCTTTCTCACCCTTTTTACCTGGTACCTGCAGACCCCTCCTTTACCTTCAGTTG
CCCACCTAGCACCTGATGCCCGTTGATCACCTGCCAGTCTGTGTCCCACCTGGGTGACTCGGGGG
CACACCGCATCCTCCTGAGATGGAGCGCAGGTCTCATTTGAGAGGGCAATCAAGGACCTGGCCAA
TCTAGGGGTCTCCCCTCTGCCCCGTTAGCCCCACCTGTGCCTGTGCTCTCTTCCCCATAATCCTC
AGTCTCAAACCCTTTTCCACCCCAGGACCTGGAGAGACTGACTCCACAACACCTAAGGCTCCTGT
AACTGGTGGGGGAGGCAGGCTTTGTTGCCTTTGTGAATAACCCCAGGGCAGGTGACTTCAAACCC
GTTTGTTCATCAGCTAAAAGGAGGTTCCACTGACAAGGGGTGTGAAAGCTCCCTGAGGGTGACCA
GAGGTAGGGGCCTTGGTCCTTGTCCCCCCCCACCATAAGACAGGCCCTTCCTCCTTCCAAAGTCA
GCTGGAAGGTCAGTGGCTCCCCCTCCCCCCTCCCCCAGTCCTGGAGAAGGAAGAAAGAAGTTACT
AAGTTACTGACTACAGCACTGCTAGTCTTTGGGGTGGGGCTTCCAATGCCCCCACCTGCATCACT
CTGGTTCTCCTGGAGGAGTAGACAAGGGCAGCCCTCTCAGTGCCCTCTGGGTGGGGTGTGTGGCT
GCTTATTGCTGGTACCCCCTGCAGCCTGTGTCTTGTCACGCCCCCTCACCCTTAGCCTACCCAGA
GGCCATGCAGCCCCGTGGCAGGTGCATTTCTGGGGGGAGCTGCAGCAAGCCCCCTGTGGCAATAG
GGAACCTCCTACAGCCTGCTCCTCCCTCTTCACACCCCCTTGGAGTATAAGGAGGGAACTGACAG
CCCAGACTCCTCGGCTCCAGAGAGGGGAAGGGAAGGGAGATTAGGCAGAAGTAGAGAGACCAGCT
TGGGGGCGGCTGCGTTTCCCCTGTCTTCTGCCCCTCCACCTGGCACACGGGGCCCAGCCATGGGG
ACCTTCAACCTGTGGACAGACTACTTGGGTTTGGCACGCCTGGTTGGGGCTCAGCGTGAAGAAGA
GGAGCCGGAGACCAGGCTGGATCGCCAGCCAGAAGCAGTGCCCGAACCGGGGGGTCAGCGACCCA
GCCCTGAATCCTCACCAGCTCCCGAGCGCCTGTGTTCTTTCTGCAAACACAACGGCGAGTCCCGG
GCCATCTACCAGTCCCACGTGCTCAAGGATGAAGCGGGCCGGGTGCTGTGCCCCATCCTCCGCGA
CTACGTGTGCCCCCAGTGCGGGGCCACCCGCGAGCGCGCCCACACCCGCCGCTTCTGCCCGCTCA
CCGGCCAGGGCTACACCTCCGTCTACAGCTACACCACCCGGAACTCGGCCGGCAAGAAGCTGGTC
CGCTCGGACAAGGCGAGGACGCAGGACCCTGGACACGGACCGCGCCGAGGAGGAGGTGCCTGTGC
AGGTTCCAAAGGTGCCAGGAAGTCTTCTGGAACTCCTCCCTCTTCCTGCTGCCCCTCAACTTCTG
CCTAAGGAGACTGGCGTGGGCAGGATGACGCCTTCACCTGGGGATGGGGACCCAGGCTCAGTGGA
GGCTGGGTTTCAGGGAAGACCCACCCTCCGAGGATCCGCCCCCTAGACGGTGCCTCCAGCCTGGG
GGCTTGGCAAAGGAGCCCGGTCTGGGACCACCGCCCAAAGCGCGCCCGCCCCTGTCACTGAAGGG
GGTGGTCCTCAGGCACCCCTGCCCTTCTTCCCCAACGCTGAGCAACCAGTCAGCGCTCAATAAAT
GTTTATGAATGGATCAGCGTCA
NCBI Reference Sequence: XM_027547964.1
>XM_027547964.1 PREDICTED:
Bos indicus x Bos taurus nanos C2HC-type zinc finger 3
(NANOS3), transcript variant X2, mRNA polynucleotide sequence
SEQ ID NO: 4
AGCCGCCCCTGGAGGGAGGGACTGGGGACCGGGTTTGAGGGTGAAGAAATGGGGAAGAGCATTAA
CGGGGTAAGCCTCGTGTAGTTATGCGCTTGGGCCCCCGTCTGATCCGACAAGGGCCCGAGTTTGG
AAGCCCGGGACCCTCTGCGATCCTCTAGCTTCGCCCTTGTCCAACCGGCAGGTGGACCCACAAGG
CGGGCTAGGCAGCGGCCCCACCTCGGGGCTCGAATTTGCAAAGTGCAGACTCAGACAACCCTCCC
CCCAACCACCTTGGGTTGTTGTGATTCATAAACCATTGTGTCCGGAACACGGTGAAGCTCACTTA
GGTATTACATTGTATTAAAATGACTTGTTTATCTCTCCGTTGCATCCATGCCCCCGGGGCCAGAA
CCACTTGGCCTCCAGACCTCTTGGGGCCTCTCGGAATCCCTCCTCTGCCTCTGCCTCTAGCTAAG
GGTGCCCTCTGTTCTGGCCTGTCTCCCAAACTGATAATTGGAAGAAATATGCACCGTTGAGGGCC
CTTTTGAGAATGCTTTGACTAAATGGGTTAGAAGCCCAGCGCCCGCTGCTGCTATATTTGCATAG
CAAAGGTGACAGAAGTATCTGCTGATATTATTACTTAGATTTATCTCCTTTTTCCCTGTCCTGGA
GCAGAGTTGGCTCCTTCCTGCTATCTGTTCCCTGACTTAATAGATTCTCTAAGTCTCTCATTCCC
TTCCCCTCCCTCACCCTACCCGGTTCCTTGACCCACCCCGCCCCCCAGCCTCCACTCCCTGCCCC
CCAAGGAGTTGCCAAGGGTTTGGGGGAACATTCAACCTGTCGGTGAGTTTGGGCAGCTCAGGCAA
ACCATCGACCGTTGAGTGGACCCCGAGGCCTGGAACTGCCGTCCACCCACCCACCCATCACGACC
CCCAACTTTCAGATCTGGGGTAGGGGCAGGGGATCCCGAACACATCCCCTCCCTTAGGCCACAGC
GAAGGTCACAATCAACATTCATTGTTGTCGGTGGGTTGTGACAGAGACCAGACCCACCGAGGGAT
GAATGTCACTGTGGCTGGGCCAGACACAATCCTGGACTCCCCCCCTCCCGCCCCCCAAAACTGCT
CAGCCAGAACCTGACCCTGACCCTGGCCTTTCACCCCTCGAGGAGGGCTGGTGTCTGGGGTACTT
AAAGACACAGGCTAGATTTGGGGGCATCAATCCTGGAGGGCTGTGGACAGGAATTACAAGTTTAG
GACTGGGCAGCTGAAAAACCTTTCTGAAAGGGATTAGGGGGCCCTGCTTCCAGAAGGCTCAGTGA
AGCTTTCTTGAATGAATGAATGAATGAGGTGTGTAGGCGGCACGTCACCTCTTCTCTGAGTTCCA
GTCTTGGGCCCTGCTTTCTCACCCTTTTTACCTGGTACCTGCAGACCCCTCCTTTACCTTCAGTT
GCCCACCTAGCACCTGATGCCCGTTGATCACCTGCCAGTCTGTGTCCCACCTGGGTGACTCGGGG
GCACACCGCATCCTCCTGAGATGGAGCGCAGGTCTCATTTGAGAGGGCAATCAAGGACCTGGCCA
ATCTAGGGGTCTCCCCTCTGCCCCGTTAGCCCCACCTGTGCCTGTGCTCTCTTCCCCATAATCCT
CAGTCTCAAACCCTTTTCCACCCCAGGACCTGGAGAGACTGACTCCACAACACCTAAGGCTCCTG
TAACTGGTGGGGGAGGCAGGCTTTGTTGCCTTTGTGAATAACCCCAGGGCAGGTGACTTCAAACC
CGTTTGTTCATCAGCTAAAAGGAGGTTCCACTGACAAGGGGTGTGAAAGCTCCCTGAGGGTGACC
AGAGGTAGGGGCCTTGGTCCTTGTCCCCCCCCACCATAAGACAGGCCCTTCCTCCTTCCAAAGTC
AGCTGGAAGGTCAGTGGCTCCCCCTCCCCCCTCCCCCAGTCCTGGAGAAGGAAGAAAGAAGTTAC
TAAGTTACTGACTACAGCACTGCTAGTCTTTGGGGTGGGGCTTCCAATGCCCCCACCTGCATCAC
TCTGGTTCTCCTGGAGGAGTAGACAAGGGCAGCCCTCTCAGTGCCCTCTGGGTGGGGTGTGTGGC
TGCTTATTGCTGGTACCCCCTGCAGCCTGTGTCTTGTCACGCCCCCTCACCCTTAGCCTACCCAG
AGGCCATGCAGCCCCGTGGCAGGTGCATTTCTGGGGGGAGCTGCAGCAAGCCCCCTGTGGCAATA
GGGAACCTCCTACAGCCTGCTCCTCCCTCTTCACACCCCCTTGGAGTATAAGGAGGGAACTGACA
GCCCAGACTCCTCGGCTCCAGAGAGGGGAAGGGAAGGGAGATTAGGCAGAAGTAGAGAGACCAGC
TTGGGGGCGGCTGCGTTTCCCCTGTCTTCTGCCCCTCCACCTGGCACACGGGGCCCAGCCATGGG
GACCTTCAACCTGTGGACAGACTACTTGGGTTTGGCACGCCTGGTTGGGGCTCAGCGTGAAGAAG
AGGAGCCGGAGACCAGGCTGGATCGCCAGCCAGAAGCAGTGCCCGAACCGGGGGGTCAGCGACCC
AGCCCTGAATCCTCACCAGCTCCCGAGCGCCTGTGTTCTTTCTGCAAACACAACGGCGAGTCCCG
GGCCATCTACCAGTCCCACGTGCTCAAGGATGAAGCGGGCCGGGTGCTGTGCCCCATCCTCCGCG
ACTACGTGTGCCCCCAGTGCGGGGCCACCCGCGAGCGCGCCCACACCCGCCGCTTCTGCCCGCTC
ACCGGCCAGGGCTACACCTCCGTCTACAGCTACACCACCCGGAACTCGGCCGGCAAGAAGCTGGT
CCGCTCGGACAAGGCGAGGACGCAGGACCCTGGACACGGACCGCGCCGAGGAGGAGGTTCCAAAG
GTGCCAGGAAGTCTTCTGGAACTCCTCCCTCTTCCTGCTGCCCCTCAACTTCTGCCTAAGGAGAC
TGGCGTGGGCAGGATGACGCCTTCACCTGGGGATGGGGACCCAGGCTCAGTGGAGGCTGGGTTTC
AGGGAAGACCCACCCTCCGAGGATCCGCCCCCTAGACGGTGCCTCCAGCCTGGGGGCTTGGCAAA
GGAGCCCGGTCTGGGACCACCGCCCAAAGCGCGCCCGCCCCTGTCACTGAAGGGGGTGGTCCTCA
GGCACCCCTGCCCTTCTTCCCCAACGCTGAGCAACCAGTCAGCGCTCAATAAATGTTTATGAATG
GATCAGCGTCA
NCBI Reference Sequence: XR_003511972.1
>XR_003511972.1 PREDICTED: Bos indicus x Bos taurus nanos C2HC-type zinc finger 3
(NANOS3), transcript variant X3, RNA polynucleotide sequence
SEQ ID NO: 5
      1 tgggaggcgg cggccgcggg ttcgagccgg cgccggagcc ccgcggtccc ctccccctgc
    61 ccgcggcctg gggagccccc gcccagcccc ggagccgcca aaatgcaatt tcccgtgccg
  121 gcgcctcgcg gctcgggggg cttttccggg cgggttttgg acagaagagg gggaaacaag
  181 gcggcggccc caaaacgagg ttccaaaggt gccaggaagt cttctggaac tcctccctct
  241 tcctgctgcc cctcaacttc tgcctaagga gactggcgtg ggc
NCBI Reference Sequence: XM_019964015.1
>XM_019964015.1 PREDICTED: Bos indicus nanos C2HC-type zinc finger 3 (NANOS3),
mRNA polynucleotide sequence
SEQ ID NO: 7
      1 tccccccctc ccgcccccca aaactgctca gccagaacct gaccctgacc ctggcctttc
    61 acccctcgag gagggctggt gtctggggta cttaaagaca caggctagat ttgggggcat
  121 caatcctgga gggctgtgga caggaattac aagtttagga ctgggcagct gaaaaacctt
  181 tctgaaaggg attagggggc cctgcttcca gaaggctcag tgaagctttc ttgaatgaat
  241 gaatgaatga ggtgtgtagg cggcacgtca cctcttctct gagttccagt cttgggccct
  301 gctttctcac ccttcttacc tggtacctgc agacccctcc tttaccttca gttgcccacc
  361 tagcacctga tgcccgttga tcacctgcca gtctgtgtcc cacctgggtg actcgggggc
  421 acaccgcatc ctcctgagat ggagcgcagg tctcatttga gagggcaatc aaggwcctgg
  481 ccaatctagg ggtctcccct ctgccccgtt agccccacct gtgcctgtgc tctcttcccc
  541 ataatcctca gtctcaaacc cttttccacc ccaggacctg gagagactga ctccacaaca
  601 cctaaggctc ctgtaactgg tgggggaggc aggctttgtt gccttcgtga ataaccccag
  661 ggcaggtgac ttcaaacccg tttgttcatc agctaaaagg aggttccact gacaaggggt
  721 gtgaaagctc cctgagggtg accagaggta ggggccttgg tccttgtccc cccccaccat
  781 aagacaggcc cttcctcctt ccaaagtcag ctggaaggtc agtggctccc cctcccccct
  841 cccccagtcc tggagaagga agaaagaagt tactaagtta ctgactacag cactgctagt
  901 ctttggggtg gggcttccaa tgcccccacc tgcatcactc tggttctcct ggaggagtag
  961 acaagggcag ccctctcagt gccctctggg tggggtgtgt ggctgcttat tgctggtacc
1021 ccctgcagcc tgtgtcttgt cacgccccct cacccttagc ctacccagag gccatgcagc
1081 cccgtggcag gtgcatttct ggggggagct gcagcaagcc ccctgtggca atagggaacc
1141 tcctacagcc tgctcctccc tcttcacacc cccttggagt ataaggaggg aactgacagc
1201 ccagactcct cggctccaga gaggggaagg gaagggagat taggcagaag tagagagacc
1261 agcttggggg cggctgcgtt tctcctgtct tctgcccctc cacctggcac acggggccca
1321 gccatgggga ccttcaacct gtggacagac tacttgggtt tggcacgcct ggttggggct
1381 cagcgtgaag aagaggagcc ggagaccagg ctggatcgcc agccagaagc agtgcccgaa
1441 ccggggggtc agcgacccag ccctgaatcc tcaccagctc ccgagcgcct gtgttctttc
1501 tgcaaacaca acggcgagtc ccgggccatc taccagtccc acgtgctcaa ggatgaagcg
1561 ggccgggtgc tgtgccccat cctccgcgac tacgtgtgcc cccagtgcgg ggccacccgc
1621 gagcgcgccc acacccgccg cttctgcccg ctcaccggcc agggctacac ctccgtctac
1681 agctacacca cccggaactc ggccggcaag aagctggtcc gctcggacaa ggcgaggacg
1741 caggaccctg gacacggacc gcgccgagga ggaggtgcct gtgcaggttc caaaggtgcc
1801 aggaagtctt ctggaactcc tccctcttcc tgctgcccct caacttctgc ctaaggagac
1861 tggcgtgggc aggatgacgc cttcacctgg ggatggggac ccaggctcag tggaggctgg
1921 gtttcaggga agacccaccc tccgaggatc cgccccctag acggtgcctc cagcctgggg
1981 gcttggcaaa ggagcccggt ctgggaccac cgcccaaagc gcgcccgccc ctgtcactga
2041 agggggtggt cctcaggcac ccctgccctt cttccccaac gctgagcaac cagtcagcgc
2101 tcaataaatg tttatg
>Bovine Nanos3 transcript (complete)
>ENSBTAT00000000513
SEQ ID NO: 9
ATGGGGACCTTCAACCTGTGGACAGACTACTTGGGTTTGGCACGCCTGGTTGGGGCTCAGCGTGA
AGAAGAGGAGCCGGAGACCAGGCTGGATCGCCAGCCAGAAGCAGTGCCCGAACCGGGGGGTCAGC
GACCCAGCCCTGAATCCTCACCAGCTCCCGAGCGCCTGTGTTCTTTCTGCAAACACAACGGCGAG
TCCCGGGCCATCTACCAGTCCCACGTGCTCAAGGATGAAGCGGGCCGGGTGCTGTGCCCCATCCT
CCGCGACTACGTGTGCCCCCAGTGCGGGGCCACCCGCGAGCGCGCCCACACCCGCCGCTTCTGCC
CGCTCACCGGCCAGGGCTACACCTCCGTCTACAGCTACACCACCCGGAACTCGGCCGGCAAGAAG
CTGGTCCGCTCGGACAAGGCGAGGACGCAGGACCCTGGACACGGACCGCGCCGAGGAGGAGGTTC
CAAAGGTGCCAGGAAGTCTTCTGGAACTCCTCCCTCTTCCTGCTGCCCCTCAACTTCTGCCTAAG
GAGACTGGCGTGGGCAGGATGACGCCTTCACCTGGGGATGGGGACCCAGGCTCAGTGGAGGCTGG
GTTTCAGGGAAGACCCACCCTCCGAGGATCCGCCCCCTAGACGGTGCCTCCAGCCTGGGGGCTTG
GCAAAGGAGCCCGGTCTGGGACCACCGCCCAAAGCGCGCCCGCCCCTGTCACTGAAGGGGGTGGT
CCTCAGGCACCCCTGCCCTTCTTCCCCAACGCTGAGCAACCAGTCAGCGCTCAATAAATGTTTAT
GAATGGATCA
```

In some embodiments, the NANOS3 polynucleotide (gene) that is modified encodes a NANOS3 polypeptide has that is 80-100 percent identical; at least 85; at least 90; at least 95; at least 96; at least 96; at least 97; at least 98; at least 99; 85-100; 90-100; 91; 92; 93; 94; 95; 96; 97; 98; 99; or 100 percent identical to any one of SEQ ID NOs: 1, 3, or 6.

>NCBI Reference Sequence: XM_027547963.1
>XM_027547963.1 PREDICTED:
Bos indicus x Bos taurus nanos C2HC-
type zinc finger 3
(NANOS3), transcript variant X1,
mRNA polypeptide sequence
SEQ ID NO: 1
MGTFNLWTDYLGLARLVGAQREEEEPETRLDRQPEAVPEPGGQRP
SPESSPAPERLCSFCKHNGESRAIYQSHVLKDEAGRVLCPILRDY
VCPQCGATRERAHTRRFCPLTGQGYTSVYSYTTRNSAGKKLVRSD
KARTQDPGHGPRRGGGACAGSKGARKSSGTPPSSCCPSTSA
>NCBI Reference Sequence:
XM_027547963.1
SEQ ID NO: 3
MGTFNLWTDYLGLARLVGAQREEEEPETRLDRQPEAVPEPGGQRP
SPESSPAPERLCSFCKHNGESRAIYQSHVLKDEAGRVLCPILRDY
VCPQCGATRERAHTRRFCPLTGQGYTSVYSYTTRNSAGKKLVRSD
KARTQDPGHGPRRGGGSKGARKSSGPPSSCCPSTSA
>NCBI Reference Sequence:
XM_019964015.1
SEQ ID NO: 6
MGTFNLWTDYLGLARLVGAQREEEEPETRLDRQPEAVPEPGGQRP
SPESSPAPERLCSFCKHNGESRAIYQSHVLKDEAGRVLCPILRDY
VCPQCGATRERAHTRRFCPLTGQGYTSVYSYTTRNSAGKKLVRSD
KARTQDPGHGPRRGGGACAGSKGARKSSGTPPSSCCPSTSA

In some embodiments, the NANOS3 gene that is modified is 80% to 100% (e.g., 80%, 81%, 82%, 83%, 84%, 85%, 86%, 87%, 88%, 89%, 90%, 91%, 92%, 93%, 94%, 95%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% to/or 100%) identical to the bovine NANOS3 gene sequence (Accession Number NC007305.5, region 10061880) in the National Center for Biotechnology Information database.

In some embodiments, the NANOS3 gene that is modified is a homologue, orthologue, or paralogue of a bovine NANOS3 gene and/or a NANOS3 sequence of the present disclosure. In some embodiments, a NANOS3 homologue is composed of or includes a nucleotide sequence that is, for example and without limitation: at least 80%; at least 85%; at least about 90%; at least about 91%; at least about 92%; at least about 93%; at least about 94%; at least about 95%; at least about 96%; at least about 97%; at least about 98%; at least about 99%; at least about 99.5%; 99.6%, 99.7%, 99.8% and/or at least about 99.9% identical to about 20 contiguous nucleotides of any one or more of the NANOS3 sequences of the present disclosure.

Site specific modification of an endogenous NANOS3 gene of a host cell and/or animal causing a disruption of the NANOS3 gene and/or gene/product and/or expression thereof can be accomplished by any suitable technique such as any of those described elsewhere herein. In general, such methods include contacting a cell with one or more genetic modifying systems described herein configured to modify a NANOS3 gene, particularly a bovine NANOS3 gene, or components thereof. In some embodiments, the systems employ the native homologues recombination pathway for site specific modification of the NANOS3 gene (such as conventional knock-in and knock-out approaches relying on homology arms to direct site specific knock in of a disruptive exogenous polynucleotide) and other such as RNA guided nucleases (e.g., CRISPR-Cas), and transposons. Examples of suitable genetic modifying systems and techniques are described in greater detail herein and will be appreciated by those of ordinary skill in the art in view of this disclosure.

In some embodiments, a CRISPR-Cas based approach is used to modify the NANOS3 gene to introduce substations, indels, or other mutations to effectively decrease or eliminate NANOS3 gene function and production of a NANOS3 gene product. Exemplary guides for CRISPR-Cas9 NANOS3 knockout are provided in at least the Working Examples below and can be designed based on the principles and description provided in this disclosure. In some embodiments the number of nucleotides modified, substituted, inserted and/or deleted can be or sum to (in the case of an indel) 1-2600 or more. In some embodiments, the NANOS3 is modified at one or more nucleotides of exon 1, exon 2, or both.

In some embodiments, the number of nucleotides modified, substituted, inserted and/or deleted can be or sum to (in the case of an indel) about 1, 2, 3, 4, 5, 6, 7, 8, 9 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, or more nucleotides of a NANOS3 gene.

In some embodiments, the NANOS3 polynucleotide modification results in about a 1 to 1000 or more fold reduction in the expression of a NANOS3 gene and/or gene product. In some embodiments, the NANOS3 modification results in about a 1 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more fold reduction in the expression of a NANOS3 gene and/or gene product. In some embodiments, the NANOS3 polynucleotide modification results in no observable or detectable amount of expression of a NANOS3 gene or gene product. In some embodiments, the NANOS3 polynucleotide modification results in reducing or completely eliminating germ cells in the animal having the modified NANOS3, where reducing results in substantially no germ cells being present in the animal having the modified NANOS3. Methods of measuring gene and gene product expression include without limitation, PCR based techniques and affinity and immune-based protein detection methods, which are generally known in the art.

In some embodiments, the modification(s) reduce expression of the NANOS3 gene or gene product by 1 to 1000 fold or more, such as about 1, 2, 3, 4, 5, 6, 7, 8, 9 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 fold or more. In some embodiments, the modification(s) reduce expression of the NANOS3 gene or gene product such that amounts are undetectable (e.g., below the limit of detection) by conventional techniques used to measure gene and/or gene product (e.g., transcript and/or protein) amounts. In some embodiments, the modification(s) reduce expression of the NANOS3 gene or gene product such that one or more functions or activities of a NANOS3 gene or gene product are insufficient for normal or wild-type NANOS3 gene or gene product function or activity, such as production of immature and mature germ cells. Thus, in some embodiments, a NANOS3 deficient animal described elsewhere herein may be germline ablated (i.e., no functional germ cells) yet have detectable NANOS3 gene or gene product expression.

### Donor Cells for Germline Complementation

### Engineered Donor Cells

In some embodiments, a genetic modifying system can be used to modify a donor cell or be used to produce a donor cell source for use in a germline complementation along with a NANOS3 deficient host to generate surrogate sires and/or dams. In some embodiments, such exogenous gene constructs are introduced into donor cells (see donor cells in e.g., FIG. 1) that can be used to complement a germline ablated NANOS3 deficient host or host cell(s) described herein.

Exemplary donor cells and cell sources include, without limitation, zygotes, developing embryos, early embryos, blastocysts, blastomeres, morulas, embryonic stem cells, primordial germ cells, primordial germ cell like cells, pluripotent stem cells (including, but not limited to those described in In some embodiments the cell is a pluripotent embryonic stem cell as described International Pat. App. Pub. WO 2019/140260), induced pluripotent stem cells (such as those reprogrammed from somatic cells), spermatogonial or oogonial stem cells, somatic cells, tissues, organs (such as testis or ovaries), any combination thereof, and/or the like.

In some embodiments the donor cells or cell source is modified to contain and/or express an exogenous engineered gene such as any of those described in U.S. Pat. App. Pub. No. 2019/165465. In some embodiments, such as where the exogenous cell source (e.g., organism) contains and/or expresses an engineered gene or gene construct that results in ablation, elimination, and/or dysfunction of undesired germline cells it will be appreciated that the germline donor cells from such a donor cell source used to complement a NANOS3 deficient host described herein will not contain the engineered construct because said construct results in ablation or renders the germ cells expressing said construct unable to fertilize or be fertilized or produce a viable zygote or embryo. Such undesired germline cells are those that carry an undesirable gene, allele, and/or chromosome. Exemplary undesirable genes, alleles, and/or chromosome include diseased genes, alleles, and/or chromosome (i.e., those that convey a genetic disease or predispose an animal to development of a disease or condition), genes, alleles, and/or chromosomes that convey an undesirable phenotype or characteristic, and/or X or Y chromosomes. In some embodiments, such donor cells are not modified prior to use in germline complementation of a NANOS3 deficient host. In some embodiments, such donor cells can be modified prior to use in germline complementation of a NANOS3 deficient host. Exemplary modifications are described below.

In some embodiments, the gene modification(s) in an engineered donor cell results in increased or reduced expression of one or more genes and/or gene products.

In some embodiments, donor source cells are modified prior to being used to complement the NANOS3 deficient host. Such modifications can include genetic modifications that can be introduced via a genetic modification or modifying system described herein. In some embodiments, a donor cell source embryo, blastomere, or ESCs are genetically modified prior to complementation (see e.g., FIG. 1). In some embodiments, donor spermatogonial (or oogonial) stem cells, pluripotent or induced pluripotent stem cells (including, but not limited to those described in International Pat. App. Pub WO 2019/140260). In some embodiments the cell is a pluripotent embryonic stem cell as described International Pat. App. Pub WO 2019/140260), primordial germ cells, or primordial germ cell-like cells are genetically modified prior to complementation (see e.g., FIG. 1).

### Exemplary Gene Modifications and Transgenes for Donor Cells

### Modifications to Generate a Polled Bovines

Genes and polynucleotides that can be modified to result in a polled (hornless) animal, such as a polled bovine.

In some embodiments the modification(s) is/are at the proximal end of the bovine chromosome 1 (BAT01), optionally corresponding to the HAS 21 region and optionally beginning at about bp 1,684,495 and ending at about bp 1,896,112. In some embodiments, the modifications are in one or more genes located at the proximal end of the bovine BATO1. In some embodiments, the genome is modified to contain a polynucleotide that is 90-100 percent identical to any one of SEQ ID NOs 1-148 of U.S. Pat. Publication 20110262909, one or more polymorphisms or SNPs associated with a polled phenotype including, but not limited to, any one or more of those set forth in Tables 1 2, or 3 of U.S. Pat. Publication 20110262909. In some embodiments, the genes IFNAR2m, SYNJ1, and C21orf63, are modified such that they contain genotype corresponding to a polled phenotype (see e.g., Table 3 of U.S. Pat. Publication 20110262909). In some embodiments the PAXBP1 gene is modified to contain one or more mutations that give rise to the polled phenotype. In some embodiments the C1H21orf62 gene is modified to contain one or more mutations that give rise to the polled phenotype. In some embodiments intron 3 of the IFGR2 gene is modified to contain one or more mutations that give rise to the polled phenotype, such as the SNP described by Glatzer et al., PLOS ONE 8:e67992 (2013). In some embodiments the FOXL2 and/or RXFP2 genes is/are modified to contain one or more mutations that give rise to the polled phenotype. In some embodiments, the ZEB2 gene is modified to contain one or more mutations that give rise to the polled phenotype (see e.g., Capitan et al., PLOS One7: e49084). In some embodiments the OLIG1 and/or OLIG2 genes are modified to introduce a polled mutation to give rise to a polled phenotype. In some embodiments, the genome is modified to introduce one or more mutations in long noncoding RNA (LNcRNA) #1 (LNcRNA #1) (which maps to annotated bovine locus LOC100848368), LNcRNA #2 which overlaps four annotated exons of the annotated LOC100848215 or regulatory molecule such that the LMcRNA #1 and/or #2 are overexpressed and results in a polled animal. See also Allais-Bonnet et al. PLoS ONE 8: e63512 (2013). In some embodiments, one or more modification are made in the annotated locus LOC100848368 and/or LOC10084821, such as one or more modification that result in decreased expression of o one or more gene products produced from one or both loci, which result in a polled phenotype.

In some embodiments, the polled phenotype is introduced by modifying the genome to introduce 3 SNPs and/or an 80 kb duplication of chromosome 1 corresponding to bp 1,909,352-1,989,480 of chromosome 1 (BTA1 (or BTAO1)) that corresponds to the PF allele identified in Friesian breeds (as described in Medugorac et al., PLOS One 7: e39477 (2012)). In some embodiments, the polled phenotype is introduced by modifying the genome such that it contains a 202 bp Insertion—deletion that is composed of a duplication of 212 bp BTA1: 1,705,834-1,706,045 bp in place of a 10 bp deletion BTA1: 1,706,051-1,706,060 bp or a 202 bp InDel that is composed of a 208 bp duplication in combination with a 6 bp deletion in the BTA1 that corresponds to the Pc allele or Pc allele variant, respectively, identified in Celtic original breeds (the Pc allele contains a 202-bp InDel resulting in polled phenotype). See also Wiedemar et al., PLoS One 9:e93435 (2014). In some embodiments, the genome is modified such that the BAT1 is modified to contain a 219 bp duplication-insertion corresponding to the P_{219ID} allele or equivalent thereof (see e.g., Medugorac et al. 2017. Nat. Genet. 49:470. doi: 10.1038/ng.3775). In some embodiments, the genome is modified such that the BAT1 is modified to contain a 7 bp deletion and 6 bp insertion corresponding to the P_{1ID} allele or equivalent thereof (see e.g., Medugorac et al. 2017. Nat. Genet. 49:470. doi: 10.1038/ng.3775). In some embodiments, the genome is modified such that the BAT1 is modified to include an approximately 110 kb duplication corresponding to the P_{G} allele. (see e.g., Stafuzza et al. (2018) PLoS One 13:e0202978 and Utsunomiya et al., (2019) Anim. Genet. 50, 187-188).

In some embodiments, the genome is modified such that it contains one or more of the sequence variants and/or SNPs of Table S1 or Table S2, of Wiedemar et al., PLoS One 9:e93435 (2014) that result in a polled phenotype. Table S2 of Wiedemar et al., PLoS One 9:e93435 (2014) is reproduced below.

TABLE S2
Sequence variants of polled associated 932 kb haplotype in Holstein. Animals carrying
the polled associated indel found in Simmental are not included. The remaining associated
variants are highlighted in italicized and the excluded variants are shown in bold and underlined.

| UMD3.1 | | reference | variant | ref/ref | ref/var | var/var | ref/ref | ref/var |
|---|---|---|---|---|---|---|---|---|
| position | variant | allele | allele | polled | polled | polled | horned | horned |
| 1′583′414º | SNP | A | G | | 84 | 4 | 204 | |
| 1′584′650 | SNP | C | T | | 1 | 2 | 55 | 1 |
| 1′587′758 | SNP | C | T | | 76 | 4 | 200 | 1 |
| 1′596′777º | SNP | G | T | | 85 | 4 | 390 | |
| 1′603′747º | SNP | C | T | | 87 | 4 | 426 | |
| 1′606′620° | SNP | C | T | | 83 | 4 | 415 | |
| 1′607′675° | SNP | G | A | | | 3* | 41 | |
| 1′618′261º | SNP | T | C | | | 3* | 43 | |
| 1′623′157º | SNP | G | A | | 11 | 2 | 77 | |
| 1′623′573 | SNP | G | C | | 77 | 4 | 368 | |
| 1′624′279° | SNP | C | T | | 82 | 2 | 417 | |
| 1′624′522° | SNP | T | G | | 85 | 2 | 411 | |
| 1′624′555° | SNP | T | C | | 81 | 1 | 408 | |
| 1′626′030° | SNP | T | C | | 84 | 4 | 425 | |
| 1′626′065° | SNP | G | C | | 84 | 4 | 421 | |
| 1′628′090° | SNP | A | G | | 60 | 4 | 384 | |
| 1′631′299° | SNP | A | G | | 86 | 4 | 424 | |
| 1′631′667º | SNP | C | T | | 87 | 4 | 429 | |
| 1′631′762° | SNP | G | A | | 87 | 4 | 429 | |
| 1′631′895° | SNP | T | C | | 87 | 4 | 427 | |
| 1′633′226° | SNP | T | A | | 77 | 4 | 420 | |
| 1′633′690° | SNP | C | T | | 79 | 4 | 422 | |
| 1′635′514° | SNP | G | C | | 76 | 4 | 414 | |
| 1′639′632° | SNP | A | G | | 82 | 3 | 396 | |
| 1′640′658° | SNP | G | T | | 86 | 4 | 432 | |
| 1′640′815° | SNP | T | C | | 85 | 3 | 420 | |
| 1642525° ** | SNP | T | A | | | 1 | 33 | |
| 1′645′083° | SNP | G | T | | 80 | 4 | 426 | |
| 1′645′296° | deletion | AT | A | | 76 | 4 | 422 | |
| 1645301° | SNP | T | A | | 75 | 4 | 423 | |
| 1′649′163 + | InDel | CGCA | TTCTCA | | 85 | 3 | 415 | |
| | | TCA | GAATAG | | | | | |
| | | | (SEQ ID | | | | | |
| | | | NO: 54) | | | | | |
| 1′651′491º | SNP | C | T | 6 | 12 | 3 | 203 | |
| 1′654′405 + | SNP | G | A | | 87 | 4 | 424 | |
| 1′655′463 + | SNP | C | T | | 86 | 4 | 429 | |
| 1′672′807 | SNP | G | A | | 2 | 2 | 216 | 1 |
| 1′676′520 | SNP | C | T | | 19 | 3 | 407 | 1 |
| 1′679′832 | SNP | C | T | | 2 | 3 | 133 | 1 |
| 1′680′646 | SNP | T | C | | 80 | 6 | 221 | 1 |
| 1′684′055° | SNP | G | C | | 85 | 4 | 416 | |
| 1′764′239º | SNP | T | C | | 85 | 4 | 409 | |
| 1′768′587 + | SNP | C | A | 4*** | 152 | 25 | 443 | |
| 1′855′898° | SNP | G | A | | 87 | 4 | 408 | |
| 1′909′354+ | 80 kbDup | | | | 105**** | | 430 | |
| 2′028′953° | SNP | C | G | | | 1 | 33 | |
| 2′248′958 | SNP | G | A | | | | 33 | |
| 2′255′019 | SNP | C | T | | | 1 | 33 | |
| 2′261′830 | SNP | T | C | | | 1 | 33 | |
| 2′263′363 | SNP | T | C | | | 1 | 33 | |
| 2′267′616 | deletion | CT | C | | | I | 33 | |
| 2′268′463 | SNP | G | A | | | 1 | 33 | |
| 2′269′541 | SNP | T | C | | | 1 | 33 | |
| 2′270′224 | SNP | C | T | | | 1 | 33 | |
| 2′271′484 | SNP | C | T | | | I | 33 | |
| 2′274′411 | SNP | C | T | | | I | 33 | |
| 2′274′700 | SNP | T | G | | | 1 | 33 | |
| 2′275′244 | SNP | G | A | | | 1 | 33 | |
| 2′275′575 | SNP | G | A | | | 1 | 33 | |
| 2′275′606 | SNP | C | G | | | 1 | 33 | |
| 2′275′689 | SNP | G | A | | | 1 | 33 | |
| 2′278′133 | SNP | A | C | | | 1 | 33 | |
| 2′279′787 | SNP | G | T | | | | 33 | |
| 2′279′861 | SNP | A | G | | | 1 | 33 | |
| 2′279′921 | SNP | T | C | | | 1 | 32 | |
| 2′279′996 | SNP | G | A | | | 1 | 32 | |
| 2′280′020 | SNP | A | G | | | 1 | 32 | |
| 2′280′142 | SNP | C | T | | | 1 | 32 | |
| 2′280′168 | SNP | G | A | | | 1 | 32 | |
| 2′280′378 | SNP | T | C | | | 1 | 32 | |
| 2′280′407 | SNP | C | T | | | 1 | 33 | |
| 2′280′428 | InDel | CCT | CTCTAC | | | 1 | 32 | |
| 2′280′522 | SNP | T | C | | | 1 | 33 | |
| 2′280′577 | SNP | C | G | | | 1 | 33 | |
| 2′280′612 | SNP | C | T | | | 1 | 33 | |
| 2′280′642 | SNP | T | C | | | 1 | 33 | |
| 2′280′673 | SNP | C | A | | | 1 | 33 | |
| 2′280′683 | SNP | A | G | | | 1 | 33 | |
| 2′280′702 | SNP | C | T | | | 1 | 33 | |
| 2′280′862 | SNP | A | G | | | 1 | 33 | |
| 2′280′879 | SNP | A | G | | | 1 | 33 | |
| 2′280′916 | SNP | T | C | | | 1 | 33 | |
| 2′280′926 | SNP | G | C | | | 1 | 33 | |
| 2′281′031 | SNP | G | A | | | 1 | 33 | |
| 2′281′058 | SNP | C | G | | | 1 | 33 | |
| 2′281′154 | SNP | A | T | | | 1 | 33 | |
| 2′281′262 | SNP | T | C | | | 1 | 33 | |
| 2′281′270 | SNP | T | A | | | 1 | 33 | |
| 2′281′440 | SNP | A | T | | | 1 | 33 | |
| 2′281′560 | SNP | A | G | | | 1 | 33 | |
| 2′281′590 | SNP | T | C | | | 1 | 33 | |
| 2′281′635 | SNP | C | T | | | 1 | 33 | |
| 2′282′082 | SNP | A | G | | | 1 | 33 | |
| 2′282′132 | SNP | G | A | | | 1 | 33 | |
| 2′282′187 | SNP | T | C | | | 1 | 33 | |
| 2′282′194 | SNP | A | T | | | 1 | 33 | |
| 2′282′315 | SNP | A | G | | | 1 | 33 | |
| 2′282′521 | SNP | T | C | | | 1 | 33 | |
| 2′282′761 | SNP | A | G | | | 1 | 33 | |
| 2′283′214 | SNP | C | T | | | 1 | 33 | |
| 2′283′224 | SNP | G | A | | | 1 | 33 | |
| 2′283′237 | SNP | C | G | | | 1 | 33 | |
| 2′283′539 | SNP | G | A | | | 1 | 33 | |
| 2′283′965 | SNP | G | A | | | 1 | 33 | |
| 2′283′992 | SNP | C | A | | | 1 | 33 | |
| 2′284′002 | SNP | C | T | | | 1 | 33 | |
| 2′284′720 | SNP | C | G | | | 1 | 33 | |
| 2′284′769 | SNP | T | C | | | 1 | 33 | |
| 2′285′147 | insertion | G | GT | | | 1 | 33 | |
| 2′285′345 | SNP | G | A | | | 1 | 33 | |
| 2′285′383 | SNP | G | A | | | 1 | 33 | |
| 2′286′301 | SNP | T | G | | | 1 | 33 | |
| 2′286′353 | SNP | G | A | | | 1 | 33 | |
| 2′286′400 | SNP | T | G | | | 1 | 33 | |
| 2′286′504 | SNP | C | G | | | 1 | 33 | |
| 2′286′523 | SNP | C | A | | | 1 | 33 | |
| 2′286′592 | SNP | T | C | | | 1 | 33 | |
| 2′286′604 | SNP | G | A | | | 1 | 33 | |
| 2′286′606 | SNP | G | A | | | 1 | 33 | |
| 2′286′988 | SNP | A | G | | | 1 | 33 | |
| 2′287′210 | SNP | C | T | | | 1 | 33 | |
| 2′287′370 | SNP | A | G | | | 1 | 33 | |
| 2′287′448 | SNP | C | G | | | 1 | 33 | |
| 2′287′529 | insertion | A | AT | | | 1 | 33 | |
| 2′287′596 | insertion | C | CT | | | 1 | 33 | |
| 2′287′746 | SNP | T | C | | | 1 | 33 | |
| 2′287′757 | SNP | C | T | | | 1 | 33 | |
| 2′287′765 | SNP | T | C | | | 1 | 33 | |
| 2′287′798 | SNP | A | G | | | 1 | 33 | |
| 2′287′996 | SNP | G | C | | | 1 | 33 | |
| 2′288′467 | SNP | A | G | | | 1 | 32 | |
| 2′288′990 | SNP | A | G | | | 1 | 32 | |
| 2′289′042 | SNP | A | G | | | 1 | 33 | |
| 2′289′346 | SNP | G | A | | | 1 | 33 | |
| 2′289′458 | SNP | C | T | | | 1 | 32 | |
| 2′289′716 | SNP | C | T | | | 1 | 33 | |
| 2′292′082 | insertion | C | CACCT | | | 1 | 33 | |
| 2′292′239 | SNP | G | A | | | 1 | 33 | |
| 2′292′304 | SNP | G | A | | | 1 | 33 | |
| 2′293′266 | SNP | T | C | | | 1 | 33 | |
| 2′293′846 | SNP | G | A | | | 1 | 33 | |
| 2′293′867 | SNP | C | A | | | 1 | 33 | |
| 2′293′870 | SNP | T | C | | | 1 | 33 | |
| 2′294′082 | SNP | G | C | | | 1 | 33 | |
| 2′294′184 | SNP | C | T | | | 1 | 33 | |
| 2′294′641 | SNP | G | T | | | 1 | 33 | |
| 2′294′647 | SNP | T | C | | | 1 | 33 | |
| 2′294′824 | SNP | G | A | | | 1 | 33 | |
| 2′294′855 | SNP | C | T | | | 1 | 33 | |
| 2′295′121 | SNP | C | G | | | 1 | 33 | |
| 2′295′487 | SNP | G | A | | | 1 | 33 | |
| 2′295′951 | SNP | G | A | | | 1 | 32 | |
| 2′297′070 | SNP | C | T | | | 1 | 33 | |
| 2′297′597 | SNP | T | A | | | 1 | 33 | |
| 2′298′042 | SNP | A | G | | | 1 | 33 | |
| 2′298′175 | SNP | G | A | | | 1 | 33 | |
| 2′298′226 | SNP | C | A | | | 1 | 33 | |
| 2′299′551 | SNP | A | G | | | 1 | 32 | |
| 2′299′669 | SNP | C | G | | | 1 | 33 | |
| 2′299′800 | SNP | C | T | | | 1 | 33 | |
| 2′299′901 | SNP | A | G | | | 1 | 33 | |
| 2′300′316 | SNP | A | G | | | 1 | 33 | |
| 2′300′391 | deletion | TTAACTCACTG | T | | | 1 | 33 | |
| | | ACAGTGAGTCG | | | | | | |
| | | ACAG (SEQ ID | | | | | | |
| | | NO: 55) | | | | | | |
| 2′300′451 | SNP | C | T | | | 1 | 33 | |
| 2′300′502 | SNP | A | G | | | 1 | 33 | |
| 2′300′761 | SNP | G | A | | | 1 | 33 | |
| 2′300′868 | SNP | C | T | | | 1 | 33 | |
| 2′300′871 | SNP | G | A | | | 1 | 33 | |
| 2′300′897 | SNP | C | T | | | 1 | 33 | |
| 2′300′924 | SNP | G | A | | | 1 | 33 | |
| 2′301′003 | SNP | G | A | | | 1 | 33 | |
| 2′301′036 | SNP | T | C | | | 1 | 33 | |
| 2′301′086 | SNP | T | C | | | 1 | 33 | |
| 2′301′104 | SNP | T | A | | | 1 | 33 | |
| 2′301′170 | SNP | A | G | | | 1 | 33 | |
| 2′301′257 | SNP | T | C | | | 1 | 33 | |
| 2′301′276 | SNP | A | G | | | 1 | 33 | |
| 2′301′300 | SNP | A | G | | | 1 | 33 | |
| 2′301′327 | SNP | T | A | | | 1 | 33 | |
| 2′301′358 | SNP | G | A | | | 1 | 33 | |
| 2′301′483 | SNP | A | G | | | 1 | 33 | |
| 2′301′520 | deletion | AGAGAGAGC | A | | | 1 | 33 | |
| 2′301′538 | SNP | T | C | | | 1 | 33 | |
| 2′301′708 | SNP | G | A | | | 1 | 32 | |
| 2′306′127 | SNP | G | A | | | 1 | 33 | |
| 2′306′341 | SNP | G | A | | | 1 | 31 | |
| 2′331′321 | SNP | G | A | | | 1 | 33 | |
| 2′335′134 | insertion | A | AT | | | 1 | 33 | |
| 2′336′578 | SNP | T | G | | | 1 | 33 | |
| 2′343′920 | SNP | G | A | | | 1 | 33 | |
| 2′354′603 | SNP | T | A | | | 1 | 33 | |
| 2′356′950 | SNP | T | C | | | 1 | 33 | |
| 2′381′155° | SNP | T | G | | | 1 | 33 | |

*Variants were excluded because obligate heterozygous Pp polled animals carried two copies of the variant allele.
** Genotyping of additional animals for this variant was technially impossible.
***One Simmental with polledness of unknown origin, two Limpurger with Scurs probably de novo mutation and one polled yak.
****Differentiation between the heterozygous (ref/var) and homozygous (var/var) genotype not performed.
°Variants identified and ruled out by Allais-Bonnet et al. [3]
+ Variants identified by Allais-Bonnet et al. [3]

In some embodiments, a modification in the genome is introduced to generate an animal which contains scurs which yields an effectively polled animal, such as a bovine. In some embodiments, the genome modification is introduced at the Scurs (Sc) locus. In some embodiments, the genome modification to generate a bovine with a scurs phenotype is introduced into chromosome 19 (BTA 19). In some embodiments, the genome modification to generate a bovine with a scurs phenotype is introduced into the TWIST1 gene. See e.g., Berryere et al. Anim Genet. 2004. 35:34-39 and Capitan et al. PLOS One 2011. 6(7): e22242.

In some embodiments, the genome is modified such that it contains one or more SNPs associated with a polled phenotype including, but not limited to, any one or more set forth in US Pat. App. Pub. 20050153328, particularly described at paragraphs [0036], [0081]-[0082], [0088], Table 1, Table 2 or any of the SNPs or regions that can be identified using the primers of Table 1 of in US Pat. App. Pub. 20050153328. In some embodiments, the genome is modified such that it contains one or more SNPs associated with a polled phenotype including, but not limited to, any one or more forth in US Pat. App. Pub. 20110195414, particularly at paragraphs [0013]-[0028], [0030], [0033], [0097], [0112], and Tables 1-5.

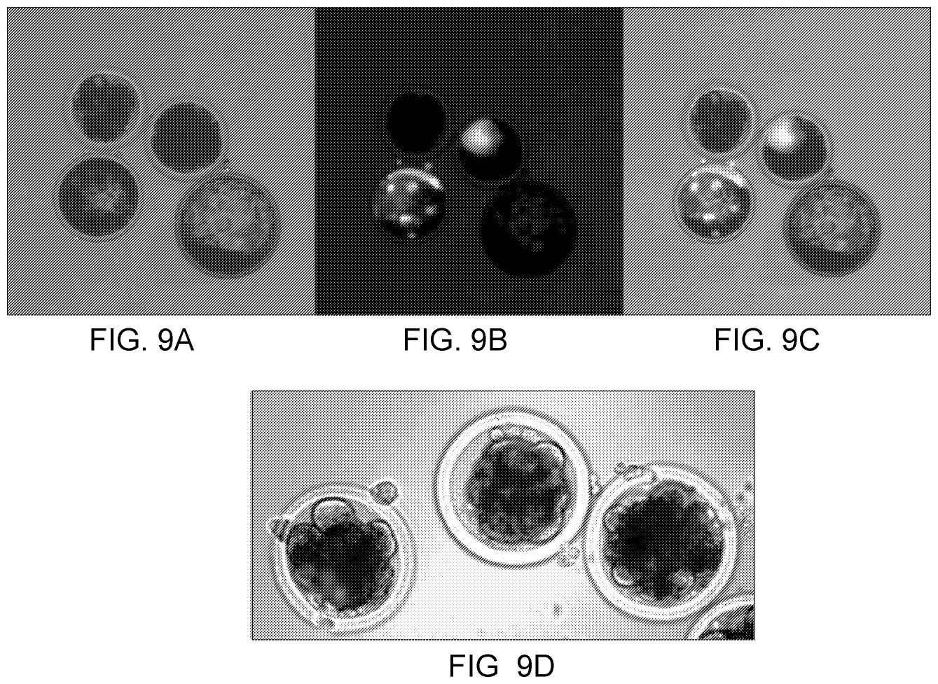

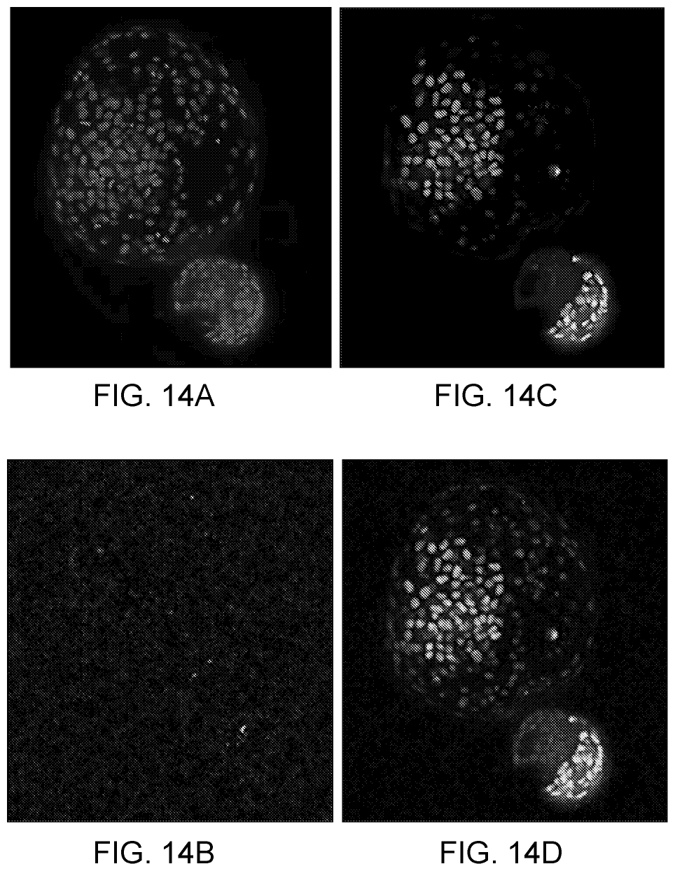

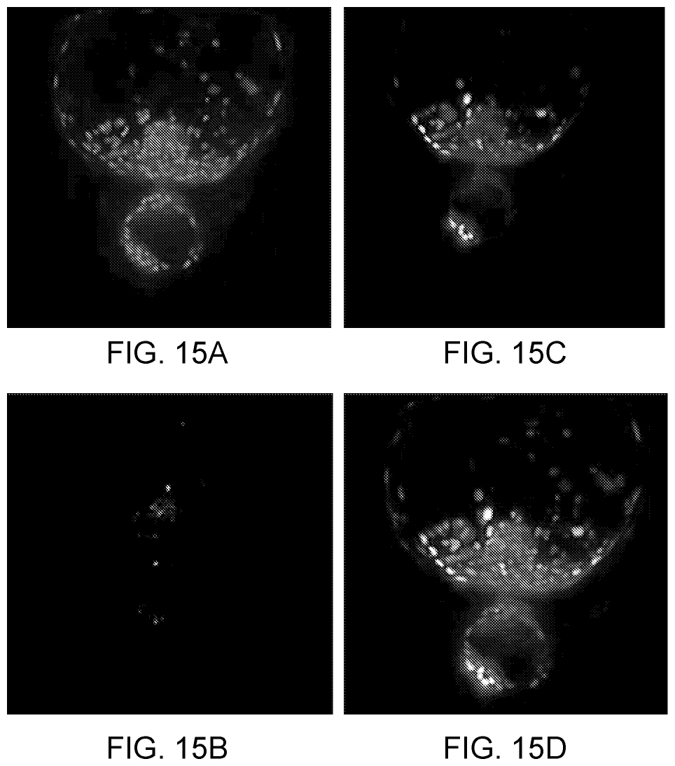

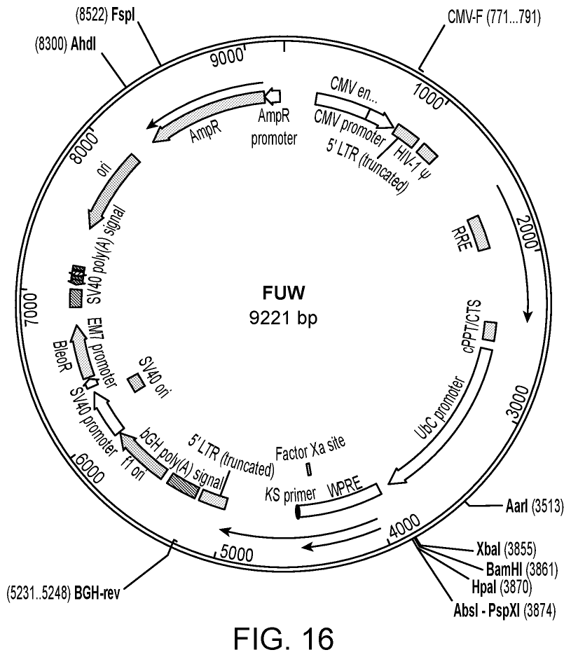

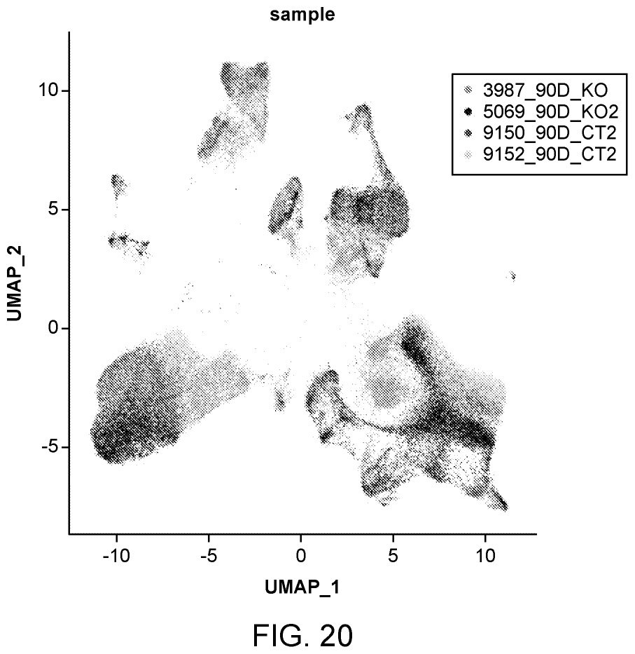

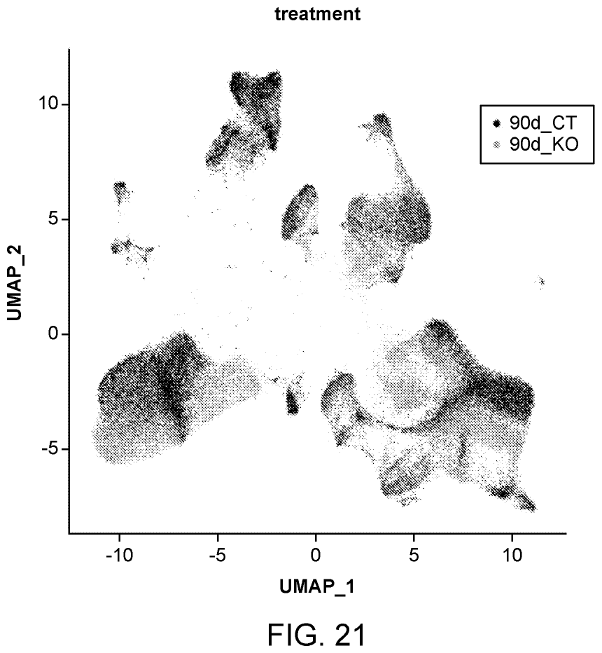

In some embodiments, the genome is modified such that it contains a SNP or haplotype corresponding to a SNP or haplotype including, but not limited to, any one or more of those described in U.S. Pat. No. 8,105,776, particularly at FIGS. 1-42, 47 and related descriptions, Col. 3 lines 19-67 (3:19-67), 4:1-67, 5:1-58, 7:19-67, 8:1-67, 9:1-12, 32:40-59, 33:15-29; 33:45-50, 33:58-67, 34:1-4, 34:37-47, 35:8-13, 35:19-29, 35:35-40, and Tables 1-7.

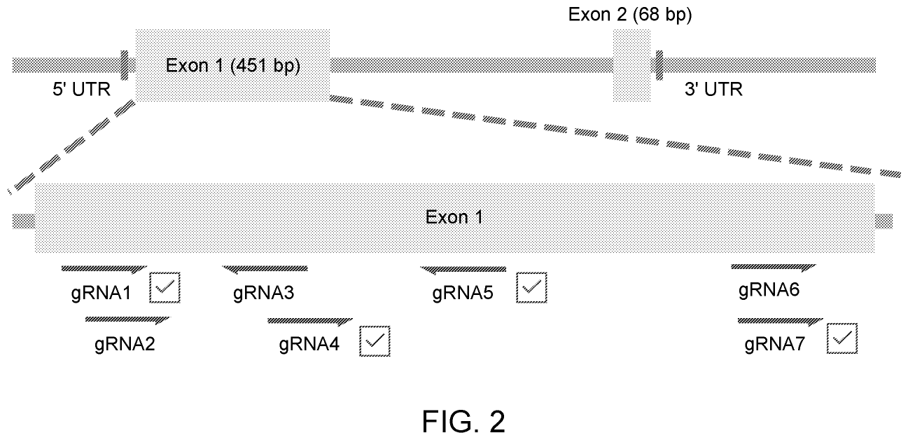

In some embodiments, the genome is modified such that it contains a modification to generate a polled phenotype, including, but not limited to, any one or more of those set forth in U.S. Pat. App. Pub. 2014/0201857, particularly at paragraphs [0026]-[0029], [0035]-[0036], [0038]-[0040], [0091]-[0098], and FIGS. 2-5.

### Example Modifications to Improve Disease Resistance or Tolerance

In some embodiments, the genome is modified such that the animal, such as a bovine, has improved disease resistance or disease tolerance. Exemplary disease in which it is advantageous for an animal to have resistance or tolerance to include, without limitation, mastitis, Johne's disease, bovine viral diarrhea-associated disease and other viral and microorganism mediated infections (e.g., tuberculosis chlamydiosis, leptospirosis, campylobacterosis, salmonellosis, listerosism yersiniosis, Pseudomonos, Aerobactor, Klebsiella, Mannhemia, Pasteurella, Histophilis, cryptosporidiosis, E. coli, rabies, anthrax, antibiotic resistant staphylococcus (e.g., MRSA), Fusobacterium necrophorum, streptococcus, corynebacterial, various fungi, and/or the like), BSE, BRD, IARS syndrome, milk fever, shipping fever, grass tetany, prussic acid poisoning, white muscle disease, IBR, foot and mouth disease, foot rot, and others.

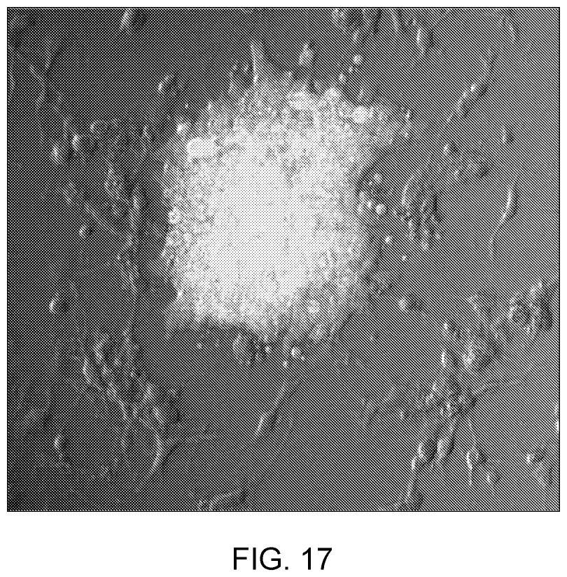

Inactive or insufficient Peg3 gene expression can result in disease intolerance and other issues. See e.g., U.S. Pat. Publication 20030018987. In some embodiments, the genome modification to improve disease resistance, such as to mastitis and other diseases includes one or more modifications in the Peg3 gene to restore healthy (non-diseased) or wild-type gene activity. In some embodiments, the modification alters a defective, inactive, or otherwise insufficient Peg3 gene to an active, functional Peg3 gene. In some embodiments, the genome modification to improve disease resistance, such as to mastitis, are in one or more genes located on the BTA5, BTA6, BTA7, BTA12, BTA13, BTA16, BTA18, BTA19, BTA20 chromosomes, such as in any of the genes and/or locations on such chromosomes as set forth in U.S. Pat. App. Publication 20150240308, particularly at paragraphs [0066]-[0104] and [0185]-[211], FIG. 17, Table 6, Table 2, Table 3, Table 4, Tables 8-26, and SEQ ID NOs: 1-3, and any of those described in U.S. Pat. App. Pub. 20110023158, including, but not limited to, any one or more of those described at paragraphs [0047]-[0051].

In some embodiments, the genome modification to improve disease resistance, such as to mastitis and/or other diseases includes one or more modifications in the beta-casein gene (CSN2), including, but not limited to, any one or more those set for the in US Pat. App. Pub. 20090013419, particularly at paragraph [0014].

In some embodiments, the genome modification to improve disease resistance, such as to tuberculosis and/or other diseases includes one or more modifications in the intergenic region of SFTPA1 and MAT1A, SP110 nucleome protein gene, the IPR1 gene and/or the intergenic region between FSCN1 and ACTB genes, such as that set forth in Chinese Patent 104293833 or Chinese Patent Application Publication CN201810813577.2A

In some embodiments, the genome modification to improve disease resistance includes one or more modifications to any of the target genes listed and/or whose gene product is a target noted in the Table beginning Col. 6 line 25 and continuing through to Col. 13 line 52 of U.S. Pat. No. 10,106,621.

In some embodiments, the genome modification to improve disease resistance, such as to bovine respiratory disease (BRD) and/or mastitis includes one or more modifications to granulocyte-colony stimulating factor gene (G-CSF), such as any of those modifications (or analogues thereof) described in U.S. Pat. No. 10,138,283, particularly at Col. 27: lines 15-45 and U.S. Pat. No. 5,416,195, particularly at Col. 2: lines 11-68, Col. 3: lines 1-16, Col. 5: lines 59-68, and Col. 6: lines 1-35.

In some embodiments, the genome modification to improve disease resistance, such as foot and mouth disease virus (FMDV), includes one or more modifications to the eIF4G gene, such as any of those set forth in U.S. Pat. No. 10,058,078, particularly at Col. 4: lines 60-67, Col. 5: lines 1-50, Col. 6: lines 16-53, Col. 7: lines 1-40, Col. 24: lines 66-67, Col. 25: lines 1-30, Table 1, Table 2, and FIG. 2.

In some embodiments, the genome modification to improve disease resistance, such as bovine spongiform encephalopathy (BSE or Mad Cow disease), includes one or more modifications such as any of those described in U.S. Pat. App. Pub. 20110023158, particularly one or more of those described at paragraphs [0057]-[0059].

In some embodiments, the genome modification to improve disease resistance, such as to mastitis and other diseases includes one or more modifications in Peg3, SOX5, ETNK1, LOC520387, PLCZ1, PIK3C2G, RERGL, LMO3, MGST1, SLC15A5, IGJ, UTP3, RUFY3, GRSF1, MOB1B, DCK, SLC4A4, GC, NPFFR2, ADAMTS3, CAD26, EDN3, RAB22A, TMEM74B, TBC1D20, DEFB129, DEFB119, DEFB117, DEFB 122a, DEFB122, DEFB123, DEFB124, ID1, XKR7, BPIFB2, BPIFB6, BPIFB3, BPIFB4, LAD1, CSRP1, MMP23B, TNFRSF4, TNFSRF18, ISG15, PLEKHN1, B3GALT6, SEC14-like protein 1, N-acetylglucosaminyltransferase, Acetylglucosaminyltransferase isozyme B, LIFR, EP4R, complement component C9, OSMR, Complement component C7 precursor, Complement component C6 precursor, beta-casein (CSN2), sFTPA1, MATA1A, SP110 nucleome protein gene, eIF4, or any combination thereof.

### Modifications to Prevent Genetic Disorders

In some embodiments, the genome is modified to contain one or more modifications that prevent one or more genetic disorders. Exemplary cattle genetic defects or disorders that can be prevented by modifying the genome include, without limitation, Alpha (a) and/or Beta (ß)-Mannosidosis, Arthrogryposis Multiplex (AM), Contractural Arachnodactyly (CA), Neuropathic Hydrocephalus (NH), Hypotrichosis (hairless calf), Idiopathic Epilepsy, Osteopetrosis, Protoporphyria, Pulmonary Hypoplasia and Anasarca (PHA), Tibial Hemimelia (TH), achondroplasia (bulldog dwarfism), alopecia, ankylosis, arthrogryposis (palate-pastern syndrome, rigid joints), brachynathia inferior (parrot mouth), cryptorchidism, dermoid, double muscling, fawn calf syndrome, hypotrichosisi (rat tail), neuraxial edema (maple syrup urine disease), oculocutaneous hypopigmentation, polydactyly, progressive bovine myeloencephalym prolonged gestation, syndactyly (mule foot), translocations, Bovine leukocyte adhesion deficiency, Complex Vertebral Malformation, freemartinism, and others. See also Ciploch et al., Genes. Genomics. 2017 39(5):461-471.

In some embodiments, the genome is modified to contain one or more modifications that can prevent dwarfism or mannosidosis, including, but not limited to, any one or more of the modifications described in U.S. Pat. App. Pub. 20110023158, particularly one or more of those described at paragraphs [0075]-[0078]. In some embodiments, the genome is modified to contain one or more modifications to prevent a bovine genetic disease such as in any one of the genes to prevent any one of the diseases described in Cieploch et al., Genes & Genomics 39: 461-471 (2017), particularly at Table 1.

### Thermotolerance

In some embodiments, it is advantageous that the animal is heat or cold tolerant. In some embodiments, the genome is modified to contain one or more modifications that confer heat and/or cold tolerance (generally referred to herein as thermotolerance) to the modified animal, such as a bovine. In some embodiments, the genome modification to improve thermotolerance, includes one or more modifications to the prolactin receptor (PRLR) gene, including, but not limited to, any of those set forth in U.S. Pat. App. Pub. 201902223417, particularly at paragraph [0008] and/or U.S. Pat. App. Pub. 20170079251, particularly at paragraphs [0006]-[0015], [0126]-[0129], [0132], [0140]-[0143], Table 1, Table 2, Table 4, Tables 7-8, and FIGS. 1-3.

### Modification of Meat or Milk Product Yield or Characteristics

In some embodiments, it is advantageous that the animal, such as a bovine, is modified such that it has modified and/or improved meat, milk, or other product yield or characteristics, such as reduced allergen content, reduced lactose content, improved nutrient profile, increased marbling, or other quality. In some embodiments, the genome is modified such that it contains one or more modifications in one or more genes such that the meat, milk, or other product has improved and/or modified yield and/or other characteristic.

### Modifications to Reduce Milk Allergens

Milk, particularly bovine milk, contains proteins that can be allergens to humans. The primary allergenic proteins in milk, particularly bovine milk, are casein (alpha S1, alphaS2, beta, kappa etc.), alpha-lactalbumin, and beta lactoglobulin. See e.g., Shoormasti et al., Iran J Allergy Asthma Immunol. 2016 April; 15(2):161-5. Other proteins in milk, such as lactoferrin, bovine IgG (e.g., IgG heavy chain), and bovine serum albumin may also be allergenic. In some embodiments, the genome is modified to contain one or more modifications to one or more genes that encode one or more of the allergenic proteins in milk, particularly bovine milk, not limited to casein (alpha S1, alphaS2, beta, kappa etc.), alpha-lactalbumin, beta lactoglobulin, lactoferrin, bovine IgG (e.g., IgG heavy chain), bovine serum albumin, or any combination thereof, such that the allergen content of milk is reduced or eliminated.

In some embodiments, the genome is modified to contain one or more modifications that reduce one or more milk allergens, the milk of a modified animal, such as a bovine, where the modification(s) are in the beta-lactoglobulin gene, such as one or more genetic mutations that confer amino acid mutations in a beta-lactoglobulin polypeptide such as C160S (single or double mutation) including, but not limited to, those set forth as in U.S. Pat. No. 6,677,433, particularly at 67:40-51. In some embodiments, such mutations result in production of milk with reduced allergen content or potential.

In some embodiments, the genome is modified to contain one or more modifications that reduce one or more milk allergens (such as proteins, lipids, fatty acids and/or the like), the milk of a modified animal, such as a bovine, such as one or more genetic mutations that confer amino acid mutations in a milk protein, lipids and/or fatty acids including, but not limited to, any one or more of the genetic mutations and/or proteins, lipids, fatty acids etc. set forth in e.g., U.S. Pat. App. Pub. 20110023158, particularly those described at paragraphs [0017]-[0039].

### Modifications to Improve Nutrient Profile of a Meat or Milk Product

In some embodiments, the genome modification(s) result in modified nutritional or nutrient profile of meat or milk produced from an animal, such as a bovine, with said genetic modification(s). Such modifications to the nutrient profile can produce an improved meat or milk with some nutritional or other health benefit(s) to one or more populations of humans or animals consuming the meat or milk product. Examples include, without limitation, milk with an altered fat content, reduced lactose or lactose free milk, and/or the like.

In some embodiments, the genome is modified to contain one or more modifications that can modify the nutritional or nutrient profile of a meat and/or milk of a modified animal, such as a bovine, including, but not limited to, one or more genetic modifications described in U.S. Pat. App. Pub. 20110023158, particularly one or more of those described at paragraphs [0036]-[0043].

In some embodiments, the genome is modified to reduce the amount of lactose in milk of a modified animal, such as a bovine, including, but not limited to, one or more genetic modifications described in U.S. Pat. App. Pub. 20110023158, particularly one or more of those described at paragraphs [0040]-[0043].

In some embodiments, the genome is modified to contain one or more modifications that can increase or modify the content of bioactive proteins in milk, including, but not limited to, any of those described in U.S. Pat. App. Pub. 20110023158, particularly one or more of those described at paragraphs [0047]-[0051].

Modifications to Increase or Modify Milk and Meat Yield and/or Quality

In some embodiments, the genome modification(s) result in an animal, such as a bovine, with increased or otherwise improved milk and/or meat yield and/or carcass qualities.

In some embodiments, the genome is modified to contain one or more modifications that can modify milk and/or meat yield in the modified animal, including, but not limited to, one or more modifications described in U.S. Pat. App. Pub. 20110023158, including, but not limited to, one or more of those described at paragraphs [0044]-[0046], [0052]-[0056], and [0059], one or more of those described in U.S. Pat. App. Pub. 20180296522, particularly at paragraph [0015], one or more in the DGAT gene such as one or more of those described in U.S. Pat. App. Pub. 20060172329, particularly at paragraphs [0007], [0009], [0012], [0016], [0077], and Tables 1-2; or any combination thereof.

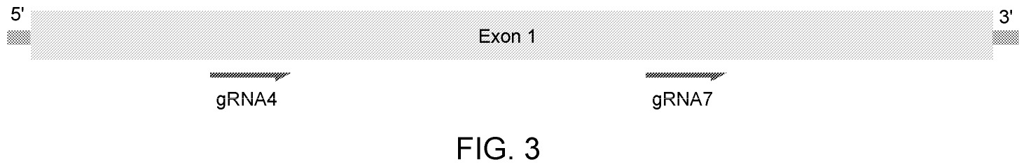

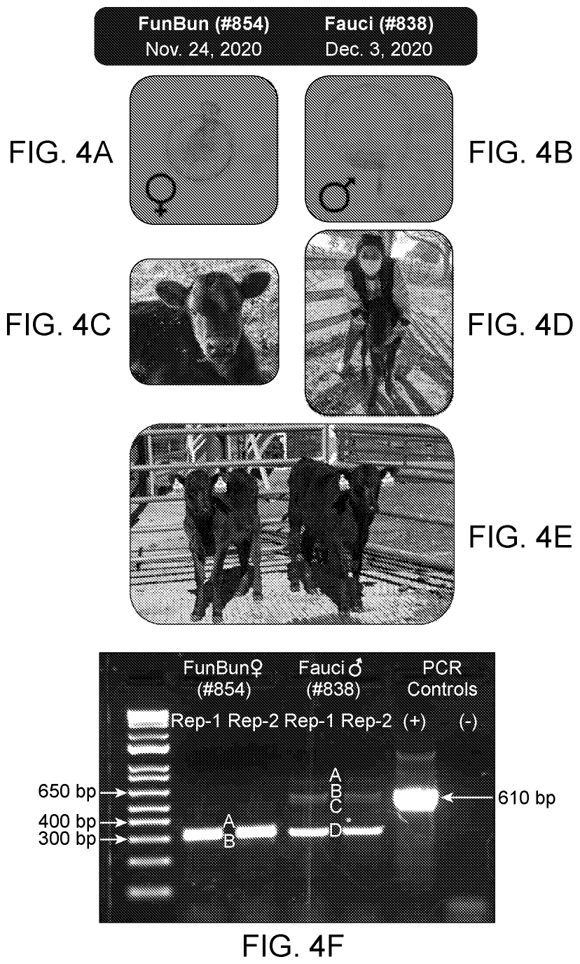

In some embodiments, the genome is modified to contain one or more modifications that can at least modify one or more growth, milk and/or meat yield, milk, and/or carcass traits and/or quality including, but not limited to, any one or more of those in the NCAPG gene as set forth in U.S. Pat. App. Pub. 20090260095 particularly at paragraph [0008], any one or more of those in an IGF-2 gene set forth in U.S. Pat. App. Pub. 20070026404 particularly at paragraphs [0103]-[105], any one or more of those in FABP4 gene as set forth in U.S. Pat. App. Pub. 20070020658 particularly at paragraph [0157], any one or more of those in the TFAM gene as set forth in U.S. Pat. App. Pub. 20070065843 particularly at paragraphs [0097]-[0100], [0130], [0155], [0164]-[0165], [0216]-[0218], Tables 2, 4-5; any one or more of those SNPs in the TFAM, TFB1M, TFB2M and/or other genes as set forth in U.S. Pat. App. Pub. 20080183394 particularly at paragraphs [0025]-[00030], [0096]-[0099], [0108]-[0109], [0129], [0182]-[0185], [0216]-[0219], [0226] Table 2, Tables 4-5, FIG. 3, FIG. 4; any one or more of those set forth in U.S. Pat. No. 6,383,751, particularly at Col. 24: line 59 through Col. 25: line 7, and Tables 5-8; any one or more of those set forth in U.S. Pat. App. Pub. 20070026404, particularly at paragraphs [0030]-[0044, [0096]-[0097], [0103], [0106]; any one or more of those in the UCN3 gene set forth in U.S. Pat. No. 7,662,567, particularly at the abstract, Col. 4:26-55, Col. 16:4-46, Col. 18:6-4, Col. 29:47-67, Col. 30:1-67, Col. 31:1-22 and Col. 47-67, 32:1-22, Table 1, and FIGS. 1-4D; any one or more of those in the CRH gene set forth in U.S. Pat. No. 7,662,567, particularly at the abstract, Col. 3: lines 25-28, Col. 4: lines 51-67, Col. 5: lines 1-22, Col. 16: lines 35-52, Col. 28: lines 33-62, Col. 31: lines 7-51, Col. 32: lines 1-52, Col. 33: lines 16-32 and 63-67, Col. 34: lines 64-67, Col. 35: lines 1-34, Table 1, and FIGS. 1, 2A-2D, 3A-3C, and 4A-4C; any one or more of those set forth in U.S. Pat. No. 8,008,011, particularly at the abstract, Col. 4: lines 3-31 and 48-64, Col. 6: lines 63-67, Col. 7: lines 1-22, Col. 8: lines 1-67, Col. 9: lines 1-2, Tables 2-4, and FIGS. 1-4; any one or more of those in the leptin and/or ob gene as set forth U.S. Pat. App. Pub 20030219819, particularly at paragraphs [0005]-[0009], [0037], and [0052]; any one or more of those in the UQCRCl gene set forth in U.S. Pat. No. 7,879,552, particularly at Col. 2: lines 40-67, Col. 3: lines 10-24, Col. 5: lines 18-21, Col. 16: lines 6-37, Col. 29: lines 13-41, Col. 30: lines 15-41, Tables 1-5, and FIGS. 3A-3D; any one or more of those in the set forth in U.S. Pat. No. 7,157,231, particularly at Col. 2: lines 6-25 and 51-67, Col. 3: lines 10-22 and 61-67, Col. 4: lines 1-19, and Tables 1-3, any one of more of the modifications, SNPs, or variants in adnectin gene, an engineered adnectin gene, or those that produce an adnectin or engineered adnectin gene product, such as any one or more of those set forth in U.S. Pat. App. Pub. 20190307855, particularly at paragraphs [0010]-[0039], Tables 1-4; or any combination thereof.

In some embodiments, the genome is modified to contain one or more modifications in any one of the genes set forth in Tables 1, 2, and/or 3 and/or corresponding to SEQ ID NO: 1-408 of U.S. Pat. No. 7,638,275.

In some embodiments, the genome is modified to contain one or more modifications in any one of the genes to improve nutrition and/or processing as set forth in Wall et al., 1997. J Dairy Sci. 80:2213-2224, particularly at TABLE 6.

Modifications in Traits that Relate to Production and/or Management

In some embodiments, the genome modification(s) result in an animal, such as a bovine, with improved characteristics associated with production and/or management, including, but not limited to, temperament, coat color, hair shedding, foot angle, growth, feed efficiency, lameness, blood pressure, and/or the like. Exemplary modifications described elsewhere herein, such as thermotolerance or disease resistance, can also improve production and/or management.

In some embodiments, the genome is modified to contain one or more modifications that can modify the coat color or other coat property (such as hair length or shedding) of the animal, such as a bovine, such as one or modifications described in U.S. Pat. App. Pub. 20110023158, particularly one or more of those described at paragraphs [0060]-[0074], and/or one or more modifications described in U.S. Pat. No. 10,716,298, particularly one or more of those described at Col. 3, lines 58-67 (3:58-67), 4:1-67, 5:1-67, 7:1-67; 8:1-18; and/or Table 1, FIG. 1. U.S. Pat. No. 10,779,518, provides several exemplary genetic markers for coat properties, particularly those related to the prolactin receptor and gene. In some embodiments, the one or more modifications can include one or more modifications such that the modified polynucleotide contains or, in the case where a marker indicates an undesirable characteristic, does not contain a genetic marker, SNP, modification, or other variant polynucleotide described in U.S. Pat. No. 10,779,518, particularly at 1:34-54, 18:16-44 and 63-67, 19:1-8, FIG. 1-5.

In some embodiments, the genome is modified to contain one or more modifications that can affect the growth rate, feed efficiency, or other aspect of growth and/or development and energy utilization, such as any one or more in any one of the genes or markers or including any one or more of the SNPs or other modifications described in U.S. Pat. App. Pub. 20080177597, particularly at [0013], [0147], [0219], Tables 1-10, 14-16, and FIGS. 1-19; any one or more of the genes, markers, SNPs, and/or modifications set forth in U.S. Pat. App. Pub. 20020142315, particularly at paragraphs [0009]-[0010], [0013], [0021], [0038]-[0044], [0095], [0097]-[0103], Tables 1-2, or any combination thereof.

In some embodiments, the genome is modified to contain one or more modifications that affect one or more characteristics associated with animal production and/or management, including, but not limited to, birth weight, calving ease, fertility, reproduction capacity, weaning weight, yearling weight, dry matter intake, etc. including, but not limited to, one or modifications in any of the genes, markers, polynucleotides and/or any one or more modifications or variations set forth in U.S. Pat. App. Pub. 20090181386, particularly in paragraphs [0011]-[0030], [0032]-[0060], [0083], [0088]-[00237], Table 2a-20j, 20k1-20k19; one or more set forth in U.S. Pat. App. Pub. 20070026404, particularly at paragraphs [0103]-[0106]; one or more set forth in U.S. Pat. App. Pub. 20060172329, particularly at paragraphs [0007], [0009], [0012], [0016], [0077], and Tables 1-2; one or more set forth in U.S. Pat. App. Pub. 20150344974, particularly at [0018]-[0023], [0029], [0080]-[0081], [0012]-[0013], and Tables 3-7; one or more as set forth in U.S. Pat. No. 7,879,552 particularly at Col. 2: lines 40-67, Col. 3: lines 10-24, Col. 4: lines 29-34, Col. 5: lines 18-21, Col. 16: lines 6-37, Col. 29: lines 13-41, Col. 30: lines 15-41, Tables 1-5, and FIGS. 3A-3D; one or more as set forth in U.S. Pat. App. Pub. 20070065843 particularly at paragraphs [0097]-[0100], [0130], [0155], [0164]-[0165], [0216]-[0218], Tables 2, 4-5; one or more set forth in U.S. Pat. App. Pub. 20100009374, particularly at paragraphs [0007]-[0013], [0036]-[0039], [0050]-[0051], [0070], [0077], and Tables 2A-2B, 4-5, 7, 9, 11, 12, or any combination thereof

In some embodiments, the genome is modified to contain one or more modifications that can affect temperament, including, but not limited to modifications in the PEG3 gene, such as any of those described in U.S. Pat. App. Publication 20030018987, particularly at paragraph [0004].

In some embodiments, the genome is modified to contain one or more modifications that can affect pulmonary arterial pressure including, but not limited to, modifications in the EPAS1 or other relevant gene, such as any of those genes and/or SNPs or other modifications described in U.S. Pat. No. 10,138,522, particularly at Col. 1: lines 58-67, Col. 2: lines 1 and 20-61, Col. 9: lines 12-15, Col. 29: lines 24-36, Col. 30: lines 30-34, Tables 1-3, and FIGS. 7-9.

### Modifications to Generate Bovine Bioreactors

In some embodiments, the genome is modified to contain one or more modifications, such as exogenous and/or heterologous genes or regulatory elements, that can render the animal, such as a bovine, a bioreactor that can produce one or more endogenous or exogenous proteins, lipids, or other biologics in e.g., a bodily fluid, that can be optionally harvested from said bodily fluid and provided to a subject in need thereof. The use of bovine as bioreactors is known in the art. See e.g., Monzani et al. Adv Exp Med Biol. 2022; 1354:299-314, Keefer et al., Council for Agricultural Science and Technology (CAST). 2007. The Role of Transgenic Livestock in the Treatment of Human Disease. Issue Paper 35. CAST, Ames, Iowa; Colman. Am J. Clin. Nut. 1996. 63:639S-645S and Wall et al., 1997. J Dairy Sci. 80:2213-2224. For example, if it is an endogenous bovine gene product that is desired, the genome can be modified to overexpress the desired endogenous bovine gene products in e.g., the milk from the animal. They can be subsequently purified from the milk to obtain the desired gene product. In other examples, it is desirable to produce a heterologous protein. In these cases, the donor cell genome can be modified to express the desired heterologous protein (e.g., by insertion of a transgene corresponding to the desired heterologous protein). In some cases, it can replace a milk protein coding region such that the endogenous milk protein promoter drives transgene production in the mammary tissue. The desired heterologous protein produced can then be purified from the milk. Other examples will be appreciated in view of the description herein. In some embodiments, the exogenous desired heterologous protein or other gene product is a therapeutic protein or other gene product.

### Gamete, Chromosome, Allele, or Gene Selection Bias

In some embodiments, the genome modification introduces one or more modifications that provides for genetic mediated selection or bias of sperm or oocytes carrying a desired genotype, allele, chromosome, and/or the like (or selection against undesired sperm or oocytes), such as those modifications, engineered genes, and/or the like set forth in U.S. Pat. App. Pub. 20210324340 (modifications of the SRY gene), one or more set forth in U.S. Pat. App. Pub. 20200399661, particularly at paragraphs [0034]-[0051], [0164], [0168], FIGS. 1A-9C, Examples 1-12.

### Other Modifications

In some embodiments, the genome is modified to contain one or more SNPs as set forth in the bovine SNP database that is publicly available at animalgenome.org/bioinfo/resources/util/q_bovsnp.html.

In some embodiments, the modification is made in any one or more genes or contains any of the modifications or SNPs as set forth in Casas and Kehrli Jr. Front. Vet. Sci., 15 Dec. 2016, doi.org/10.3389/fvets.2016.00113, particularly at Table 1, any of those set forth in Ma et al., Agriculture 2021, 11, 1018. doi.org/10.3390/agriculture11101018, particularly at Tables 1-3, any one or more as set forth in Keogh et al., Animal Volume 15, Issue 1, January 2021, 100011, particularly at Table 4, 5; any one or more as set forth in Costilla et al., Genetics Selection Evolution volume 52, Article number: 51 (2020); any one or more as set forth as in Dyle et al., Genetics Selection Evolution Vol. 52, Article No. 2 (2020), any one or more set forth in Shao et al. 2021, Front Genet. 12: 617128; Ortega 2018, Anim Reprod, vol. 15, nSupplement 1, p. 923-932, dx.doi.org/10.21451/1984-3143-AR2018-0018; Halli et al. 2021, PLoS ONE 16(10): e0258216, doi.org/10.1371/journal.pone.0258216; Thomson et al., Canadian Journal of Animal Science, 2013. 93(3): 295-306, doi.org/10.4141/cjas2012-136; Sweett et al., 2020 Scientific Reports, Vol. 10, Article No. 20102; Hirwa et al. 2011, Asian J Anim Sci 5(1):34-45; Marete et al. 2018, PloS ONE 13(7): e0199931, doi.org/10.1371/journal.pone.0199931; Paredes-Sanchez et al. 2020, Rev Mex Cienc Pecu 11(3):894-904, doi.org/10.22319/rmcp.v11i3.5279; Lim et al., 2014. Int. J Genomic Article ID 708562, doi.org/10.1155/2014/708562; Weikard et al. 2005, Comp. Genomics 21(1); Buzanskas et al. 2017, Journal of Animal Science and Biotechnology 8:67; Bouwman et al. 2018, Meta-analysis of genome-wide association studies for cattle stature identifies common genes that regulate body size in mammals. Nature Genetics 50:362-367, DOI: 10.1038/s41588-018-0056-5; Ali et al. 2020, Ann. Anim. Sci., Vol. 20, No. 2: 409-423; De Leon et al. 2019, Genet. Mol. Res. 18(3): gmr18373; Sun et al. 2021, Journal of Dairy Research 88(3): 247-252; Baqir et al. 2015, J App. Animal. Res. 44(1):380-383, doi.org/10.1080/09712119.2015.1091333; Alvarenga et al. 2021, Animals 11(3): 715. doi.org/10.3390/anil 1030715; Fang et al. 2020, Genome Res. 30: 790-801; Dunner et al. 2013, Livestock Science 154 (1-3): 34-44; Li et al. 2011, DNA and Cell Biology 30(1):247-254; or any combination thereof.

### Non-Engineered Donor Cells

In some embodiments, the donor cells are derived from a donor cell source are not genetically modified prior to being used to complement a NANOS3 deficient host. In some embodiments, such cells can be from a desired breed, lineage, or specific sire or dam. In some embodiment, such non-engineered donor cells have or be said to contain “elite genetics” or are otherwise derived or obtained from “genetically elite animals”. The phrase “elite genetics” or “genetically elite” is a term of art that refers to the genetic makeup an animal, such as bovine, or cell(s) thereof that represents that such an animal (or cell(s) thereof) are superior genetic outliers (i.e., top or bottom, depending on trait, phenotype, genotype, etc. 0.0001 to 10% such as 0.0001% to 0.001%, 0.001% to 0.01%, 0.01% to 0.1%, 0.1% to 1.0%, 1% to 2%, 2% to 3%, 3% to 4%, 4% to 5%, 5% to 6%, 6%-7%, 7%-8%, 8%-9%, 9%-10%, or any value or range of values therein) of a contemporary population) for a desired trait(s), phenotype(s) and/or genotype(s) at one or more loci, alleles, genes, and/or the like.

### Generating Engineered Host and Donor Cells for Germline Complementation

Engineered host cells (e.g., NANOS3 deficient cells) and/or engineered donor cells can be modified using any suitable genetic modification technique or system. Exemplary systems, techniques and strategies are described below and elsewhere herein. Other suitable systems and approaches will be appreciated by one of ordinary skill in the art in view of the description herein and are within the scope of the present description. Engineered host cells and/or host animals (i.e., NANOS3 deficient cells and/or animals) can be generated using appropriate techniques used to make genetically modified organisms, such as bovine. These include without limitation, somatic cell nuclear transfer, genetic modification of various pluripotent, totipotent, or other stem cells, including but not limitation embryonic stem cells, primordial germ cells, primordial germ cell-like cells, spermatogonial or oogonial stem cells, induced pluripotent stem cells, zygotes, blastocycts, blastomeres, etc. Exemplary bovine cells for genome modification to produce host cells, donor cells, and/or animals, such as bovine, are described in e.g., Bogliotti et al. PNAS Feb. 27, 2018, 115 (9) 2090-2095 and WO 2019/140260 (bovine primed pluripotent embryonic stem cells), Zhao et al., PNAS Apr. 13, 2021 118 (15) e2018505118; doi.org/10.1073/pnas.2018505118 (bovine expanded potential stem cells); Su et al., Int. J. Mol. Sci. 2021, 22(19), 10489; doi.org/10.3390/ijms221910489 (bovine induced pluripotent stem cells); Bressan et al. Stem Cell Res. Ther. 2020. 11:247 (bovine induced pluripotent stem cells); Pillai et al., Biol. Open. 2021 Oct. 15; 10(10):bio058756. doi: 10.1242/bio.058756 (bovine induced pluripotent stem cells); Kawaguchi et al. 2015. PLoS ONE 10(8): e0135403. doi.org/10.1371/journal.pone.0135403 (naïve bovine induced pluripotent stem cells); Yuan. PNAS 2018. Feb. 27:115(9):1962-1962 (bovine pluripotent embryonic cells); Wei et al., Cell Stem Cell. 2021 Mar. 4; 28(3):550-567.e12. doi: 10.1016/j.stem.2020.11.003 (intermediate pluripotent stem cells); Owen et al., 12;22(1):118. doi: 10.1186/s12864-021-07418-3 (bovine zygotes); Soto et al. 2021. Sci Rep. 11:11045. (bovine embryonic stem cells); Linher et al., 2009. PLOS One 4(12): e8263. doi.org/10.1371/journal.pone.0008263 (bovine primordial germ cell-like cells), Souza et al., Reprod Domest Anim. 2017; 52(2):243-250 (bovine ovarian stem cells); Kim et al., Reprod. Fertil. Dev. 2016, 28, 1762-1780 (embryonic pluripotent cells); Van Stekelenburg-Hamers, et al. Mol. Reprod. Dev. 1995.40:444-454 (inner cell mass pluripotent cells); Mitalipova et al., Cloning. 2001, 3, 59-67 (bovine embryonic pluripotent cells); Stice et al., Biol. Reprod. 1996. 54:100-110 (embryonic pluripotent cells), Lim et al. Therio. 2011. 76:133-142 (bovine embryonic pluripotent cells); Wu et al. Sci. Rep. 2016, 6, 1-12 (bovine embryonic pluripotent cells); Park et al., Anim. Reprod. Sci. 2015 (bovine pluripotent cells); Saito et al., Biol. 1992, 201, 134-141; Iwasaki et al., Biol. Reprod. 2000, 62, 470-475; Jin et al., Cytotechnology 2012, 64, 379-389 (bovine blastocyst derived pluripotent cells); Huang et al., PLoS ONE 2011, 6, e24501 (bovine induced pluripotent cells); Sumer et al. J. Anim. Sci. 2011, 89, 2708-2716 (bovine induced pluripotent cells); Xiang et al. FEBS J. 2021, 1-18 (bovine expanded potential stem cells); Han et al. Cell Res. 2011, 21, 1509-1512 (bovine induced pluripotent stem cells); Cao et al., Int. J. Biol. Sci. 2012, 8, 498-511 (bovine iPSCs); Wang et al. Cell Death Dis. 2013. 4(e907) (bovine testicular iPSCs); Talluri et al., Cell. Reprogram. 2015. 17, 131-140 (bovine iPSCs); Heo et al., Stem Cells Dev. 2015, 24, 393-402 (bovine iPSCs); Malaver-Ortega et al., Stem Cells Int. 2016, 2016, 1-11 (bovine iPSCs); Zhao et al., Tissue Cell 2017, 49, 521-527. (single cell derived bovine iPSCs); Kawaguchi et al., PLoS ONE 2015 10, e0135403. (bovine iPSCs); Furusawa et al., Biol. Repro. 2013. 89:2(1):1-12 (bovine inner cell mass derived cells); Aponte et al. 2008. Reprod. November 136(5):543-547 (bovine spermatogonial stem cells); Tajik et al., Iran J Vet Res. 2017. 18(2):113-118 (bovine spermatogonial stem cells); Zheng et al. Reprod. 2014. 147(3). doi.org/10.1530/REP-13-0466 (bovine spermatogonial stem cells); Oatley et al. 2004. Biol. Rep. 71(3):942-947 (bovine spermatogonial stem cells); Lei et al., 2017. J. Integr. Agri. 16(11):2547-2557 (bovine male germline stem cells); McLean, Z., B. Oback, and G. Laible. 2020. Embryo-mediated genome editing for accelerated genetic improvement of livestock. Frontiers of Agricultural Science and Engineering 7(2):148-160 (bovine stem cells); Giassetti et al. 2019. Spermatogonial Stem Cell Transplantation: Insights and Outlook for Domestic Animals. Annual Review of Animal Biosciences 7(1):385-401 (SSCs); Ciccarelli et al., 2020. Donor-derived spermatogenesis following stem cell transplantation in sterile NANOS2 knockout males. Proceedings of the National Academy of Sciences 117(39):24195-24204 (SSCs); Kinoshita et al., 2021, Pluripotent stem cells related to embryonic disc exhibit common self-renewal requirements in diverse livestock species. Development 148, dev199901 (embryonic disc stem cells); Zhi et al., 2022, Cell Research. 32:383-400 (epiblast stem cells); Yu et al., 2021 (intermediate or formative PSCs); Xiang et al., 2021. LCDM medium supports the derivation of bovine extended pluripotent stem cells with embryonic and extra embryonic potency in bovine-mouse chimeras from iPSCs and bovine fetal fibroblasts. FEBS Journal 288: 4394-4411; and elsewhere herein (see e.g., exemplary techniques for generating genetically modified and cloned bovine below and Working Examples herein). Where the donor cells are gametes or embryonic in nature, suitable techniques, such as embryo transfer, invitro fertilization etc. can be used to obtain an adult engineered animal.

Exemplary techniques for generating genetically modified and cloned bovine are described in e.g., See e.g., Tan, W., et al. (2016) Transgenic Res, 2016 June; 25(3):273-287, Yum et al., J Anim Sci Biotechnol. 2018; 9: 16, Monzani et al., Bioengineered. 2016 May-June; 7(3): 123-131, Chan et al., PNAS Nov. 24, 1998 95 (24) 14028-14033; Laible and Wells. (2006) Transgenic Cattle Applications: The Transition from Promise to Proof, Biotechnology and Genetic Engineering Reviews, 22:1, 125-150, DOI: 10.1080/02648725.2006.10648068; Wall et al., 1997. J Dairy Sci. 80:2213-2224, Ross and Cibelli. Methods Mol Biol. 2010; 636:155-77. doi: 10.1007/978-1-60761-691-7_10; Beyhan Z, et al. Dev Biol. 2007. PMID: 17359962; lager A E, et al. Cloning Stem Cells. 2008. PMID: 18419249; Ross P J, et al. Reproduction. 2009. PMID: 19074500; Wang K, et al. Cloning Stem Cells. 2009. PMID: 19196039; Arias M E, et al. Biol Res. 2013. PMID: 24510147; Bogliotti Y S, et al. J Vis Exp. 2016; Bogliotti Y S, et al. Proc Natl Acad Sci USA. 2018; Daigneault B W, et al. Sci Rep. 2018; Daigneault B W, et al. Biotechniques. 2018 November; 65(5):281-283. doi: 10.2144/btn-2018-0051; Goszczynski et.al. Biol Reprod. 2019 Apr. 1; 100(4):885-895. doi: 10.1093/biolre/ioy256; Soto and Ross. Transgenic Resm. 2016 June; 25(3):289-306. doi: 10.1007/s11248-016-9929-5; Goszczynski et al., Reprod Domest Anim. 2019 October; 54 Suppl 4:22-31. doi: 10.1111/rda.13503; Soto et al., Sci Rep. 2021 May 26; 11(1):11045. doi: 10.1038/s41598-021-90422-0; Owen et al., BMC Genomics. 2021 Feb. 12; 22(1):118. doi: 10.1186/s12864-021-07418-3; Henning et al., Sci Rep. 2020 Dec. 18; 10(1):22309. doi: 10.1038/s41598-020-78264-8; Camargo et al., Front. Genet. 2020. 7:11:570069; Ferre et al., Animal. 2020 May; 14(5):991-1004. doi:10.1017/S1751731119002775; Navarro et al., Reprod Fertil Dev. 2019 January; 32(2):11-39. doi: 10.1071/RD19272; Owen et al., Sci Rep. 2020 Sep. 29; 10(1):16031. doi: 10.1038/s41598-020-72902-x; Young et al., Nat Biotechnol. 2020 February; 38(2):225-232. doi: 10.1038/s41587-019-0266-0; Hennig et al., Sci. Rep. 2022. 8:12:2067, Giassetti et al. 2019. Spermatogonial Stem Cell Transplantation: Insights and Outlook for Domestic Animals. Annual Review of Animal Biosciences 7(1):385-401 (SSCs); Ciccarelli et al., 2020. Donor-derived spermatogenesis following stem cell transplantation in sterile NANOS2 knockout males. Proceedings of the National Academy of Sciences 117(39):24195-24204 (SSCs) and the references cited therein, which are incorporated by reference in their entireties and can be adapted for use with the present disclosure.

In embodiments, the method includes delivering a genetic modifying system and/or other optional exogenous cargo polynucleotide and/or polypeptide and/or components thereof to a cell or cells to be modified. Delivery can occur in vivo, in vitro, ex vivo, or in situ. Exemplary delivery compositions, systems, and techniques are further described below and elsewhere herein. In some embodiments, the cells that are modified are bovine cells, such as bovine embryonic stem cells, bovine primordial germ cells, bovine primordial germ cell-like cells, bovine pluripotent stem cells, bovine totipotent stem cells, bovine oogonial stem cells, bovine oogonia, bovine spermatogonial stem cells, bovine spermatogonia, bovine germ cells, bovine zygotes, bovine blastocyst cells, bovine blastomeres, bovine induced pluripotent stem cells (such as those reprogrammed from somatic cells and/or the like.

In some embodiments, a genetic modifying system can be used to introduce an exogenous or heterologous gene (such as a gene native to another species or organism). In some embodiments introduce an exogenous an engineered gene construct to a cell. In some embodiments introduce an exogenous an engineered gene construct to a cell. In some embodiments, the exogenous engineered gene construct is an engineered gene construct capable of selective germ cell ablation, destruction, or otherwise renders selected germ cells or germ cell progenitor cells incapable of fertilization. In some embodiments, the genetic modification system can be used to perform gene editing.

The engineered host and donor cells can be used, inter alia, in a germline complementation approach with the engineered donor being introduced to the germline depleted host to generate surrogate sires and damns that can be used in a conventional mating scheme to produce offspring of the donor cell source.

### Exemplary Genetic Modification Systems

In certain embodiments, the genetic modification system includes a programmable nuclease system (e.g., a CRISPR (or CRISPR-Cas) system), a zinc finger nuclease (ZFN) system, a TALEN, a meganuclease), an RNAi system, transposon system, or a combination thereof. Various genetic modification systems have been used to modify bovine cells and/or generate modified bovines, including CRISPR-Cas systems, ZFNs, TALENs, and transposon systems. See e.g., Owen et al., BMC Genomics volume 22, Article number: 118 (2021); Yum S Y, Lee S J, Kim H M, Choi W J, Park J H, Lee W W, et al. Efficient generation of transgenic cattle using the DNA transposon and their analysis by next-generation sequencing. Sci Rep. 2016; 6(27185); Garrels W, Talluri T R, Apfelbaum R, Carratala Y P, Bosch P, Potzsch K, et al. One-step multiplex Transgenesis via sleeping beauty transposition in cattle. Sci Rep. 2016; 6(21953); Ding S, Wu X, Li G, Han M, Zhuang Y, Xu T. Efficient transposition of the piggyBac (PB) transposon in mammalian cells and mice. Cell. 2005; 122:473-83; Li T, Shuai L, Mao J, Wang X, Wang M, Zhang X, et al. Efficient production of fluorescent transgenic rats using the piggyBac transposon. Sci Rep. 2016; 6(33225); Alessio A P, Fili A E, Garrels W, Forcato D O, Olmos Nicotra M F, Liaudat A C, et al. Establishment of cell-based transposon-mediated transgenesis in cattle. Theriogenology. 2016; 85:1297-311. e2; Kim S, Saadeldin I M, Choi W J, Lee S J, Lee W W, Kim B H, et al. Production of transgenic bovine cloned embryos using piggybac transposition. J Vet Med Sci. 2011; 73:1453-7; Liu et al., Nature Communications volume 4, Article number: 2565 (2013); Sun et al., Scientific Reports Vol. 8, Article No. 15430 (2018); Luo et al., 2014. Efficient Generation of Myostatin (MSTN) Biallelic Mutations in Cattle Using Zinc Finger Nucleases. PLoS ONE 9(4): e95225, doi.org/10.1371/journal.pone.0095225; U.S. Pat. Pub 20110023158; Wang et al., Efficient TALEN-mediated gene knockin at the bovine Y chromosome and generation of a sex-reversal bovine. Cellular and Molecular Life Sciences volume 78, pages 5415-5425 (2021); Moghaddassi et al. (2014) TALEN-Mediated Modification of the Bovine Genome for Large-Scale Production of Human Serum Albumin. PLoS ONE 9(2): e89631, doi.org/10.1371/journal.pone.008963; and US Pat. Pub. 20170099813, which are all incorporated by reference herein as if expressed in their entireties and can be adapted for use with the present disclosure. These and other suitable genetic modifying systems for bovine genetic modification are described in greater below and in e.g., the Working Examples herein.

### CRISPR-Cas Systems

In some embodiments, the NANOS3 gene is modified using a CRISPR-Cas system. An exemplary use of a CRISPR-Cas system to generate a NANOS3 deficient cell and organisms is shown in the Working Examples herein. In general, a CRISPR-Cas or CRISPR system as used in herein and in documents, such as WO 2014/093622, refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “guide RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008. CRISPR-Cas systems can be used to edit one or more nucleotides, remove one or more nucleotides, and/or delete one or more nucleotides.

Any suitable CRISPR-Cas system can be used in the context of the present disclosure to modify a NANOS3 polynucleotide in a host cell or any target polynucleotide of a donor cell. In some embodiments, the CRISPR-Cas system is a Class 2 system.

### Class 1 Systems

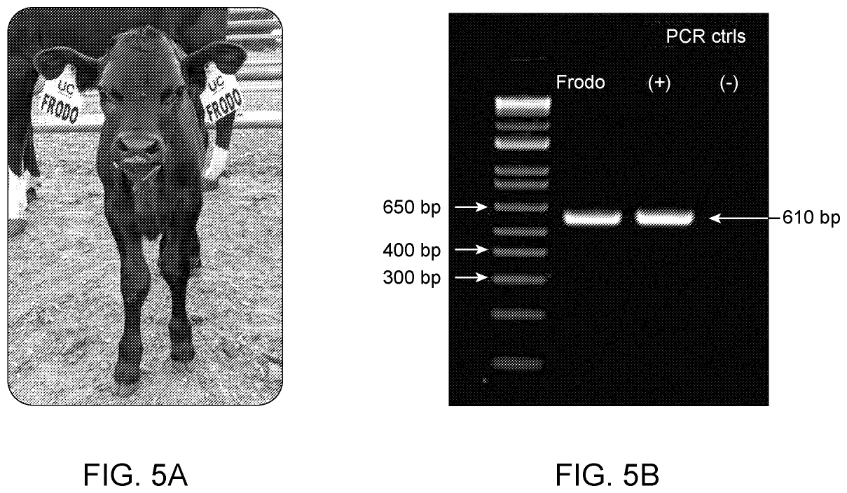

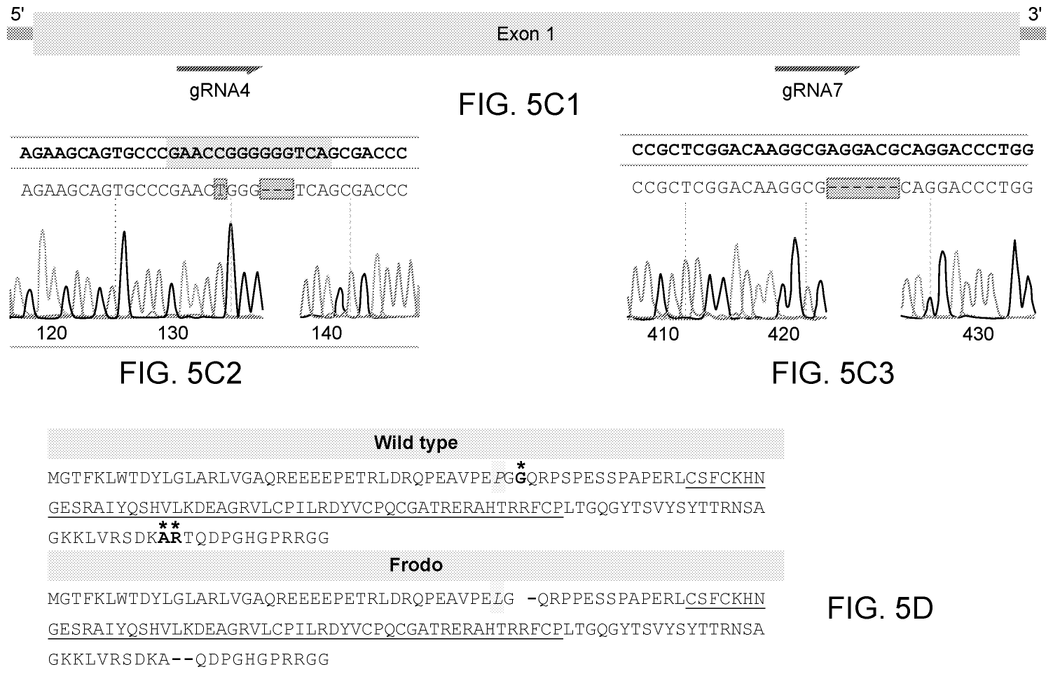

In some embodiments, the CRISPR-Cas system is a Class 1 CRISPR-Cas system. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326; Koonin E V, Makarova K S. 2019 Origins and evolution of CRISPR-Cas systems. Phil. Trans. R. Soc. B 374: 20180087, DOI: 10.1098/rstb.2018.0087, particularly at FIGS. 1 and 2. In some embodiments, the Class 1 CRISPR-Cas system is a subtype Type I-A, I—B, I—C, I-U, I-D, I-E, and I-F, Type IV-A and IV-B, and Type III-A, III-D, III-C, and III-B system. In some embodiments, the Class 1 CRISPR-Cas system is a variant system, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

In some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system is a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. In some embodiments, the CRISPR-Cas system is a Type II subtype, such as II-A, II-B, II-C1, or II-C2 system. In some embodiments, the Type II CRISPR-Cas system is a Cas9 system. In some embodiments, the CRISPR-Cas system is a Type V subtype, such as V-A, V-B1, V-B2, V-C, V-D, V-E, V-F1, V-F1(V-U3), V-F2, V-F3, V-G, V-H, V-I, V-K (V-U5), V-U1, V-U2, or V-U4 system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ. In some embodiments, the CRISPR-Cas system is a Type VI subtype, such as a VI-A, VI-B1, VI-B2, VI-C, or VI-D system. In some embodiments, the Type VI CRISPR-Cas system includes a Cas13a (C2c2), Cas13b (Group 29/30), Cas13c, and/or Cas13d.

### Guide RNAs

The CRISPR-Cas system described herein includes one or more guide RNAs (also referred interchangeably herein as “guide molecules” “guide polynucleotides” and “guide sequences”). The terms guide molecule, guide sequence and guide polynucleotide refer to polynucleotides capable of guiding Cas to a target genomic locus and are used interchangeably as in foregoing cited documents such as International Patent Publication No. WO 2014/093622. In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide. The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

The guide molecules can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. Target sequences are further discussed below.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sea sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments, the guide RNA (capable of guiding Cas to a target locus) can include (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and within the spirit and scope of this disclosure. Various modifications may be used to increase the specificity of binding to the target sequence and/or increase the activity of the Cas protein and/or reduce off-target effects. Example guide sequence modifications are described in International Patent Application WO2020033601, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or including the target sequence. Likewise, a “target polynucleotide” as used in this context herein refers to a polynucleotide sequence being or including the target sequence for a guide polynucleotide. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide can be DNA. The target polynucleotide can be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins.

Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including | |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

Sequences are provided using IUPAC nucleotide single letter codes

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programming of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. 2014 Nat Biotechnol. 2014 December; 32(12):1262-7 created a pool of sgRNAs, tiling across all possible target sites of a panel of six endogenous mouse and three endogenous human genes and quantitatively assessed their ability to produce null alleles of their target gene by antibody staining and flow cytometry. Doench et al. can demonstrate that optimization of the PAM improved activity and also provided an on-line tool for designing sgRNAs. Such approaches can be adapted for use with the present disclosure.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Nuclear Targeting and Transportation Sequences

For modification of nuclear located polynucleotides including, but not limited to, genomic DNA one or more components of the CRISPR-Cas system can include one or more sequences or signals for nucleus targeting and/or transportation. Although these are discussed with specific reference to CRISPR-Cas systems, such sequences and signals can be applied to other genetic modification systems or components thereof discussed elsewhere herein.

Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 12) or PKKKRKVEAS (SEQ ID NO: 13); the NLS from nucleoplasmin (e.g., the nucleoplasmin bipartite NLS with the sequence KRPAATKKAGQAKKKK (SEQ ID NO: 14)); the c-myc NLS having the amino acid sequence PAAKRVKLD (SEQ ID NO: 15) or RQRRNELKRSP (SEQ ID NO: 16); the hRNPA1 M9 NLS having the sequence NQSSNFGPMKGGNFGGRSSGPYGGGGQYFAKPRNQGGY (SEQ ID NO: 17); the sequence RMRIZFKNKGKDTAELRRRRVEVSVELRKAKKDEQILKRRNV (SEQ ID NO: 18) of the IBB domain from importin-alpha; the sequences VSRKRPRP (SEQ ID NO: 19) and PPKKARED (SEQ ID NO: 20) of the myoma T protein; the sequence PQPKKKPL (SEQ ID NO: 21) of human p53; the sequence SALIKKKKKMAP (SEQ ID NO: 22) of mouse c-abl IV; the sequences DRLRR (SEQ ID NO: 23) and PKQKKRK (SEQ ID NO: 24) of the influenza virus NS1; the sequence RKLKKKIKKL (SEQ ID NO: 25) of the Hepatitis virus delta antigen; the sequence REKKKFLKRR (SEQ ID NO: 26) of the mouse Mxl protein; the sequence KRKGDEVDGVDEVAKKKSKK (SEQ ID NO: 27) of the human poly(ADP-ribose) polymerase; and the sequence RKCLQAGMNLEARKTKK (SEQ ID NO: 28) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In CRISPR-Cas systems including a deaminase, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In some embodiments, a component of the CRISPR-Cas system includes a one or more nuclear export signals (NES), one or more one or more nuclear localization signals (NLS), or any combinations thereof. In some cases, the NES may be an HIV Rev NES. In certain cases, the NES may be MAPK NES. When the component is a protein, the NES or NLS may be at the C terminus of component. In some embodiments, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Donor Templates

In some embodiments the CRISPR-Cas system includes a donor nucleic acid such as a donor template, e.g., a recombination template, as discussed elsewhere in this disclosure. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some embodiments, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149). The strategy and techniques Of Suzuki et al. can be adapted for use with the present disclosure.

### Specialized Cas-Based Systems

Dead Cas (dCas) Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423, which can be adapted for use with the present disclosure.

### Split-Cas Systems

In some embodiments, the CRISPR-Cas system is a split CRISPR-Cas system. See e.g., Zetche et al., 2015. Nat. Biotechnol. 33(2): 139-142 and International Patent Publication WO2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wildtype Cas allows other methods of delivery of the systems to the cells, such as the use of cell penetrating peptides as described herein.

### DNA and RNA Base Editing Systems

In some embodiments, a polynucleotide of the present disclosure described elsewhere herein is modified using a base editing system. For example, in some embodiments, a genomic edit is made using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471, which can be adapted for use with the present disclosure.

Other Example Type V base editing systems are described in International Patent Publication Nos. WO2018/213708, WO2018/213726, WO2019126709, WO2019126716, and WO2019126762, each of which is incorporated herein by reference and can be adapted for use with the present disclosure.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO2019/005886, and WO2019/071048, WO2019126709, which are incorporated herein by reference and can be adapted for use with the present disclosure. An example FnCas9 system that may be adapted for RNA base editing purposes is described in International Patent Publication No. WO2016/106236, which is incorporated herein by reference and can be adapted for use with the present disclosure.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference and can be adapted for use with the present disclosure.

### Prime Editor Systems

In some embodiments, a polynucleotide of the present disclosure described elsewhere herein is modified using a prime editing system. See e.g., Anzalone et al. 2019. Nature. 576: 149-157. For example, in some embodiments, a genomic edit is made using a prime editing system. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

In some embodiments, a variant prime editing system is used to modify a polynucleotide of the present disclosure. In some embodiments, the variant prime editing system is a system for programmable addition via site-specific targeting elements (PASTE), such as a system described in Yarnall et al., Nature Biotechnology (2022). https://doi.org/10.1038/s41587-022-01527-4.

### CRISPR Associated Transposase (CAST) Systems

In some embodiments, a polynucleotide of the present disclosure described elsewhere herein can be modified using a CRISPR Associated Transposase (“CAST”) system. The CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference and can be adapted for use with the present disclosure.

### TALE Nucleases

In some instances, the site-directed nuclease is a TALE polypeptide. In some embodiments, a TALE nuclease or TALE nuclease system can be used to modify a polynucleotide, such as a NANOS3 gene or a polynucleotide in a donor cell in a complementation system described herein. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IJPAC single letter code for amino acids. A general representation of a TALE monomer which is comprised within the DNA binding domain is X₁₋₁₁—(X₁₂X₁₃)—X₁₄₋₃₃ or X₃₄ or X₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁—(X₁₂X₁₃)—X₄₋₃₃ or X₃₄ or X₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN, and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine, and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

In some embodiments, the TALEs can include N- and/or C-terminal capping regions, which can increase TALE polypeptide binding efficiency (see e.g., Zhang et al., Nature Biotechnology 29:149-153 (2011). Such “capping regions” can be directly N-terminal and/or C-terminal of the DNA binding region of a TALE. Exemplary amino acid sequence of a N-terminal capping region and C-terminal capping regions are generally known in the art.

As used herein, the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides described herein.

In some embodiments, the entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain an N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In some embodiments, the C-terminal capping region includes only or at least the 68 C-terminal amino acids, which enhance binding activity equal to the full- length capping region. See e.g., Zhang et al., Nature Biotechnology 29:149-153 (2011). In some embodiments, the C-terminal capping region includes only or at least the 20 C-terminal amino acids, which have about 50% or greater the efficacy of the full-length capping region. See e.g., Zhang et al., Nature Biotechnology 29:149-153 (2011).

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

A variety of additional TALEN-based systems have been described in the art, and modifications thereof are regularly reported; see, e.g., Boch, Science 326(5959):1509-12 (2009); Mak et al., Science 335(6069):716-9 (2012); and Moscou et al., Science 326(5959):1501 (2009). The use of TALENs based on the “Golden Gate” platform, or cloning scheme, has been described by multiple groups; see, e.g., Cermak et al., Nucleic Acids Res. 39(12):e82 (2011); Li et al., Nucleic Acids Res. 39(14):6315-25 (2011); Weber et al., PLoS One. 6(2):e16765 (2011); Wang et al., J Genet Genomics 41(6):339-47, Epub 2014 May 17 (2014); and Cermak T et al., Methods Mol Biol. 1239:133-59 (2015), any of which can be adapted for use with the present disclosure.

### Zinc Finger Nucleases

In some embodiments, the site-directed nuclease is a zinc finger protein. In some embodiments, a polynucleotide, such as a NANOS3 polynucleotide or a donor cell polynucleotide, is modified using a zinc finger system. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. These and any other ZFN systems can be used to modify the genome, such as the NANOS3 gene. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference and whose systems and methods can be adapted for use with the present disclosure to generate a NANOS3 deficient cell and/or organism.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96(6):2758-63 (1999); Dreier B et al., J Mol Biol. 303(4):489-502 (2000); Liu Q et al., J Biol Chem. 277(6):3850-6 (2002); Dreier et al., J Biol Chem 280(42):35588-97 (2005); and Dreier et al., J Biol Chem. 276(31):29466-78 (2001).

### Homing Endonucleases

In some embodiments, the genetic modifying system is or includes one or more homing endonucleases. Homing endonucleases (HEs) are sequence-specific endonucleases that have long recognition sequences (14-44 base pairs) and cleave DNA with high specificity-often at sites unique in the genome. There are at least six known families of HEs as classified by their structure, including GIY-YIG, His-Cis box, H-N-H, PD-(D/E)xK, and Vsr-like that are derived from a broad range of hosts, including eukaryotes, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24(8):663-80 (2014); Belfort and Bonocora, Methods Mol Biol. 1123:1-26 (2014); Hafez and Hausner, Genome 55(8):553-69 (2012); and references cited therein, which can be adapted for use with the present disclosure.

### Meganucleases and Hybrid Meganucleases

In some embodiments, the site-directed nuclease is a meganuclease or a hybrid mega nuclease. In some embodiments, a meganuclease, a hybrid mega nuclease, or system thereof can be used to modify a polynucleotide, such as a NANOS3 polynucleotide or donor cell polynucleotide. Meganucleases are endodeoxyribonucleases that are characterized by a large recognition site (double-stranded DNA sequences of 12 to 40 base pairs). Exemplary meganucleases and methods for using meganucleases can be found in U.S. Pat. Nos. 8,163,514, 8,133,697, 8,021,867, 8,119,361, 8,119,381, 8,124,369, and 8,129,134, which are specifically incorporated herein by reference. Such methods can be adapted for use to generate a NANOS3 deficient cell and/or organism.

Exemplary hybrid meganucleases include, without limitation, the MegaTal system and Tev-mTALEN systems, which use a fusion of TALE DNA binding domains and catalytically active HEs, taking advantage of both the tunable DNA binding and specificity of the TALE, as well as the cleavage sequence specificity of the HE; see, e.g., Boissel et al., NAR 42: 2591-2601 (2014); Kleinstiver et al., G3 4:1155-65 (2014); and Boissel and Scharenberg, Methods Mol. Biol. 1239: 171-96 (2015). Other exemplary hybrid meganucleases include, without limitation, the MegaTev system, which includes fusion of a meganuclease (Mega) with the nuclease domain derived from the GIY-YIG homing endonuclease I-TevI (Tev) where two active sites are positioned about 30 bp apart on a DNA substrate and generate two DSBs with non-compatible cohesive ends; see, e.g., Wolfs et al., NAR 42, 8816-29 (2014). RNAi

In certain embodiments, the genetic modification system is an interfering RNA (RNAi) system or agent (e.g., shRNA). As used herein, “gene silencing” or “gene silenced” in reference to an activity of an RNAi molecule or system, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA system or molecule, including but not limited to, siRNAi, shRNAi, endogenous microRNA, long non-coding RNA, and artificial microRNA. For instance, it includes sequences previously identified as siRNA, regardless of the mechanism of up- or down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

RNAi molecules can be delivered as the final active RNAi molecule or via a DNA polynucleotide or vector that encodes the RNAi molecule.

In some embodiments, the RNAi molecule or system targets a NANOS3 RNA molecule, such a NANOS3 mRNA. In some embodiments, the RNAi molecule or system produces an RNAi molecule that binds to and results in the degradation of a NANOS3 RNA and/or inhibition of translation of a NANOS3 mRNA. In some embodiments, the amount of NANOS3 RNA is reduced below detectable levels and/or reduces the amount of a NANOS3 protein so as to effectively eliminate the function of NANOS3. In some embodiments, organisms expressing a NANOS3 targeting RNAi system lack germ cells.

In some embodiments, the RNAi molecule or system targets one or more RNA molecules in a donor cell(s) to target a gene product of interest to generate a cell or organism having a desired phenotype. Exemplary genes whose expression can be modified by an RNAi system described herein in a donor cell are described in greater detail elsewhere herein.

### Transposon Systems

In some embodiments, the NANOS3 polynucleotide in a host cell or a target polynucleotide in a donor cell is modified using a transposon system. Exemplary transposons systems that can be utilized for modifying a polynucleotide are described herein and will be appreciated by those of ordinary skill in the art in view of this disclosure. In some embodiments, the transposon system is a Class I transposon system polypeptide. In some embodiments, the transposon system is a Class II transposon system polypeptide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Suitable Class I transposon systems include any of those in, without limitation, LTR and non-LTR retrotransposon systems. Exemplary Class I transposon systems include, without limitation, CRE, R2, R4, L1, RTE, Tad, R1, LOA, I, Jockey, CR1 polypeptides. See e.g., Proc Natl Acad Sci USA. 2006 Nov. 21; 103(47):17602-7; Eickbush T H et al., Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Suitable Class II transposon systems include any of those in, without limitation, the following transposon systems: Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof. In some embodiments, the Class II transposon system is a DD[E/D]transposon or transposon polypeptide. In some embodiments, the Class II transposon system is a Tel/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized in the context of the present invention include and are not limited to those described in e.g., Han et al., 2013. BMC Genomics. 14:71, doi: 10.1186/1471-2164-14-71, Lopez and Garcia-Perez. 2010. Curr. Genomics. 11(2):115-128; Wessler. 2006. PNAS. 103(47): 176000-17601; Gao et al., 2017. Marine Genomics. 34:67-77; Bradic et al. 2014. Mobile DNA. 5(12) doi:10.1186/1759-8753-5-12; Li et al., 2013. PNAS. 110(25)E2279-E2287; Kebriaei et al. 2017. Trends in Genetics. 33(11): 852-870); Miskey et al. 2003. Nucleic Acid res. 31(23):6873-6881; Nicolas et al. 2015. Microbiol Spectr. 3(4) doi: 10.1128/microbiolspec.MDNA3-0060-2014); W. S. Reznikoff. 1993. Annu Rev. Microbiol. 47:945-963; Rubin et al. 2001. Genetics. 158(3): 949-957; Wicker et al. 2003. Plant Physiol. 132(1): 52-63; Majumdar and Rio. 2015. Microbiol. Spectr. 3(2) doi: 10.1128/microbiolspec.MDNA3-0004-2014; D. Lisch. 2002. Trends in Plant Sci. 7(11): 498-504; Sinzelle et al. 2007. PNAS. 105(12): 4715-4720; Han et al. 2014; Genome Biol. Evol. 6(7):1748-1757; Grzebelus et al. 2006; Mol. Genet. Genomics. 275(5):450-459; Zhang et al. 2004. Genetics. 166(2):971-986; Chen and Li. 2008. Gene. 408(1-2):51-63; and C. Feschotte. 2004. Mol. Biol. Evol. 21(9):1769-1780.

### Recombinase Systems

In some embodiments, the genetic modification system to modify a genome is a recombinase system. Generally, recombinases are enzymes that catalyze site-specific recombination events, and recombination systems employ such enzymes to achieve site-specific polynucleotide integration or disruption. Many recombinase systems for gene knock-in, gene knock-out, and other genome or polynucleotide modifications are generally known in the art since their introduction several decades ago (see e.g., Sauer, B. Mol Cell Biol 7(6):2087-2096 (1987)) and can be used in the context of the present disclosure to introduce a transgene of the present disclosure and/or one or more components of another genetic modifying system described herein and/or generally known to a genome of a cell or another polynucleotide. Exemplary systems include without limitations, Cre-lox and FLP-FRT systems (see e.g., Maizels et al., J. Immunol. 2013. 161(1): doi:10.4049/jimmunol.1301241; Graham et al., Biotech J. 2009. 4(1):108-118; Chen et al. Animal. 4(5):767-771 (2010); Kalds et al. Front. Genet. 2019, doi.org/10.3389/fgene.2019.00750; Gurusinghe et al., J Cell Biochem. 2017. 118(5):1201-1215; and Wang et al., Plant Cell Rep (2011) 30:267-285), which are each incorporated by reference as if expressed in their entirety and can be adapted for use with the present disclosure.

### Delivery of Polynucleotides and Polypeptides

The gene modification system or component thereof (including any polynucleotides, vectors, vector systems, and/or the like) can be delivered to a cell or cell population using any suitable delivery composition, system, or technique.

### Physical Delivery

In some embodiments, the genetic modifying system or component thereof, may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded and purified if needed), and introduced to cells by a physical delivery method or technique.

### Microinjection

Microinjection of the genetic modifying system or component thereof directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids comprising coding sequences for Cas or other genetic modifying system effector proteins and/or any associated polynucleotides (e.g., guide RNAs, mRNAs, and/or guide RNAs), may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas or other effector protein-encoding mRNA to the cytoplasm, e.g., facilitating translation and shuttling of Cas or other effector protein to the cell nucleus.

Microinjection may be used to generate genetically modified animals. For example, gene modification systems or components thereof may be injected into zygotes, blastomeres, blastocysts, embryonic stem cells, pluripotent stem cells, induced pluripotent stem cells, primordial germ cells, primordial germ cell like-cells, and/or the like to allow for gene medication, such as germline modification.

Microinjection and nuclear transfer of bovine zygotes, blastocysts, and other cells has been described and used to generate genetically modified bovine. See e.g., Behboodi et al., J Dairy Sci. 1993 November; 76(11):3392-9; Bogliotti et al., J Vis Exp. 2016; (116): 54465; Galli et al., Cloning and Stem Cells. September 2002.189-196, doi.org/10.1089/15362300260339476; Krisher et al., Transgenic Research volume 3, pages 226-231 (1994); Yum et al., Scientific Reports volume 6, Article number: 27185 (2016); Krimpenfort et al., Biotechnology (N Y). 1991 September; 9(9):844-7. doi: 10.1038/nbt0991-844, Krishner et al., 1995. J Dairy Sci. 78:1282-1288; Otero et al., Indian J Sci Tech Vol 11(31), DOI: 10.17485/ijst/2018/v11i31/130839, August 2018; Kubisch et al., 1995. J Rep. Fert. 104:133-139, which are incorporated by reference herein as if expressed in their entireties and can be adapted for use with the present disclosure.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

Electroporation has been used to deliver exogenous polynucleotides and/or polypeptides to bovine zygotes. See e.g., Lin and Van Eenennaam. Front Genet. 2021; 12: 648482, doi.org/10.3389/fgene.2021.648482, particularly at supplementary table 1. In some embodiments, the voltage and number of pulses for delivery of an exogenous polynucleotide to a bovine cell, such as a zygote or blastocyst, via electroporation is 10-20 V/mm and 2-6 pulses, 10-20V/mm and 2-3 pulses, 15-20V/mm and 2-3 pulses, 15V/mm and 6 pulses See e.g., Tanihara, F., Hirata, M., Morikawa, S., Nguyen, N. T., Le, Q. A., Hirano, T., et al. (2019). The effects of electroporation on viability and quality of in vivo-derived bovine blastocysts. J. Reprod. Dev. 65, 475-479. doi: 10.1262/jrd.2019-049; Namula, Z., Wittayarat, M., Hirata, M., Hirano, T., Nguyen, N. T., Le, Q. A., et al. (2019). Genome mutation after the introduction of the gene editing by electroporation of Cas9 protein (GEEP) system into bovine putative zygotes. In Vitro Cell. Dev. An. 55, 598-603; Miao, D., Giassetti, M. I., Ciccarelli, M., Lopez-Biladeau, B., and Oatley, J. M. (2019). Simplified pipelines for genetic engineering of mammalian embryos by CRISPR-Cas9 electroporation dagger. Biol. Reprod. 101, 177-187; Ciccarelli, M., Giassetti, M. I., Miao, D., Oatley, M. J., Robbins, C., Lopez-Biladeau, B., et al. (2020). Donor-derived spermatogenesis following stem cell transplantation in sterile NANOS2 knockout males. Proc. Natl. Acad. Sci. U.S.A 117, 24195-24204; Camargo, L. S. A., Owen, J. R., Van Eenennaam, A. L., and Ross, P. J. (2020). Efficient one-step knockout by electroporation of ribonucleoproteins into zona-intact bovine embryos. Front. Genet. 11:570069; and Wei, J., Gaynor, P., Cole, S., Brophy, B., Oback, B., and Laible, G. (2018). “Developing the laboratory conditions for bovine zygote-mediated genome editing by electroporation” in Proceedings of the World Congress on Genetics Applied to Livestock Production, which are incorporated by reference herein and can be adapted for use with the present invention.

### Hydrodynamic Delivery

Hydrodynamic delivery may also be used for delivering the gene modification system, e.g., for in vivo delivery. In some examples, hydrodynamic delivery may be performed by rapidly pushing a large volume (8-10% body weight) solution containing the gene modification system into the bloodstream of a subject (e.g., a bovine). As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered genetic modification system or components may be enriched in ovaries and/or testis.

### Transfection

The cargos, e.g., nucleic acids and/or polypeptides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid. Nucleic acids and vectors and vector systems that can encode a genetic modifying system and/or components thereof are described in greater detail else wherein herein. Transfection has been used to deliver nucleic acid constructs to bovine cells. See e.g., Tajik et al., Iran J Vet Res. 2017 Spring; 18(2): 113-118; Jafarnejad et al., S African J Anim Sci, Vol. 48 No. 1 (2018) DOI: 10.4314/sajas.v48i1.13; Duarte et al., Anim Biotechnol. 2020 Dec. 30; 1-11. doi: 10.1080/10495398.2020.1862137; and Osorio Gene. 2017 Aug. 30; 626:200-208, which are incorporated by reference as if expressed in their entireties herein and can be adapted for use with the present disclosure.

### Transduction

The genetic modifying systems and/or components thereof, e.g., nucleic acids and/or polypeptides, can be introduced to cells by transduction by a viral, pseudoviral, and/or virus like particle. Methods of packaging the genetic modifying systems and/or components thereof in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral, pseudoviral, and/or virus like particle. After packaging in a viral, pseudoviral, and/or virus like particle. the viral particles can be exposed to cells (e.g., in vitro, ex vivo, or in vivo) where the viral, pseudoviral, and/or virus like particle infects the cell and delivers the cargo to the cell via transduction. Viral, pseudoviral, and/or virus like particles can be optionally concentrated prior to exposure to target cells. In some embodiments, the virus titer of a composition containing viral and/or pseudoviral particles can be obtained and a specific titer be used to transduce cells. Viral vectors and systems and generation of viral (or pseudoviral, and/or virus like particle) delivery particles is described in greater detail elsewhere herein. Viral transduction has been used to deliver exogenous nucleic acid constructs to bovine cells. See e.g., Hoffmann et al., Biology of Reproduction, Volume 71, Issue 2, 1 Aug. 2004, Pages 405-409, doi.org/10.1095/biolreprod.104.028472; Yu et al., (2014) Expression of Intracellular Interferon-Alpha Confers Antiviral Properties in Transfected Bovine Fetal Fibroblasts and Does Not Affect the Full Development of SCNT Embryos. PLoS ONE 9(7): e94444, doi.org/10.1371/journal.pone.0094444; and Wu et al., Scientific Reports volume 6, Article number: 28343 (2016), which are incorporated by reference as if expressed in their entireties herein and can be adapted for use with the present disclosure.

### Biolistics

The genetic modifying systems and/or components thereof, e.g., nucleic acids and/or polypeptides, can be introduced to cells using a biolistic method or technique. The term of art “biolistic”, as used herein refers to the delivery of nucleic acids to cells by high-speed particle bombardment. In some embodiments, the genetic modifying systems and/or components thereof can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a genetic modifying systems and/or components thereof described herein. Various implantable devices are described in the art, and include any device, graft, or other composition that can be implanted into a subject, such as a bovine.

### Delivery Vehicles

Polynucleotides and/or polypeptides of the present disclosure, such as a genetic modifying system, can be delivered (e.g., to a target cell to be modified) via one or more delivery vehicles. The delivery vehicles can deliver a cargo, such as a polynucleotide or polypeptide of the present disclosure (such as a genetic modifying system) into cells, tissues, organs, or organisms (e.g., animals or plants). In some embodiments, delivery vehicles are sued to deliver a cargo, such as a genetic modifying system or component thereof or other polynucleotide or polypeptide of the present disclosure to a target bovine cell. The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles, pseudoviral particles, or virus like particles), non-viral vehicles (e.g., exosomes, liposomes, etc.), and other delivery reagents described herein and those appreciated by one of ordinary skill in the art in view of the present disclosure.

The delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

### Particles

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to cells, as described in US20130185823, WO2008042156, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (e.g., one or more components of a genetic modifying system (e.g., a CRISPR-Cas system or component(s) thereof) and can include additional carriers and/or excipients) to provide particles of an optimal size for delivery for any in vitro, ex vivo and/or in vivo application of the present disclosure. In some embodiments, particle dimension (e.g., diameter) characterization is based on measurements using dynamic laser scattering (DLS). See also e.g., U.S. Pat. Nos. 8,709,843; 6,007,845; 5,855,913; 5,985,309; 5,543,158; and Dahlman et al. Nature Nanotechnology (2014), doi:10.1038/nnano.2014.84, describes particles, methods of making and using them, and measurements thereof which can be adapted for use with the present disclosure.

### Vectors and Vector Systems

In some embodiments the delivery vehicle is a vector or vector system or particle, such as a virus or viral like particle, produced from such a vector or vector system. As such, also provided herein are vectors that can contain one or more of the genetic modifying system polynucleotides described herein. In certain embodiments, the vector can contain one or more polynucleotides encoding one or more elements of a genetic modifying system described herein. The vectors can be useful in producing bacterial, fungal, yeast, plant cells, animal cells, and transgenic animals that can express one or more components of the genetic modifying system described herein, and as such, contain a genetic modification or be rendered capable of producing particles (e.g., viral or viral like particles) that can be used to deliver a genetic modifying system described herein to a cell, such as a bovine cell.

Within the scope of this disclosure are vectors containing one or more of the polynucleotide sequences described herein, such as those relevant to introducing a NANOS3 modification to generate a host cell or a modification to a donor cell polynucleotide. One or more of the polynucleotides that are part of a genetic modifying system can be included in a vector or vector system. The vectors and/or vector systems can be used, for example, to express one or more of the polynucleotides in a cell, such as a producer cell, to produce a genetic modifying system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the genetic modifying system described herein. In some embodiments, expression of elements of the genetic modifying system described herein can be driven by the CBh promoter or other ubiquitous promoter. Where the element of the genetic modifying system is an RNA, its expression can be driven by a Pol III promoter, such as a U6 promoter. In some embodiments, the two are combined.

### Cell-Based Vector Amplification and Expression

Vectors may be introduced and propagated in a prokaryotic cell or eukaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the genetic modifying system described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell. In some embodiments the host cell is a cell to be modified by a genetic modifying system. In some embodiments the host cell is a producer cell capable of producing particles (e.g., virus particles, virus like particles, exosomes, and/or the like) that can be used to deliver a genetic modifying system or component thereof to a cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990). In some embodiments, the suitable host cell is a bovine cell, including but not limited to, bovine embryonic stem cells, bovine induced pluripotent stem cells, bovine blastocyst cells, bovine spermatogonia stem cells, bovine oogonial cells, bovine primordial germ cells, bovine primordial germ cell like cells, bovine totipotent cells, or other bovine cell described elsewhere herein.

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is provided elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif (1990) 60-89).

In some embodiments, one or more vectors driving expression of one or more elements of a genetic modifying system described herein are introduced into a host cell such that expression of the elements of the delivery system described herein direct formation of a genetic modifying system complex (e.g., a CRISPR-Cas complex) at one or more target sites at on a target polynucleotide, such as in a target cell or target cell genome. For example, a CRISPR-Cas effector protein described herein and a nucleic acid component (e.g., a guide polynucleotide) can each be operably linked to separate regulatory elements on separate vectors. RNA(s) of different elements of a genetic modifying (e.g., CRISPR-Cas) system can be delivered to an animal, plant, microorganism or cell thereof to produce an animal (e.g., a mammal, such as a bovine)), that constitutively, inducibly, or conditionally expresses different elements of the genetic modifying (e.g., CRISPR-Cas) system described herein that incorporates one or more elements of the genetic modifying system (e.g., a CRISPR-Cas system) described herein or contains one or more cells that incorporates and/or expresses one or more elements of the genetic modifying (e.g., CRISPR-Cas) system described herein.

### Cell-Free Vector and Polynucleotide Expression

In some embodiments, the polynucleotide encoding one or more features of the genetic modifying system or other polynucleotide described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg²⁺, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus or other particle (e.g., viral like particle or exosome) produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the genetic modifying system polynucleotides described herein) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization or export signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the 3-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Exemplary promoters also include bovine U6 (bU6) and bovine 7SK (b7SK), and other bovine PolII promoters (see e.g., Lambeth et al., Anim Genet. 2006 August; 37(4):369-72), bovine papillomavirus-1 promoters (BPV-1) (Linz and Baker. J Virol. 1988 August; 62(8):2537-43. doi: 10.1128/JVI.62.8.2537-2543.1988), the bovine SIX1 gene promoter (see e.g., Wei et al. Scientific Reports volume 7, Article number: 12599 (2017)), bovine growth hormone promoter (see e.g., Jiang et al., Nuc Acid Prot Syn Mol Gen. 1999. 274(12): 7893-7900), bovine pyruvate carboxylase (see e.g., Hazelton et al. J. Dairy Sci. 91:91-99), a bidirectional promoter (see e.g., Meersserman et al. DNA Research, Volume 24, Issue 3, June 2017, Pages 221-233), a bovine Akt3 promoter (see e.g., Farmanullah et al. Journal of Genetic Engineering and Biotechnology (2021) 19:164), bovine alpha-lactalbumin promoter (see e.g., FEBS Lett. 1991 Jun. 17; 284(1):19-22), bovine beta-casein promoter (see e.g., Cerdan et al., Mol Reprod Dev. 1998 March; 49(3):236-45), any combination thereof.

In some embodiments, the regulatory sequence can be a regulatory sequence described in U.S. Pat. No. 7,776,321, U.S. Pat. Pub. No. 2011/0027239, or International Patent Publication No. WO 2011/028929, the contents of which are incorporated by reference herein in their entireties. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, P-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. As used herein, “regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer1l4), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. myostatin, Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more elements of the CRISPR-Cas system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments where multiple elements are to be expressed from the same vector or within the same vector system, different promoters or regulatory elements can be used for each element to be expressed to avoid or limit loss of expression due to competition between promoters and/or other regulatory elements.

In some embodiments, the polynucleotide, vector or system thereof can include one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 29) and others described elsewhere herein), endoplasmic reticulum localization/retention signals (e.g., KDEL (SEQ ID NO: 30), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria targeting signals (see e.g., Chin, R. M., et al, 2018, Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430), and peroxisome targeting signals (e.g. (S/A/C)-(K/R/H)-(L/A), SLK, (R/K)-(L/V/I)-XXXXX-(H/Q)-(L/A/F)). Suitable protein targeting motifs can also be designed or identified using any suitable database or prediction tool, including but not limited to Minimotif Miner (minimotifminer.org, mitominer.mrc-mbu.cam.ac.uk/release-4.0/embodiment.do?name=Protein%20MTS), LocDB (see above), PTSs predictor, TargetP-2.0 www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/). Selectable Markers and Tags

One or more of the polynucleotides described herein, such as those of or encoding a genetic modifying system and/or exogenous gene can be operably linked, fused to, or otherwise modified to include a polynucleotide that encodes or is a selectable marker or tag, which can be a polynucleotide or polypeptide. In some embodiments, the polypeptide encoding a polypeptide selectable marker is incorporated in the genetic modifying system polynucleotide or other polynucleotide of the present disclosure such that the selectable marker polypeptide, when translated, is inserted between two amino acids between the N- and C- terminus of the genetic modifying system polypeptide (or other polypeptide of the present disclosure) or at the N- and/or C-terminus of the genetic modifying system polypeptide (or other polypeptide of the present disclosure). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the genetic modifying system (or other polynucleotide) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the genetic modifying system (or other polypeptide) described herein via suitable linker, such as a glycine or glycine serine linkers as short as GS or GG up to (GGGGG)₃ (SEQ ID NO: 31) or (GGGGS)₃ (SEQ ID NO: 32). Other suitable linkers are described elsewhere herein.

### Targeting Moieties

The vector or vector system (or other polynucleotide) can include one or more polynucleotides that are or encode one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the genetic modifying system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated genetic modifying system polynucleotide(s) to specific cells, tissues, organs, etc. In some embodiments, the targeting moieties can target integrins on cell surfaces. Optionally, the binding affinity of the targeting moiety is in the range of 1 nM to 1 μM.

Exemplary targeting moieties that can be included are described elsewhere herein. See description related to “Targeted Delivery” and/or “Responsive Delivery” herein.

### Codon Optimization

As described elsewhere herein, the polynucleotide encoding one or more embodiments of the genetic modifying system or other polypeptides (such as those to be delivered to a target cell) of the present disclosure described herein can be codon optimized. In some embodiments, one or more polynucleotides contained in a vector (“vector polynucleotides”) described herein that are in addition to an optionally codon optimized polynucleotide encoding embodiments of the genetic modifying system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000,” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type, such as a bovine cell. In some embodiments, a codon optimized sequence is a sequence optimized for expression in a eukaryote, e.g., bovines (i.e., being optimized for expression in a bovine or bovine cell), or for another eukaryote, such as another animal (e.g., an ovine). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a mammal, including but not limited to human, or non-human eukaryote or animal or mammal as discussed herein, e.g., a bovine, ovine, camelid, and/or the like.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations, and components thereof for expression of one or more elements of a genetic modifying system or other polynucleotides described herein are as used in the foregoing documents, such as International Patent Publication No. WO 2014/093622 and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a genetic modifying system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the genetic modifying system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of the genetic modifying system, including but not limited to a Cas effector), is external to the capsid or virus particle. In the sense that it is not inside the capsid (enveloped or encompassed with the capsid) but is externally exposed so that it can contact the target genomic DNA. In some embodiments, the viral vector is configured such that all the cargo(s) are contained within the capsid after packaging.

### Split Viral Vector Systems

When the viral vector or vector system (be it a retroviral (e.g., AAV) or lentiviral vector) is designed so as to position the cargo(s) (e.g., one or more CRISPR-Cas system components) at the internal surface of the capsid once formed, the cargo(s) will fill most or all of internal volume of the capsid. In other embodiments, the genetic modifying effector (e.g., Cas) (or other exogenous gene or protein) may be modified or divided so as to occupy a less of the capsid internal volume. Accordingly, in certain embodiments, the genetic modifying system or component thereof (e.g., a Cas effector protein) or other exogenous gene or protein can be divided in two portions, which can be packaged in separate viral or viral like particles. In certain embodiments, by splitting the genetic modifying system or component thereof in two (or more) portions, space is made available to link one or more heterologous domains to one or both genetic modifying system component (e.g., Cas protein) or other protein portions. Such systems can be referred to as “split vector systems”. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the genetic modifying system (e.g., a CRISPR-Cas) system that can be achieved with a split system or split protein design.

Split CRISPR proteins or other exogenous proteins whose encoding polynucleotides can be incorporated into the viral or other vectors described herein are set forth elsewhere herein and in documents incorporated herein by reference in further detail herein. In certain embodiments, each part of a split protein is attached to a member of a specific binding pair, and when bound with each other, the members of the specific binding pair maintain the parts of the spit protein in proximity. In certain embodiments, each part of a split protein is associated with an inducible binding pair. An inducible binding pair is one which is capable of being switched “on” or “off” by a protein or small molecule that binds to both members of the inducible binding pair. In general, according to the invention, some proteins may preferably split between domains, leaving domains intact. Where the cargo is a Cas protein, non-limiting examples of such Cas proteins include, without limitation, Cas protein, and orthologues. Non-limiting examples of split points include, with reference to SpCas9: a split position between 202A/203S; a split position between 255F/256D; a split position between 310E/311I; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099. Corresponding positions in other Cas proteins can be appreciated in view of these positions made with reference to SpCas9.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the delivery of a cargo (e.g., a genetic modifying systems or other exogenous polynucleotide) can include, but are not limited to, those vectors based upon murine leukemia virus (MuLV), gibbon ape leukemia virus (GaLV), Simian immunodeficiency virus (SIV), human immunodeficiency virus (HIV), equine infections anemia (EIA), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); WO1994026877). Other exemplary retroviral vectors are described elsewhere herein.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Exemplary lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. See e.g., Balagaan, J Gene Med 2006; 8: 275-285; Binley et al., HUMAN GENE THERAPY 23:980-991 (September 2012)), which can be modified for use with the present disclosure.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to deliver a genetic modifying system or exogenous polynucleotide of the present disclosure.

In some embodiments, the pseudotype and infectivity or tropism of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859).

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envelope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Various exemplary lentiviral vectors, such as those used in the treatment of Parkinson's disease, ocular diseases, delivery to the brain, are described in e.g., US Patent Publication No. 20120295960, 20060281180, 20090007284, US20110117189; US20090017543; US20070054961, US20100317109, US20110293571; US20110293571, US20040013648, US20070025970, US20090111106, and US Patent Nos. U.S. Pat. Nos. 7,259,015, 7,303,910 and 7,351,585. Any of these systems can be used or adapted to deliver a genetic modifying system polynucleotide or other exogenous polynucleotide of the present disclosure.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, the viral vector is a Cocal vesiculovirus envelope pseudotyped retroviral or lentiviral vector particles are contemplated (see, e.g., US Patent Publication No. 20120164118). Cocal virus is in the Vesiculovirus genus and is a causative agent of vesicular stomatitis in mammals, and as such vectors based on this virus can be used to deliver cells to a wide variety of animals, including insects, cattle, and horses (see e.g., Jonkers et al., Am. J. Vet. Res. 25:236-242 (1964) and Travassos da Rosa et al., Am. J. Tropical Med. & Hygiene 33:999-1006 (1984)). In some embodiments, Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be any suitable serotype, such as serotype 2, 5, 8, 9, and others. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more exogenous polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the CRISPR-Cas system polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use to deliver a polynucleotide or system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the CRISPR-Cas system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the CRISPR-Cas system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. In some embodiments, utilizing homologs of the Cas effector protein that are shorter can be utilized, such for example those in Table 2.

TABLE 2
Exemplary shorter Cas effector homologs.

| Species | Cas9 Size (nt) |
|---|---|
| Corynebacter diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Campylobacter jejuni | 2952 |
| Streptococcus thermophilus LMD-9 | 3396 |

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors.

The AAV vector or system thereof can be configured to produce AAV particles having a specific serotype.

AAV particles, packaging polynucleotides encoding compositions of the present disclosure may comprise or be derived from any natural or recombinant AAV serotype. According to the present disclosure, the AAV particles may utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-lb, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK 11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments s, the AAV serotype may be, or have, a mutation in the AAV9 sequence as described by N Pulicherla et al. (Molecular Therapy 19(6):1070-1078 (2011)), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the serotype may be AAVDJ or a variant thereof, such as AAVDJ8 (or AAV-DJ8), as described by Grimm et al. (Journal of Virology 82(12): 5887-5911 (2008). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772 may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence as described in International Publication No. WO2015121501, such as, but not limited to, true type AAV (ttAAV) (SEQ ID NO: 2 of WO2015121501), “UPenn AAV10” (SEQ ID NO: 8 of WO2015/121501), “Japanese AAV10” (SEQ ID NO: 9 of WO2015/121501), or variants thereof.

According to the present disclosure, AAV capsid serotype selection or use may be from a variety of species. In one example, the AAV may be an avian AAV (AAAV). The AAAV serotype may be, or have, a sequence as described in U.S. Pat. No. 9,238,800, such as, but not limited to, AAAV (SEQ ID NO: 1, 2, 4, 6, 8, 10, 12, and 14 of U.S. Pat. No. 9,238,800), or variants thereof.

In one example, the AAV may be a bovine AAV (BAAV). The BAAV serotype may be, or have, a sequence as described in U.S. Pat. No. 9,193,769, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, such as, but not limited to, BAAV (SEQ ID NO: 5 and 6 of U.S. Pat. No. 7,427,396), or variants thereof.

In one example, the AAV may be a caprine AAV. The caprine AAV serotype may be, or have, a sequence as described in U.S. Pat. No. 7,427,396, such as, but not limited to, caprine AAV (SEQ ID NO: 3 of U.S. Pat. No. 7,427,396), or variants thereof.

In other examples the AAV may be engineered as a hybrid AAV from two or more parental serotypes. In one example, the AAV may be AAV2G9 which comprises sequences from AAV2 and AAV9. The AAV2G9 AAV serotype may be, or have, a sequence as described in United States Patent Publication No. US2016/0017005.

In one example, the AAV may be a serotype generated by the AAV9 capsid library with mutations in amino acids 390-627 (VP1 numbering) as described by Pulicherla et al. (Molecular Therapy 19(6):1070-1078 (2011). The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one example, the AAV may be a serotype including at least one AAV capsid CD8+ T-cell epitope. As a non-limiting example, the serotype may be AAV1, AAV2 or AAV8.

In one example, the AAV may be a variant, such as PHP.A or PHP.B as described in Deverman. 2016. Nature Biotechnology. 34(2): 204-209.

AAV vector serotypes can be matched to target cell types. For example, the following exemplary cell types can be transduced by the indicated AAV serotypes among others.

In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted, e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008) and in Table 3.

TABLE 3
Tissue/Cell Types and Exemplary Serotypes

| | |
|---|---|
| liver | AAV 3, AAV5, AAV8, AAV9 |
| Skeletal muscle | AAV1, AAV7, AAV6, AAV8, AAV9 |
| Central nervous system | AAV5, AAV1, AAV4, AAV9 |
| RPE | AAV5, AAV4 |
| Lung | AAV9 |
| Heart | AAV8 |
| Pancreas | AAV8 |
| Kidney | AAV2, AAV8 |
| Hematopoietic Stem Cells | AAV6 |

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the genetic modifying system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more components of a genetic modifying system or other exogenous polynucleotide to be delivered to a cell. Specific cassette configuration for delivery of a genetic modifying system and/or other exogenous polynucleotide(s) will be appreciated by one of ordinary skill in the art in view of the description herein.

In some embodiments, one or more components of a genetic modifying system or other polypeptides and/or polynucleotides are associated with Adeno Associated Virus (AAV), e.g., an AAV comprising a polypeptide of the genetic modification system or exogenous polypeptide as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid as described herein. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a Cas protein or other exogenous polypeptide. Likewise, these can be fusions, with the protein, e.g., large payload protein such as a Cas protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as with other aspects of the present disclosure, and through this description herein one knows now that large payload proteins can be fused to the AAV capsid. Accordingly, the approaches described herein are also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1.

In some embodiments, a genetic modifying system polypeptide or other exogenous polypeptide is external to the capsid or virus particle, such as an AAV capsid. Although this approach is discussed in the context of AAVs, such an approach is applicable to other viral systems or viral like particle systems where capsids are formed. In these embodiments, the cargo polypeptide is not inside the capsid (enveloped or encompassed with the capsid) but is externally exposed so that it can contact the target genomic or other target DNA or RNA). In some embodiments, the cargo polypeptide is associated with the AAV VP2 domain by way of a fusion protein. In some embodiments, the association may be considered to be a modification of the VP2 domain. In some embodiments, the AAV VP2 domain may be associated (or tethered) to the cargo polypeptide via a connector protein, for example using a system such as the streptavidin-biotin system. Also provided herein are polynucleotides encoding a cargo polypeptide (e.g., a genetic modifying polypeptide or other exogenous cargo polypeptide) and associated AAV VP2 domain. In some preferred embodiments, the cargo polypeptide is fused or tethered (e.g., via linker) to the VP2 domain so that, a non-naturally occurring modified AAV having a VP2-cargo polypeptide fusion or otherwise modified capsid protein is formed. In some embodiments, where the cargo is tethered via a linker, the cargo can be distanced from the remainder of the AAV (or other viral or viral like particle). The fusion or tether can be at the N-terminus, C-terminus, or both of the capsid polypeptide. In some embodiments, an NLS and/or a linker (such as a GlySer linker) or other tether is positioned between the C-terminal end of the cargo and the N-terminal end of the capsid domain. In some embodiments, an NLS and/or a linker (such as a GlySer linker) or other tether is positioned between the N-terminal end of the cargo and the C-terminal end of the capsid domain. In some embodiments, the capsid polypeptide that is modified with a cargo polypeptide is truncated or contains a loss of one or more internal amino acids with the N- and C-terminal amino acids (e.g., the first (or last) 2-10 amino acids of the capsid domain intact. In these embodiments, the cargo polypeptide can be inserted between the intact N- and/or C-terminal amino acids via a fusion (e.g., an in-frame fusion) or linker or other tether (such as a streptavidin/biotin system or other adaptor molecule such as MS2). In some embodiments where a linker is used, the linker can be a branched linker, which can allow for more distance between the cargo polypeptide and capsid. A cargo polypeptide can be incorporated into other capsid domains of the AAV (e.g., VP1 and/or VP3) in a similar fashion as described with respect to VP2. Likewise, similar approaches (e.g., fusion or tethered) can be used to modified non-AAV capsids of other viral and viral-like delivery systems described herein.

### Herpes Simplex Viral Vectors

In some embodiments, the vector is a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments the cargo polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the present disclosure.

### Poxvirus Vectors

In some embodiments, the vector can be a poxvirus vector or system thereof. In some embodiments, the poxvirus vector can result in cytoplasmic expression of one or more cargo polynucleotides of the present disclosure. In some embodiments the capacity of a poxvirus vector or system thereof can be about 25 kb or more. In some embodiments, a poxvirus vector or system thereof can include one or more cargo polynucleotides described herein.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a genetic modifying system polynucleotide or other polynucleotide of the present disclosure), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ or more particles/mL.

Lentiviruses may be prepared from any lentiviral vector or vector system described herein. In one example embodiment, after cloning a polynucleotide to be delivered into a suitable lentiviral vector (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

See also Merten et al., 2016. “Production of lentiviral vectors.” Mol. Ther. 3: 10617 for additional methods and techniques for lentiviral vector and particle production, which can be adapted for use with the present disclosure.

### AAV Particle Production

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In general, there are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the cargo polynucleotide to be packaged and delivered by the resulting AAV particle (e.g., the genetic modifying system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a cargo polynucleotide (e.g., the CRISPR-Cas system polynucleotide(s)) between 2 ITRs; (2) a vector that carries the AAV Rep-Cap encoding polynucleotides; and (helper polynucleotides). One of skill in the art will appreciate various methods and variations thereof that are both helper and -helper free and as well as the different advantages of each system. See also Kimur et al., 2019. Sci. Rep. 6:13601; Shin et al., Meth. Mol Biol. 2012. 798:267-284; Negrini et al., 2020. Curr. Prot. Neurosci. 93:e103; Dobrowsky et al., 2021. Curr. Op. Biomed. Eng. 20:100353 for additional methods and techniques for AAV vector and particle production, which can be adapted for use with the present disclosure.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating cargo polynucleotide(s) and delivering said cargo polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more polynucleotides of the present disclosure described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the cargo polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the cargo polynucleotide(s). In some embodiments, the naked polynucleotide can contain other nucleic acids and/or polynucleotides in addition to the cargo polynucleotide(s). The naked polynucleotides can include one or more elements of a transposon system. Transposons and system thereof are described in greater detail elsewhere herein.

### Non-Viral Polynucleotide Vectors

In some embodiments, one or more of the polynucleotides of the present disclosure can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more cargo polynucleotides) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the cargo polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the cargo polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the cargo polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include without limitation Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Non-Vector Delivery Vehicles

The delivery vehicles may comprise non-vector vehicles. In general, methods and vehicles capable of delivering nucleic acids and/or proteins may be used for delivering the systems compositions herein. Examples of non-vector vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles can include or be composed of lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Lipid nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of a cargo polypeptide) and/or RNA molecules (e.g., mRNA of encoding a cargo polypeptide and/or other RNA cargos such as gRNAs). In certain cases, LNPs may be use for delivering RNP complexes of e.g., Cas/gRNA.

Components in LNPs may comprise cationic lipids 1,2-dilineoyl-3-dimethylammonium-propane (DLinDAP), 1,2-dilinoleyloxy-3-N,N- dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing cargo polypeptides or polynucleotides. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037.; Altmoğlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing cargo polypeptide(s) and/or polynucleotide(s). In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., cshprotocols.cshlp.org/content/2010/4/pdb.prot5407.long, the teachings of which can be applied and/or adapted to generated and/or deliver the genetic modifying systems and/or other cargo polypeptides or polynucleotides described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US 20160129120; US 20160244761; US 20120251618; WO 2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles contain or are composed entirely of stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the cargos described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles contain or be composed entirely of lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Østergaard et al., Bioconjugate Chem., 2015, 26 (8), pp 1451-1455.

### Cell Penetrating Peptides

In some embodiments, the delivery vehicles contain or are composed entirely of cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles contain or are composed entirely of DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles contain or are composed entirely of metal nanoparticles. In some embodiments, the delivery vehicles contain or are composed entirely of gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal nanoparticles include, without limitation, tungsten, palladium, rhodium, platinum, and iridium particles. Other non-limiting, exemplary metal nanoparticles suitable for delivery vehicles are described in US 20100129793.

iTOP

In some embodiments, the delivery vehicles contain or are composed entirely of iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles contain or are composed entirely of polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642. Other exemplary and non-limiting polymeric particles suitable for delivery vehicles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles can contain or be composed entirely of streptolysin 0 (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles can contain or be composed entirely of multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles can contain or be composed entirely of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles can contain or be composed entirely of inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles can contain or be composed entirely of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome forms a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29: 341; El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

Spherical nucleic acids (SNA) are three-dimensional arrangements of nucleic acids, with densely packed and radially arranged oligonucleotides on a central nanoparticle core. In its simplest form the SNA is composed of oligonucleotides and a core. In some embodiments, the delivery vehicle can contain or be composed entirely of SNAs. SNAs are three dimensional nanostructures that can be composed of densely functionalized and highly oriented nucleic acids that can be covalently attached to the surface of spherical nanoparticle cores. The core may be a hollow core which is produced by a 3-dimensional arrangement of molecules which form the outer boundary of the core. For instance, the molecules may be in the form of a lipid layer or bilayer which has a hollow center. In other embodiments, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

The oligonucleotides to be delivered can be associated with the core of an SNP. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors, or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, oligonucleotides are attached to nanoparticles by terminating the polynucleotide with a 5′ or 3′ thionucleoside. In other instances, an aging process is used to attach oligonucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123. In some embodiments, the core is a metal core. In some embodiments, the core is an inorganic metal core. In some embodiments, the core is a gold core.

In some instances, the oligonucleotide is attached or inserted in the SNA. A spacer sequence can be included between the attachment site and the oligonucleotide. In some embodiments, a spacer sequence comprises or consists of an oligonucleotide, a peptide, a polymer or an oligoethylene glycol. In a preferred embodiment, the spacer is oligoethylene glycol and more preferably, hexaethyleneglycol.

Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., U.S. Pat. App. Pub. US20210002640 and US20200188521.

### Self-Assembling Nanoparticles

In some embodiments, the delivery vehicle contains or is composed entirely of a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle contains or is composed entirely of supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle is configured for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as on its surface. Exemplary targeting moieties include, without limitation, small molecule, polypeptide, and/or polynucleotide ligands for cell surface molecules, antibodies, affibodies, aptamers, or any combination thereof. In some embodiments, a targeted delivery vehicle can be generated by coupling, conjugating, attaching, or otherwise associating a targeting moiety with a delivery vehicle described elsewhere herein. In some embodiments, multiple targeting moieties with different targets are coupled to a delivery vehicle. In some embodiments a multivalent approach can be employed. Multivalent presentation of targeting moieties (e.g., antibodies) can also increase the uptake and signaling properties of targeting moiety fragments. In some embodiments, targeted delivery can be to one cell type or to multiple cell types. Methods of coupling conjugating, attaching, or otherwise associating a targeting moiety with a delivery vehicle are generally known in the art.

In some embodiments, the targeting moiety is an aptamer. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets.

Targeted delivery includes intracellular delivery. Delivery vehicles that utilize the endocytic pathway are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH can be included in the delivery vehicle. Such lipids or peptides can include amines, which are protonated at an acidic pH and cause endosomal swelling and rupture by a buffer effect, pore-forming protein listeriolysin O, histidine-rich peptides have the ability to fuse with the endosomal membrane, resulting in pore formation, and can buffer the proton pump causing membrane lysis, and/or unsaturated dioleoylphosphatidylethanolamine (DOPE) that readily adopt an inverted hexagonal shape at a low pH and causes fusion of liposomes to the endosomal membrane. Inclusion of such molecules can result in an efficient endosomal release and/or may provide an endosomal escape mechanism to increase cargo delivery by the vehicle.

In some embodiments, the delivery vehicle is or includes modified CPP(s) that can facilitate intracellular delivery via macropinocytosis followed by endosomal escape. CPPs are described in greater detail elsewhere herein.

In some embodiments, targeted delivery is organelle-specific targeted delivery. A delivery vehicle can be surface-functionalized with a targeting moiety that can direct organelle specific delivery, such as a nuclear localization sequence, ribosomal entry sequence, mitochondria specific moiety, and/or the like. The invention further comprehends a lipid entity of the invention targeting the nucleus, e.g., via a DNA-intercalating moiety.

In some embodiments, the targeted delivery is multifunctional targeted delivery that can be accomplished by attaching more than one targeting moiety to the surface of the delivery vehicle. In some embodiments, such an enhances accumulation in a desired site and/or promotes organelle-specific delivery and/or target a particular type of cell and/or respond to the local environmental stimuli such as temperature (e.g., elevated), pH (e.g., acidic or basic), respond to targeted or localized externally applied stimuli such as a magnetic field, light, energy, heat or ultrasound (e.g., responsive delivery, which is described in greater detail elsewhere herein) and/or promote intracellular delivery of the cargo.

Exemplary targeting moieties are generally known in the art, and include without limitation, those described in e.g., in e.g., Deshpande et al, “Current trends in the use of liposomes for tumor targeting,” Nanomedicine (Lond). 8(9), doi:10.2217/nnm.13.118 (2013), International Patent Publication No. WO 2016/027264, Lorenzer et al, “Going beyond the liver: Progress and challenges of targeted delivery of siRNA therapeutics,” Journal of Controlled Release, 203: 1-15 (2015); Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), Zhao et al., 2020. Cell 181:151-167, particularly at Tables 1-5; Liu et al., Front. Bioeng. Biotechnol. 2021. 9:701504. doi: 10.3389/fbioe.2021.701504; US20210379192 (describes exemplary skeletal muscle cell targeting moieties), Snow-Lisy et al., Drug. Deliv. Transl. Res. 1:351(2011); US20060263336 (describes exemplary progenitor cell targeting moieties) each of which and the documents cited therein are hereby incorporated herein by reference.

Other exemplary targeting moieties are described elsewhere herein, such as epitope tags, reporter and selectable markers, and/or the like which can be configured for and/or operate in some embodiments as targeting moieties.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, a targeting moiety is responsive to an external stimuli and facilitate responsive delivery. In other embodiments, responsiveness is determined by a non-targeting moiety component of the delivery vehicle.

In some embodiments, the responsive delivery is stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

In some embodiments, the responsive delivery is temperature-triggered delivery. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

In some embodiments, the responsive delivery is redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria, and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 33)) can be incorporated into a linker, and can have antibody targeting, e.g., antibody 2C5.

In some embodiments, the responsive delivery is light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Responsive delivery to the testis has been described. See e.g., He et al., 2015. Oncol. Rep. 34(5)-2318 (describes ultrasound microbubble-mediated delivery to the testis); Li et al., Curr. Drug. Deliv. 2020 17(5):438-446 (describes heat stress and pulsed unfocused ultrasound delivery into testicular seminiferous tubules), which can be adapted for use with the present disclosure to provide responsive delivery to the testis or testicular cell.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Guide RNA (gRNA) Design and In Vitro Testing for Bovine NANOS3 Gene Knockout

NANOS3 is known to be critical for normal germline development in several organisms, but the role has not yet been reported for male bovine germ cells (Tsuda et al., 2003, Julaton and Reijo Pera, 2011, Ideta et al., 2016). Bovine NANOS3 is a 2,633 bp gene with two exons (FIG. 1A). The larger exon 1 (451 bp) was targeted as it contains the coding region for the critical zinc finger binding domain (Suzuki et al., 2014).

Guide RNAs (gRNAs) targeting the bovine NANOS3 exon 1 were designed and screened for potential off-target sites based on the bovine reference genome using online bioinformatic tools, sgRNA Scorer 2.0 (Chari et al., 2017) and Cas-OFFinder (Bae et al., 2014), respectively. Based on a systematic analysis of CRISPR-Cas9 mismatch tolerance (Anderson et al., 2015) and testing in bovine zygotes (Hennig et al., 2020) only gRNAs that met specific mismatch parameters were selected for testing. A mismatch is defined as a discrepancy between a base of the gRNA and the predicted off-target site. The gRNA selection criterion was 1) at least 3 total mismatches and 2) at least 1 of the mismatched bases was located in the seed region (8-10 bp proximal to the PAM site) of the gRNA. Based on this criterion, we selected 7 gRNAs for testing in the lab.

First, the gRNAs were tested using an in vitro cleavage assay. Each gRNA was incubated with Cas9 protein and polymerase chain reaction (PCR) amplified genomic NANOS3 bovine DNA in a buffer for 1 hour at 37° C. and ran the resulting product on a 2% agarose gel. It was observed that 4 of the gRNAs (gRNA #1, #4, #5, #7) successfully cut the target region in vitro. Next, the four successful gRNAs were further tested in vivo to determine blastocyst development rate and mutation efficiency of each gRNA. Guide sequences are shown in Table 4. FIGS. 2-3 diagram the target positions of the gRNAs within exon 1 of NANOS3.

TABLE 4

| Guide Sequence | Sequence | SEQ ID |
|---|---|---|
| Reference | (20 nt gRNA + PAM) | NO: |
| 1 (gRNA1) | GTGGACAGACTACTTGGGTTTGG | 34 |
| 2 (gRNA2) | CTACTTGGGTTTGGCACGCCTGG | 35 |
| 3 (gRNA3) | GTTCGGGCACTGCTTCTGGCTGG | 36 |
| 4 (gRNA4) | AGAAGCAGTGCCCGAACCGGGGG | 37 |
| 5 (gRNA5) | CGCTTCATCCTTGAGCACGTGGG | 38 |
| 6 (gRNA6) | TGGTCCGCTCGGACAAGGCGAGG | 39 |
| 7 (gRNA7) | CTCGGACAAGGCGAGGACGCAGG | 40 |

### Example 2—In Vivo Guide RNA testing for Bovine NANOS3 Gene Knockout

### Bovine Embryo Production

In order to produce embryos for in vivo testing, bovine ovaries were collected from a local slaughterhouse and transported to the laboratory at 35-37° C. in sterile saline (Hennig et al., 2020, Owen et al., 2020b). Cumulus-oocyte-complexes (COCs) were aspirated from follicles and groups of 50 COCs were transferred to 4-well dishes containing 400 μL of maturation media (IVF Bioscience, Falmouth, United Kingdom). COCs were incubated for 18-22 hr at 38.5° C. in a humidified 5% CO₂ incubator. Approximately 25 COCs per drop were fertilized in 60 uL drops of SOF-IVF media (Bakhtari and Ross, 2014) with 2×10⁶ sperm per mL and incubated for 6 hr at 38.5° C. in a humidified 5% CO₂ incubator (Hennig et al., 2020, Owen et al., 2020b). Six hours post insemination (hpi), presumptive zygotes were denuded by light vortex in SOF-HEPES medium for 5 min (Bakhtari and Ross, 2014, Hennig et al., 2020, Owen et al., 2020b). Zygotes were incubated in 50 uL drops (n=25 per drop) or in 400 μL wells (n=50-200 per well) of culture media (IVF Bioscience, Falmouth, United Kingdom) at 38.5° C. in a humidified atmosphere of 5% CO₂, 5% O₂, and 90% N₂ for 7 days (Bakhtari and Ross, 2014, Hennig et al., 2020, Owen et al., 2020b).

In Vivo Validation of Single gRNAs (sgRNAs) for Bovine NANOS3 Knockout

To determine the mutation rate for each guide, presumptive zygotes (6 hours post insemination (hpi)) were microinjected (see e.g., laser-assisted cytoplasmic injection (Bogliotti et al., 2016)) with 6 μL of a solution containing 67 ng/μL of a gRNA (Synthego, Menlo Park, CA) alongside 167 ng/μL of Cas9 protein (PNA Bio, Inc., Newbury Park, CA), which had been incubated together at room temperature for 30 minutes prior to microinjection. A group of 30 embryos were microinjected for each single gRNA (sgRNA) tested and control non-microinjected groups of embryos (n=120) were also included in each trial (Table 5). Microinjected embryos were incubated for 7-8 days.

To determine blastocyst development rate, day 7 embryos were scored for developmental stage reached. All microinjected groups resulted in a blastocyst development rate (≥20%; Table 5), which was similar to previous microinjected bovine embryo experiments (Hennig et al., 2020, Owen et al., 2020b).

For mutation analysis, all microinjected embryos and 10 randomly selected control non-microinjected embryos that reached blastocyst stage were individually collected for DNA extraction. The target region was amplified by two rounds of nested polymerase chain reaction (PCR) using primers (Eurofins Genomics, Louisville, KY) developed using an online bioinformatic tool, Primer3 (Untergasser et al., 2012). PCR products were visualized on a 1% agarose gel, purified using the QIAquick Gel Extraction Kit (Qiagen, Inc., Valencia, CA) and Sanger sequenced (GeneWiz, South Plainfield, NJ). Three of the four sgRNAs (#4, #5 and #7) resulted in an over 60% total mutation rate (Table 5).

TABLE 5
Comparison of blastocyst rates and mutation efficiencies of tested NANOS3 gRNAs.

| | Location | Blastocyst | | Total | | Knockout | > 5% wt | |
|---|---|---|---|---|---|---|---|---|
| gRNA | (Exon 1) | Rate | | Mutation | | (0% wt) | Mono-allelic | Mosaic |
| Control | — | 42/120 | (35%) | 0/10 | (0%) | — | — | — |
| 1 | 5′ | 7/30 | (23%) | 1/7 | (14%) | 0/7 (0%) | 0/7 (0%) | 1/7 (14%) |
| 4 | 5′ | 7/30 | (23%) | 5/6 | (83%) | 4/6 (67%) | 0/6 (0%) | 1/6 (17%) |
| 5 | Center | 6/30 | (20%) | 5/6 | (83%) | 2/6 (33%) | 0/6 (0%) | 3/6 (50%) |
| 7 | 3′ | 8/30 | (27%) | 5/8 | (63%) | 2/8 (25%) | 2/8 (25%) | 1/8 (13%) |

In Vivo Validation of Dual gRNAs (dgRNAs) for Bovine NANOS3 Knockout

After sgRNA testing, we tested a dual gRNA system, which has been shown to be an efficient method for complete gene disruption, or knockout, in livestock species (Vilarino et al., 2017, Wu et al., 2017). To determine the dgRNA knockout efficiency for bovine NANOS3, presumptive zygotes (6 hpi) were microinjected as described before with 6 μL of a solution containing 67 ng/μL of sgRNA #4 and 67 ng/μL of sgRNA #7 (Synthego, Menlo Park, CA). SgRNA #4 and #7 were selected based on their high individual mutation efficiencies (Table 5) and genomic location, so that when microinjected together they would introduce a large deletion of 297 bp (FIG. 3A-3D). An added benefit of using a dgRNA system, is that it allows initial evaluation of mutation efficiency by gel electrophoresis of the PCR products without the need for Sanger sequencing (Vilarino et al., 2017, Wu et al., 2017). The dgRNA_4+7 microinjections resulted in a similar blastocyst development rate (19%) and >75% knockout (i.e., 0 wild-type alleles) rate (n=28, 4 replicates) (Table 6). Therefore, the dgRNA_4+7 system was used for objective 1.2 to generate NANOS3−/− cattle.

TABLE 6
Blastocyst rates and mutation efficiencies of NANOS3 dgRNA_4 + 7 in vivo testing

| | | | | | Knockout | | >5% wt | | | |
|---|---|---|---|---|---|---|---|---|---|---|
| Rep. | Blastocyst Rate | | Total Mutation | | (0% wt) | | Mono-allelic | | Mosaic | |
| 1 | 6/30 | (20%) | 6/6 | (100%) | 4/6 | (67%) | 1/6 | (17%) | 1/6 | (17%) |
| 2 | 6/30 | (20%) | 4/6 | (67%) | 3/6 | (50%) | 1/6 | (17%) | 0/6 | (0%) |
| 3 | 5/40 | (13%) | 5/5 | (100%) | 5/5 | (100%) | 0/5 | (0%) | 0/5 | (0%) |
| 4 | 11/50 | (22%) | 11/11 | (100%) | 10/11 | (91%) | 0/11 | (0%) | 1/11 | (9%) |
| | 28/150 | (19%) | 26/28 | (93%) | 22/28 | (79%) | 2/28 | (7%) | 2/28 | (7%) |

### Example 3—Generation CRISPR-Cas9 NANOS3−/− Bovines

### Recipient Synchronization

Recipient females were synchronized following the “Select Synch+CIDR®” (controlled internal drug release; intravaginal progesterone insert) protocol. On day 0, recipients were given an injection of gonadotropin releasing hormone (GnRH) and were administered a CIDR®. After 7 days, the CIDR® was removed and recipients were given a prostaglandin (PG) injection. Recipients were observed for heat (i.e., signs of estrus) using heat patches or activity monitoring collars and confirmed with visual observation. Additionally, a veterinarian confirmed corpus luteum (CL) development via rectal palpation. Only recipients with an observed heat and ≥15 mm CL were considered as eligible recipients.

### Embryo Transfer (ET)

Bovine embryos were in vitro produced and microinjected at 6 hpi with the dgRNA_4+7 system, as described above. On day 7 of culture, all embryos were scored for developmental stage reached. Based on the number of available synchronized recipients, microinjected blastocysts were selected and loaded into embryo transfer (ET) straws (Table 7). The selected embryos were transferred into the recipient's uterine horn that was ipsilateral to the ovary harboring a CL 7 days post heat detection. A total of 26 presumptive NANOS3 knockout embryos have been transferred by collaborating veterinarians into 26 synchronized recipients (Table 8).

### Established Pregnancies

Twenty-one days after ET (28-day fetus), initial pregnancy rates were determined via a blood progesterone test (n=10/26, 38%). Approximately, one week later (≥35-day fetus), pregnancies were confirmed via ultrasound (n=8/26, 31%). Furthermore, near the end of the first trimester (50-70 day gestation) the sex of the developing fetuses were determined via ultrasound. As a result of these 8 pregnancies, 3 live calves were born (1 male and 1 female; FIGS. 4A-4F and 5A-5D).

TABLE 7
ET and pregnancy rates for CRISPR-Cas9 NANOS3 targeted bovine embryos

| | | | | Pregnancies | | | | | | | | |
|---|---|---|---|---|---|---|---|---|---|---|---|---|
| | | | | | | | | | | Male | Female | |
| Rep | ET Date | Rep KO¹ rate | | 28-day | | 35-day | | 70-day | | (#) | (#) | Due Date |
| 1 | Feb. 27, 2020 | 26/26 | (100%) | 3/8 | (38%) | 2/8 | (25%) | 2/8 | (25%) | 1 | 1 | Dec. 2, 2020 |
| 2 | May 27, 2020 | n/a² | | 0/6 | (0%) | 0/6 | (0%) | 0/6 | (0%) | 0 | 0 | — |
| 3 | Jun. 17, 2020 | n/a² | | 2/5 | (40%) | 1/5 | (20%) | 1/5 | (20%) | 1 | 0 | Mar. 24, 2021 |
| 4C³ | Dec. 16, 2020 | 10/12 | (83%) | 3/4 | (75%) | 3/4 | (75%) | 3/4 | (75%) | 3 | 0 | Sep. 25, 2021 |

| | | | | | | | | | | | |
|---|---|---|---|---|---|---|---|---|---|---|---|
| 4Y⁴ | Dec. 16, 2020 | 2/4 | (50%) | 2/3 | (67%) | 2/3 | (67%) | n/a | 2 | 0 | n/a |

| | | | | | | | | | |
|---|---|---|---|---|---|---|---|---|---|
| 38/42 | (90%) | 10/26 | (38%) | 8/26 | (31%) | 6/24 | (25%) | 7 | 1 |

¹Knockout rate (i.e., 0% wt) of blastocysts from the same replicate that were not selected for ET.
²Due to low development, all blastocysts from that replicate were used for ET.
³4C: Embryos in replicate 4C were in vitro fertilized using conventional semen but all resulting pregnancies are male.
⁴4Y: Embryos in replicate 4Y were in vitro fertilized using male-sex-sorted semen and the 2 male presumptive NANOS3 knockout fetuses were collected on day 42 of gestation.

NANOS3^{-/-} bovine fetuses

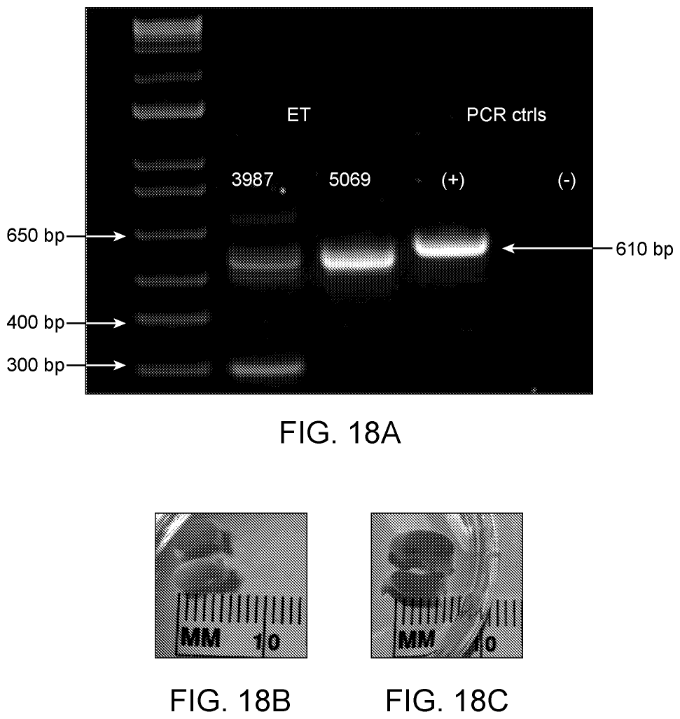

To evaluate NANOS3−/− fetal gonad development to better understand the role of NANOS3, two male presumptive NANOS3 knockout fetuses were collected and analyzed. Tissue was collected from the fetuses and DNA was extracted for NANOS3 genotyping via PCR and Sanger sequencing. Both 90-day fetuses were NANOS3 knockouts (i.e., no wild type DNA was present) (FIG. 18A). 90-day fetus, #3987 was a NANOS3 mosaic knockout with 4+ alleles, including 1 large deletion, and no wildtype alleles. 90-day fetus, #5069 was also a NANOS3 mosaic knockout with 3+ alleles, and no wildtype alleles.

Phenotyping NANOS3^{-/-} bovine fetuses

Fetal testes were isolated from the two 90-day NANOS3 knockout fetuses and preserved (i.e., slow-frozen) for analysis via single-cell RNA sequencing (scRNA-Seq) (e FIGS. 18B-18C). 90-day, male, wildtype (i.e., NANOS3+/+) testes samples were also collected and preserved in the same way for comparison. Fetal gonads were dissociated into single cell suspensions and then fixed and permeabilized using the “Cell Fixation Kit” Parse Biosciences, Seattle, WA). Fixed cells were submitted on ice to the UC Davis DNA Technologies & Expression Analysis Core for library preparation and sequencing. Libraries were prepared using a split-pool combinatorial barcoding method (“Single Cell Whole Transcriptome—100 k cells/nuclei, up to 48 samples” kit, Parse Biosciences, Seattle, WA). Sequencing was performed on an Illumina NovaSeq 6000 platform as 150 base paired-end. Reads were mapped to the bovine reference genome (ARS-UCD1.2.105). Data was analyzed using the Parse Biosciences pipeline (version 0.9.6p) and the R package Seurat (v4.1.0). Dimensionality reduction was performed by uniform manifold approximation and projection (UMAP) and differential gene expression was performed using non-parametric Wilcoxon rank sum test, which is part of the standard Seurat pipeline.

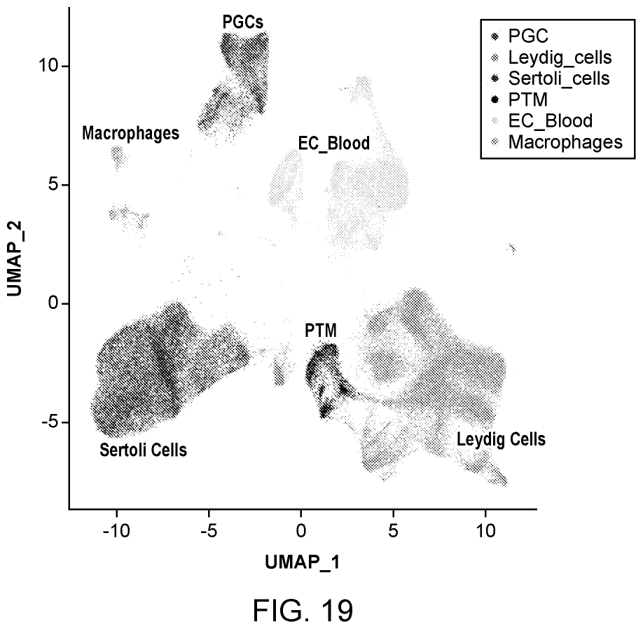

The scRNA-Seq analysis demonstrated a complete loss of primordial germ cells (PGCs) in the NANOS3 knockout 90-day fetal testes, but all other somatic cell populations (e.g., Sertoli and Leydig cells) were present and comparable to 90-day wildtype control samples (FIGS. 19-21, 22A-22F, and 23A-23B).

### NANOS3^{-/-} Live Calves

Blood samples were collected from the calves, DNA was extracted, and PCR was performed to determine NANOS3 genotypes. Based on PCR and Sanger Sequence analysis, the first male (Fauci) is a mosaic NANOS3 knockout carrying at least 4 different knockout alleles, including 1 allele with a large (298 bp) deletion and >3 alleles with small indels at one or both guides' cut sites. The heifer (FunBun) is a bi-allelic, compound heterozygote carrying 2 unique knockout alleles, both with large indels (291-297 bp). The mutations present in these two calves are predicted to completely disrupt NANOS3. The third healthy calf, named Frodo (male), carries an allele (bi-allelic, homozygous) with in-frame deletions (i.e., small deletions that are multiples of 3). The allele results in an amino acid substitution and a deletion of 3 amino acids total (FIG. 5D). The amino acid substitution and deletions are all outside of the highly conserved Zinc Finger binding domain. Based on literature, it is unknown if these deleted amino acids are necessary for NANOS3 protein function or not. However, the exact amino acid sequence that is predicted to result from the allele Frodo is carrying was not found in any other species when a protein BLAST (Basic Local Alignment Search Tool) was conducted. Therefore, Frodo will be further evaluated for germ cell production, as described below.

### Phenotyping NANOS3^{-/-} Live Bovines

Table 8 shows a record of monthly blood collections, body weights, and scrotal circumference (SC) measurements for the first two live, NANOS3−/− calves (FunBun, Fauci, Frodo).

TABLE 8

| | FunBun (female, | Fauci (male, | | Frodo (male, | |
|---|---|---|---|---|---|
| Age | #854) | #838) | | #3964) | |
| (mo) | Wt (lbs) | Wt (lbs) | SC (cm) | Wt (lbs) | SC (cm) |
| 1 | 170 | 205 | — | 163 | — |
| 2 | 240 | 271 | — | 215 | — |
| 3 | 287 | 341 | — | —* | — |
| 4 | 306 | 370 | — | 316 | — |
| 5 | 340 | 407 | 13.4 | 439 | 16.0 |
| 6 | 416 | 467 | 14.2 | 546 | 20.5 |
| 7 | 462 | 522 | 19.5 | | |
| 8 | 556 | 640 | 19.5 | | |
| 9 | 638 | 716 | 22.0 | | |
| 10 | 685 | 802 | 22.6 | | |
| 11 | 730 | 884 | 26.2 | | |
| 12 | —* | —* | 27.0 | | |
| 13 | 776 | 950 | 27.5 | | |
| 14 | 874 | 1085 | 27.5 | | |
| 15 | 946 | 1200 | 27.5 | | |

*No measurement - broken scale

A one year breeding soundness examination was carried out on the first male knockout bull (#838). The examination indicated the knockout bull had an anatomically normal reproductive tract (i.e., accessorcy sex glands and penis) and normal testicular development, although the scrotal circumference (27 cm) was smaller than expected for age and breed matched controls. Microscope evaluation of an ejaculate obtained via electroejaculation revealed seminal plasma only with no spermatozoa present. These results were confirmed with a second breeding soundness exam one month later (13-months-old).

### Example 4—Functional Characterization of CRISPR/Cas9 NANOS3 Knockout Bovine Testes Using Single Cell RNA-Sequencing—Follow-on to Example 3

Guide RNA (gRNA) Design and In Vitro and In Vivo Testing

CRISPR-Cas9 guide RNAs targeting Exon 1 of bovine NANOS3 were optimized as previously described. Briefly, guide RNAs (gRNAs) targeting the bovine NANOS3 exon 1 were designed and screened for potential off-target sites based on the bovine reference genome (ARS-UCD1.2) using sgRNA Scorer 2.0 (Chard et al., 2017, ACS Synth Biol. 6(5):902-4) and Cas-OFFinder (Bae et al., 2014, Bioinformatics. 30(10):1473-5), respectively. Selected gRNAs had three or more total mismatches in the guide sequence for predicted off-target sites, and at least one mismatch in the seed region (8-10 bp proximal to the protospacer adjacent motif (PAM) site) of the gRNA (Hennig et al., 2020, Sci. Rep. 10(1):22309). gRNA (Synthego, Menlo Park, CA, USA) cleavage efficiency was assessed by an in vitro cleavage assay, as previously described (Hennig et al., 2020, Sci. Rep. 10(1):22309). (See also e.g., Example 1, FIG. 2).

gRNA In Vivo Testing

To determine the mutation rate for each single guide (sgRNA) and for dual guide (dgRNA) combinations, 6-hpi zygotes were microinjected (e.g., laser-assisted cytoplasmic injection (Bogliotti et al., 2016)) with 6 μL of a solution containing 167 ng/μL of Cas9 protein (PNA Bio, Inc., Newbury Park, CA, USA), and 67 ng/μL of a sgRNA or 67 ng/μL each of 2 gRNAs. The Cas9 protein and gRNA(s) were incubated together at room temperature for 30 minutes to form a ribonucleic protein (RNP) prior to microinjection. Microinjected embryos were incubated for 7-8 days and on day 7 embryos were scored for developmental stage reached. Blastocysts were individually collected in lysis buffer for DNA extraction. The NANOS3 target region was amplified by two rounds of nested PCR using primers (Eurofins Genomics, Louisville, KY, USA). PCR products were visualized on a 1% agarose gel, purified using the QIAquick Gel Extraction Kit (Qiagen, Inc., Valencia, CA, USA) and Sanger sequenced (GeneWiz, South Plainfield, NJ, USA).

### Bovine Embryo Production

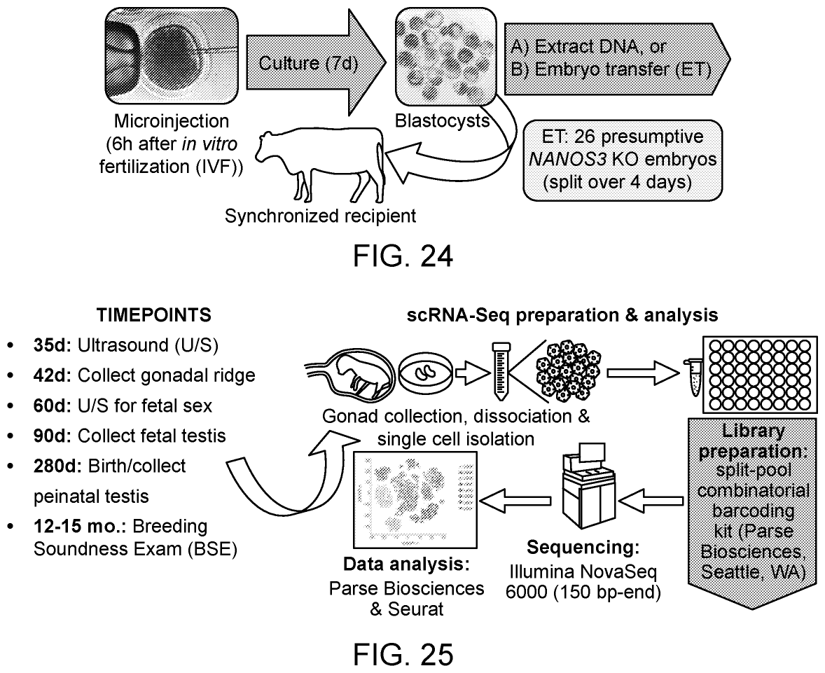

NANOS3 knock out embryos were produced in vitro using an in vitro embryo production (IVP) and CRISPR/Cas9 microinjection, which is diagramed in FIG. 24 and adapted from Henning S.L., et al 2020, Sci. Rep. 10(1):22309. Briefly, bovine ovaries were obtained from a local processing plant. Cumulus-oocyte complexes (COCs) were aspirated from follicles and transferred to maturation media (IVF Bioscience, Falmouth, UK) for 21-24 hr. COC's were fertilized in SOF-IVF (Bakhtari and Ross, 2014, Epigenetics. 9(9):1271-9) with 2×10⁶ sperm per mL for 6 hr. In vitro maturation and fertilization incubations were at 38.5° C. in a humidified 5% CO₂ incubator. Six hours post insemination (hpi), presumptive zygotes were denuded by light vortex in SOF-HEPES (Bakhtari and Ross, 2014) for 5 min. Zygotes were in vitro cultured (IVF Bioscience, Falmouth, UK) at 38.5° C. in a humidified atmosphere of 5% CO₂, 5% O₂, and 90% N2 for 7-8 days.

### Embryo Transfer (ET) & Pregnancy Monitoring.

All animal procedures were approved by the Institutional Animal Care and Use Committee (IACUC) of the University of California (UC), Davis (#21513). Recipient females were synchronized following the “Select Synch+CIDR®” protocol. Bovine embryos were in vitro produced and microinjected at 6-hpi with a dgRNA system, as described above. On day 7, microinjected blastocysts were selected and loaded individually into ET straws and transferred (one per recipient) into the recipient's uterine horn that was ipsilateral to the ovary harboring a CL 7 days post heat detection. Pregnancy rates were determined via blood progesterone tests and pregnancies were monitored through periodic transrectal ultrasounds.

### Live Animal Evaluation

Blood samples were collected from new-born calves, DNA was extracted using a DNeasy® Blood & Tissue Kit (Qiagen, Inc., Valencia, CA, USA), and the NANOS3 target region was amplified by PCR. PCR products were visualized on a 1% agarose gel, purified, and Sanger sequenced. Once the confirmed NANOS3 edited males reached reproductive age (˜12 months), a breeding soundness exam (BSE) was conducted by UC Davis veterinarians. The BSE included a general physical examination, inspection of genital organs, and semen collection via electroejaculation.

scRNA-Seq Analysis

Gonad samples were collected and prepared for scRNA-Seq analysis as shown in FIG. 25. The fetal testis from 90d KO samples (n=2) and from 2 age-matched wildtype controls were isolated and prepared for scRNA-seq analysis. Single cells were isolated from whole bovine testes, libraries were prepared using a split-pool combinatorial barcoding kit (Parse Biosciences, Seattle, WA), and sequenced on an Illumina NovaSeq 6000 instrument (150 base paired-end). The sequencing data was analyzed using a Parse Biosciences pipeline and the Seurat package in R.

### Results and Discussion

Bovine NANOS3 is a 2,633 bp gene with two exons (FIG. 2). Seven gRNAs were designed to target the larger exon one (451 bp), as it contains the coding region for the critical zinc finger binding domain. Four gRNAs (#1, #4, #5, #7) successfully cut the target region in vitro, so they were further tested in vivo. To determine blastocyst development rate and mutation efficiency each gRNA was independently microinjected alongside Cas9 protein in groups of 30 zygotes, 6-hpi. Groups of 30 non-injected embryos were cultured as controls. All injected groups resulted in a blastocyst development rate (≥20%), which was similar to other microinjected bovine embryo experiments (Hennig et al., 2020, Sci. Rep. 10(1):22309), and three sgRNAs (#4, #5 and #7) resulted in an over 60% total mutation rate.

Next, a dgRNA system was tested. gRNA #4 and #7 were selected based on their high individual mutation efficiencies (83% and 63%, respectively), and genomic locations, so that when co-injected they could introduce a large deletion of 297 bp (FIG. 2). The dgRNA_4+7 microinjections resulted in a similar blastocyst development rate (19%) and >75% KO rate (i.e., 0 wild-type alleles) (n=28, 4 replicates).

For ET, in vitro produced bovine embryos were microinjected at 6-hpi with the dgRNA_4+7. On day 7 of in vitro culture, eight presumptive NANOS3 KO blastocysts were selected and transferred into synchronized recipients. Twenty-one days after ET (28-day foetus), initial pregnancy rates were determined via a blood progesterone test (n=3/8, 38%). Ultrasound was used approximately one week later (35-day foetus) to confirm pregnancies (n=2/8, 25%) and around 60-days of gestation to determine foetal sex (one male and one female).

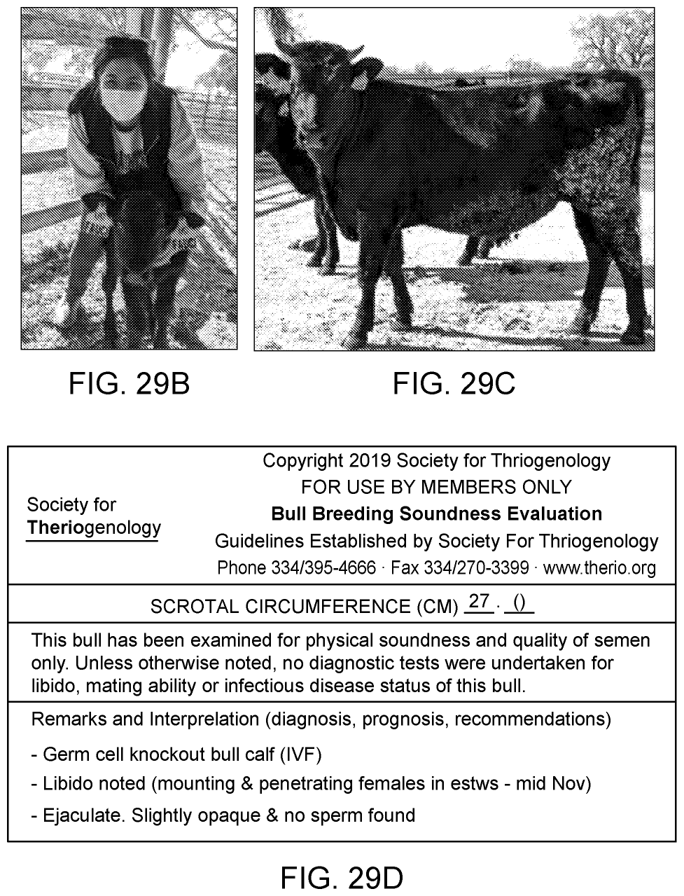

Two healthy calves one male (#838) and one female (#854) were born unassisted at the UC Davis Beef Barn in the fall of 2020; however, this paper will only focus on the resulting male (FIG. 29A). PCR and Sanger Sequence analysis of DNA extracted from blood indicated that #838 is a mosaic NANOS3 KO, carrying at least four different KO alleles (FIG. 29A). One allele has a large (298 bp) deletion and ≥3 alleles have small indels at one or both gRNA cut sites. The mutations present in #838 were predicted to disrupt NANOS3, which Applicant hypothesized would result in a complete loss of germ cells in his gonads. To evaluate the potential for NANOS3 KO cattle to serve as hosts for donor-derived gamete production, the reproductive development and capability of bull #838 was documented. The bull demonstrated normal libido and a 12-month BSE revealed that #838 had a normal reproductive tract (i.e., accessory sex glands and penis) and normal testicular development, although the scrotal circumference (27 cm) was smaller than expected for age and breed matched controls. Upon electroejaculation, #838 produced a slightly opaque seminal fluid, and under microscopic observation there was no evidence of the presence of any sperm.

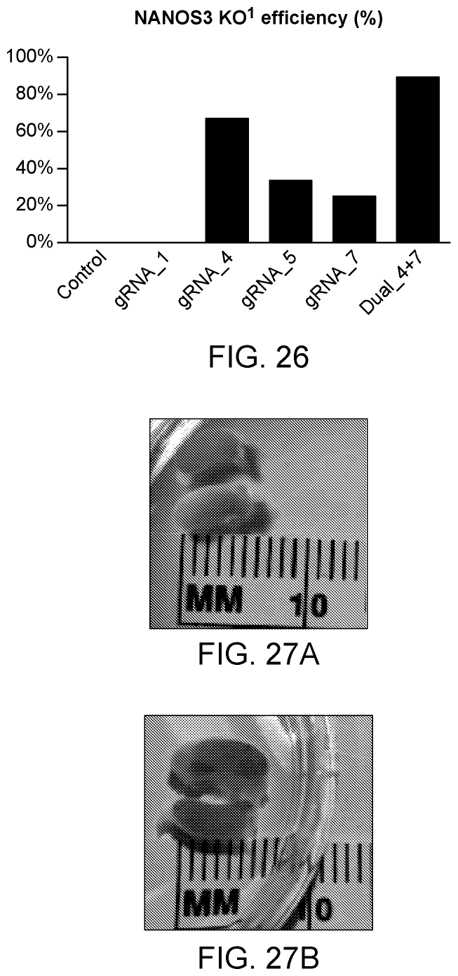

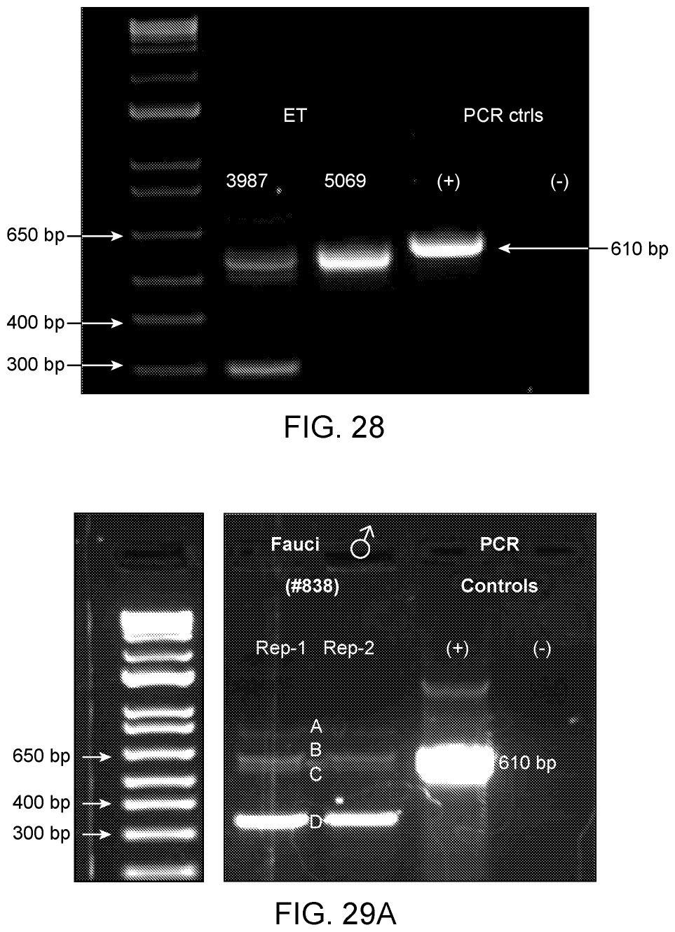

Results from the production of a CRISPR-Cas9 NANOS3 KO cattle are shown in FIG. 26. Dual gRNA 4+7 resulted in an 89% NANOS3 KO rate in developing embryos (n =74/83, 7 replicates). There was a 31% pregnancy rate for 35d ultrasound (n=8/26). KO in FIG. 26 was defined as 0% wildtype (wt), or non-mutated, NANOS3 alleles based on PCR and Sanger Sequence analysis of the target region. FIG. 27A-27B shows images of 90d fetal testes from two fetuses (3987, FIG. 27A; 5069, FIG. 27B). PCR for NANOS3 was performed on DNA extracted from blood from the two fetuses (FIG. 28). #3987 was a mosaic KO (4+ alleles) and included one large deletion and no wildtype. #5069 was a mosaic KO (3+ alleles), and no wildtype.

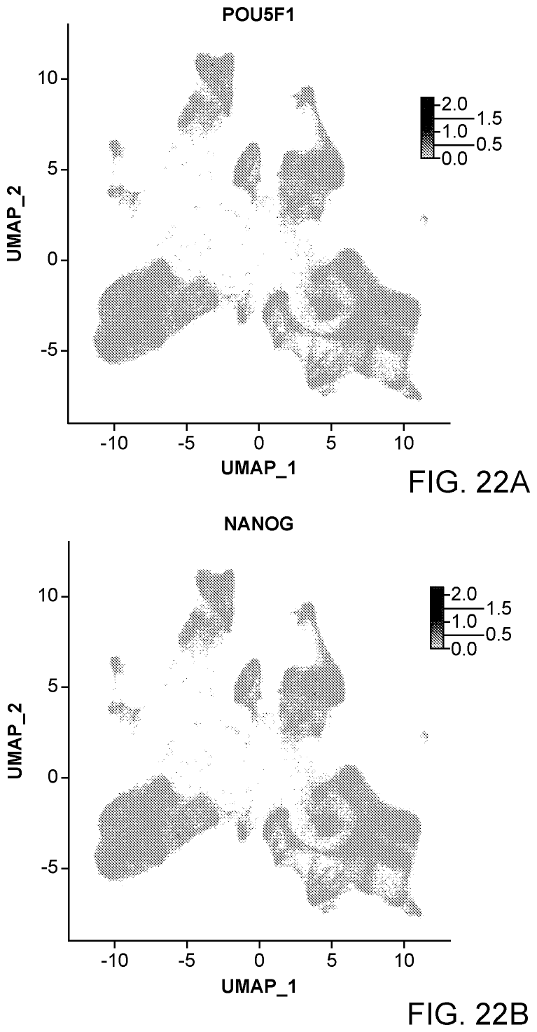

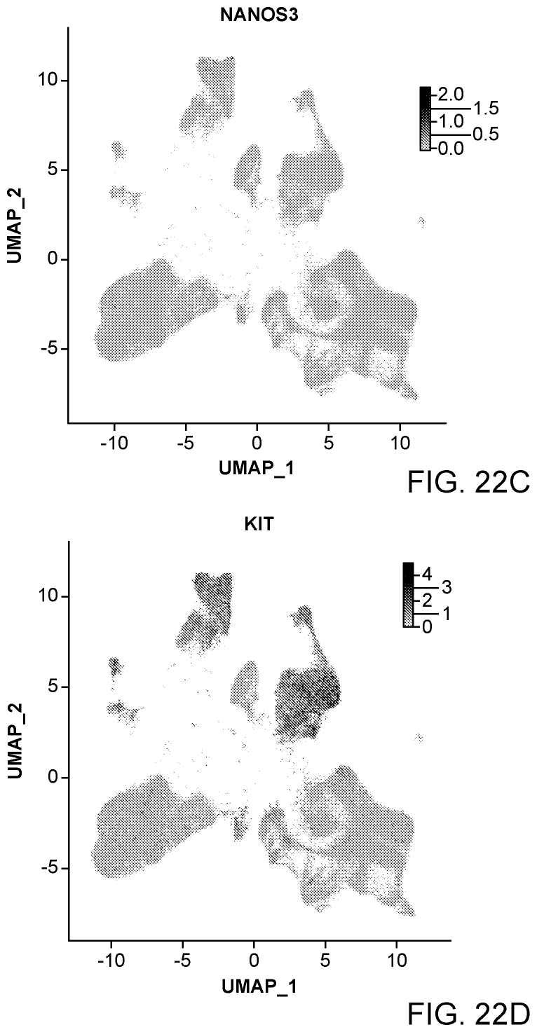

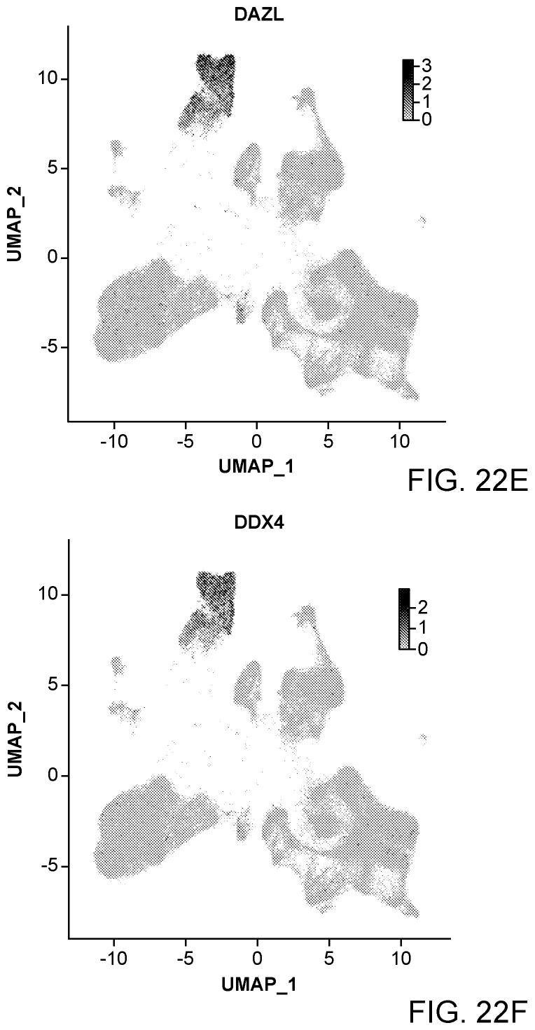

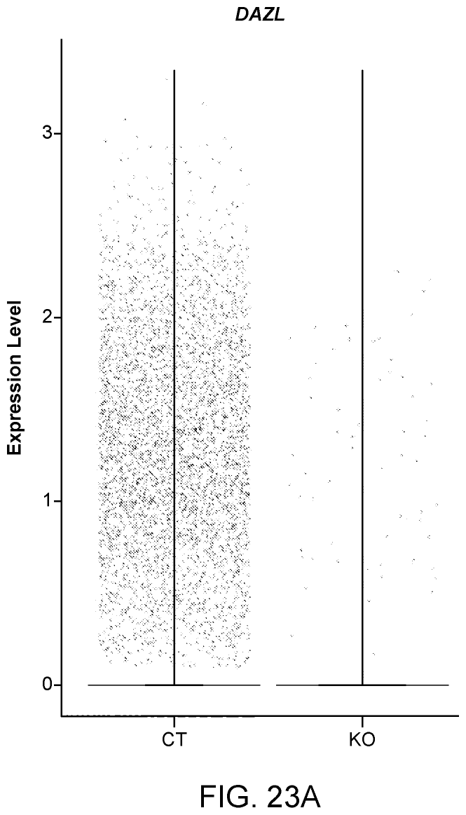

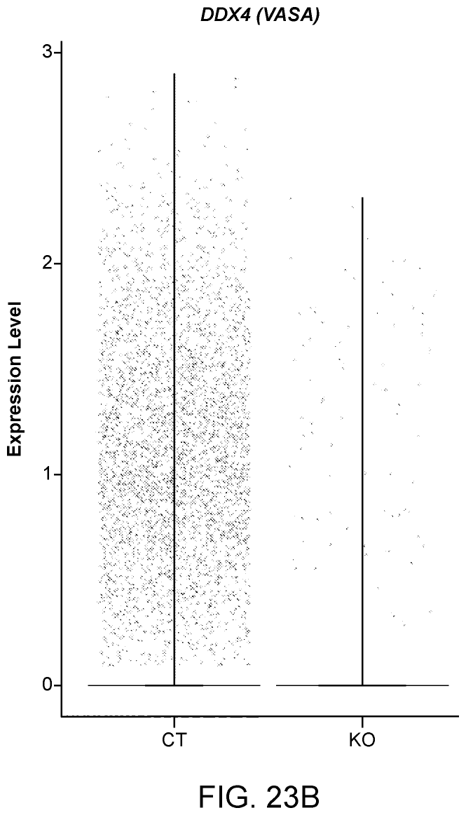

scRNA-seq was performed and demonstrated a complete loss of primordial germ cells (PGCs) in CRISPR/Cas9 NANOS3 KO 90d fetal testes and all other somatic cell subpopulations were present and comparable to 90d wildtype control samples. scRNA-seq results are shown in FIG. 22A-22F. PGC expression markers were further evaluated. Results are sown in FIG. 23A-23B.

NANOS3 KO bull (#838) was germline ablated, but otherwise had normal reproductive development (FIG. 29A-29D). BSE results noted that libido, reproductive tract anatomy were all normal. Microscopic evaluation of an ejaculate obtained via electroejaculation revealed seminal plasma only with no spermatozoa present, indicating successful germline ablation.

Applicant optimized a gene knockout approach using co-injection of two selected gRNA/Cas9 ribonucleoprotein complexes into bovine zygotes (6 hours after IVF) to achieve a high NANOS3 KO rate in developing embryos. Subsequent embryo transfers resulted in 8 pregnancies, including the successful production of a live bull calf with a targeted gene KO of NANOS3. Although both of the 90d samples and the live bull were mosaic at the NANOS3 target locus, no wildtype sequences remained, and all mutations resulted in NANOS3 loss of function. scRNA-seq analysis showed a complete loss of PGCs in CRISPR/Cas9 NANOS3 KO fetal testis, but all other somatic cell populations were present and comparable to age matched wt control samples. Additionally, the lack of spermatozoa in the ejaculate of the live bull at sexual maturity supports the hypothesis that inactivation of NANOS3 in male cattle will result in complete germline ablation. Importantly the bull had normal libido and an anatomically normal reproductive tract. This example at least demonstrates that NANOS3 KO bulls have a phenotype that would be well suited to serve as hosts for germline complementation.

### Example 5—Physiological Characterization of Live CRISPR/CAS9 NANOS3 Edited Cattle

In order to evaluate the potential for NANGS3−/− cattle to serve as hosts for donor-derived gamete production, Applicant analyzed the NANGS3−/− live animals' reproductive development and capabilities. Monthly, Applicant recorded body weight, measured scrotal circumference (males only), and collected blood to measure steroid hormone levels (e.g., testosterone and estrogen) as the animals approached and progress through puberty (Table 9). Additionally, when the NANOS3 edited males reach reproductive age (˜12 months), breeding soundness exams (BSE) have/will be conducted by UC Davis veterinarians. The BSEs follow the standards set forth by the Society for Theriogenology and include a general physical examination, inspection of reproductive organs, and semen collection via electroejaculation. See BSE test discussed in Example 4. Ultimately, the three NANOS3 edited animals were/will be slaughtered (˜15-months) to enable collection and comprehensive analysis of their reproductive tracts, with specific focus on the gonads.

TABLE 9
Record of monthly blood collection dates, body weights,
and scrotal circumference (SC) measurements for the
live, NANOS3−/− calves (#854, #838, and #3964).

| | #854 | | | | |
|---|---|---|---|---|---|
| | (FunBun) | #838 (Facui) (male) | | #3964 (Frodo) (male) | |
| | (female) | | Scrotal | | Scrotal |
| Age | Weight | Weight | circumference | Weight | circumference |
| (mo) | (lbs) | (lbs | (SC) (cm) | (lbs) | (SC) (cm) |
| 1 | 170 | 205 | — | 163 | — |
| 2 | 240 | 271 | — | 215 | — |
| 3 | 287 | 341 | — | —* | — |
| 4 | 306 | 370 | — | 316 | — |
| 5 | 340 | 407 | 13.4 | 439 | 16.0 |
| 6 | 416 | 467 | 14.2 | 546 | 20.5 |
| 7 | 462 | 522 | 19.5 | 618 | 22.5 |
| 8 | 556 | 640 | 19.5 | 594 | 26 |
| 9 | 638 | 716 | 22.0 | 796 | 31 |
| 10 | 685 | 802 | 22.6 | 800 | 31 |
| 11 | 730 | 884 | 26.2 | 846 | 32 |
| 12 | —* | —* | 27.0 | 897 | 32 |
| 13 | 776 | 950 | 27.5 | 954 | 32 |
| 14 | 874 | 1085 | 27.5 | 1010 | 32 |
| 15 | 946 | 1200 | 27.5 | — | — |

*Scale was broken due to excessive rain.

### Bull #838 (Fauci) Characterization

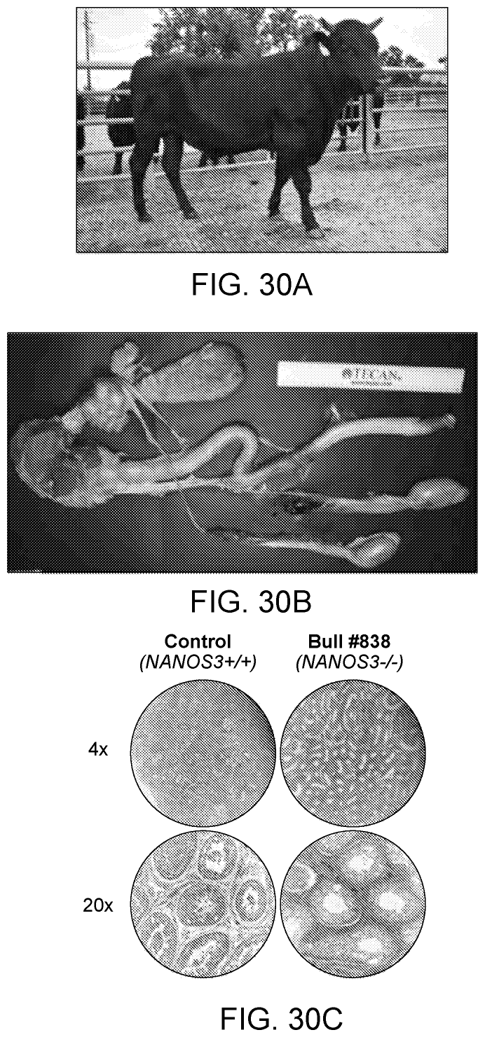

Bull #838 (named, Fauci (FIG. 30A)) was a mosaic KG, with at least 4 mutated alleles, including an allele with 1 large deletion, and no wildtype alleles. Due to these knockout mutations, we hypothesized that there would be a complete loss of germ cells in bull #838, but otherwise normal gonadal development (Tsuda et al., 2003). At 12-months of age, bull #838 demonstrated normal libido and a BSE found that he had an anatomically normal reproductive tract (i.e., accessory sex glands and penis) and normal testicular development, although the scrotal circumference (27 cm) was smaller than expected for age and breed matched controls. However, microscopic evaluation of an ejaculate obtained via electroejaculation revealed seminal plasma only with no spermatozoa present. These results were repeated and confirmed with BSE's at 13 and 15-months-old. bull #838 was harvested and we completed a full analysis of his reproductive tract. Bull #838's reproductive tract was anatomically normal with all accessory sex glands present (FIG. 30B). Additionally, cross-sections of bull #838's testis were processed for H&E analysis (FIG. 30C). Compared to an age matched, wildtype (NANOS3+/+) bull, bull #838 (NANOS3−/−) lacks any spermatogenesis, but still has Sertoli cells lining the seminiferous tubules. The lack of spermatozoa in the ejaculate of bull #838 and his germ cell deficient testis support the hypothesis that inactivation of NANOS3 in male cattle will result in complete germline ablation (i.e., functionally sterile). Furthermore, this Example at least demonstrates that NANOS3 KO bulls have a phenotype that would be well suited to serve as hosts for germline complementation studies.

### Bull #3964 (Frodo) Characterization

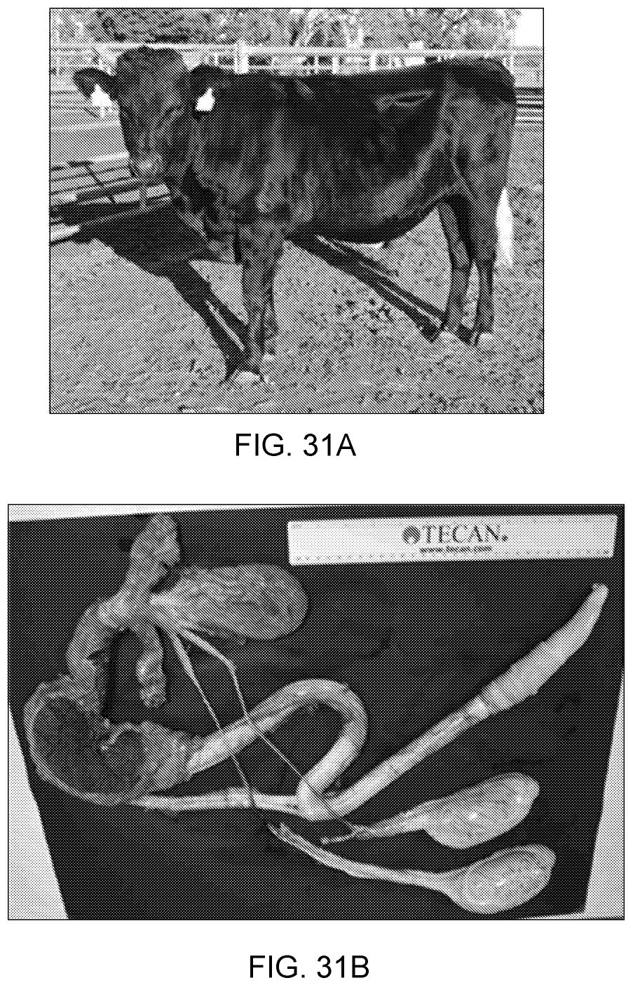

Bull #3964 (Frodo) (FIG. 31A) was carrying 3 mutated alleles and no wild type alleles. The majority (70%) of Bull #3964's alleles had large deletions (1.3-1.5 kb). However, he was also carrying one allele (30%) that had in-frame deletions (i.e., small deletions that are multiples of 3). The allele results in an amino acid substitution and a deletion of 3 amino acids total and the mutations are all outside of the highly conserved Zinc Finger binding domain. Based on literature, it is unknown if these deleted amino acids are necessary for NANOS3 protein function or not. However, Applicant hypothesized that these in-frame deletions could result in a functional NANOS3 protein and thus an intact germline.

At 12-months of age, bull #3964 demonstrated normal libido and passed a BSE. Bull #3964 was found to have an anatomically normal reproductive tract, normal testicular development with adequate scrotal circumference (32 cm), and produced a satisfactory ejaculate for his age (30% motility, 78% normal cells, 11% head abnormalities, 11% tail abnormalities, 0% tail abnormalities). Bull #3964 was harvested around 15 months of age for further evaluation of his reproductive tract. Bull #3964's reproductive tract was anatomically normal with all accessory sex glands present and adequate scrotal circumference (FIG. 31B). Additionally, cross-sections of bull #3964's testis were collected and are currently being processed for H&E analysis to further confirm the BSE results.

### Heifer #854 (FunBun) Phenotype

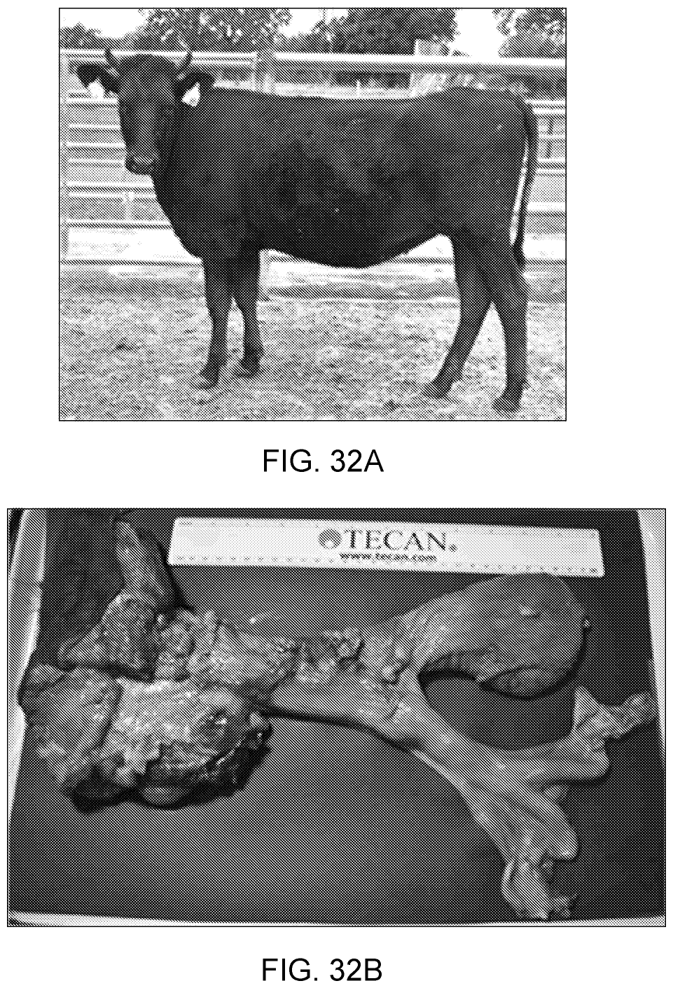

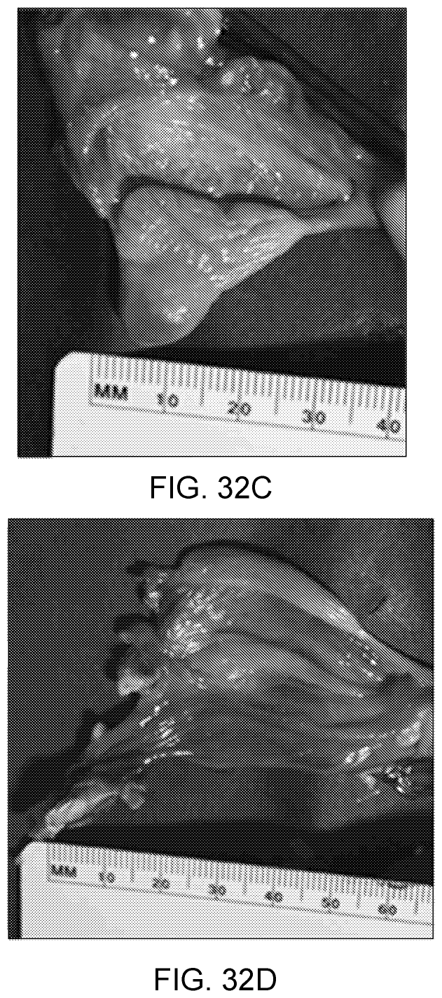

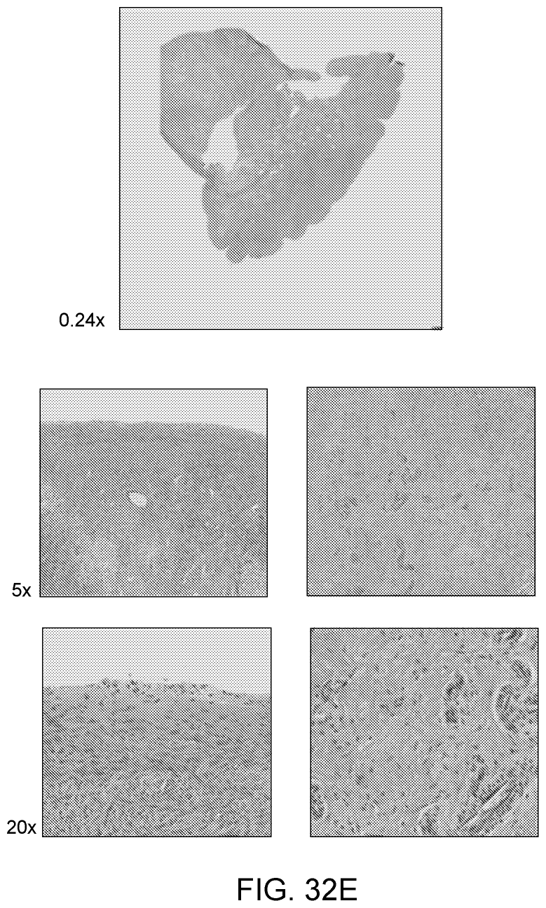

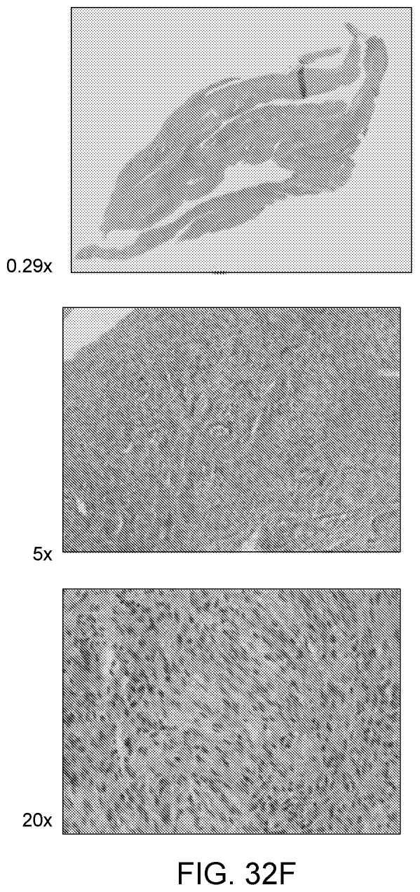

Heifer #854 (named, FunBun (FIG. 32A)) was a mosaic KO, with 5 mutated alleles and no wildtype alleles. All of heifer #854's alleles had targeted dual gRNA_4+7 indels (291-298 bp; Table 9). Due to these knockout mutations, Applicant hypothesized that there would be a complete loss of germ cells in heifer #854, but otherwise normal gonadal development.

Heifer #854 was observed through puberty until 15-months of age and never showed signs of estrus. UC Davis veterinarians performed a reproductive exam on heifer #854, around 14 months of age. A small, involuted, and hypoplastic reproductive tract, with a small cervix and flaccid uterine horns, was observed during palpation, which are similar characteristics of a juvenile or freemartin female. The right ovary was unable to be imaged with ultrasound and no structures could be identified. The left ovary was small (<1 cm) and no structures or follicular development were observed with ultrasound.

Heifer #854 was harvested and Applicant completed a full analysis of her reproductive tract. Heifer #854's reproductive tract was observed to be anatomically abnormal, with a small clitoris, long anterior vagina and a putative primitive streak on the right side (FIGS. 32B-32D). Additionally, cross-sections of the left ovary and right primitive streak were processed for H&E analysis, which showed a complete lack of oogenesis (FIGS. 32E-32F). The lack of oogenesis in heifer #854's ovaries confirm the hypothesis that inactivation of NANOS3 will result in complete germline ablation in an adult female bovine (i.e., functionally sterile).

### Example 6—Generate Pregnancies Using ESC Chimeric Embryos and Examine Early-Stage Fetuses to Determine the Extent to which ESC-Derived Cells can Contribute to Fetal Chimerism

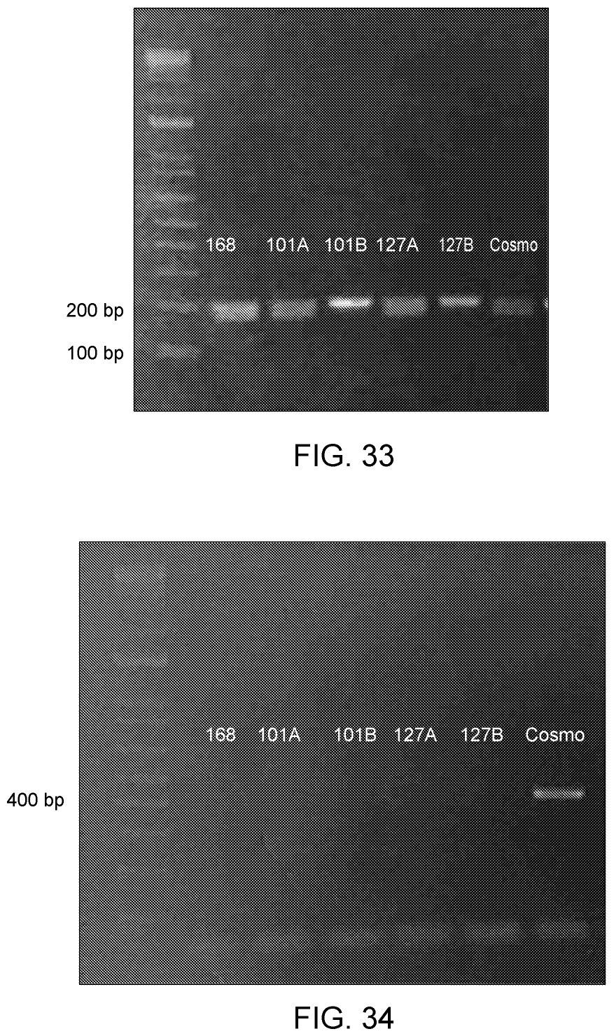

Applicant used previously developed procedures (Bogliotti et al (2018) Proceedings of the National Academy of Sciences, 115, 2090-2095) to obtain >75% of the embryos with ESC contribution to ICM. By applying those conditions, Applicant produced chimeric embryos utilizing stem cells derived from the Cosmo bESC cell line. These cells have a unique DNA sequence (GFP) to trace the lineage of ESCs in the developing embryo. A total of 20 presumptive bESC complemented embryos were loaded in 10 straws, with 2 embryos per straw and were transferred into 10 synchronized recipients. Six (60%) heifers carrying presumptive bESC complemented embryos were detected pregnant. Four recipients remained pregnant at ultrasound ˜10 days later. Two (#0159, #0168) were carrying singletons with a confirmed heartbeat, and two (#0127, #0101) were carrying twins. #0159 lost her pregnancy before we took the fetuses at day 90 of gestation. Pregnancies were interrupted at day 90 of gestation and 5 fetuses were taken. Recipients with twins carried a female and a male fetus in both cases, and the singleton pregnancy resulted in a male. Fetuses were identified as #101A, #101B, #127A and #127B, and #168. DNA was extracted from all samples using a Qiagen extraction kit and DNA concentration was measured by Nanodrop. PCR technique using three sets of primers DDX3, was utilized to confirm sex of the fetus After PCR analysis and gel electrophoresis, sex was confirmed with 3 male and 2 female fetuses (FIG. 33).

Samples of brain, liver, kidney, heart, cotyledons (fetal placental tissue) and gonads were taken from fetus #168, #101A, #101B, #127A and #127B. After PCR and an extensive qPCR investigation in all the tissues from each fetus, no presence of GFP was detected in any tissue analyzed of any of the five fetuses. Representative image of PCR and gel electrophoresis of samples taken from the fetuses' tail (FIG. 34).

### Example 7—Genotype Analysis of CRISPR/CAS9 NANOS3 Edited Bovine Samples Through Next Generation Sequencing

Further genotype analysis was completed for all eight CRISPR/CAS9 NANOS3 edited bovine samples to provide data on the types and proportion of edits that were introduced by the dgRNA_4+7 editing approach.

### Long-Range PCR of Bovine NANOS3

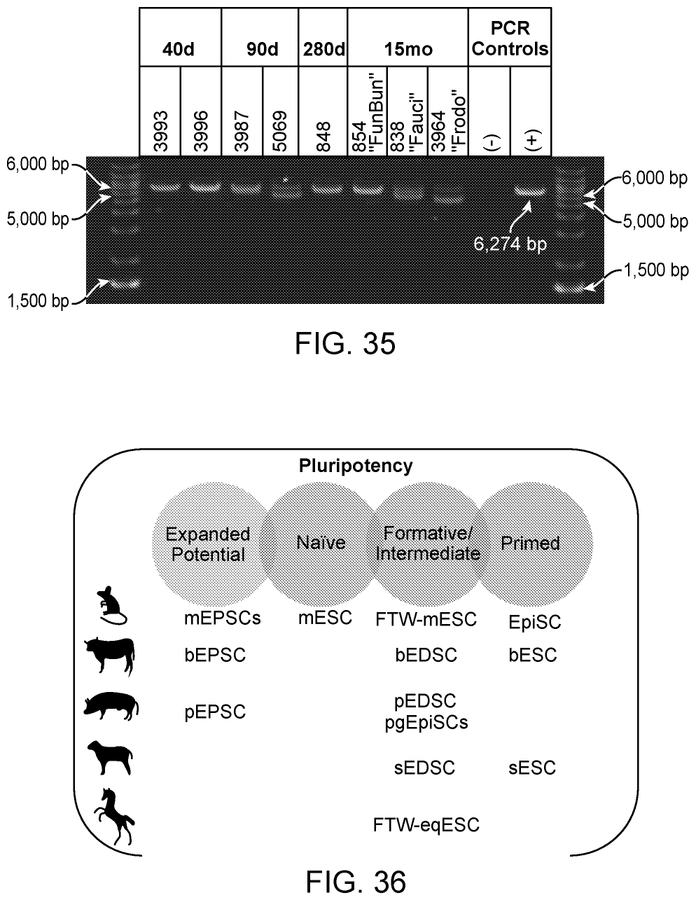

A 6,274 bp region centered around the NANOS3 dgRNA_4+7 target location was amplified by one round of long-range PCR using primers (N3-6kb_2F: CCTCAACTGACGGGGAAGTC (SEQ ID NO: 46), N3-6kb_2R: TTGTTGTCGGTGGGTTGTGA (SEQ ID NO: 47); Integrated DNA Technologies) developed using the online bioinformatic tools, Primer-Blast (Ye et al., 2012, BMC Bioinformatics 13:134) and Primer3 (Untergasser et al., 2012, Nucleic Acids Res. 40(15):e115-e115.). The long-range PCR products were visualized on a 2% agarose gel. This long-range PCR method allowed for further evaluation of the NANOS3 target site to detect large (>500 bp) indels. Three of the samples (90d #5069, live male #838, and live male #3964) were observed to carry potentially large (>500 bp) deletions, as indicated by the presence of bands smaller than the wild type control sample (FIG. 35).

### NANOS3 Long-Amplicon Library Preparation and Next Generation Sequencing (NGS)

Long-range PCR products were purified using a AMPure PB Kit (Pacific Biosciences of California, Inc, (“PacBio”) Menlo Park, CA). SMRTbell libraries were prepared with PacBio barcoded overhang adapters, which allowed for pooling of the samples (SMRTbell® Express Template Prep Kit 2.0 and Barcoded overhang adapter kit 8A, PacBio, Menlo Park, CA). Sequencing was performed on a PacBio Sequel II system by the UC Davis DNA Technologies & Expression Analysis Core. HiFi reads (reads generated with Circular Consensus Sequencing (CCS) analysis whose quality value is equal to or greater than 20) were sorted by barcode and BAM files were converted to individual FASTQ files for each sample using SMRT Link v11.0.0.146107. HiFi reads were aligned to a reference FASTA file corresponding to the 6,274 bp target region of bovine NANOS3 (ARS-UCD1.2.108:7:11,805,072-11,811,345) using BWA MEM2 v2.2.1. SAM files were converted to BAM files, sorted, and indexed using SAMtools v1.15. The proportion and types of alleles were determined for each sample using AlleleProfileR (Bruyneel et al., 2019).

The NANOS3 long-amplicon NGS data revealed a variety of alleles present in the CRISPR/CAS9 NANOS3 targeted bovines, with indels ranging from 1 bp up to 1.5 kb (Table 10). Seven of the eight NANOS3 targeted bovines (87.5%) were successfully edited (i.e., 0% wild type alleles). One NANOS3 targeted bovine, 40d_3996, was not edited (100% wildtype). Out of the seven edited NANOS3 targeted bovines, five (71%) were mosaic (i.e., carried more than 2 different alleles). Six of the seven NANOS3 edited bovines (85.7%) carried only knockout allele(s). A knockout allele is defined as having either a frameshift-inducing indel (i.e., small indels that are not multiples of three) or a moderate sized indel (>21 bp) in a protein-coding region that are predicted to generate a complete loss-of-function mutation. One of the seven NANOS3 edited bovines, 15mo_3964, carried an allele (#2; 30% of the reads) with only in-frame deletions. However, the long-amplicon analysis revealed that 15mo_3964 also carried 2 alleles (total of 70% of the reads) each with large deletions.

Overall, the NANOS3 long-amplicon NGS data 1) confirmed the results observed in the initial small-amplicon PCR and Sanger Sequencing analysis, 2) enabled identification and measurement of the proportion of unique alleles present in the mosaic samples, and 3) revealed alleles with large deletions (>500 bp) that were not detected with previous methods. Ultimately, this analysis shows a 75% (6/8) total knockout rate was achieved with our dgRNA_4+7 editing approach (Table 10).

TABLE 10

| | | | gRNA4¹ | | gRNA7² | | dgRNA_4 + 7 | | |
|---|---|---|---|---|---|---|---|---|---|
| | | | | Indel | | Indel | | Indel | |
| | Allele | Allele description | Indel | size | Indel | size | Indel | size | Proportion |
| Sample | # | (indel size category)¹ | start⁴ | (bp) | start⁴ | (bp) | start⁴ | (bp) | of reads |
| 40d_3993 | 1 | gRNA 4 & 7: small | 118 | −1 | 409 | −8 | | | 100% |
| 40d_3996 | 1 | wild type | | | | | | | 100% |
| 90d_3987 | 1 | gRNA4: moderate & gRNA7: small | 83 | −51 | 409 | −8 | | | 37% |
| | 2 | gRNA 4 & 7: moderate | 115 | −27 | 410 | −32 | | | 36% |
| | 3 | dgRNA_4 + 7: targeted | | | | | 112 | −298 | 28% |
| 90d_5069 | 1 | dgRNA_4 + 7: large | | | | | −331 | −960 | 36% |
| | 2 | dgRNA_4 + 7: large | | | | | −662 | −1122 | 32% |
| | 3 | gRNA4: small & gRNA7: moderate | 107 | −20 | 393 | −27 | | | 26% |
| | 4 | gRNA 4 & 7: moderate | 115 | −27 | 367 | −55 | | | 5% |
| 280d_848 | 1 | gRNA4: moderate & gRNA7: small | 116 | −273 | 409 | −8 | | | 64% |
| | 2 | dgRNA_4 + 7: targeted | | | | | 117 | −297 | 36% |
| | | | | | | | | (−298, +1) | |
| 15mo_854 | 1 | dgRNA_4 + 7: targeted | | | | | 119 | −297 | 61% |
| “Fun Bun” | 2 | dgRNA_4 + 7: targeted | | | | | 119 | −291 | 26% |
| | | | | | | | | (−297, +6) | |
| | 3 | dgRNA_4 + 7: targeted | | | | | 117 | −298 | 9% |
| | 4 | dgRNA_4 + 7: targeted | | | | | 117 | −293 | 2% |
| | | | | | | | | (−298, +5) | |
| | 5 | dgRNA_4 + 7: targeted | | | | | 119 | −294 | 2% |
| | | | | | | | | (−297, +3) | |
| 15mo_838 | 1 | gRNA4: large & gRNA7: moderate | −714 | −979 | 410 | −32 | | | 59% |
| “Fauci” | | | | (−1000, +21) | | | | | |
| | 2 | dgRNA_4 + 7: targeted | | | | | 117 | −298 | 15% |
| | 3 | gRNA4: large & gRNA7: moderate | −715 | −1000 | 410 | −32 | | | 10% |
| | 4 | gRNA4: moderate & gRNA7: small | 97 | −30 | 412 | −7 | | | 8% |
| | 5 | dgRNA_4 + 7: targeted | | | | | 114 | −298 | 8% |
| | | | | | | | | (−301, +3) | |
| 15mo_3964 | 1 | dgRNA_4 + 7: large | | | | | 107 | −1326 | 64% |
| “Frodo” | 2 | gRNA 4 & 7: small⁵ | 119 | −3 | 410 | −6 | | | 30% |
| | 3 | dgRNA_4 + 7: large | | | | | 59 | −1502 | 6% |

¹The gRNA4 cut site is position 118, relative to the start of exon 1.
²The gRNA7 cut site is position 415, relative to the start of exon 1.
³Description of the indel(s) present in the allele based on indel size and start location. All indels are predicted to knockout bovine NANOS3 (i.e., either a frameshift-inducing indel or a moderate sized indel in a protein-coding region that are predicted to generate a complete loss-of-function mutation), unless otherwise noted. Size categories: small <21 bp, moderate = 21-500 bp, targeted = 297 ± 5 bp, large >500 bp.
⁴Starting position of the indel, relative to the start of exon 1.
⁵Allele #2 present in 15mo_3964 has only in-frame deletions (i.e., small deletions that are multiples of 3), which results in an amino acid substitution and a deletion of 3 amino acids total.

### Example 8—Guide RNA Design and In Vitro Testing of Guide RNAs for Knockout of Ovine NANOS3

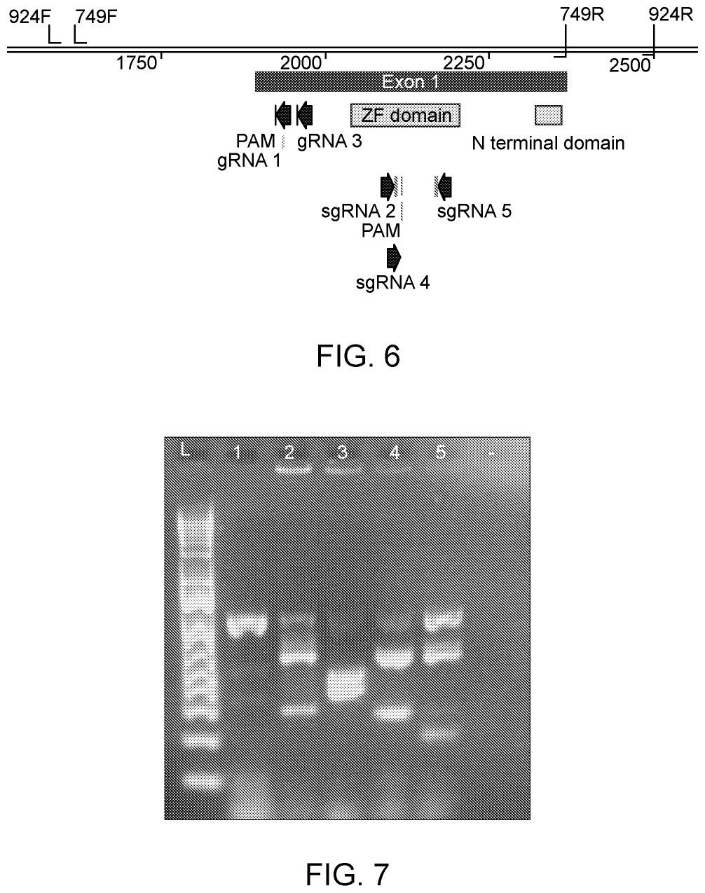

The ovine NANOS3 transcript is 543 bp and consists of two exons. We chose to target exon 1, as it is larger (475 bp) and contains the coding region for the C2HC-type zinc finger binding and N-terminal domains (Hashimoto et al., 2010) that are critical for NANOS3 function (Beer and Draper, 2013) (FIG. 6).

Guide RNAs (gRNAs) targeting exon 1 of the ovine NANOS3 gene were designed using the CHOPCHOP version 3 web tool based on the ovine Oarv_3.1 reference genome (Labun et al., 2019). Table 11 shows the guide RNA sequences tested. The potential off-target binding sites were identified by CHOPCHOP for the Oarv_3.1 reference genome and by Cas-OFFinder for the Oarv_3.1 and ARS-UI_Ramb_v2.0 reference genomes (Bae et al., 2014). The criteria used to identify off-targets with Cas-OFFinder was off-target genomic sequences with up to 3 mismatches (discrepancies between a base of the gRNA and the predicted off-target site) and no RNA or DNA bulges (unpaired stretches of nucleotides).

Four out of the five gRNAs selected had no potential off-target binding sites and the fifth gRNA, gRNA #5, will not be analyzed for off-target effects because it had poor in vitro cleavage efficiency, so it was not used for the in vivo knockout. Based on a systematic analysis of CRISPR-Cas9 mismatch tolerance (Anderson et al., 2015) and testing in bovine zygotes (Hennig et al., 2020) all of the gRNAs selected for in vivo testing are highly unlikely to cause off-target editing.

TABLE 11

| Guide Sequence | Sequence | SEQ ID |
|---|---|---|
| Reference | (20 nt gRNA + PAM) | NO: |
| 1 (gRNA1) | ACTGGACACGGATCGCGCCGAGG | 41 |
| 2 (gRNA2) | ACTGGGGGCACACGTAGTCGCGG | 42 |
| 3 (gRNA3) | TGGTCCGCTCGGACAAGGCGAGG | 43 |
| 4 (gRNA4) | ACACGTAGTCGCGGAGGATGGGG | 44 |
| 5 (gRNA5) | GCAAACACAACGGCGAGTCCCGG | 45 |

After using bioinformatic tools to select guides with a low chance of off-target mutagenesis, the gRNAs were tested using an in vitro cleavage assay. Each gRNA was incubated with 150 ng of Cas9 protein, 60 ng of polymerase chain reaction (PCR) amplified genomic NANOS3 ovine DNA (749 bp), 100 ng of single gRNA (sgRNA), 1 NEB3.1 buffer for 1 hour at 37° C. and ran the resulting product on a 1% agarose gel. It was observed that 3 of the gRNAs, gRNA #2, #3, and #4, successfully cut the target region in vitro with high efficiency and gRNA #2 cut the target region with medium-low efficiency (FIG. 7). The resulting DNA was sequenced using Sanger sequencing (GeneWiz, South Plainfield, NJ) and confirmed that gRNAs #2-5 cut at the expected location, between the 3^{rd} and 4^{th} base pairs from the start of the PAM site. Next, gRNA #2 and #3 were further tested in vivo to determine the blastocyst development rate and mutation efficiency of a dual gRNA approach to NANOS3 KO (FIG. 3).

### Example 9—In Vivo Guide RNA Validation of Ovine NANOS3

### Ovine Embryo Production

In order to produce embryos for in vivo testing, ovine ovaries were collected from a local slaughterhouse and transported to the laboratory at 35-37° C. in sterile saline (Hennig et al., 2020, Owen et al., 2020b). Cumulus-oocyte-complexes (COCs) were aspirated from follicles and groups of 50 COCs were transferred to 4-well dishes containing 500 μL of maturation media (IVF Bioscience, Falmouth, United Kingdom). COCs were incubated for 22-24 hr at 38.5° C. in a humidified 5% CO₂ incubator. Approximately 25 COCs per drop were fertilized in 50 uL drops of SOF-IVF media (Bakhtari and Ross, 2014) with 1×10⁶ sperm per mL and incubated for 6 hr at 38.5° C. in a humidified 5% CO₂ incubator (Hennig et al., 2020, Owen et al., 2020b). Six hours post insemination (hpi), presumptive zygotes were denuded by pipetting in SOF-HEPES medium for 5 min (Bakhtari and Ross, 2014, Hennig et al., 2020, Owen et al., 2020b). Zygotes were incubated in in 500 μL wells (n=50-200 per well) of in vitro culture media (IVF Bioscience, Falmouth, United Kingdom) at 38.5° C. in a humidified atmosphere of 5% C02, 5% 02, and 90% N₂ for 7 days (Bakhtari and Ross, 2014, Hennig et al., 2020, Owen et al., 2020b). See also, FIGS. 6 and 7.

Single gRNA (sgRNA) In Vivo Validation of Ovine NANOS3

To determine the mutation rate for each guide, presumptive zygotes (6 hpi) were electroporated using a Nepa21 electroporator with 20 μL of a solution containing 100 ng/μL of sgRNA 2 and sgRNA 3 (Synthego, Menlo Park, CA) alongside 200 ng/μL of Cas9 protein (PNA Bio, Inc., Newbury Park, CA), and 8 ul of Opti-MEM (Thermo Fisher) which had been incubated together at room temperature for 10 minutes prior to electroporation. A group of 90 presumptive zygotes were electroporated at 40 volts, 3.5 ms pulse length, 50 ms pulse interval, 2 bipolar pulses. The control embryos (n=41) were also included the trial (Table 12).

To determine blastocyst development rate, day 7 embryos were scored for developmental stage reached. The electroporation treatment did not appear to harm blastocyst development rate with the control blastocyst rate of 20% (8/41) and electroporated blastocyst rate of 19% (17/90). For mutation analysis, all electroporated blastocysts and 2 randomly selected control blastocysts were individually collected for DNA extraction with lysis buffer (Epicentre). The target region was amplified by two rounds of nested polymerase chain reaction (PCR) using primers (Integrated DNA Technologies) developed using the online bioinformatic tools, Primer-Blast (Ye et al., 2012) and Primer3 (Untergasser et al., 2012). PCR products were visualized on a 1% agarose gel, purified using the QIAquick Gel Extraction Kit (Qiagen, Inc., Valencia, CA) and Sanger sequenced (GeneWiz, South Plainfield, NJ). The dual sgRNA approach resulted in an over 60% total mutation rate (Table 12).

TABLE 12
Comparison of blastocyst rates and mutation
efficiencies of tested ovine NANOS3 gRNAs

| | Location | Blastocyst | | Total | | Knockout | >5% wt | |
|---|---|---|---|---|---|---|---|---|
| gRNA | (Exon 1) | Rate | | Mutation | | (0% wt) | Mosaic | Unedited |
| Control | — | 8/41 | (20%) | 0/2 | (0%) | — | — | — |
| 2 + 3 | 2 | 17/90 | (19%) | 9/17 | (14%) | 1/17 (6%) | 13/17 (76%) | 3/17 (18%) |
| 2 + 3 | 3 | 17/90 | (19%) | 12/17 | (83%) | | | |

### Example 10—Optimization of Bovine Embryonic Stem Cell Culture

### Development of ESC Lines

To determine the best conditions to achieve embryo chimerism, different bovine ESCs lines were developed, to track the cells inside of the embryo.

### Bovine ESC Line 1

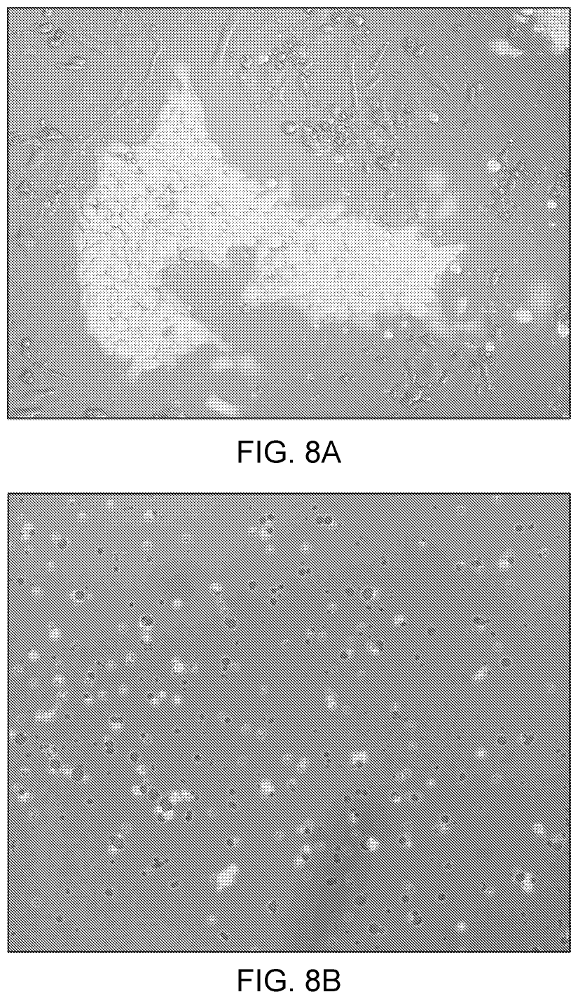

First, a male line of ESCs from a pure-bred Jersey embryo using conditions described in Bogliotti et al., 2018; WO 2019/140260 was developed. This line of cells was cultured in N2B27 media containing IWR-1 (WNT inhibitor; Sigma-Aldrich), Y27632 (ROCK inhibitor; Enzo Life Sciences) and Activin A (R&D Systems) on mouse embryonic fibroblast (MEF) feeder cells (Invitrogen). EGFP coding sequences were cloned into the multiple cloning site of the lentiviral vector, to express EGFP under control of the UbC promoter. The lentivirus was produced using a third-generation packaging system (Invitrogen's ViralPower). Male Jersey ESCs were plated onto vitronectin (Invitrogen) coated plates 24 h prior to transduction with EGFP lentivirus. Media was changed and, 24 h post-transduction and 48 h post transduction, cells were plated across a 96 well plate seeded with MEF. One week later, wells with bright green colonies of cells were harvested using TrypLE and diluted to 1 cell/200 ul and plated into a new 96 well plate seeded with MEF, adding 100 ul/well. A week after one well was identified as having colonies of bright EGFP positive cells and these were expanded on MEF. Following isolation of this EGFP-ESC line the culture tested positive for Mycoplasma. Plasmocure (Invivogen) was used to eliminate the mycoplasma contamination from this cell line. Following removal of mycoplasma, a line with bright green expression was established (FIGS. 8A-8B) and kept frozen (−196 C.°) at approximately. 1×10⁶ cell/ml in N2B27 media with 10% DMSO as cryoprotectant, until using. Before using, a vial containing frozen cells were thawed in a water bath at 37 C.°, plated in one well of a 48 wells plate covered in MEF and passaged every 2-3 days using the methodology described above.

### Bovine ESC Line 2

The second ESCs line was derived from a female embryo at the early blastocyst stage and cultured in N2B27 media previously described (see e.g., International Patent Application Pub. No. WO 2019/140260, which is incorporated by reference as if expressed in its entirety herein), these cells were platted in a different matrix, vitronectin, free from mouse embryonic fibroblast (MEF) feeder cells, with the aim of obtain a pure line of bovine cells.

### Bovine ESC Line 3

A third line of ESCs have been derived from a targeted knock-in of SRY at the safe-harbor H11 locus. The hemizygous SRY XY was accomplished using the CRISPR-Cas9 system in bovine zygotes ((Owen et al., 2020a)). Genomic analyses revealed no wildtype sequence at the H11 target site, but rather a 26 bp insertion allele with no SRY, and a complex 38 kb knock-in allele with seven copies of the SRY:GFP template and a single copy of the donor plasmid backbone. Semen was collected from the SRY bull at sexual maturity and used to carry out in vitro fertilization utilizing oocytes collected from cow ovaries obtained from a local slaughterhouse and matured in vitro. After reaching blastocyst stage, embryos were utilized to derive ESCs. One male ESC line was obtained and cultured in MEF and latter transitioned to vitronectin. Although, these ESCs in culture were negative to green fluorescence, they tested positive to GFP after PCR analysis. This provides a unique sequence to track the donor cells using PCR.

### Expanded Potential Stem Cells (EPSCs)

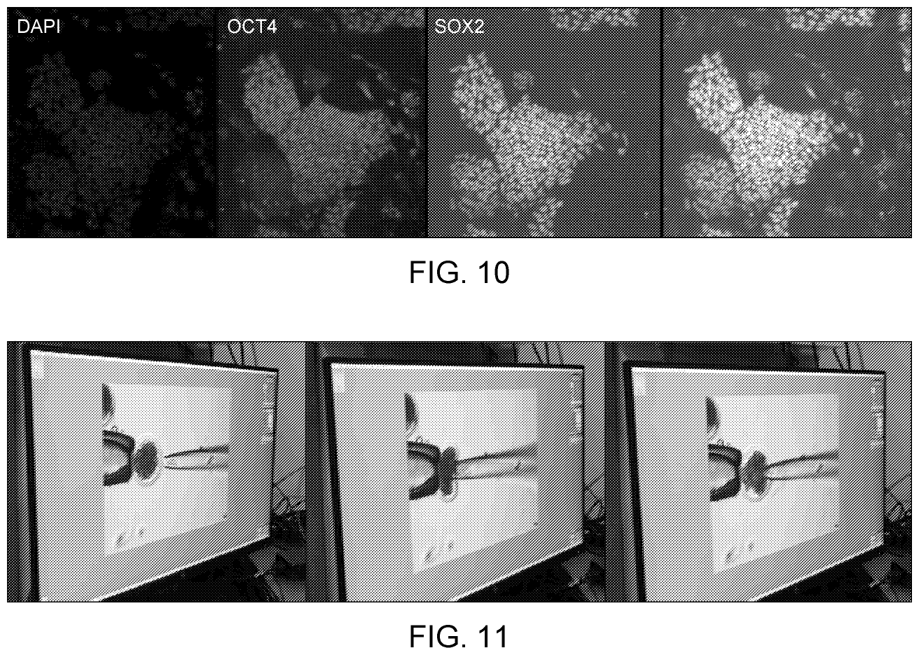

Another type of ESCs named expanded potential stem cells (EPSCs) have been characterized to have broader developmental potential to generate embryonic and extraembryonic cell lineages in bovine (Zhao et al., 2021), porcine and mouse (Gao et al., 2019). EPSCs express high levels of pluripotency genes, propagate robustly in feeder-free culture, and are genetically stable in long-term culture. Cells also have enriched transcriptomic features of early preimplantation embryos and differentiate in vitro to cells of the three somatic germ layers and, in chimeras, contribute to both the embryonic (fetal) and extraembryonic cell lineages. An EPSCs have been generated and in every cell passage, cells from all derived lines were tested by immunofluorescence for expression of the embryonic stem cell markers OCT4 and SOX2. The expression of these two markers (FIG. 10) indicates that the cells have pluripotency, the ability to generate a multitude of cell types and tissues (Wu and Izpisua Belmonte, 2015).

### Example 11—Evaluation of Embryo Chimerism and Optimization of Embryo Complementation Conditions

The cell lines developed were used to perform experiments to determine the best conditions to achieve embryo chimerism, the green ESC were expected to incorporate into the inner cell mass of the embryo (ICM).

To evaluate the capacity of the ESC to incorporate into the ICM of the embryo, or develop a chimera, we injected in vitro produced embryos at different developmental stages. We have injected embryos at day 3, 4, 5 and 6 after in vitro fertilization with 5 to 10 ESCs from different cells lines. The number of ESC injected was adjusted depending on the developmental stage of embryos. Embryos injected at day 3 and 4 days after in vitro fertilization were injected with 5 cells per embryo, embryos injected at day 5 after in vitro fertilization were injected with 8-10 cells and finally, embryos injected at day 6 after in vitro fertilization were injected with 10 cells per embryo.

To perform injections, first cells were dissociated into single cells and kept in ice during the embryo manipulation. Embryos with high quality were selected and transferred to a drop of (4-(2-hydroxyethyl)-1-piperazineethanesulfonic acid) (HEPES) in a dish containing an aliquot of ESC and placed on an inverted microscope. One by one the embryos were immobilized with a holding pipette and through a hole in the zona pellucida created by a single laser pulse, the injection needle containing the cells was introduced into the embryo and cells were placed close to the ICM (FIG. 11). After microinjection, the embryos were washed in HEPES and cultured in the confocal imaging system until the blastocyst stage, to evaluate the incorporation of the cells into the ICM. Embryos were culture in a media composed by half volume of bovine culture media (IVF Bioscience®) and half volume of N2B27 media. Culturing in this media resulted in a higher blastocyst rate as compared to culturing only in bovine culture media.

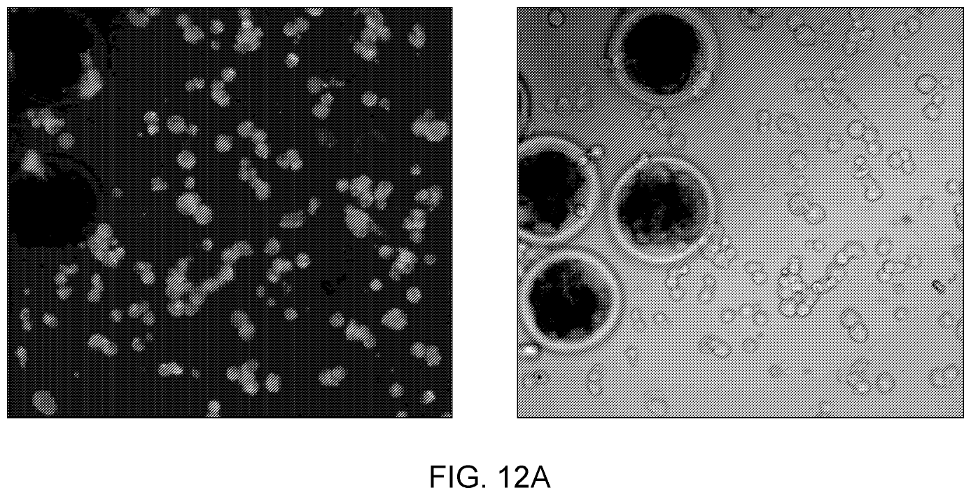

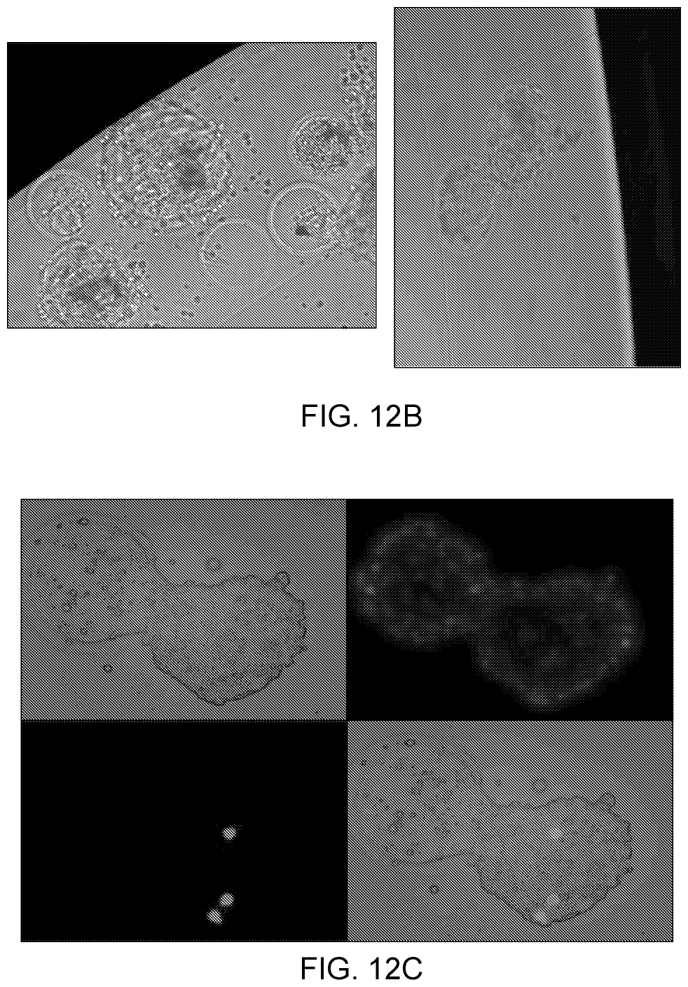

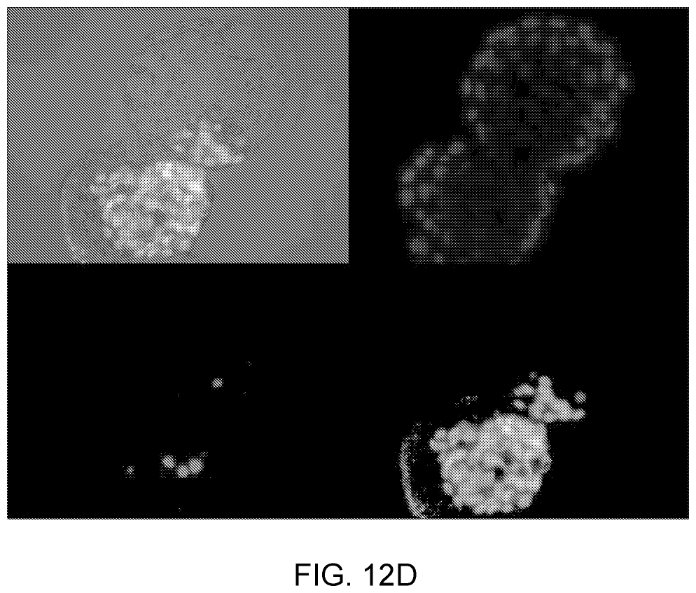

From these studies, the optimal conditions for injection, manipulation time, laser power and best diameter of injection needle without affecting the development of the embryo were determined. The capacity of the different ESC to incorporate into the embryo ICM and create a chimera might be affected by the cell line, pluripotency state and passage number, besides other factors inherent to the embryo, as its quality, developmental stage and factors related to the technic. To track ESCs not expressing GFP inside of the embryo after injection and evaluate their complementation, utilized PKH26 red fluorescent dye was utilized, which stains the cytoplasm (FIGS. 12A-12D). This allowed the use of the derived ESC lines with a low passage number.

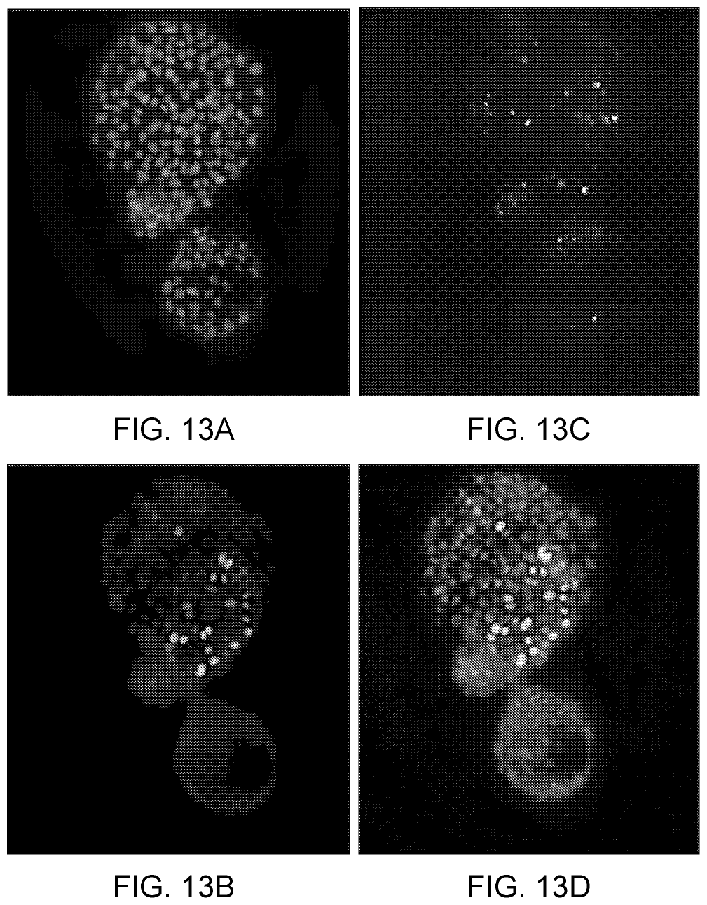

After injecting day 6 (after in vitro fertilization) embryos with 10 ESCs, multiplication of the cells inside of embryos (FIGS. 13A-13D, 14A-14D, and 15A-15D) was observed. After the end of the incubation period, day 8 after fertilization, the hatching blastocysts were fixed with 4% paraformaldehyde and stained with DAPI (blue-fluorescent DNA stain) and a green fluorescence SOX2 marker (a transcription factor that is essential for maintaining pluripotency of undifferentiated embryonic stem cells) to evaluate the pluripotency of the injected ESC. At least 50% of the injected cells were positive for both red and green colors, indicating pluripotency of the injected ESCs, and the presence of red cells in both the inner cell mass (future embryo) and the trophectoderm (not pluripotent and so does not stain green) indicating the incorporation of some of the donor ESCs in the developing host embryo.

These optimized conditions can be used in the context of e.g., complementation of embryos for germline complementation.

### Example 12—Derivation of Expanded Potential Stem Cells from Sexed Semen and Establishment of a Reporter Cell Line

There are different pluripotent states termed “naive” and “primed” (FIG. 36). Mouse ESCs are the gold standard of naive pluripotency, while human as well as bovine ESCs exist in the developmentally more advanced primed pluripotent state. In general, primed ESC have poor single-cell clonality, which is undesirable for gene editing and lower derivation efficiencies. Importantly, naive ESCs efficiently contribute to chimera formation, are capable of germline transmission after blastocyst injection and can generate an entire adult organism when injected into embryos.

Recently, Zhao et al. (2021) published a new type of bovine ESC named expanded potential stem cells (bEPSCs). This bEPSCs expressed high levels of pluripotency genes, propagated robustly in feeder-free culture, and were genetically stable in long-term culture. bEPSCs have enriched transcriptomic features of early preimplantation embryos and were capable of differentiate in vitro into cells of the three somatic germ layers and, in chimeras, contributed to both the embryonic (fetal) and extraembryonic cell lineages. Also, precise gene editing was efficiently achieved in bEPSCs, and genetically modified bEPSCs were used as donors in somatic cell nuclear transfer. Stem cells reside in a different pluripotency state, from naïve to primed and the potential to integrate in the ICM of an embryo after injection is reduced as cells move from the naïve to primed states. Cells derived following Bogliotti's protocol (2018), in our case bESC seems to reside in a primed state.

EPSC seem to reside in a naïve state, according to the ability to integrate into the host embryo observed by Zhao et al. (2021). When bovine chimeras were evaluated (at blastocyst stage and at day 38, 40 and 70 of gestation), the expanded potential stem cells were found to be present in the complemented ICM (Zhao et al., 2021). In order to produce bovine chimeras with germline transmission and to compare the complementation rate with the embryos complemented with bESC, Applicant derived and established a line of expanded potential stem cells (bEPSC) from bovine blastocysts fertilized with Y sorted sperm. The cell line was derived, tested for pluripotency markers expression, mycoplasma free and confirmed to be male sex through PCR analysis of the cell's DNA.

In order to have a reporter gene, the derived bEPSC were transduced with a lentivirus vector with tdTomato under the control of EF1 promoter. After multiple passages and clonal expansion, the potential expanded stem cells showed 100% red florescent under UV light, due to the expression of the fluorescent tdTomato transgene. This approach will allow us to evaluate the rate of complementation and to trace cell lineage during embryonic and fetal development and afterbirth.

### Example 13—Production of NANOS3^{-/-} Knockout Chimeric Embryos with Expanded Potential Stem Cells (bEPSC) and Transfer

Using the red fluorescent bEPSC as donor cells (Example 12), Applicant followed the previously mentioned protocol to obtain NANOS3 −/− knockouts embryos. Six hours post insemination (hpi), presumptive zygotes were denuded by light vortex in SOF-HEPES medium for 5 min and embryos were injected with a NANOS3 dual gRNA, 6 μL of a solution containing 67 ng/μL of gRNA #4 and 67 ng/μL of sgRNA #7 (Synthego, Menlo Park, CA). After CRISPR/Cas9 injection, zygotes were incubated in drops of IVC culture media (IVF Bioscience, Falmouth, United Kingdom) at 38.5° C. in a humidified atmosphere of 5% CO₂, 5% O₂, and 90% N2 for 5 days. Applicant produced chimeric embryos following the methodology detailed previously, by injecting 41 presumptive NANOS3-knockout 5d morulas with 10 red bEPSC, 5 of those embryos were produced with Y sorted sperm. Applicant also injected 10 red bEPSC in 5 embryos control embryos that were not presumptive NANOS3−/− knockouts.

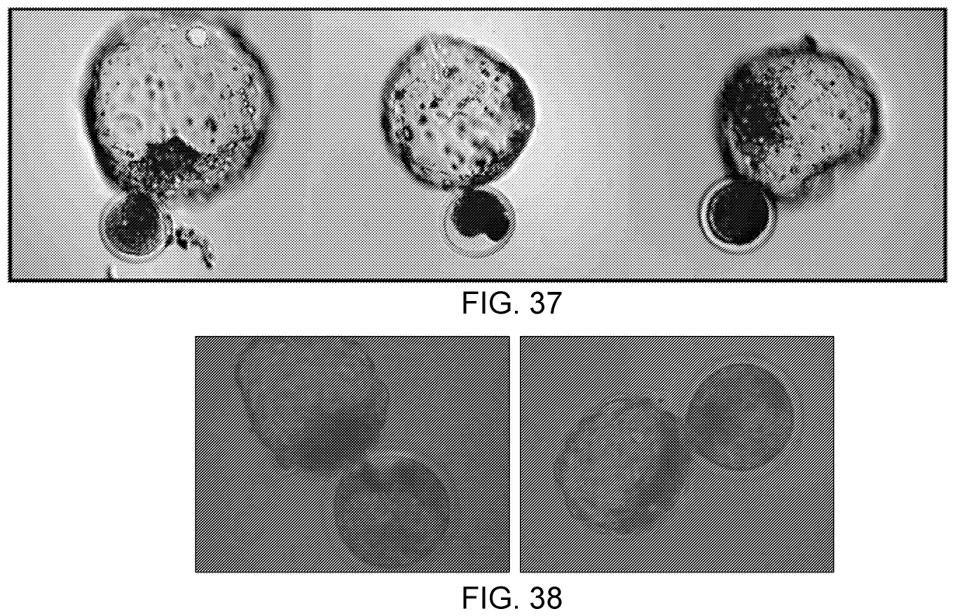

After 2 days of in vitro culture, 21 good quality blastocysts were obtained. 18 presumptive NANOS3-knockout and 3 no injected previously with Crispr/Cas9. 15 presumptive NANOS3-knockout and 3 not injected with Crispr/Cas9 blastocyst were selected and loaded into straws for embryo transfer. A total of 13 straws were loaded, 8 straws with one embryo each, 7 of them with presumptive NANOS3-knockout-bEPSC complemented embryos, and 1 straw with a single embryo not treated with Crispr/Cas9. The remaining 5 straws were loaded with 2 embryos each, 4 straws with presumptive NANOS3-knockout-bEPSC complemented embryos and 1 straw with a complemented embryo no treated with Crispr/Cas9. Selected embryos were transferred into the recipient's uterine horn that was ipsilateral to the ovary harboring a CL, 7 days post heat detection (Apr. 15, 2022). Those embryos that were not transferred embryos were fixed, stained with DAPI and incubated with an antibody anti SOX2 to confirm pluripotency and allow visualization of the injected red cells (FIG. 37).

Six of thirteen (46%) of recipients transferred with one presumptive NANOS3-knockout-bEPSC complemented embryo were detected pregnant.

### Example 14—Production of Chimeric Embryos with Expanded Potential Stem Cells (bEPSC), Transfer and Embryo Recovery at Elongation Stage

Applicant collected ovaries and produced embryos to generate NANOS3^{-/-} knockout chimeric embryos with expanded potential stem cells (bEPSC) to be transferred and recovered from recipients by flushing at around 16 days (9 days after ET). Applicant aimed to recover multiple embryos at their elongation stage, to rapidly evaluate the complementation.

Applicant produced embryos using male sexed semen and at day 5 of development (morula stage) embryos were injected with 10 embryonic stem cells (ESC) each. Applicant used the same cells implemented in the previous experiment. Cells were derived using the media to produce Expanded Potential Stem Cells (Zhao et al., 2021, Proceedings of the National Academy of Sciences 118, 9) and have been proven to complement both mouse and bovine blastocysts after injection. Cells were previously transfected with lentivirus to express the tdTomato reporter gene as a fluorescent marker and clonally selected so they were 100% red. Immediately before the injection of the ESC into the embryos, ESC were stained with a red dye to allow visualization during injection and localization of the cells in the blastocyst before the embryo transfer. Applicant evaluated the blastocyst rate of injected embryos and degree of complementation with the injected ESC and the best quality blastocysts were selected to be transferred to synchronized recipients that same day. FIG. 38 shows representative 7-day blastocysts with red-stained (as represented in greyscale) ESCs on the day of embryo transfer.

Two of the recipient cows received one single blastocyst each and the other two recipients received 8 blastocysts each. These latter two cows were then flushed ˜9-10 days later to recover the elongated embryos at day 16 of development and analyze the contribution of the ESC.

Three of 8 embryos were recovered in one of the recipients and 7 of 8 in the other recipient (n=10). Recipients with one embryo each were not disturbed until day 30 of transfer and were determined not to be pregnant based on blood tests.

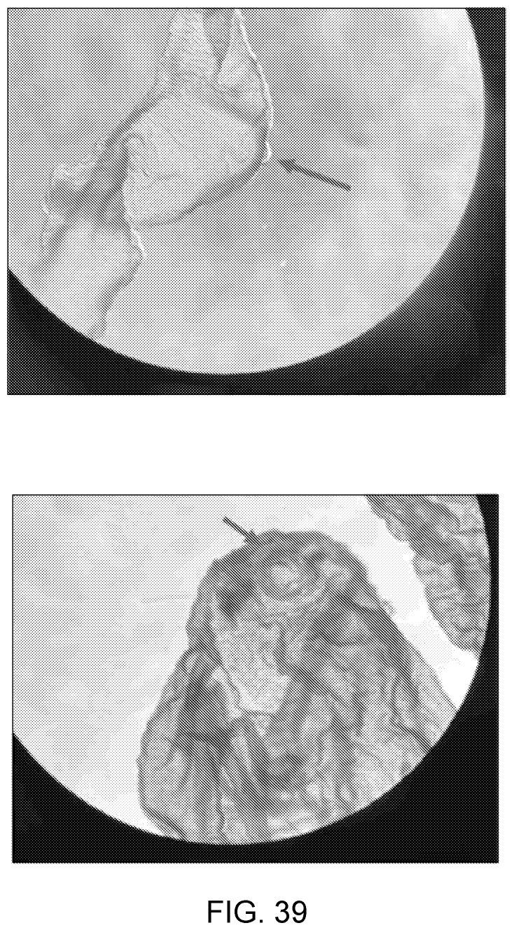

Recovered embryos were visualized under a stereoscope to identify the embryonic disc, that will form the embryo proper. Five of the recovered embryos had a clear embryonic disc (see e.g., FIG. 39)

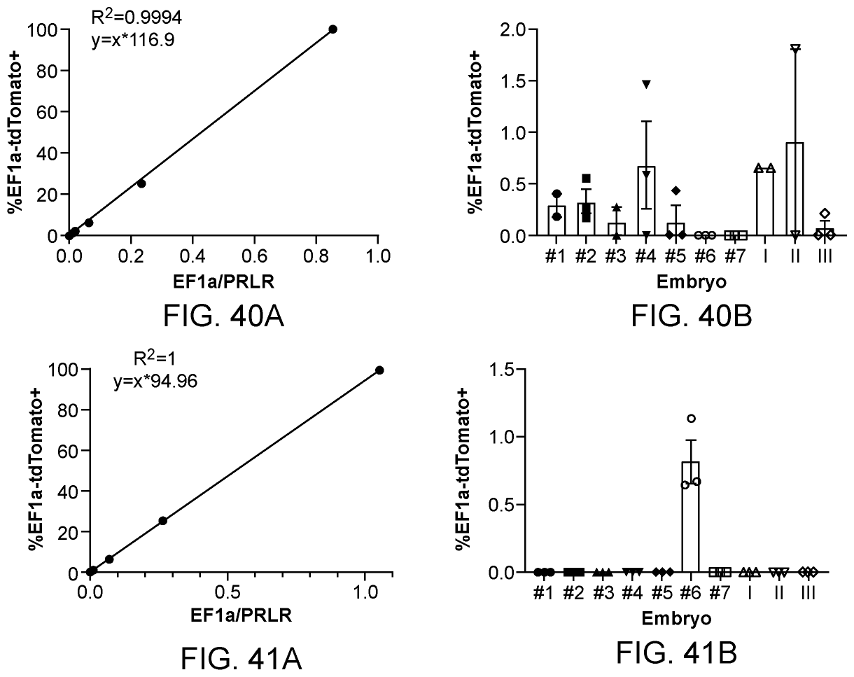

In order to detect the presence of the ESC in the recovered embryos Applicant performed qPCR assays using probe and primers to detect the exogenous promoter (human elongation factor 1alpha [EF1a]) driving the expression of tdTomato in the injected cells. A housekeeping control gene was also detected using probe and primers specific to bovine prolactin receptor (PRLR). To determine the percent of cells containing the tdTomato construct in the fetal DNA, Applicant used DNA prepared from the tdTomato expressing ESC and diluted it with wild type bovine DNA to give a standard curve ranging from 0.0244% to 100% tdTomato ESC DNA. The EF1a and PRLR qPCR reactions were multiplexed so Applicant were detecting both targets in the same well. A standard curve prepared from 4-fold dilutions of tdTomato ESC DNA was used to calculate a relative quantity for both EF1a and PRLR. Then the ratio EF1a/PRLR was plotted against % tdTomato ESC DNA to give a standard curve from which we could measure % tdTomato ESC DNA in the embryo DNA (FIG. 40A, FIG. 41A).

Genomic DNA was extracted from 25 mg of placental tissue cut from each of the 10 elongated embryos that were recovered (I, II, II from one cow. #1-#7 from the second cow). Applicant found low-level contribution by tdTomato+ ESC to the placental tissues from 8/10 elongated embryos evidencing complementation in these embryos (FIG. 40B). To confirm these results, Applicant did a second genomic DNA extraction from a second 25 mg piece of placental tissue from the 10 elongated embryos. This time Applicant found low-level contribution by tdTomato+ ESC to the placental tissues from 1/10 elongated embryos (FIG. 41B). Altogether 9/10 elongated embryos had detectable evidence of tdTomato+ ESC DNA in 1 out of 2 pieces of placenta. The level of tdTomato+ ESC contribution ranged from 1 in 110 cells to 1 in 1395 cells. Although this a low-level contribution, it does provide evidence of ESC contribution to the developing trophectoderm and complementation with donor cells.

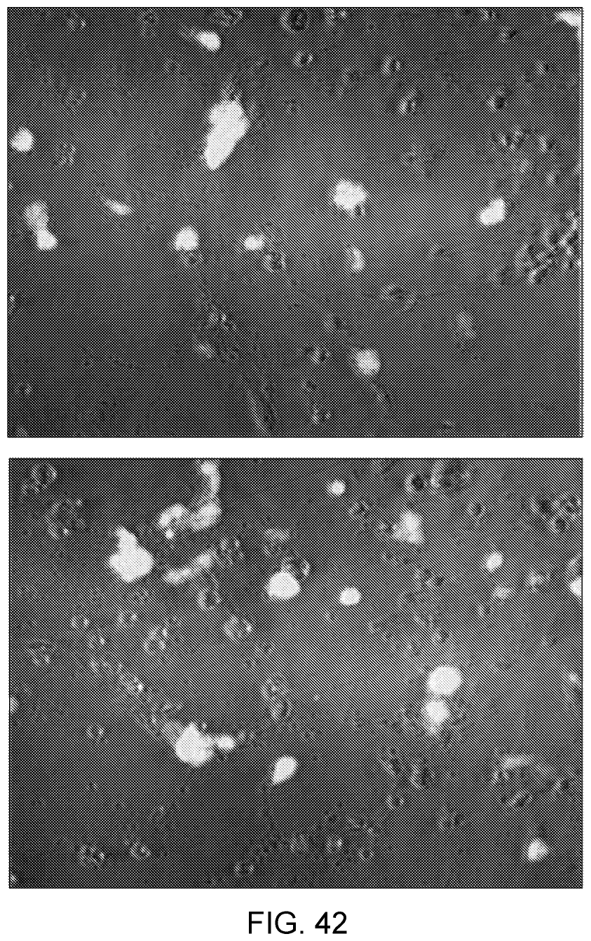

At present Applicant is testing further for the presence of tdTomato using immunofluorescence with an anti-tdTomato protein antibody. Applicant have tested the specificity of the anti-tdTomato antibody in cultured ESCs utilized in the embryonic injections (FIG. 42).

Applicant also produced embryos at the lab that were injected at morula stage with ESC to evaluate their complementation. This time injected cells were treated at plating and passaging with the CEPT (chroman 1, emricasan, polyamines, and ISRIB) cocktail. Applicant injected two different lines of cells. A group of embryos was injected with ESC expressing TdTomato and another group with ESC derived from Cosmo (male carrying GFP marker). Immediately before injections both types of cells were stained with a red dye to facilitate visualization. Both lines of cells were derived in expanded potential stem cell media, are male sexed, mycoplasma free, and expressed pluripotent markers.

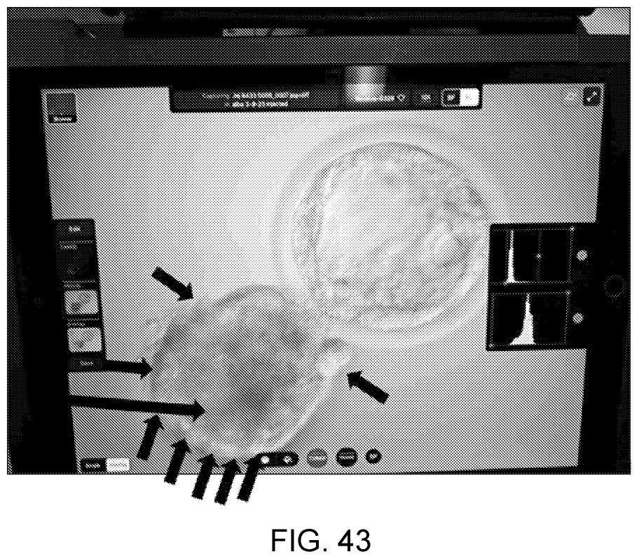

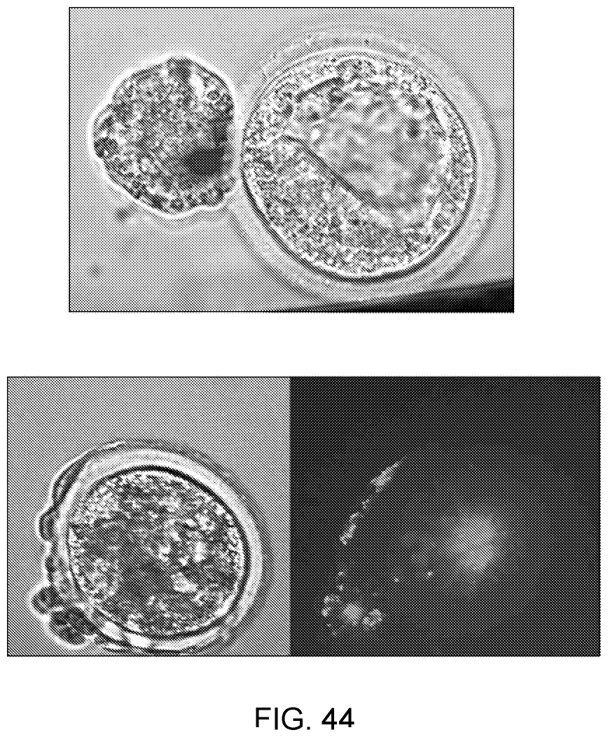

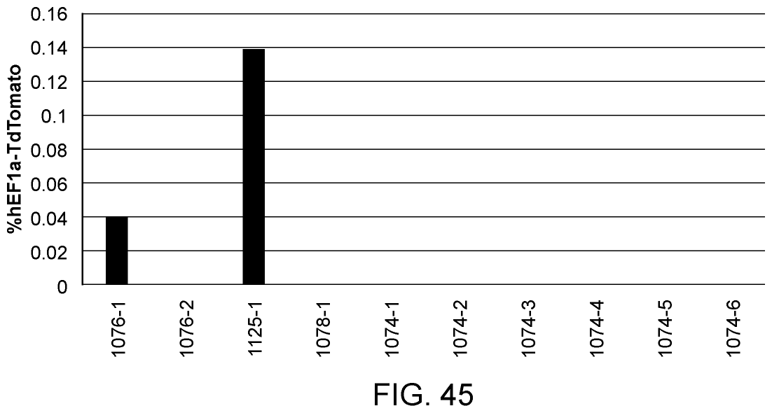

To examine contribution of ESCs to the ICM, 5 ESC were injected in morulas. After injections, embryos were cultured in a 50:50 mix of medias composed of the expanded potential stem cell media and CEPT anti-apoptotic cocktail. At day 7 expanded, hatching blastocysts were examined (FIG. 43). There is clear evidence of an increase in the number of ESCs beyond 5, and location in the ICM. After 2 days in culture, Applicant evaluated the blastocyst rate of injected embryos and degree of complementation with the injected ESC and the best quality blastocysts were selected to be transferred to synchronized recipients that same day (FIG. 43). Five synchronized recipients Applicant transferred, recipients #1125 and #1076 received 7 and 5 embryos respectively, injected with ESC expressing TdTomato. Recipients #1074, #1078 and #J024 received 10, 10 and 20 embryos respectively, injected with ESC derived from Cosmo. About 5 days later, recipient's uteruses were collected and flushed/opened to recover transferred embryos at day 18 of embryonic development. Embryos were recovered from all the recipients except #J024. Applicant took small pieces of each embryo to extract DNA and to evaluate presence of GFP or tdTomato in the cells of the trophoblast through real time PCR. FIG. 44 shows the results of this qPCR analysis. qPCR detected the presence of tdTomato in placental tissue from two embryos injected with the ESCs carrying the EF1a-tdTomato marker indicating complementation from donor ESCs.

### REFERENCES AS CITED IN THE EXAMPLES

Anderson, E. M., et al. 2015. Systematic analysis of CRISPR-Cas9 mismatch tolerance reveals low levels of off-target activity. J. Biotechnol. 211:56-65.
Bae, S., et al. 2014. Cas-OFFinder: a fast and versatile algorithm that searches for potential off-target sites of Cas9 RNA-guided endonucleases. Bioinformatics 30(10):1473-1475.
Bakhtari, A. and P. J. Ross. 2014. DPPA3 prevents cytosine hydroxymethylation of the maternal pronucleus and is required for normal development in bovine embryos. Epigenetics 9(9):1271-1279.
Beer, R. L. and B. W. Draper. 2013. nanos3 maintains germline stem cells and expression of the conserved germline stem cell gene nanos2 in the zebrafish ovary. Developmental biology 374(2):308-318.
Bishop, T.F. and A.L. Van Eenennaam. 2020. Genome editing approaches to augment livestock breeding programs. The Journal of Experimental Biology 223 (Suppl 1):jeb207159.
Bogliotti, Y. S., et al. 2016. Laser-assisted Cytoplasmic Microinjection in Livestock Zygotes. Journal of Visualized Experiments 5(116).
Chari, R., N. et a.. 2017. sgRNA Scorer 2.0: A Species-Independent Model To Predict CRISPR/Cas9 Activity. ACS Synth Biol 6(5):902-904.
Ciccarelli, M., et al. 2020. Donor-derived spermatogenesis following stem cell transplantation in sterile NANOS2 knockout males. Proceedings of the National Academy of Sciences 117(39):24195-24204.
Gao, X., et al. 2019. Establishment of porcine and human expanded potential stem cells. Nature Cell Biology 21(6):687-699.
Giassetti, M. I., et al. 2019. Spermatogonial Stem Cell Transplantation: Insights and Outlook for Domestic Animals. Annual Review of Animal Biosciences 7(1):385-401.
Gottardo, P., et al. 2019. A Strategy To Exploit Surrogate Sire Technology in Livestock Breeding Programs. G3: Genes|Genomes|Genetics 9(1):203-215.
Hashimoto, H., et al. 2010. Crystal structure of zinc-finger domain of Nanos and its functional implications. EMBO Rep 11(11):848-853.
Hayashi, K., et al. 2011. Reconstitution of the Mouse Germ Cell Specification Pathway in Culture by Pluripotent Stem Cells. Cell 146(4):519-532.
Hennig, S. L., et al. 2020. Evaluation of mutation rates, mosaicism and off target mutations when injecting Cas9 mRNA or protein for genome editing of bovine embryos. Scientific Reports 10(1):22309.
Ideta, A., et al. 2016. Generation of exogenous germ cells in the ovaries of sterile NANOS3-null beef cattle. Scientific Reports 6:24983.
Johnson, S., et al. 2010. Protocols for synchronization of estrus and ovulation. Proceedings Applied Reproductive Strategies in Beef Cattle. San Antonio, TX.
Julaton, V. T. and R.A. Reijo Pera. 2011. NANOS3 function in human germ cell development. Hum. Mol. Genet. 20(11):2238-2250.
Labun, K., et al. 2019. CHOPCHOP v3: expanding the CRISPR web toolbox beyond genome editing. Nucleic Acids Research 47(W1):W171-W174.
McFarlane, G. R., et al. 2019. On-Farm Livestock Genome Editing Using Cutting Edge Reproductive Technologies. Frontiers in Sustainable Food Systems 3(106).
McLean, Z., et al. 2020. Embryo-mediated genome editing for accelerated genetic improvement of livestock. Frontiers of Agricultural Science and Engineering 7(2):148-160.
McLean, Z. L., et al. 2021. Testes of DAZL null neonatal sheep lack prospermatogonia but maintain normal somatic cell morphology and marker expression. Mol. Reprod. Dev. 88(1):3-14.
Miao, D., et al. 2019. Simplified pipelines for genetic engineering of mammalian embryos by CRISPR-Cas9 electroporationt. Biol. Reprod. 101(1):177-187.
Owen, J. R., et al. 2020a. One-step generation of a targeted knock-in calf using the CRISPR-Cas9 system in bovine zygotes. Genome Biology Under Review.
Owen, J. R., et al. 2020b. Harnessing endogenous repair mechanisms for targeted gene knock-in of bovine embryos. Scientific Reports 10(1):16031.
Park, K.-E., et al. 2017. Generation of germline ablated male pigs by CRISPR/Cas9 editing of the NANOS2 gene. Scientific Reports 7:40176.
Richardson, T. E., et al. 2009. Sterile testis complementation with spermatogonial lines restores fertility to DAZL-deficient rats and maximizes donor germline transmission. PLoS One 4(7):e6308.
Suzuki, A., et al. 2014. Interaction of NANOS2 and NANOS3 with different components of the CNOT complex may contribute to the functional differences in mouse male germ cells. Biology Open 3(12):1207-1216.
Taylor, L., et al. 2017. Efficient TALEN-mediated gene targeting of chicken primordial germ cells. Development 144(5):928-934.
Tsuda, M., et al. 2003. Conserved Role of nanos Proteins in Germ Cell Development. Science 301(5637):1239-1241.
Untergasser, A., et al. 2012. Primer3-new capabilities and interfaces. Nucleic Acids Res. 40(15):e115-e115.
Vilarino, M., et al. 2017. CRISPR/Cas9 microinjection in oocytes disables pancreas development in sheep. Scientific Reports 7(1):17472.
Wu, J. and J. C. Izpisua Belmonte. 2015. Dynamic Pluripotent Stem Cell States and Their Applications. Cell Stem Cell 17(5):509-525.
Wu, J., et al. 2017. CRISPR-Cas9 mediated one-step disabling of pancreatogenesis in pigs. Scientific Reports 7(1):10487.
Ye, J., et al. 2012. Primer-BLAST: a tool to design target-specific primers for polymerase chain reaction. BMC Bioinformatics 13:134.
Zhao, L., et al. 2021. Establishment of bovine expanded potential stem cells. Proceedings of the National Academy of Sciences 118(15):e2018505118.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A complemented non-human animal or embryo comprising:
a first population of cells comprising one or more cells, wherein the first population of cells consists of an engineered non-human animal cell or population thereof comprising a NANOS3 gene modification, wherein the NANOS3 gene modification reduces or eliminates the expression of a NANOS3 gene product;
a second population of cells comprising one or more cells, wherein the second population cells are not an engineered non-human cell or population thereof comprising a comprising a NANOS3 gene modification, wherein the NANOS3 gene modification reduces or eliminates the expression of a NANOS3 gene product.

2. The complemented non-human animal or embryo of aspect 1, wherein the second population of cells comprises one or more engineered cells comprising one or more genetic modifications in one or more target genes and wherein the one or more target genes are not NANOS3.

3. The complemented non-human animal or embryo of aspect 1, wherein second population of cells does not comprise an engineered cell.

4. The complemented non-human animal or embryo of any one of aspects 1-3, wherein the second population of cells comprises an elite genome, a genomically selected genome, or both.

5. The complemented non-human animal or embryo of any one of aspects 1-4, wherein the second population of cells comprises one or more
a. embryonic cells, optionally a zygote or inner cell mass cells;
b. stem cells, optionally embryonic stem cells or induced pluripotent stem cells;
c. spermatogonial stem cells or oogonial stem cells;
d. primordial germ cells; or
e. primordial germ cell like cells.

6. The complemented non-human animal or embryo of any one of aspects 1-5, wherein the second population of cells are self-renewing cells.

7. The complemented non-human animal or embryo of any one of aspects 1-6, wherein the second population of cells is pluripotent, totipotent, or multipotent.

8. The complemented non-human animal or embryo of any one of aspects 1-7, wherein the second population of cells is germline competent.

9. The complemented non-human animal or embryo of any one of aspects 1-8, wherein the complemented embryo is a preimplantation embryo, optionally a zygote, 2 cell, 4 cell, an 8 cell, 16 cell, a blastocyst, or a morula.

10. The complemented non-human animal or embryo of any one of aspects 1-9, wherein the first population of cells makes up a percentage of cells of the complemented non-human animal or embryo ranging from about 25 percent to any percent up to but not including 100 percent.

11. The complemented non-human animal or embryo of any one of aspects 1-10, wherein the complemented non-human animal or embryo comprises at least one cell of the second population of cells, optionally wherein the second population of cells makes up a percentage of cells of the engineered non-human animal or embryo ranging from any non-zero percent to about 75 percent.

12. The complemented non-human animal or embryo of any one of the aspects 1-11, wherein the complemented embryo is a day 3 post fertilization embryo, a day 4 post fertilization embryo, a day 5 post fertilization embryo, or a day 6 post fertilization day embryo.

13. The complemented non-human animal or embryo of aspect 12, wherein
a. the day 3 post fertilization complemented embryo comprises about 5 cells from the second population of cells;
b. the day 4 post fertilization complemented embryo comprises about 5 cells from the second population of cells;
c. the day 5 post fertilization complemented embryo comprises about 8-10 cells from the second population of cells; and/or
d. the day 6 post fertilization complemented embryo comprises about 10-20 cells from the second population of cells.

14. The complemented non-human animal or embryo of any one of aspects 11-13, wherein the complemented embryo is a morula.

15. The complemented non-human animal or embryo of any one of aspects 1-14, wherein the complemented non-human animal or embryo is a male.

16. The complemented non-human animal embryo of any one of aspects 1-14, wherein the complemented non-human animal or embryo is a female.

17. The complemented non-human animal or embryo of any one of aspects 1-16, wherein (a) the complemented non-human animal or embryo is a bovine, equine, porcine, ovine, caprine, camelid, cervine, canine, feline, murine, leporine, or cavine; (b) wherein the engineered non-human animal cell or population thereof is a bovine, equine, porcine, ovine, caprine, camelid, cervine, canine, feline, murine, leporine, or a cavine cell; (c) the first population of cells comprising one or more cells is a bovine, equine, porcine, ovine, caprine, camelid, cervine, canine, feline, murine, leporine, or a cavine population of cells; (d) the second population of cells comprising one or more cells is a bovine, equine, porcine, ovine, caprine, camelid, cervine, canine, feline, murine, leporine, or a cavine population of cells; or (e) any combination of (a)-(d).

18. The complemented non-human animal or embryo of any one of aspects 1-17, wherein the NANOS3 gene modification is
a. an insertion of one or more nucleotides;
b. a deletion of one or more nucleotides;
c. a substitution of one or more nucleotides; or
d. any combination of (a)-(c).

19. The complemented non-human animal or embryo of any one of aspects 1-18, wherein the NANOS3 gene modification is in exon 1 of the NANOS3 gene, optionally in the zinc finger domain of the NANOS3 gene.

20. The complemented non-human animal or embryo of any one of aspects 1-19, wherein the engineered non-human animal cell or population thereof is monoallelic or is biallelic for the NANOS3 gene modification.

21. The complemented non-human animal or embryo of any one of aspects 1-20, wherein the engineered non-human animal cell or population thereof does not express a functional NANOS3 gene or gene product.

22. The complemented non-human animal or embryo of any one of aspects 1-21, wherein the engineered non-human animal cell is heterozygous or homozygous for the NANOS3 gene modification, wherein the NANOS3 gene modification is optionally a NANOS3 gene knockout.

23. The complemented non-human animal or embryo of any one of aspects 1-22, wherein the engineered non-human animal cell or population thereof is an engineered male cell or population thereof.

24. The complemented non-human animal or embryo of any one of aspects 1-22, wherein the engineered non-human animal cell is an engineered female cell or cell population.

25. The complemented non-human animal or embryo of any one of aspects 1-24, wherein the engineered non-human animal cell or population thereof is an engineered somatic cell or population thereof.

26. The complemented non-human animal or embryo of any one of aspects 1-25, wherein the engineered non-human animal cell or population thereof is an engineered germ cell or population thereof.

27. The complemented non-human animal or embryo of aspect 26, wherein the engineered germ cell or population thereof is an engineered gamete or population thereof.

28. The complemented non-human animal or embryo of aspect 27, wherein the engineered gamete or population thereof is an engineered spermatozoon or population thereof or an engineered ovum or population thereof.

29. The complemented non-human animal or embryo of aspect 26, wherein the engineered germ cell or population thereof is an engineered immature germ cell or population thereof.

30. The complemented non-human animal or embryo of aspect 29, wherein the engineered immature germ cell or population thereof is an engineered spermatid or population thereof or an engineered oocyte or population thereof.

31. The complemented non-human animal or embryo of any one of aspects 1-25, wherein the engineered non-human animal cell is an engineered embryonic cell population thereof, optionally wherein the engineered embryonic cell is a zygote.

32. The complemented non-human animal or embryo of any one of aspects 1-25, or 31, wherein the engineered non-human animal cell population thereof is an engineered blastocyst cell or population thereof, optionally an engineered inner cell mass cell or population thereof.

33. The complemented non-human animal or embryo of any one of aspects 1-25, or 31-32, wherein the engineered non-human animal cell or population thereof is an engineered stem cell or population thereof, optionally an engineered embryonic stem cell or population thereof or an induced pluripotent stem cell or population thereof.

34. The complemented non-human animal or embryo of any one of aspects 26 or 33, wherein the engineered non-human animal cell or cell population is an engineered spermatogonial stem cell or population thereof or an engineered oogonial stem cell or population thereof.

35. The complemented non-human animal or embryo of any one of aspects 1-25, wherein the engineered non-human animal cell or cell population cell is a primordial germ cell or population thereof or an engineered primordial germ cell-like cell or population thereof.

36. The complemented non-human animal or embryo of any one of aspects 1-25, or 31-35, wherein the engineered non-human animal cell or population thereof is an engineered self-renewing cell or population thereof.

37. The complemented non-human animal or embryo of any one of aspects 1-25, or 31-36, wherein the engineered non-human animal cell is pluripotent, totipotent, or multipotent.

38. A non-human animal developed or generated from the complemented non-human animal or embryo of any one of aspects 1-37.

39. The non-human animal of aspect 38, wherein one or more germ cells of the engineered animal originated from the second population of cells.

40. The non-human animal of any one of aspects 38-39, wherein about 0.001 percent to

100 percent of the germ cells originated from the second population of cells.

41. The non-human animal of any one of aspects 38-40, wherein the non-human animal is a male or a female.

42. A progeny of one or more complemented non-human animals or non-human animals of any one of aspects 1-41.

43. An engineered non-human animal cell or population thereof comprising:
a NANOS3 gene modification, wherein the NANOS3 gene modification reduces or eliminates the expression of a NANOS3 gene product.

44. The engineered non-human animal cell or population thereof of aspect 43, wherein the NANOS3 gene modification is
a. an insertion of one or more nucleotides;
b. a deletion of one or more nucleotides;
c. a substitution of one or more nucleotides;
d. or any combination of (a)-(c).

45. The engineered non-human animal cell or population thereof of any one of aspects 43-44, wherein the NANOS3 gene modification is in exon 1 of the NANOS3 gene, optionally in the zinc finger domain of the NANOS3 gene.

46. The engineered non-human animal cell or population thereof of any one of aspects 43-45, wherein the engineered non-human animal cell or population thereof is a bovine, equine, porcine, ovine, caprine, camelid, cervine, canine, feline, murine, leporine, or a cavine cell.

47. The engineered non-human animal cell or population thereof of any one of aspects 43-46, wherein one or both of the NANOS3 alleles are modified.

48. The engineered non-human animal cell or population thereof of any one of aspects 43-47, wherein the engineered non-human animal cell or population thereof is monoallelic or biallelic for the NANOS3 gene modification.

49. The engineered non-human animal cell or population thereof of any one of aspects 43-48, wherein the engineered non-human animal cell population thereof does not express a functional NANOS3 gene or gene product.

50. The engineered non-human animal cell or population thereof of any one of aspects 43-49, wherein the engineered non-human animal cell is heterozygous or homozygous for the NANOS3 gene modification, wherein the NANOS3 gene modification is optionally a NANOS3 gene knockout.

51. The engineered non-human animal cell or population thereof of any one of aspects 43-50, wherein the engineered non-human animal cell or population thereof is an engineered male cell or population thereof.

52. The engineered non-human animal cell or population thereof of any one of aspects 43-51, wherein the engineered non-human animal cell is an engineered female cell or cell population.

53. The engineered non-human animal cell or population thereof of any one of aspects 43-52, wherein the engineered non-human animal cell or population thereof is an engineered somatic cell or population thereof.

54. The engineered non-human animal cell or population thereof of any one of aspects 43-53, wherein the engineered non-human animal cell or population thereof is an engineered germ cell or population thereof.

55. The engineered non-human animal cell or cell population of aspect 54, wherein the engineered germ cell or population thereof is an engineered gamete or population thereof.

56. The engineered non-human animal cell or cell population of aspect 55, wherein the engineered gamete or population thereof is an engineered spermatozoon or population thereof or an engineered ovum or population thereof.

57. The engineered non-human animal cell or population thereof of aspect 54, wherein the engineered germ cell or population thereof is an engineered immature germ cell or population thereof.

58. The engineered non-human animal cell or population thereof of aspect 57, wherein the engineered immature germ cell or population thereof is an engineered spermatid or population thereof or an engineered oocyte or population thereof.

59. The engineered non-human animal cell or population thereof of any one of aspects 43-52, wherein the engineered non-human animal cell is an engineered embryonic cell population thereof, optionally wherein the engineered embryonic cell is a zygote.

60. The engineered non-human animal cell or population thereof of any one of aspects 43-52 or 59, wherein the engineered non-human animal cell population thereof is an engineered blastocyst cell or population thereof, optionally an engineered inner cell mass cell or population thereof.

61. The engineered non-human animal cell or population thereof of any one of aspects 43-52 or 58-59, wherein the engineered non-human animal cell or population thereof is an engineered stem cell or population thereof, optionally an engineered embryonic stem cell or population thereof or an induced pluripotent stem cell or population thereof.

62. The engineered non-human animal cell or cell population of any one of aspects 51-52 or 61, wherein the engineered non-human animal cell or cell population is an engineered spermatogonial stem cell or population thereof or an engineered oogonial stem cell or population thereof.

63. The engineered non-human animal cell or population thereof of any one of aspects 43-52, wherein the engineered non-human animal cell or cell population cell is a primordial germ cell or population thereof or an engineered primordial germ cell-like cell or population thereof.

64. The engineered non-human animal cell or population thereof of any one of aspects 43-52 or 61-63, wherein the engineered non-human animal cell or population thereof is an engineered self-renewing cell or population thereof.

65. The engineered non-human cell of any one of aspects 43-52 or 61-64, wherein the engineered non-human animal cell is pluripotent, totipotent, or multipotent.

66. An engineered non-human animal, embryo, or progeny thereof comprising an engineered non-human animal cell or population thereof as in any one of aspects 43-65.

67. The engineered non-human animal, embryo, or progeny thereof of aspect 66, wherein the engineered non-human animal, embryo, or progeny thereof is a chimera.

68. The engineered non-human animal, embryo, or progeny thereof of aspect 66, wherein the engineered non-human animal, embryo, or progeny thereof is a mosaic.

69. The engineered non-human animal, embryo, or progeny thereof of aspect 66, wherein the engineered non-human animal, embryo, or progeny thereof is not chimeric.

70. The engineered non-human animal, embryo, or progeny thereof of aspect 66 or 67, is not a mosaic.

71. The engineered non-human animal, embryo, or progeny thereof of aspect 66, wherein at least 1 cell of or at least 0.0001 percent to 100 percent of all cells of the engineered non-human animal, embryo, or progeny thereof is an engineered non-human animal cell as in any one of aspects 43-65.

72. The engineered non-human animal, embryo, or progeny thereof of any one of aspects 66-71, wherein the engineered non-human animal, embryo, or progeny thereof is a male.

73. The engineered non-human animal, embryo, or progeny thereof of any one of aspects 66-71, wherein the engineered non-human animal, embryo, or progeny thereof is a female.

74. The engineered non-human animal, embryo, or progeny thereof of any one of aspects 66-73, wherein the engineered non-human animal is a bovine, equine, porcine, ovine, caprine, camelid, cervine, canine, feline, murine, leporine, or a cavine.

75. The engineered non-human animal, embryo, or progeny thereof of any one of aspects 66-74, further comprising a second population of cells comprising one or more cells, wherein the second population of cells does not comprise engineered non-human animal cells of any one of aspects 43-65 and wherein the second population of cells are germline competent cells, germ cells, or gametes.

76. The engineered non-human animal, embryo, or progeny thereof of aspect 75, wherein the second population of cells comprises or consists of one or more
a. embryonic cells, optionally a zygote or inner cell mass cells;
b. stem cells, optionally embryonic stem cells or induced pluripotent stem cells;
c. spermatogonial stem cells or oogonial stem cells;
d. primordial germ cells; or
e. primordial germ cell like cells.

77. The engineered non-human animal, embryo, or progeny thereof of aspect 75, wherein the second population of cells comprises or consists of one or more spermatids or one or more oocytes.

78. The engineered non-human animal, embryo, or progeny thereof of aspect 75, wherein the second population of cells comprises or consists of spermatozoa or ova.

79. The engineered non-human animal, embryo, or progeny thereof of any one of aspects 75-78, wherein the second population of cells comprises or consists of one or more engineered cells comprising one or more genetic modifications in one or more target genes and wherein the one or more target genes are not NANOS3.

80. The engineered non-human animal, embryo, or progeny thereof of any one of aspects 75-78, wherein the second population of cells do not comprise or consist of an engineered cell or population thereof.

81. The engineered non-human animal, embryo, or progeny thereof of aspect 80, wherein the second population of cells comprises or consists of an elite genome, a genomically selected genome, or both.

82. A method of generating a NANOS3 modified non-human animals or embryos, the method comprising:
introducing one or more NANOS3 gene modifications to a non-human animal cell, wherein the NANOS3 gene modification reduces or eliminates the expression of a NANOS3gene product; and
one or more of the following techniques: somatic cell nuclear transfer, oocyte pronuclear DNA microinjection, zygote microinjection, or embryo microinjection, intracytoplasmic sperm injection, in vitro fertilization, embryo transfer, in vitro embryo culture, or any combination thereof.

83. The method of aspect 82, wherein the NANOS3 gene modification is
a. an insertion of one or more nucleotides;
b. a deletion of one or more nucleotides;
c. a substitution of one or more nucleotides; or
d. any combination of (a)-(c).

84. The method of any one of aspects 82-83, wherein the NANOS3 gene modification is in exon 1 of the NANOS3 gene, optionally in the zinc finger domain of the NANOS3 gene.

85. The method of any one of aspects 82-84, wherein one or both of the NANOS3 alleles are modified.

86. The method of any one of aspects 82-85, wherein the non-human animal or embryo is monoallelic or biallelic for the NANOS3 gene modification.

87. The method of any one of aspects 82-86, wherein the engineered non-human animal or embryo does not express a functional NANOS3 gene or gene product.

88. The method of any one of aspects 82-87, wherein the non-human animal or embryo is a heterozygous or homozygous NANOS3 gene knockout.

89. The method of any one of aspects 82-88, wherein the non-human animal or embryo is germline ablated.

90. The method of any one of aspects 82-89, wherein the non-human animal or embryo is a bovine, equine, porcine, ovine, caprine, camelid, cervine, canine, feline, murine, leporine, or a cavine.

91. The method of any one of aspects 82-90, wherein the non-human animal or embryo is a male.

92. The method of any one of aspects 82-90, wherein the non-human animal or embryo is a female.

93. The method of any one of aspects 82-92, wherein introducing one or more NANOS3 gene modifications to the non-human animal cell comprises CRISPR-Cas mediated gene modification, Zinc Finger Nuclease gene modification, TALEN mediated gene modification, recombinase mediated gene modification, prime editing mediated gene modification, meganuclease mediated gene modification, transposase/transposon mediated gene modification, or any combination thereof.

94. The method of aspect 93, wherein introducing one or more NANOS3gene modifications to the non-human animal cell comprises use of a CRISPR-Cas system and wherein the guide RNA for the CRISPR-Cas system targets exon 1 of the NANOS3 gene, optionally in the zinc finger region, and are optionally selected from any one of SEQ ID NOs: 39-45 or any combination thereof.

95. A method of non-human animal embryo complementation comprising:
introducing a self-renewing exogenous population of cells into a non-human animal preimplantation embryo, optionally at about day 3, 4, 5, or 6 post fertilization;
optionally washing the non-human animal preimplantation embryo in HEPES or other suitable buffer; and
culturing the non-human preimplantation embryo in a suitable culture media optionally consisting of a 1:1 ratio by volume of a suitable bovine culture media that is at least supplemented with N2, B27, FGF, and IWR-1.

96. The method of aspect 95, wherein the number of exogenous cells introduced is about 1 to about 25 cells or about 30-50 percent of the total number of cells present in the embryo prior to introducing the exogenous cells.

97. The method of any one of aspects 95-96, wherein
a. the number of exogenous cells introduced at 3 days or 4 days post fertilization is about 5 cells;
b. wherein the number of exogenous cells introduced at 5 days post fertilization is about 8, 9 cells, or 10 cells; or
c. the number of exogenous cells introduced at 6 days post fertilization is about 10-20 cells.

98. The method of non-human animal embryo complementation of any one of aspects 95-97, wherein the self-renewing exogenous cells are embryonic stem cells, expanded embryonic stem cells, pluripotent stem cells, induced pluripotent stem cells, multipotent stem cells, totipotent stem cells, primordial germ cells, primordial germ cell-like cells, totipotent cells, or a combination thereof.

99. The method of non-human animal embryo complementation of any one of aspects 95-98, wherein the non-human animal embryo is genetically germline ablated.

100. The method of non-human animal embryo complementation of any one of aspects 95-99, wherein the non-human animal embryo comprises or consists of one or more engineered cells of any one of aspects 43-65.

101. The method of non-human animal embryo complementation of any one of aspects 95-100, wherein the self-renewing exogenous cells are germline competent.

102. The method of non-human animal embryo complementation of any one of aspects 95-101, wherein the self-renewing exogenous cells are engineered cells comprising one or more gene modifications in one or more target genes and wherein the one or more target genes are not NANOS3.

103. The method of non-human animal embryo complementation of any one of aspects 95-102, wherein the self-renewing exogenous cells are not genetically modified.

104. The method of non-human animal embryo complementation of any one of aspects 95-103, wherein the self-renewing exogenous cells comprise an elite genome, a genomically selected genome, or both.

105. The method of non-human animal embryo complementation of any one of aspects 95-104, wherein the non-human animal embryo is a bovine, equine, porcine, ovine, caprine, camelid, cervine, canine, feline, murine, leporine, or a cavine.

106. A complemented non-human embryo produced from a method of embryo complementation of any one of aspects 95-105.

107. A non-human animal produced from the embryo of aspect 107 and progeny thereof.

1. A complemented non-human animal or embryo comprising:
a first population of cells comprising one or more cells, wherein the first population of cells consists of an engineered non-human animal cell or population thereof comprising a NANOS3 gene modification, wherein the NANOS3 gene modification reduces or eliminates the expression of a NANOS3 gene product;
a second population of cells comprising one or more cells, wherein the second population of cells are not an engineered non-human cell or population thereof comprising a comprising a NANOS3 gene modification, wherein the NANOS3 gene modification reduces or eliminates the expression of a NANOS3 gene product.

2. The complemented non-human animal or embryo of claim 1,
wherein the second population of cells comprises one or more engineered cells comprising one or more genetic modifications in one or more target genes and wherein the one or more target genes are not NANOS3 or wherein the second population of cells does not comprise an engineered cell.

3. (canceled)

4. The complemented non-human animal or embryo of claim 1,
(i) wherein the second population of cells comprises an elite genome, a genomically selected genome, or both
(ii) wherein the second population of cells comprises one or more (a) embryonic cells, preferably a zygote or inner cell mass cells; (b) stem cells, preferably embryonic stem cells or induced pluripotent stem cells; (c) spermatogonial stem cells or oogonial stem cells; (d) primordial germ cells; or (e) primordial germ like cells;
(iii) wherein the second population of cells are self-renewing cells;
(iv) wherein the second population of cells is pluripotent, totipotent, or multipotent;
(v) wherein the second population of cells is germline competent;
(vi) wherein the complemented embryo is a preimplantation embryo, preferably a zygote, 2 cell, 4 cell, an 8 cell, 16 cell, a blastocyst, or a morula;
(vii) wherein the first population of cells makes up a percentage of cells of the complemented non-human animal or embryo ranging from about 25 percent to any percent up to but not including 100 percent;
(viii) wherein the complemented non-human animal or embryo comprises at least one cell of the second population of cells, preferably wherein the second population of cells makes up a percentage of cells of the engineered non-human animal or embryo ranging from any non-zero percent to about 75 percent;
(ix) wherein the complemented embryo is a day 3 post fertilization embryo, a day 4 post fertilization embryo, a day 5 post fertilization embryo, or a day 6 post fertilization day embryo, preferably wherein (a) the day 3 post fertilization complemented embryo comprises about 5 cells from the second population of cells; (b) the day 4 post fertilization complemented embryo comprises about 5 cells from the second population of cells, (c) the day 5 post fertilization complemented embryo comprises about 8-10 cells from the second population of cells, and/or (d) the day 6 post fertilization complemented embryo comprises about 10-20 cells from the second population of cells;
(x) wherein the complemented embryo is a morula;
(xi) wherein the complemented embryo is a male or female;
(xii) wherein (a) the complemented non-human animal or embryo is a bovine, equine, porcine, ovine, caprine, camelid, cervine, canine, feline, murine, leporine, or cavine; (b) wherein the engineered non-human animal cell or population thereof is a bovine, equine, porcine, ovine, caprine, camelid, cervine, canine, feline, murine, leporine, or a cavine cell; (c) the first population of cells comprising one or more cells is a bovine, equine, porcine, ovine, caprine, camelid, cervine, canine, feline, murine, leporine, or a cavine population of cells; (d) the second population of cells comprising one or more cells is a bovine, equine, porcine, ovine, caprine, camelid, cervine, canine, feline, murine, leporine, or a cavine population of cells; or (e) any combination of (a)-(d);
(xiii) wherein the engineered non-human animal cell or population thereof is an engineered male cell or population thereof;
(xiv) wherein the engineered non-human animal cell or population thereof an engineered female cell or population thereof; or
(xv) any combination of (i)-(xiv).

5.-17. (canceled)

18. The complemented non-human animal or embryo of claim 1, wherein the NANOS3 gene modification is
a. an insertion of one or more nucleotides;
b. a deletion of one or more nucleotides;
c. a substitution of one or more nucleotides; or
d. any combination of (a)-(c).

19. The complemented non-human animal or embryo of claim 1, wherein the NANOS3 gene modification is in exon 1 of the NANOS3 gene, optionally in the zinc finger domain of the NANOS3 gene.

20. The complemented non-human animal or embryo of claim 1, wherein
(i) the engineered non-human animal cell or population thereof is monoallelic or is biallelic for the NANOS3 gene modification;
(ii) the engineered non-human animal cell or population thereof does not express a functional NANOS3 gene or gene product;
(iii) the engineered non-human animal cell is heterozygous or homozygous for the NANOS3 gene modification, wherein the NANOS3 gene modification is optionally a NANOS3 gene knockout: or
(iv) any combination of (i)-(iii).

21.-24. (canceled)

25. The complemented non-human animal or embryo of claim 1, wherein the engineered non-human animal cell or population thereof is (i) an engineered somatic cell or population thereof or (ii) an engineered germ cell or population thereof.

26. (canceled)

27. The complemented non-human animal or embryo of claim 25, wherein
(a) the engineered germ cell or population thereof is an engineered gamete or population thereof;
(b) the engineered germ cell or population thereof is engineered gamete or population thereof and is an engineered spermatozoon or population thereof or an engineered ovum or population thereof;
(c) the engineered germ cell or population thereof is an engineered immature germ cell or population thereof;
(d) the engineered germ cell or population thereof is an engineered immature germ cell or population thereof and where the immature germ cell or population thereof is an engineered spermatid or population thereof or an engineered oocyte or population thereof;
(e) the engineered non-human animal cell or cell population cell is a primordial germ cell or population thereof or an engineered primordial germ cell-like cell or population thereof;
(f) the engineered non-human animal cell or cell population is an engineered spermatogonial stem cell or population thereof or an engineered oogonial stem cell or population thereof;
(g) the engineered non-human animal cell or population thereof is an engineered self-renewing cell or population thereof;
(h) the engineered self-renewing cell or population thereof is pluripotent, totipotent, or multipotent: or
(i) any combination of (a)-(i).

28.-30. (canceled)

31. The complemented non-human animal or embryo of claim 1, wherein
(i) the engineered non-human animal cell is an engineered embryonic cell population thereof;
(ii) the engineered embryonic cell is a zygote;
(iii) the engineered non-human animal cell population thereof is an engineered blastocyst cell or population thereof;
(iv) the engineered non-human animal cell population thereof is an engineered inner cell mass cell or population thereof;
(v) the engineered non-human animal cell or population thereof is an engineered stem cell or population thereof;
(vi) the engineered non-human animal cell or population thereof is an engineered embryonic stem cell or population thereof or an induced pluripotent stem cell or population thereof;
(vii) the engineered non-human animal cell or cell population is an engineered spermatogonial stem cell or population thereof or an engineered oogonial stem cell or population thereof;
(viii) the engineered non-human animal cell or population thereof is an engineered self-renewing cell or population thereof;
(ix) the engineered non-human animal cell or population thereof is pluripotent, totipotent, or multipotent; or
(x) any combination of (i)-(ix).

32.-37. (canceled)

38. A non-human animal developed or generated from the complemented non-human animal or embryo of claim 1 or a progeny of the non-human animal.

39. The non-human animal of claim 38 or progeny of non-human animal, wherein
(i) one or more germ cells of the engineered animal originated from the second population of cells;
(ii) about 0.001 percent to 100 percent of the germ cells originated from the second population of cells;
(iii) the non-human animal is a male;
(iv) the non-human animal is a female; or
(v) any combination of (i)-(iv).

40.-42. (canceled)

43. An engineered non-human animal cell or population thereof comprising:
a NANOS3 gene modification, wherein the NANOS3 gene modification reduces or eliminates the expression of a NANOS3 gene product, wherein
the NANOS3 gene modification is
(i) an insertion of one or more nucleotides;
(ii) a deletion of one or more nucleotides;
(iii) a substitution of one or more nucleotides; or
(iv) any combination of (i)-(iii).

44. (canceled)

45. The engineered non-human animal cell or population thereof of claim 43, wherein the NANOS3 gene modification is in exon 1 of the NANOS3 gene, optionally in the zinc finger domain of the NANOS3 gene.

46. The engineered non-human animal cell or population thereof of claim 43, wherein
(a) the engineered non-human animal cell or population thereof is a bovine, equine, porcine, ovine, caprine, camelid, cervine, canine, feline, murine, leporine, or a cavine cell or population thereof;
(b) one or both of the NANOS3 alleles are modified;
(c) the engineered non-human animal cell or population thereof is monoallelic or biallelic for the NANOS3 gene modification;
(d) the engineered non-human animal cell population thereof does not express a functional NANOS3 gene or gene product;
(e) the engineered non-human animal cell is heterozygous or homozygous for the NANOS3 gene modification;
(f) the NANOS3 gene modification is a NANOS3 gene knockout;
(g) the engineered non-human animal cell or population thereof is an engineered male cell or population thereof;
(h) the engineered non-human animal cell is an engineered female cell or cell population;
(i) the engineered non-human animal cell or population thereof is an engineered somatic cell or population thereof;
(j) the engineered non-human animal cell or population thereof is an engineered germ cell or population thereof;
(k) the engineered non-human animal cell is an engineered embryonic cell or population thereof;
(l) the engineered non-human animal cell is an engineered embryonic stem cell or population thereof or an induced pluripotent stem cell or population thereof;
(m) the engineered non-human animal cell or cell population is an engineered spermatogonial stem cell or population thereof;
(n) the engineered non-human animal cell or cell population is an engineered oogonial stem cell or population thereof
(o) the engineered non-human animal cell is a zygote;
(p) the engineered non-human animal cell population thereof is an engineered blastocyst cell or population thereof;
(q) the engineered non-human animal cell population thereof is an engineered inner cell mass cell or population thereof;
(r) the engineered non-human animal cell population thereof is an engineered gamete or population thereof;
(s) the engineered non-human animal cell population thereof is an engineered spermatozoon or population thereof or an engineered ovum or population thereof;
(t) the engineered non-human animal cell population thereof is an is an engineered immature germ cell or population thereof;
(u) the engineered non-human animal cell population thereof is an engineered spermatid or population thereof or an engineered oocyte or population thereof;
(v) the engineered non-human animal cell or cell population cell is a primordial germ cell or population thereof or an engineered primordial germ cell-like cell or population thereof;
(w) the engineered non-human animal cell or population thereof is an engineered self-renewing cell or population thereof;
(x) the engineered non-human animal cell is pluripotent, totipotent, or multipotent.

47.-65. (canceled)

66. An engineered non-human animal, embryo, or progeny thereof comprising an engineered non-human animal cell or population thereof of claim 43.

67. The engineered non-human animal, embryo, or progeny thereof of claim 66,
(i) wherein (a) the engineered non-human animal, embryo, or progeny thereof is a chimera; (b) the engineered non-human animal, embryo, or progeny thereof is a mosaic; (c) the engineered non-human animal, embryo, or progeny thereof is not chimeric; or (d) is not a mosaic;
(ii) wherein at least 1 cell of or at least 0.0001 percent to 100 percent of all cells of the engineered non-human animal, embryo, or progeny thereof is an engineered non-human animal cell comprising a NANOS3 gene modification, wherein the NANOS3 gene modification reduces or eliminates the expression of a NANOS3 gene product;
(iii) wherein the engineered non-human animal, embryo, or progeny thereof is a male;
(iv) wherein the engineered non-human animal, embryo, or progeny thereof is a female;
(v) wherein the engineered non-human animal is a bovine, equine, porcine, ovine, caprine, camelid, cervine, canine, feline, murine, leporine, or a cavine; or
(vi) any combination of (i)-(v).

68.-74. (canceled)

75. The engineered non-human animal, embryo, or progeny thereof of claim 66, further comprising a second population of cells comprising one or more cells, wherein the second population of cells does not comprise engineered non-human animal cells of comprising a NANOS3 gene modification, wherein the NANOS3 gene modification reduces or eliminates the expression of a NANOS3 gene product and wherein the second population of cells are germline competent cells, germ cells, or gametes.

76. The engineered non-human animal, embryo, or progeny thereof of claim 75, wherein
(i) the second population of cells comprises or consists of one or more (a) embryonic cells, preferably a zygote or inner cell mass cells (b) stem cells, preferably embryonic stem cells or induced pluripotent stem cells; (c) spermatogonial stem cells or oogonial stem cells; (d) primordial germ cells; or (e) primordial germ cell like cells;
(ii) the second population of cells comprises or consists of one or more spermatids or one or more oocytes;
(iii) the second population of cells comprises or consists of spermatozoa or ova
(iv) the second population of cells comprises or consists of one or more engineered cells comprising one or more genetic modifications in one or more target genes and wherein the one or more target genes are not NANOS3;
(v) the second population of cells do not comprise or consist of an engineered cell or population thereof,
(vi) the second population of cells comprises or consists of an elite genome, a genomically selected genome, or both;
(vii) (iv) in combination with any one or more of (i)-(iii); or
(viii) (v) in combination with any one or more of (i)-(iii).

77.-81. (canceled)

82. A method of generating a NANOS3 modified non-human animals or embryos, the method comprising:
introducing one or more NANOS3 gene modifications to a non-human animal cell, wherein the NANOS3 gene modification reduces or eliminates the expression of a NANOS3gene product; and
one or more of the following techniques: somatic cell nuclear transfer, oocyte pronuclear DNA microinjection, zygote microinjection, or embryo microinjection, intracytoplasmic sperm injection, in vitro fertilization, embryo transfer, in vitro embryo culture, or any combination thereof.

83. The method of claim 82, wherein the NANOS3 gene modification is
a. an insertion of one or more nucleotides;
b. a deletion of one or more nucleotides;
c. a substitution of one or more nucleotides; or
d. any combination of (a)-(c).

84. The method of claim 82, wherein the NANOS3 gene modification is in exon 1 of the NANOS3 gene, optionally in the zinc finger domain of the NANOS3 gene.

85. The method of claim 82, wherein
(i) one or both of the NANOS3 alleles are modified;
(ii) the non-human animal or embryo is monoallelic or biallelic for the NANOS3 gene modification;
(iii) the engineered non-human animal or embryo does not express a functional NANOS3 gene or gene product;
(iv) the non-human animal or embryo is a heterozygous or homozygous NANOS3 gene knockout;
(v) the non-human animal or embryo is germline ablated;
(vi) the non-human animal or embryo is a bovine, equine, porcine, ovine, caprine, camelid, cervine, canine, feline, murine, leporine, or a cavine;
(vii) the non-human animal or embryo is a male;
(viii) the non-human animal or embryo is a female;
(ix) introducing one or more NANOS3 gene modifications to the non-human animal cell comprises CRISPR-Cas mediated gene modification, Zinc Finger Nuclease gene modification, TALEN mediated gene modification, recombinase mediated gene modification, prime editing mediated gene modification, meganuclease mediated gene modification, transposase/transposon mediated gene modification, or any combination thereof;
(x) introducing one or more NANOS3gene modifications to the non-human animal cell comprises use of a CRISPR-Cas system and wherein the guide RNA for the CRISPR-Cas system targets exon 1 of the NANOS3 gene, optionally in the zinc finger region, and are optionally selected from any one of SEQ ID NOs: 39-45 or any combination thereof; or
(xi) any combination of (i)-(xi).

86.-94. (canceled)

95. A method of non-human animal embryo complementation comprising:
introducing a self-renewing exogenous population of cells into a non-human animal preimplantation embryo, optionally at about day 3, 4, 5, or 6 post fertilization;
optionally washing the non-human animal preimplantation embryo in HEPES or other suitable buffe; and
culturing the non-human preimplantation embryo in a suitable culture media optionally consisting of a 1:1 ratio by volume of a suitable bovine culture media that is at least supplemented with N2, B27, FGF, and IWR-1.

96. The method of claim 95, wherein
(i) the number of exogenous cells introduced is about 1 to about 25 cells or about 30-50 percent of the total number of cells present in the embryo prior to introducing the exogenous cells,
(ii) (a) the number of exogenous cells introduced at 3 days or 4 days post fertilization is about 5 cells, (b) the number of exogenous cells introduced at 5 days post fertilization is about 8, 9 cells, or 10 cells, or (c) the number of exogenous cells introduced at 6 days post fertilization is about 10-20 cells;
(iii) wherein the self-renewing exogenous cells are embryonic stem cells, expanded embryonic stem cells, pluripotent stem cells, induced pluripotent stem cells, multipotent stem cells, totipotent stem cells, primordial germ cells, primordial germ cell-like cells, totipotent cells, or a combination thereof;
(iv) the non-human animal embryo is genetically germline ablated;
(v) the non-human animal embryo comprises or consists of one or more engineered non-human animal cells of comprising a NANOS3 gene modification, wherein the NANOS3 gene modification reduces or eliminates the expression of a NANOS3 gene product;
(vi) the self-renewing exogenous cells are germline competent;
(vii) the self-renewing exogenous cells are germline competent;
(viii) the self-renewing exogenous cells are engineered cells comprising one or more gene modifications in one or more target genes and wherein the one or more target genes are not NANOS3;
(ix) wherein the self-renewing exogenous cells are not genetically modified;
(x) the self-renewing exogenous cells comprise an elite genome, a genomically selected genome, or both;
(xi) the non-human animal embryo is a bovine, equine, porcine, ovine, caprine, camelid, cervine, canine, feline, murine, leporine, or a cavine; or
(xii) any combination of (i)-(xi).

97.-105. (canceled)

106. A complemented non-human embryo produced from a method of embryo complementation of claim 95.

107. A non-human animal produced from the embryo of claim 106 and progeny thereof.
